(19) United States
(12) Patent Application Publication
(10) Pub. No.: US 2025/0313840 A1
(43) Pub. Date: Oct. 9, 2025

(54) MUCIN-5B (MUC5B) TARGETED SIRNA AND ANTISENSE OLIGONUCLEOTIDES AND METHODS OF USE THEREOF

(71) Applicant: Vertex Pharmaceuticals Incorporated, Boston, MA (US)

(72) Inventor: Diego D'Astolfo, Boston, MA (US)

(73) Assignee: Vertex Pharmaceuticals Incorporated, Boston, MA (US)

(21) Appl. No.: 19/084,446

(22) Filed: Mar. 19, 2025

Related U.S. Application Data
(60) Provisional application No. 63/567,742, filed on Mar. 20, 2024.

(51) Int. Cl. C12N 15/113; A61P 11/00

(52) U.S. Cl. CPC C12N 15/113; A61P 11/00; C12N 2310/11; C12N 2310/14; C12N 2310/315; C12N 2310/321; C12N 2310/322; C12N 2310/3515; C12N 2320/32

(57) ABSTRACT

Disclosed herein are double stranded ribonucleic acids (dsRNAs) and synthetic antisense oligonucleotides (ASOs) for use in inhibiting the expression of MUC5B and in the treatment of a subject having a disease or disorder characterized by overexpression of MUC5B, including in subjects having lung disease or in a subject at risk of developing lung disease, for example, in a subject having or at risk of developing idiopathic pulmonary fibrosis.

### CROSS REFERENCE TO RELATED APPLICATIONS

This application claims priority to U.S. Provisional Application No. 63/567,742, filed Mar. 20, 2024, the disclosure of which is hereby incorporated by reference in its entirety.

### SEQUENCE LISTING

The instant application contains a “lengthy” Sequence Listing which has been submitted via DVD-R in lieu of a printed paper copy, and is hereby incorporated by reference in its entirety. Said DVD-R, recorded on Mar. 7, 2025, is labeled “CRF”, and contains one file, named 01245-0060-00PCT_generic_SL.xml, which is 170,611,341 bytes in size.

### INTRODUCTION AND SUMMARY

Pulmonary fibrosis (PF) is a disease that affects older adults and causes a decline in lung function, which can lead to a decreased quality of life and even death. The disease can be caused by various factors, including idiopathic (the most common type), connective-tissue disease-related, autoimmune, and exposure-related factors. A significant portion of PF cases are driven by the rs35705950-T mutation in the Mucin-5B (MUC5B) promoter, which is present in over 50% of patients with idiopathic pulmonary fibrosis (IPF) (50 k of 100 k in the US), rheumatoid-arthritis associated-ILD (10 k of 20K in the US), nonspecific interstitial pneumonia (10 k of 20 k), and chronic hypersensitivity pneumonitis (5 k of 10K in the US).

MUC5B is a member of the mucin family of proteins, which are highly glycosylated macromolecular components of mucus secretions. This family member is expressed in club cells in normal lung epithelium. The MUC5B protein is the major gel-forming mucin in mucus, which is secreted by submucosal glands, salivary glands, nasal mucosa, gallbladder, submucosal glands in the trachea, and esophagus. It is a major contributor to the lubricating and viscoelastic properties of whole saliva, normal lung mucus, and cervical mucus, and, in the lung, the MUC5B protein plays a key role in mucociliary transport and mucociliary clearance (MCC) and host defense responsible for trapping and clearing inhaled particles.

Increased expression of MUC5B is associated with the development of lung diseases, e.g., pulmonary fibrosis, cystic fibrosis, and/or chronic obstructive pulmonary disease (COPD). For example, mice overexpressing Muc5b exhibit increased mucosal depth, reduced ciliary beat frequency, and reduced mucociliary transport rate, and bleomycin-treated mice overexpressing Muc5b in distal airways and alveoli have decreased survival and increased lung fibrosis. Furthermore, increased MUC5B expression has been observed in the distal airways of subjects having IPF.

The genetic variation rs35705950-T results in abnormal MUC5B expression in small airways and alveoli epithelial cells. MUC5B is an essential large mucus glycoprotein that lubricates and protects epithelial cells, playing a crucial role in mucociliary clearance. An increased amount of MUC5B in the upper airway may protect against infections, but the de novo expression of MUC5B in the distal lung causes ER stress and cell injury.

Current therapies (e.g., Pirfenidone, Nintedanib) are mainly supportive and may slow the progression of the disease but have little impact on overall survival. There remains a need for additional and alternative effective gene editing strategies for treating lung diseases, e.g., pulmonary fibrosis, e.g., IPF; cystic fibrosis, and/or chronic obstructive pulmonary disease (COPD).

Provided herein are compositions that can selectively and effectively inhibit the expression of a target MUC5B gene and methods for treating lung diseases.

Accordingly, the following non-limiting embodiments are provided.

Embodiment 1. A double stranded ribonucleic acid (dsRNA) targeting Mucin-5B (MUC5B), wherein the dsRNA comprises a sense strand and an antisense strand forming a double stranded region, wherein the antisense strand comprises a region of complementarity to an mRNA encoding MUC5B, and wherein the antisense strand comprises a nucleotide sequence selected from SEQ ID NOs: 178-354.

Embodiment 2. The dsRNA of embodiment 1, wherein the sense strand comprises a nucleotide sequence selected from SEQ ID NOs: 1-177.

Embodiment 3. The dsRNA of any one of embodiments 1-2, wherein the sense and antisense strands are selected from any one of the sense and antisense pairs in Table 2 or Table 3.

Embodiment 4. A double stranded ribonucleic acid (dsRNA) targeting Mucin-5B (MUC5B), wherein the dsRNA comprises a sense strand and an antisense strand forming a double stranded region, wherein the antisense strand comprises a region of complementarity to an mRNA encoding MUC5B, and wherein the antisense strand comprises a nucleotide sequence differing in sequence by no more than 1 nucleotide from any one of SEQ ID NOs: 179-192, 194-328, and 332-354.

Embodiment 5. A double stranded ribonucleic acid (dsRNA) targeting Mucin-5B (MUC5B), wherein the dsRNA comprises a sense strand and an antisense strand forming a double stranded region, wherein the antisense strand comprises a region of complementarity to an mRNA encoding MUC5B, and wherein the antisense strand comprises a nucleotide sequence comprising at least 22 contiguous nucleotides differing in sequence by no more than 1 nucleotide from any one of SEQ ID NOs: 179-187, 189, 190, 192, 194-200, 202, 204-311, 313, 315-328, 332-334, and 336-354.

Embodiment 6. The dsRNA of embodiment 5, wherein the antisense strand comprises a nucleotide sequence comprising at least 22 contiguous nucleotides from any one of SEQ ID NOs: 179-187, 189, 190, 192, 194-200, 202, 204-311, 313, 315-328, 332-334, and 336-354.

Embodiment 7. A double stranded ribonucleic acid (dsRNA) targeting Mucin-5B (MUC5B), wherein the dsRNA comprises a sense strand and an antisense strand forming a double stranded region, wherein the antisense strand comprises a region of complementarity to an mRNA encoding MUC5B, and wherein the antisense strand comprises a nucleotide sequence comprising at least 21 contiguous nucleotides differing in sequence by no more than 1 nucleotide from any one of SEQ ID NOs: 181-187, 189, 190, 192, 194-200, 202, 204-229, 231-243, 246-310, 313, 315-328, 333-334, and 336-354.

Embodiment 8. The dsRNA of embodiment 7, wherein the antisense strand comprises a nucleotide sequence comprising at least 21 contiguous nucleotides from any one of SEQ ID NOs: 181-187, 189, 190, 192, 194-200, 202, 204-229, 231-243, 246-310, 313, 315-328, 333-334, and 336-354.

Embodiment 9. A double stranded ribonucleic acid (dsRNA) targeting Mucin-5B (MUC5B), wherein the dsRNA comprises a sense strand and an antisense strand forming a double stranded region, wherein the antisense strand comprises a region of complementarity to an mRNA encoding MUC5B, and wherein the antisense strand comprises a nucleotide sequence comprising at least 20 contiguous nucleotides differing in sequence by no more than 1 nucleotide from any one of SEQ ID NOs: 181-187, 190, 192, 194-200, 205-210, 212-229, 231-242, 246-249, 253-258, 260-266, 268-285, 287, 289-294, 296, 298-310, 315-328, 333-334, 336-346, 348, and 350-354.

Embodiment 10. The dsRNA of embodiment 9, wherein the antisense strand comprises a nucleotide sequence comprising at least 20 contiguous nucleotides from any one of SEQ ID NOs: 181-187, 190, 192, 194-200, 205-210, 212-229, 231-242, 246-249, 253-258, 260-266, 268-285, 287, 289-294, 296, 298-310, 315-328, 333-334, 336-346, 348, and 350-354.

Embodiment 11. A double stranded ribonucleic acid (dsRNA) targeting Mucin-5B (MUC5B), wherein the dsRNA comprises a sense strand and an antisense strand forming a double stranded region, wherein the antisense strand comprises a region of complementarity to an mRNA encoding MUC5B, and wherein the antisense strand comprises a nucleotide sequence comprising at least 19 contiguous nucleotides differing in sequence by no more than 1 nucleotide from any one of SEQ ID NOs: 181-187, 190, 192, 194-200, 205-210, 213, 215-228, 231-237, 239-242, 246-248, 253-258, 261-266, 268-269, 271, 273-285, 287, 289-294, 296, 298-303, 305-310, 315-328, 334, 336-338, 340-346, 348, and 350-354.

Embodiment 12. The dsRNA of embodiment 11, wherein the antisense strand comprises a nucleotide sequence comprising at least 19 contiguous nucleotides from any one of SEQ ID NOs: 181-187, 190, 192, 194-200, 205-210, 213, 215-228, 231-237, 239-242, 246-248, 253-258, 261-266, 268-269, 271, 273-285, 289-294, 296, 298-303, 305-310, 315-328, 334, 336-338, 340-346, 348, and 350-354.

Embodiment 13. A double stranded ribonucleic acid (dsRNA) targeting Mucin-5B (MUC5B), wherein the dsRNA comprises a sense strand and an antisense strand forming a double stranded region, wherein the antisense strand comprises a region of complementarity to an mRNA encoding MUC5B, and wherein the antisense strand comprises a nucleotide sequence comprising at least 18 contiguous nucleotides differing in sequence by no more than 1 nucleotide from any one of SEQ ID NOs: 181-187, 190, 192, 194-197, 205-210, 213, 215-228, 231-237, 239-242, 253-254, 261-266, 268-269, 271, 273-281, 283-285, 290, 292-294, 298-303, 305-310, 315-322, 324, 326-328, 334, 336-338, 340-346, and 351-354.

Embodiment 14. The dsRNA of embodiment 13, wherein the antisense strand comprises a nucleotide sequence comprising at least 18 contiguous nucleotides from any one of SEQ ID NOs: 181-187, 190, 192, 194-197, 205-210, 213, 215-228, 231-237, 239-242, 253-254, 261-266, 268-269, 271, 273-281, 283-285, 290, 292-294, 298-303, 305-310, 315-322, 324, 326-328, 334, 336-338, 340-346, and 351-354.

Embodiment 15. A double stranded ribonucleic acid (dsRNA) targeting Mucin-5B (MUC5B), wherein the dsRNA comprises a sense strand and an antisense strand forming a double stranded region, wherein the antisense strand comprises a region of complementarity to an mRNA encoding MUC5B, and wherein the antisense strand comprises a nucleotide sequence comprising at least 17 contiguous nucleotides differing in sequence by no more than 1 nucleotide from any one of SEQ ID NOs: 181-187, 190, 192, 194-197, 205-210, 215-228, 232, 234-237, 239-242, 253-254, 261-266, 268-269, 274, 276-278, 284-285, 290, 292-294, 298-303, 305-310, 315-322, 324, 326-328, 334, 336-338, 341-346, and 351-354.

Embodiment 16. The dsRNA of embodiment 15, wherein the antisense strand comprises a nucleotide sequence comprising at least 17 contiguous nucleotides from any one of SEQ ID NOs: 181-187, 190, 192, 194-197, 205-210, 215-228, 232, 234-237, 239-242, 253-254, 261-266, 268-269, 274, 276-278, 284-285, 290, 292-294, 298-303, 305-310, 315-322, 324, 326-328, 334, 336-338, 341-346, and 351-354.

Embodiment 17. A double stranded ribonucleic acid (dsRNA) targeting Mucin-5B (MUC5B), wherein the dsRNA comprises a sense strand and an antisense strand forming a double stranded region, wherein the antisense strand comprises a region of complementarity to an mRNA encoding MUC5B, and wherein the antisense strand comprises a nucleotide sequence comprising at least 16 contiguous nucleotides differing in sequence by no more than 1 nucleotide from any one of SEQ ID NOs: 181, 183-187, 190, 192, 194-197, 207-210, 215-228, 232, 234-237, 239-242, 253-254, 261-266, 268-269, 274, 276-278, 284-285, 293, 299, 301-303, 305-307, 315-321, 327, 334, 336-338, 341-346, and 351-354.

Embodiment 18. The dsRNA of embodiment 17, wherein the antisense strand comprises a nucleotide sequence comprising at least 16 contiguous nucleotides from any one of SEQ ID NOs: 181, 183-187, 190, 192, 194-197, 207-210, 215-228, 232, 234-237, 239-242, 253-254, 261-266, 268-269, 274, 276-278, 284-285, 293, 299, 301-303, 305-307, 315-321, 327, 334, 336-338, 341-346, and 351-354.

Embodiment 19. A double stranded ribonucleic acid (dsRNA) targeting Mucin-5B (MUC5B), wherein the dsRNA comprises a sense strand and an antisense strand forming a double stranded region, wherein the antisense strand comprises a region of complementarity to an mRNA encoding MUC5B, and wherein the antisense strand comprises a nucleotide sequence comprising at least 15 contiguous nucleotides differing in sequence by no more than 1 nucleotide from any one of SEQ ID NOs: 181, 183-187, 190, 192, 194-197, 207-210, 215-222, 224, 226-228, 234-237, 240-242, 253-254, 261-266, 268-269, 276-278, 284-285, 293, 299, 301-303, 315-317, 319, 321, 327, 334, 336-338, 341-346, and 351-354.

Embodiment 20. The dsRNA of embodiment 19, wherein the antisense strand comprises a nucleotide sequence comprising at least 15 contiguous nucleotides from any one of SEQ ID NOs: 181, 183-187, 190, 192, 194-197, 207-210, 215-222, 224, 226-228, 234-237, 240-242, 253-254, 261-266, 268-269, 276-278, 284-285, 293, 299, 301-303, 315-317, 319, 321, 327, 334, 336-338, 341-346, and 351-354.

Embodiment 21. A double stranded ribonucleic acid (dsRNA) targeting Mucin-5B (MUC5B), wherein the dsRNA comprises a sense strand and an antisense strand forming a double stranded region, wherein the antisense strand comprises a region of complementarity to an mRNA encoding MUC5B, and wherein the antisense strand comprises a nucleotide sequence comprising at least 15 contiguous nucleotides differing in sequence by no more than 2 nucleotides from any one of SEQ ID NOs: 185-187, 192, 194, 207-210, 216-222, 224, 226-228, 235-237, 240-242, 253-254, 261-266, 268-269, 276-278, 284-285, 301-303, 315-317, 319, 321, 334, 336-338, 341-346, and 352-354.

Embodiment 22. The dsRNA of embodiment 21, wherein the antisense strand comprises a nucleotide sequence comprising at least 15 contiguous nucleotides differing in sequence by no more than 1 nucleotide from any one of 185-187, 192, 194, 207-210, 216-222, 224, 226-228, 235-237, 240-242, 253-254, 261-266, 268-269, 276-278, 284-285, 301-303, 315-317, 319, 321, 334, 336-338, 341-346, and 352-354.

Embodiment 23. The dsRNA of embodiment 21, wherein the antisense strand comprises a nucleotide sequence comprising at least 15 contiguous nucleotides from any one of 185-187, 192, 194, 207-210, 216-222, 224, 226-228, 235-237, 240-242, 253-254, 261-266, 268-269, 276-278, 284-285, 301-303, 315-317, 319, 321, 334, 336-338, 341-346, and 352-354.

Embodiment 24. A double stranded ribonucleic acid (dsRNA) targeting Mucin-5B (MUC5B), wherein the dsRNA comprises a sense strand and an antisense strand forming a double stranded region, wherein the antisense strand comprises a region of complementarity to an mRNA encoding MUC5B, and wherein the antisense strand comprises a nucleotide sequence comprising at least 15 contiguous nucleotides differing in sequence by no more than 3 nucleotides from any one of SEQ ID NOs: 186-187, 192, 194, 208, 216-222, 226-228, 236, 240-242, 253-254, 268-269, 276-278, 284-285, 301-303, 316, 334, 336-338, 341-346, and 352-354.

Embodiment 25. The dsRNA of embodiment 24, wherein the antisense strand comprises a nucleotide sequence comprising at least 15 contiguous nucleotides differing in sequence by no more than 2 nucleotides from any one of SEQ ID NOs: 186-187, 192, 194, 208, 216-222, 226-228, 236, 240-242, 253-254, 268-269, 276-278, 284-285, 301-303, 316, 334, 336-338, 341-346, and 352-354.

Embodiment 26. The dsRNA of embodiment 24, wherein the antisense strand comprises a nucleotide sequence comprising at least 15 contiguous nucleotides differing in sequence by no more than 1 nucleotide from any one of SEQ ID NOs: 186-187, 192, 194, 208, 216-222, 226-228, 236, 240-242, 253-254, 268-269, 276-278, 284-285, 301-303, 316, 334, 336-338, 341-346, and 352-354.

Embodiment 27. The dsRNA of embodiment 24, wherein the antisense strand comprises a nucleotide sequence comprising at least 15 contiguous nucleotides from any one of SEQ ID NOs: 186-187, 192, 194, 208, 216-222, 226-228, 236, 240-242, 253-254, 268-269, 276-278, 284-285, 301-303, 316, 334, 336-338, 341-346, and 352-354.

Embodiment 28. A double stranded ribonucleic acid (dsRNA) targeting Mucin-5B (MUC5B), wherein the dsRNA comprises a sense strand and an antisense strand forming a double stranded region, wherein the antisense strand comprises a region of complementarity to an mRNA encoding MUC5B, wherein
a. the sense strand comprises the nucleotide sequence of SEQ ID NO: 1, and the antisense strand comprises the nucleotide sequence of SEQ ID NO: 178;
b. the sense strand comprises a nucleotide sequence comprising at least 20 contiguous nucleotides differing in sequence by no more than 1 nucleotide from SEQ ID NO: 2, and the antisense strand comprises a nucleotide sequence comprising at least 22 contiguous nucleotides differing in sequence by no more than 1 nucleotide from SEQ ID NO: 179;
c. the sense strand comprises a nucleotide sequence comprising at least 20 contiguous nucleotides differing in sequence by no more than 1 nucleotide from any one of SEQ ID NO: 3, and the antisense strand comprises a nucleotide sequence comprising at least 22 contiguous nucleotides differing in sequence by no more than 1 nucleotide from SEQ ID NO: 180;
d. the sense strand comprises a nucleotide sequence comprising at least 15 contiguous nucleotides differing in sequence by no more than 1 nucleotide from SEQ ID NO: 5, and the antisense strand comprises a nucleotide sequence comprising at least 17 contiguous nucleotides differing in sequence by no more than 1 nucleotide from SEQ ID NO: 182;
e. the sense strand comprises a nucleotide sequence comprising at least 15 contiguous nucleotides differing in sequence by no more than 3 nucleotides from SEQ ID NO: 4, and the antisense strand comprises a nucleotide sequence comprising at least 15 contiguous nucleotides differing in sequence by no more than 1 nucleotide from SEQ ID NO: 181;
f. the sense strand comprises a nucleotide sequence comprising at least 15 contiguous nucleotides differing in sequence by no more than 3 nucleotides from SEQ ID NO: 6, and the antisense strand comprises a nucleotide sequence comprising at least 15 contiguous nucleotides differing in sequence by no more than 1 nucleotide from SEQ ID NO: 183;
g. the sense strand comprises a nucleotide sequence comprising at least 15 contiguous nucleotides differing in sequence by no more than 3 nucleotides from SEQ ID NO: 7, and the antisense strand comprises a nucleotide sequence comprising at least 15 contiguous nucleotides differing in sequence by no more than 1 nucleotide from SEQ ID NO: 184;
h. the sense strand comprises a nucleotide sequence comprising at least 15 contiguous nucleotides differing in sequence by no more than 3 nucleotides from SEQ ID NO: 8, and the antisense strand comprises a nucleotide sequence comprising at least 15 contiguous nucleotides differing in sequence by no more than 2 nucleotides from SEQ ID NOs: 185;
i. the sense strand comprises a nucleotide sequence comprising at least 15 contiguous nucleotides differing in sequence by no more than 3 nucleotides from SEQ ID NO: 9, and the antisense strand comprises a nucleotide sequence comprising at least 15 contiguous nucleotides differing in sequence by no more than 3 nucleotides from SEQ ID NO: 186;
j. the sense strand comprises a nucleotide sequence comprising at least 15 contiguous nucleotides differing in sequence by no more than 3 nucleotides from SEQ ID NO: 10, and the antisense strand comprises a nucleotide sequence comprising at least 15 contiguous nucleotides differing in sequence by no more than 3 nucleotides from SEQ ID NO: 187.
k. the antisense strand comprises a nucleotide sequence selected from any one of SEQ ID NOs: 18,793-36,683, and the sense strand comprises a nucleotide sequence selected from any one of SEQ ID NOs: 902-18,792;
l. the antisense strand comprises a nucleotide sequence selected from any one of SEQ ID NOs: 54,575-72,465, and the sense strand comprises a nucleotide sequence selected from any one of SEQ ID NOs: 36,684-54,574; or
m. the antisense strand comprises a nucleotide sequence selected from any one of SEQ ID NOs: 90,357-108,247, and the sense strand comprises a nucleotide sequence selected from any one of SEQ ID NOs: 72,466-90,356.

Embodiment 29. The dsRNA of any one of embodiments 1-28, wherein the sense strand or the antisense strand:
a. is conjugated to one or more lipophilic moieties;
b. is packaged in a lipid nanoparticle (LNP); and/or
c. comprises at least one modification.

Embodiment 30. The dsRNA of any one of embodiments 1-29, wherein each of the sense strand and the antisense strand:
a. is conjugated to one or more lipophilic moieties;
b. is packaged in a lipid nanoparticle (LNP); and/or
c. comprises at least one modification.

Embodiment 31. The dsRNA of any one of embodiments 29-30, comprising one or more lipophilic moieties that are each conjugated to one or more positions in the double stranded region of the dsRNA.

Embodiment 32. The dsRNA of any one of embodiments 1-31, wherein the dsRNA comprises at least one or two modified nucleotides.

Embodiment 33. The dsRNA of any one of embodiments 1-32, wherein
a. at least 1, 2, 3, 4, 5, 6, 7, 8, 9, 10, 11, 12, 13, 14, 15, 16, 17, 18, 19, or 20 nucleotides of the sense strand are modified; and/or
b. at least 1, 2, 3, 4, 5, 6, 7, 8, 9, 10, 11, 12, 13, 14, 15, 16, 17, 18, 19, or 20 nucleotides of the antisense strand are modified; or
c. each nucleotide in the sense strand is modified; and/or
d. each nucleotide in the antisense strand is modified.

Embodiment 34. The dsRNA of any one of embodiments 1-33, wherein the antisense strand comprises a nucleotide sequence selected from any one of SEQ ID NOs: 532-708.

Embodiment 35. The dsRNA of embodiment 34, wherein the sense strand comprises a nucleotide sequence selected from any one of SEQ ID NOs: 355-531.

Embodiment 36. The dsRNA of any one of embodiments 1-35, wherein the sense and antisense strands are selected from any one of the sense and antisense pairs in Table 2 or Table 3.

Embodiment 37. The dsRNA of any one of embodiments 1-36, wherein each of the nucleotides of the sense strand comprises a modification.

Embodiment 38. The dsRNA of any one of embodiments 1-37, wherein each of the nucleotides of the antisense strand comprises a modification.

Embodiment 39. The dsRNA of any one of embodiments 1-38, wherein the dsRNA comprises at least one modification, wherein the modification comprises a 2′O-methyl, a 2′fluoro, or a phosphorothioate linkage, or a combination of a 2′O-methyl or 2′fluoro and a phosphorothioate linkage.

Embodiment 40. The dsRNA of any one of embodiments 1-39, wherein each strand is no more than 30 nucleotides in length.

Embodiment 41. The dsRNA of any one of embodiments 1-40, wherein at least one strand comprises a 3′ overhang of at least 1 nucleotide.

Embodiment 42. The dsRNA of embodiment 41, wherein at least one strand comprises a 3′ overhang of at least 2 nucleotides.

Embodiment 43. The dsRNA of any one of embodiments 1-42, wherein the double stranded region is 19-21 nucleotide pairs in length.

Embodiment 44. The dsRNA of any one of embodiments 1-43, wherein the sense strand is 21 nucleotides in length and the antisense strand is 23 nucleotides in length.

Embodiment 45. The dsRNA of any one of embodiments 1-44, wherein the dsRNA is a small-interfering RNA (siRNA).

Embodiment 46. A double stranded ribonucleic acid (dsRNA) targeting Mucin-5B (MUC5B), wherein the dsRNA comprises a sense strand and an antisense strand forming a double stranded region, wherein the antisense strand comprises a region of complementarity to an mRNA encoding MUC5B, and wherein the antisense strand comprises a nucleotide sequence selected from SEQ ID NOs: 535, 538, 540, 541, 533, 536, or 186,538-186,624.

Embodiment 47. The dsRNA of embodiment 46, wherein the sense strand comprises a nucleotide sequence selected from SEQ ID NOs: 356, 358, 359, 361, 363, 364, or 186,502-186,537.

Embodiment 48. The dsRNA of embodiments 45-47, wherein the sense and antisense strands are selected from any one of the sense and antisense pairs in Table 9.

Embodiment 49. The dsRNA of embodiment 48, wherein:
a. the sense strand comprises the nucleotide sequence of SEQ ID NO: 358, and the antisense strand comprises the nucleotide sequence of SEQ ID NO: 186,538;
b. the sense strand comprises the nucleotide sequence of SEQ ID NO: 358, and the antisense strand comprises the nucleotide sequence of SEQ ID NO: 186,539;
c. the sense strand comprises the nucleotide sequence of SEQ ID NO: 358, and the antisense strand comprises the nucleotide sequence of SEQ ID NO: 186,540;
d. the sense strand comprises the nucleotide sequence of SEQ ID NO: 186,502, and the antisense strand comprises the nucleotide sequence of SEQ ID NO: 186,541;
e. the sense strand comprises the nucleotide sequence of SEQ ID NO: 186,503, and the antisense strand comprises the nucleotide sequence of SEQ ID NO: 535;
f. the sense strand comprises the nucleotide sequence of SEQ ID NO: 186,504, and the antisense strand comprises the nucleotide sequence of SEQ ID NO: 535;
g. the sense strand comprises the nucleotide sequence of SEQ ID NO: 186,505, and the antisense strand comprises the nucleotide sequence of SEQ ID NO: 535;
h. the sense strand comprises the nucleotide sequence of SEQ ID NO: 186,506, and the antisense strand comprises the nucleotide sequence of SEQ ID NO: 186,542;
i. the sense strand comprises the nucleotide sequence of SEQ ID NO: 186,507, and the antisense strand comprises the nucleotide sequence of SEQ ID NO: 186,543;
j. the sense strand comprises the nucleotide sequence of SEQ ID NO: 358, and the antisense strand comprises the nucleotide sequence of SEQ ID NO: 186,544;
k. the sense strand comprises the nucleotide sequence of SEQ ID NO: 358, and the antisense strand comprises the nucleotide sequence of SEQ ID NO: 186,545;
l. the sense strand comprises the nucleotide sequence of SEQ ID NO: 358, and the antisense strand comprises the nucleotide sequence of SEQ ID NO: 186,546;
m. the sense strand comprises the nucleotide sequence of SEQ ID NO: 358, and the antisense strand comprises the nucleotide sequence of SEQ ID NO: 186,547;
n. the sense strand comprises the nucleotide sequence of SEQ ID NO: 186,507, and the antisense strand comprises the nucleotide sequence of SEQ ID NO: 186,548;
o. the sense strand comprises the nucleotide sequence of SEQ ID NO: 186,507, and the antisense strand comprises the nucleotide sequence of SEQ ID NO: 186,549;
p. the sense strand comprises the nucleotide sequence of SEQ ID NO: 186,508, and the antisense strand comprises the nucleotide sequence of SEQ ID NO: 186,550;
q. the sense strand comprises the nucleotide sequence of SEQ ID NO: 186,508, and the antisense strand comprises the nucleotide sequence of SEQ ID NO: 186,551;
r. the sense strand comprises the nucleotide sequence of SEQ ID NO: 358, and the antisense strand comprises the nucleotide sequence of SEQ ID NO: 186,552;
s. the sense strand comprises the nucleotide sequence of SEQ ID NO: 358, and the antisense strand comprises the nucleotide sequence of SEQ ID NO: 186,553;
t. the sense strand comprises the nucleotide sequence of SEQ ID NO: 358, and the antisense strand comprises the nucleotide sequence of SEQ ID NO: 186,554;
u. the sense strand comprises the nucleotide sequence of SEQ ID NO: 358, and the antisense strand comprises the nucleotide sequence of SEQ ID NO: 186,555;
v. the sense strand comprises the nucleotide sequence of SEQ ID NO: 361, and the antisense strand comprises the nucleotide sequence of SEQ ID NO: 186,556;
w. the sense strand comprises the nucleotide sequence of SEQ ID NO: 361, and the antisense strand comprises the nucleotide sequence of SEQ ID NO: 186,557;
x. the sense strand comprises the nucleotide sequence of SEQ ID NO: 361, and the antisense strand comprises the nucleotide sequence of SEQ ID NO: 186,558;
y. the sense strand comprises the nucleotide sequence of SEQ ID NO: 186,509, and the antisense strand comprises the nucleotide sequence of SEQ ID NO: 186,559;
z. the sense strand comprises the nucleotide sequence of SEQ ID NO: 186,510, and the antisense strand comprises the nucleotide sequence of SEQ ID NO: 538;
aa. the sense strand comprises the nucleotide sequence of SEQ ID NO: 186,511, and the antisense strand comprises the nucleotide sequence of SEQ ID NO: 538;
bb. the sense strand comprises the nucleotide sequence of SEQ ID NO: 186,512, and the antisense strand comprises the nucleotide sequence of SEQ ID NO: 538;
cc. the sense strand comprises the nucleotide sequence of SEQ ID NO: 186,513, and the antisense strand comprises the nucleotide sequence of SEQ ID NO: 186,560;
dd. the sense strand comprises the nucleotide sequence of SEQ ID NO: 186,514, and the antisense strand comprises the nucleotide sequence of SEQ ID NO: 186,561;
ee. the sense strand comprises the nucleotide sequence of SEQ ID NO: 361, and the antisense strand comprises the nucleotide sequence of SEQ ID NO: 186,562;
ff. the sense strand comprises the nucleotide sequence of SEQ ID NO: 361, and the antisense strand comprises the nucleotide sequence of SEQ ID NO: 186,563;
gg. the sense strand comprises the nucleotide sequence of SEQ ID NO: 361, and the antisense strand comprises the nucleotide sequence of SEQ ID NO: 186,564;
hh. the sense strand comprises the nucleotide sequence of SEQ ID NO: 361, and the antisense strand comprises the nucleotide sequence of SEQ ID NO: 186,565;
ii. the sense strand comprises the nucleotide sequence of SEQ ID NO: 186,514, and the antisense strand comprises the nucleotide sequence of SEQ ID NO: 186,566;
jj. the sense strand comprises the nucleotide sequence of SEQ ID NO: 186,514, and the antisense strand comprises the nucleotide sequence of SEQ ID NO: 186,567;
kk. the sense strand comprises the nucleotide sequence of SEQ ID NO: 186,515, and the antisense strand comprises the nucleotide sequence of SEQ ID NO: 186,568;
ll. the sense strand comprises the nucleotide sequence of SEQ ID NO: 186,515, and the antisense strand comprises the nucleotide sequence of SEQ ID NO: 186,569;
mm. the sense strand comprises the nucleotide sequence of SEQ ID NO: 361, and the antisense strand comprises the nucleotide sequence of SEQ ID NO: 186,570;
nn. the sense strand comprises the nucleotide sequence of SEQ ID NO: 361, and the antisense strand comprises the nucleotide sequence of SEQ ID NO: 186,571;
oo. the sense strand comprises the nucleotide sequence of SEQ ID NO: 361, and the antisense strand comprises the nucleotide sequence of SEQ ID NO: 186,572;
pp. the sense strand comprises the nucleotide sequence of SEQ ID NO: 361, and the antisense strand comprises the nucleotide sequence of SEQ ID NO: 186,573;
qq. the sense strand comprises the nucleotide sequence of SEQ ID NO: 363, and the antisense strand comprises the nucleotide sequence of SEQ ID NO: 186,574;
rr. the sense strand comprises the nucleotide sequence of SEQ ID NO: 363, and the antisense strand comprises the nucleotide sequence of SEQ ID NO: 186,575;
ss. the sense strand comprises the nucleotide sequence of SEQ ID NO: 363, and the antisense strand comprises the nucleotide sequence of SEQ ID NO: 186,576;
tt. the sense strand comprises the nucleotide sequence of SEQ ID NO: 186,516, and the antisense strand comprises the nucleotide sequence of SEQ ID NO: 540;
uu. the sense strand comprises the nucleotide sequence of SEQ ID NO: 186,517, and the antisense strand comprises the nucleotide sequence of SEQ ID NO: 540;
vv. the sense strand comprises the nucleotide sequence of SEQ ID NO: 186,518, and the antisense strand comprises the nucleotide sequence of SEQ ID NO: 540;
ww. the sense strand comprises the nucleotide sequence of SEQ ID NO: 186,519, and the antisense strand comprises the nucleotide sequence of SEQ ID NO: 540;
xx. the sense strand comprises the nucleotide sequence of SEQ ID NO: 186,520, and the antisense strand comprises the nucleotide sequence of SEQ ID NO: 186,577;
yy. the sense strand comprises the nucleotide sequence of SEQ ID NO: 363, and the antisense strand comprises the nucleotide sequence of SEQ ID NO: 186,578;
zz. the sense strand comprises the nucleotide sequence of SEQ ID NO: 363, and the antisense strand comprises the nucleotide sequence of SEQ ID NO: 186,579;
aaa. the sense strand comprises the nucleotide sequence of SEQ ID NO: 363, and the antisense strand comprises the nucleotide sequence of SEQ ID NO: 186,580;
bbb. the sense strand comprises the nucleotide sequence of SEQ ID NO: 363, and the antisense strand comprises the nucleotide sequence of SEQ ID NO: 186,581;
ccc. the sense strand comprises the nucleotide sequence of SEQ ID NO: 363, and the antisense strand comprises the nucleotide sequence of SEQ ID NO: 186,582 (usAfscga(C3)auccaugGfaCfuuguasgsu);
ddd. the sense strand comprises the nucleotide sequence of SEQ ID NO: 363, and the antisense strand comprises the nucleotide sequence of SEQ ID NO: 186,583;
eee. the sense strand comprises the nucleotide sequence of SEQ ID NO: 363, and the antisense strand comprises the nucleotide sequence of SEQ ID NO: 186,584;
fff. the sense strand comprises the nucleotide sequence of SEQ ID NO: 364, and the antisense strand comprises the nucleotide sequence of SEQ ID NO: 186,585;
ggg. the sense strand comprises the nucleotide sequence of SEQ ID NO: 364, and the antisense strand comprises the nucleotide sequence of SEQ ID NO: 186,586;
hhh. the sense strand comprises the nucleotide sequence of SEQ ID NO: 364, and the antisense strand comprises the nucleotide sequence of SEQ ID NO: 186,587;
iii. the sense strand comprises the nucleotide sequence of SEQ ID NO: 186,521, and the antisense strand comprises the nucleotide sequence of SEQ ID NO: 186,588;
jjj. the sense strand comprises the nucleotide sequence of SEQ ID NO: 186,522, and the antisense strand comprises the nucleotide sequence of SEQ ID NO: 541;
kkk. the sense strand comprises the nucleotide sequence of SEQ ID NO: 186,523, and the antisense strand comprises the nucleotide sequence of SEQ ID NO: 541;
lll. the sense strand comprises the nucleotide sequence of SEQ ID NO: 186,524, and the antisense strand comprises the nucleotide sequence of SEQ ID NO: 541;
mmm. the sense strand comprises the nucleotide sequence of SEQ ID NO: 186,525, and the antisense strand comprises the nucleotide sequence of SEQ ID NO: 186,589;
nnn. the sense strand comprises the nucleotide sequence of SEQ ID NO: 186,526, and the antisense strand comprises the nucleotide sequence of SEQ ID NO: 186,590;
ooo. the sense strand comprises the nucleotide sequence of SEQ ID NO: 364, and the antisense strand comprises the nucleotide sequence of SEQ ID NO: 186,591;
ppp. the sense strand comprises the nucleotide sequence of SEQ ID NO: 364, and the antisense strand comprises the nucleotide sequence of SEQ ID NO: 186,592;
qqq. the sense strand comprises the nucleotide sequence of SEQ ID NO: 364, and the antisense strand comprises the nucleotide sequence of SEQ ID NO: 186,593;
rrr. the sense strand comprises the nucleotide sequence of SEQ ID NO: 364, and the antisense strand comprises the nucleotide sequence of SEQ ID NO: 186,594;
sss. the sense strand comprises the nucleotide sequence of SEQ ID NO: 186,526, and the antisense strand comprises the nucleotide sequence of SEQ ID NO: 186,595;
ttt. the sense strand comprises the nucleotide sequence of SEQ ID NO: 186,526, and the antisense strand comprises the nucleotide sequence of SEQ ID NO: 186,596;
uuu. the sense strand comprises the nucleotide sequence of SEQ ID NO: 186,527, and the antisense strand comprises the nucleotide sequence of SEQ ID NO: 186,597;
vvv. the sense strand comprises the nucleotide sequence of SEQ ID NO: 186,527, and the antisense strand comprises the nucleotide sequence of SEQ ID NO: 186,598;
www. the sense strand comprises the nucleotide sequence of SEQ ID NO: 364, and the antisense strand comprises the nucleotide sequence of SEQ ID NO: 186,599;
xxx. the sense strand comprises the nucleotide sequence of SEQ ID NO: 364, and the antisense strand comprises the nucleotide sequence of SEQ ID NO: 186,600;
yyy. the sense strand comprises the nucleotide sequence of SEQ ID NO: 364, and the antisense strand comprises the nucleotide sequence of SEQ ID NO: 186,601;
zzz. the sense strand comprises the nucleotide sequence of SEQ ID NO: 364, and the antisense strand comprises the nucleotide sequence of SEQ ID NO: 186,602;
aaaa. the sense strand comprises the nucleotide sequence of SEQ ID NO: 356, and the antisense strand comprises the nucleotide sequence of SEQ ID NO: 186,603;
bbbb. the sense strand comprises the nucleotide sequence of SEQ ID NO: 356, and the antisense strand comprises the nucleotide sequence of SEQ ID NO: 186,604;
cccc. the sense strand comprises the nucleotide sequence of SEQ ID NO: 356, and the antisense strand comprises the nucleotide sequence of SEQ ID NO: 186,605;
dddd. the sense strand comprises the nucleotide sequence of SEQ ID NO: 186,528, and the antisense strand comprises the nucleotide sequence of SEQ ID NO: 533;
eeee. the sense strand comprises the nucleotide sequence of SEQ ID NO: 186,529, and the antisense strand comprises the nucleotide sequence of SEQ ID NO: 533;
ffff. the sense strand comprises the nucleotide sequence of SEQ ID NO: 186,530, and the antisense strand comprises the nucleotide sequence of SEQ ID NO: 533;
gggg. the sense strand comprises the nucleotide sequence of SEQ ID NO: 186,531, and the antisense strand comprises the nucleotide sequence of SEQ ID NO: 533;
hhhh. the sense strand comprises the nucleotide sequence of SEQ ID NO: 186,532, and the antisense strand comprises the nucleotide sequence of SEQ ID NO: 186,606;
iiii. the sense strand comprises the nucleotide sequence of SEQ ID NO: 356, and the antisense strand comprises the nucleotide sequence of SEQ ID NO: 186,607;
jjjj. the sense strand comprises the nucleotide sequence of SEQ ID NO: 356, and the antisense strand comprises the nucleotide sequence of SEQ ID NO: 186,608;
kkkk. the sense strand comprises the nucleotide sequence of SEQ ID NO: 356, and the antisense strand comprises the nucleotide sequence of SEQ ID NO: 186,609;
llll. the sense strand comprises the nucleotide sequence of SEQ ID NO: 356, and the antisense strand comprises the nucleotide sequence of SEQ ID NO: 186,610;
mmmm. the sense strand comprises the nucleotide sequence of SEQ ID NO: 356, and the antisense strand comprises the nucleotide sequence of SEQ ID NO: 186,611 (usAfsccg(C3)aauuuggUfcAfaacagsgsa);
nnnn. the sense strand comprises the nucleotide sequence of SEQ ID NO: 356, and the antisense strand comprises the nucleotide sequence of SEQ ID NO: 186,612;
oooo. the sense strand comprises the nucleotide sequence of SEQ ID NO: 356, and the antisense strand comprises the nucleotide sequence of SEQ ID NO: 186,613;
pppp. the sense strand comprises the nucleotide sequence of SEQ ID NO: 359, and the antisense strand comprises the nucleotide sequence of SEQ ID NO: 186,614;
qqqq. the sense strand comprises the nucleotide sequence of SEQ ID NO: 359, and the antisense strand comprises the nucleotide sequence of SEQ ID NO: 186,615;
rrrr. the sense strand comprises the nucleotide sequence of SEQ ID NO: 359, and the antisense strand comprises the nucleotide sequence of SEQ ID NO: 186,616;
ssss. the sense strand comprises the nucleotide sequence of SEQ ID NO: 186,533, and the antisense strand comprises the nucleotide sequence of SEQ ID NO: 536;
tttt. the sense strand comprises the nucleotide sequence of SEQ ID NO: 186,534, and the antisense strand comprises the nucleotide sequence of SEQ ID NO: 536;
uuuu. the sense strand comprises the nucleotide sequence of SEQ ID NO: 186,535, and the antisense strand comprises the nucleotide sequence of SEQ ID NO: 536;
vvvv. the sense strand comprises the nucleotide sequence of SEQ ID NO: 186,536, and the antisense strand comprises the nucleotide sequence of SEQ ID NO: 536;
wwww. the sense strand comprises the nucleotide sequence of SEQ ID NO: 186,537, and the antisense strand comprises the nucleotide sequence of SEQ ID NO: 186,617;
xxxx. the sense strand comprises the nucleotide sequence of SEQ ID NO: 359, and the antisense strand comprises the nucleotide sequence of SEQ ID NO: 186,618;
yyyy. the sense strand comprises the nucleotide sequence of SEQ ID NO: 359, and the antisense strand comprises the nucleotide sequence of SEQ ID NO: 186,619;
zzzz. the sense strand comprises the nucleotide sequence of SEQ ID NO: 359, and the antisense strand comprises the nucleotide sequence of SEQ ID NO: 186,620;
aaaaa. the sense strand comprises the nucleotide sequence of SEQ ID NO: 359, and the antisense strand comprises the nucleotide sequence of SEQ ID NO: 186,621;
bbbbb. the sense strand comprises the nucleotide sequence of SEQ ID NO: 359, and the antisense strand comprises the nucleotide sequence of SEQ ID NO: 186,622 (usAfsgug(C3)cggguggAfaCfaaagcsusc);
ccccc. the sense strand comprises the nucleotide sequence of SEQ ID NO: 359, and the antisense strand comprises the nucleotide sequence of SEQ ID NO: 186,623; or
ddddd. the sense strand comprises the nucleotide sequence of SEQ ID NO: 359, and the antisense strand comprises the nucleotide sequence of SEQ ID NO: 186,624.

Embodiment 50. The dsRNA of embodiment 48 or 49, wherein:
a. the sense strand comprises the nucleotide sequence of SEQ ID NO: 186,507, and the antisense strand comprises the nucleotide sequence of SEQ ID NO: 186,543;
b. the sense strand comprises the nucleotide sequence of SEQ ID NO: 186,512, and the antisense strand comprises the nucleotide sequence of SEQ ID NO: 538;
c. the sense strand comprises the nucleotide sequence of SEQ ID NO: 186,514, and the antisense strand comprises the nucleotide sequence of SEQ ID NO: 186,561;
d. the sense strand comprises the nucleotide sequence of SEQ ID NO: 361, and the antisense strand comprises the nucleotide sequence of SEQ ID NO: 186,562;
e. the sense strand comprises the nucleotide sequence of SEQ ID NO: 361, and the antisense strand comprises the nucleotide sequence of SEQ ID NO: 186,563;
f. the sense strand comprises the nucleotide sequence of SEQ ID NO: 364, and the antisense strand comprises the nucleotide sequence of SEQ ID NO: 186,593;
g. the sense strand comprises the nucleotide sequence of SEQ ID NO: 364, and the antisense strand comprises the nucleotide sequence of SEQ ID NO: 186,600;
h. the sense strand comprises the nucleotide sequence of SEQ ID NO: 356, and the antisense strand comprises the nucleotide sequence of SEQ ID NO: 186,603;
i. the sense strand comprises the nucleotide sequence of SEQ ID NO: 356, and the antisense strand comprises the nucleotide sequence of SEQ ID NO: 186,605;
j. the sense strand comprises the nucleotide sequence of SEQ ID NO: 186,532, and the antisense strand comprises the nucleotide sequence of SEQ ID NO: 186,606;
k. the sense strand comprises the nucleotide sequence of SEQ ID NO: 356, and the antisense strand comprises the nucleotide sequence of SEQ ID NO: 186,607;
l. the sense strand comprises the nucleotide sequence of SEQ ID NO: 359, and the antisense strand comprises the nucleotide sequence of SEQ ID NO: 186,614;
m. the sense strand comprises the nucleotide sequence of SEQ ID NO: 186,536, and the antisense strand comprises the nucleotide sequence of SEQ ID NO: 536; or
n. the sense strand comprises the nucleotide sequence of SEQ ID NO: 186,537, and the antisense strand comprises the nucleotide sequence of SEQ ID NO: 186,617.

Embodiment 51. The modified dsRNA of any one of embodiments 46-50, wherein the dsRNA has any one or more of the following characteristics compared to the corresponding unmodified dsRNA or to the dsRNA sequence used as the starting dsRNA:
a. Improved efficacy;
b. Improved potency;
c. Improved stability; and/or
d. Reduced off-target effects.

Embodiment 52. The dsRNA of any one of embodiments 45-51, wherein the sense strand or the antisense strand:
a. is conjugated to one or more lipophilic moieties;
b. is packaged in a lipid nanoparticle (LNP); and/or
c. comprises at least one modification.

Embodiment 53. The dsRNA of any one of embodiments 45-52, wherein each of the sense strand and the antisense strand:
a. is conjugated to one or more lipophilic moieties;
b. is packaged in a lipid nanoparticle (LNP); and/or
c. comprises at least one modification.

Embodiment 54. The dsRNA of any one of embodiments 52-53, comprising one or more lipophilic moieties that are each conjugated to one or more positions in the double stranded region of the dsRNA.

Embodiment 55. A synthetic antisense oligonucleotide (ASO) targeting MUC5B, wherein the oligonucleotide comprises a nucleotide sequence:
a. having at least 90% nucleotide sequence identity to any one of SEQ ID NOs: 709-802;
b. comprising at least 15, 16, or 17 contiguous nucleotides differing in sequence by no more than 3 nucleotides from any one of SEQ ID NO: 709-802;
c. comprising at least 15, 16, or 17 contiguous nucleotides differing in sequence by no more than 3 nucleotides from any one of SEQ ID NO: 803-896; or
d. comprising at least 15, 16, or 17 contiguous nucleotides differing in sequence by no more than 3 nucleotides from any one of SEQ ID NO: 147,339-186,429.

Embodiment 56. The synthetic antisense oligonucleotide of embodiment 55 comprising a nucleotide sequence selected from any one of SEQ ID NOs: 709-802.

Embodiment 57. The synthetic antisense oligonucleotide of any one of embodiments 55-56, wherein the oligonucleotide:
a. is conjugated to one or more lipophilic moieties;
b. is packaged in a lipid nanoparticle (LNP); and/or
c. comprises at least one modified nucleotide.

Embodiment 58. The synthetic antisense oligonucleotide of any one of embodiments 55-57, comprising a nucleotide sequence selected from any one of SEQ ID NOs: 803-896.

Embodiment 59. The synthetic antisense oligonucleotide of any one of embodiments 55-58, wherein each of the nucleotides of the oligonucleotide comprises a modification.

Embodiment 60. The dsRNA of embodiment 29, or the synthetic antisense oligonucleotide of embodiment 57, wherein at least one of the nucleotides is modified, wherein the modification is selected from a deoxy-nucleotide, a 3′-terminal deoxy-thymine (dT) nucleotide, a 2′-O-methyl modified nucleotide (2′-OMe), a 2′-fluoro modified nucleotide (2′-F), a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a 5′-phosphorothioate group, a phosphorothioate linkage, a nucleotide comprising a 5′-methylphosphonate group, a nucleotide comprising a 5′ phosphate or 5′ phosphate mimic, a nucleotide comprising vinyl phosphonate, a nucleotide comprising adenosine-glycol nucleic acid (GNA), a nucleotide comprising thymidine-glycol nucleic acid (GNA) S-Isomer, a nucleotide comprising 2-hydroxymethyl-tetrahydrofurane-5-phosphate, a nucleotide comprising 2′-deoxythymidine-3′ phosphate, a nucleotide comprising 2′-deoxyguanosine-3′-phosphate, a 2′-O hexadecyl nucleotide, a nucleotide comprising a 2′-phosphate, a cytidine-2′-phosphate nucleotide, a guanosine-2′-phosphate nucleotide, a 2′-O-hexadecyl-cytidine-3′-phosphate nucleotide, a 2′-O-hexadecyl-adenosine-3′-phosphate nucleotide, a 2′-O-hexadecyl-guanosine-3′-phosphate nucleotide, a 2′-O-hexadecyl-uridine-3′-phosphate nucleotide, a 5′-vinyl phosphonate (VP/(e.g., (vin-u)), a 2′-deoxyadenosine-3′-phosphate nucleotide, a 2′-deoxycytidine-3′-phosphate nucleotide, a 2′-deoxyguanosine-3′-phosphate nucleotide, a 2′-deoxythymidine-3′-phosphate nucleotide, a 2′-deoxyuridine nucleotide, a terminal nucleotide linked to a cholesteryl derivative, a dodecanoic acid bisdecylamide group and a C3 spacer; and combinations thereof.

Embodiment 61. The synthetic antisense oligonucleotide of any one of embodiments 55-60, wherein the synthetic antisense oligonucleotide comprises 14-25 or 17-22 nucleotides.

Embodiment 62. The synthetic antisense oligonucleotide of any one of embodiments 55-61, wherein the synthetic antisense oligonucleotide comprises at least one locked nucleotide at both the 5′ and 3′ end.

Embodiment 63. The synthetic antisense oligonucleotide of any one of embodiments 55-62, wherein the synthetic antisense oligonucleotide comprises 17 nucleotides and three locked nucleotides at both the 5′ and 3′ end.

Embodiment 64. The synthetic antisense oligonucleotide of any one of embodiments 55-63, wherein the synthetic antisense oligonucleotide comprises at least one phosphorothioate linkage between nucleotides.

Embodiment 65. The synthetic antisense oligonucleotide of any one of embodiments 55-64, wherein the synthetic antisense oligonucleotide comprises a phosphorothioate linkage between each nucleotide.

Embodiment 66. The synthetic antisense oligonucleotide of any one of embodiments 55-65, wherein the synthetic antisense oligonucleotide is:
a. conjugated to one or more lipophilic moieties
b. is packaged in a lipid nanoparticle (LNP); and/or
c. comprises at least one modified nucleotide.

Embodiment 67. The dsRNA of any one of embodiments 29-31, or 62-64, or the synthetic antisense oligonucleotide of embodiment 60, wherein the dsRNA or oligonucleotide is conjugated to one or more lipophilic moieties, wherein one or more lipophilic moieties is an aliphatic, alicyclic, or polyalicyclic compound.

Embodiment 68. The dsRNA of any one of embodiments 1-54 or the synthetic antisense oligonucleotide of any one of embodiments 55-67, further comprising a targeting ligand that targets a lung tissue.

Embodiment 69. The dsRNA or the synthetic antisense oligonucleotide of embodiment 68, wherein the targeting ligand is a 2′-O-hexadecyl (C16) conjugate.

Embodiment 70. A pharmaceutical composition comprising the dsRNA of any one of embodiments 1-54 or the synthetic antisense oligonucleotide of any one of embodiments 55-69, further comprising a lipid delivery vehicle.

Embodiment 71. The pharmaceutical composition of embodiment 70, wherein the lipid delivery vehicle is a lipid nanoparticle (LNP).

Embodiment 72. The pharmaceutical composition of embodiment 71, wherein the lipid nanoparticle comprises one or more of a cationic lipid, a non-cationic lipid, a cholesterol-based lipid, a PEG-modified lipid, an amphiphilic block copolymer and/or a polymer, or a combination thereof.

Embodiment 73. A vector comprising one or more of the dsRNA of any one of embodiments 1-54 or the synthetic antisense oligonucleotide of any one of embodiments 55-69.

Embodiment 74. A cell containing the dsRNA of any one of embodiments 1-54 or the synthetic antisense oligonucleotide of any one of embodiments 55-69.

Embodiment 75. A pharmaceutical composition comprising the dsRNA of any one of embodiments 1-54 or the synthetic antisense oligonucleotide of any one of embodiments 55-69 and a diluent or excipient.

Embodiment 76. The pharmaceutical composition of embodiment 75, wherein the composition is formulated for intrapulmonary administration, inhalation or intranasal administration.

Embodiment 77. A device for inhalation administration comprising the dsRNA of any one of embodiments 1-54 or the synthetic antisense oligonucleotide of any one of embodiments 55-69.

Embodiment 78. The device of embodiment 77, wherein the device is selected from the group consisting of a nebulizer, a metered-dose inhaler, and a dry powder inhaler.

Embodiment 79. A method of inhibiting expression of a MUC5B gene in a cell, the method comprising: contacting the cell with the dsRNA of any one of embodiments 1-54 or the synthetic antisense oligonucleotide of any one of embodiments 55-69, or the pharmaceutical composition of any one of embodiments 75-76.

Embodiment 80. The method of embodiment 79, wherein the cell is within a subject.

Embodiment 81. The method of embodiment 80, wherein the subject is a human.

Embodiment 82. The method of any one of embodiments 79-81, wherein expression of the MUC5B gene is inhibited by at least 50%.

Embodiment 83. A method for treating a subject having a lung disease or a subject at risk of developing a lung disease, comprising administering to the subject in need thereof a therapeutically effective amount of the dsRNA of any one of embodiments 1-54 or the synthetic antisense oligonucleotide of any one of embodiments 55-69, or the pharmaceutical composition of embodiment 75 or embodiment 76.

Embodiment 84. The method of embodiment 83, wherein the subject is a human.

Embodiment 85. The method of any one of embodiments 83-84, wherein the lung disease is associated with overexpression of MUC5B, optionally wherein overexpression of MUC5B is associated with one or more of reduced mucociliary function, reduced alveolar repair, and increased lung fibrosis.

Embodiment 86. The method of any one of embodiments 83-85, wherein the lung disease is one or more of pulmonary fibrosis, cystic fibrosis, chronic obstructive pulmonary disease (COPD), asthma, Primary Ciliary Dyskinesia, or bronchiectasis.

Embodiment 87. The method of embodiment 86, wherein the pulmonary fibrosis is idiopathic pulmonary fibrosis (IPF).

Embodiment 88. The method of any one of embodiments 79-87, wherein treating comprises amelioration of at least symptom of the disease.

Embodiment 89. The method of any one of embodiments 79-88, wherein the dsRNA, the synthetic antisense oligonucleotide, or the pharmaceutical composition is administered to the subject subcutaneously, orotracheally, via oral inhalation, or via intranasal administration.

Embodiment 90. The method of any one of embodiments 79-89, further comprising administering to the subject an additional agent or a therapy suitable for treatment or prevention of a lung disease.

Embodiment 91. The method of embodiment 90, wherein the additional therapeutic agent is selected from the group consisting of an anti-inflammatory agent, an immune modulator, a phosphodiesterase-5 inhibitor, a tyrosine kinase inhibitor, an antifibrotic agent and a combination of any of the foregoing.

### BRIEF DESCRIPTION OF THE DRAWINGS

FIGS. 1A and 1B present a schematic of the reporter systems for measuring on-target (FIG. 1A) and off-target (FIG. 1B) performance of dsRNAs in A549 cells as described in example 4.

FIG. 2 shows exemplary modifications to a dsRNA duplex of the EEL backbone. Nucleotides are numbered from 5′ to 3′ for the sense and antisense strands. An exemplary EEL 21/23mer is depicted. For EEL 19/21mer duplexes, position 12 of the sense strand is an unmodified RNA nucleotide (not shown in Figure).

FIG. 3 shows exemplary modifications to a dsRNA duplex of the DV22 backbone. Nucleotides are numbered from 5′ to 3′ for the sense and antisense strands.

### DETAILED DESCRIPTION

Reference will now be made in detail to certain embodiments of the invention. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biolog, published by Blackwell Science Ltd., 1994 (ISBN 0-632-02182-9); and Robert A. Meyers (ed.), Molecular Biolog and Biotechnology: a Comprehensive Desk Reference, published by VCH Publishers, Inc., 1995 (ISBN 1-56081-569-8). Unless otherwise explained, all technical and scientific terms used herein have the same meaning as commonly understood by one of ordinary skill in the art to which this disclosure belongs.

Before describing the present teachings in detail, it is to be understood that the disclosure is not limited to specific compositions or process steps, as such may vary. It should be noted that, as used in this specification and the appended claims, the singular form “a,” “an,” and “the” include plural references unless the context clearly dictates otherwise. Thus, for example, reference to “a guide” includes a plurality of guides, and reference to “a cell” includes a plurality of cells and the like. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

Further, ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise). Any concentration range, percentage range, ratio range, or integer range is to be understood to include the value of any integer within the recited range and, when appropriate, fractions thereof (such as one-tenth and one-hundredth of an integer), unless otherwise indicated. Also, any number range recited herein relating to any physical feature, such as polymer subunits, size, or thickness, is to be understood to include any integer within the recited range unless otherwise indicated. As used herein, unless otherwise indicated, “about” or “consisting essentially of” mean ±20% of the indicated range, value, or structure. As used herein, the terms “include” and “comprise” are open-ended and used synonymously.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### I. Definitions

Unless stated otherwise, the following terms and phrases as used herein are intended to have the following meanings:

“Nucleotide,” “nucleic acid,” and “nucleic acid molecule” are used herein to refer to a multimeric compound comprising nucleosides or nucleoside analogs that have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O′-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion, see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases, and linkages or can include both conventional components and substitutions (e.g., conventional bases with 2′ methoxy linkages or polymers containing both conventional bases and one or more base analogs). Nucleic acid includes “locked nucleic acid” (LNA), an analog containing one or more LNA nucleotide monomers with a bicyclic furanose unit locked in an RNA-mimicking sugar conformation, which enhances hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Mucin 5B” (“MUC5B”) refers to the well-known gene and polypeptide, also known in the art as also referred to as “Mucin 5B, Oligomeric Mucus/Gel-Forming,” “High Molecular Weight Salivary Mucin MG1,” “Mucin 5, Subtype B, Tracheobronchial,” “Sublingual Gland Mucin,” “Mucin-5B,” “MUC-5B,” “MUC5,” “MG1,” “Mucin-5 Subtype B, Tracheobronchial,” “Cervical Mucin MUC5B,” “Cervical Mucin,” or “MUC9.” The term “MUC5B” includes human MUC5B, the amino acid and nucleotide sequences of which may be found in, for example, GenBank Accession No. NM_002458.3 (GI: 1519244536; SEQ ID NO: 897); mouse MUC5B, the amino acid and nucleotide sequences of which may be found in, for example, GenBank Accession No. NM_028801.2 (GL: 147905739; SEQ ID NO: 898); and rat MUC5B, the amino acid and nucleotide sequences of which may be found in, for example, GenBank Accession No.: XM_006230608.2 (GI: 672039062; SEQ ID NO: 899).

“MUC5B” also includes Macaca mulatta MUC5B, the amino acid and nucleotide sequences of which may be found in, for example, GenBank Accession No. XM_028833012.1 (GL: 1622861542; SEQ ID NO: 900) and Macaca fascicularis MUC5B, the amino acid and nucleotide sequences of which may be found in, for example, GenBank Accession No. XM_015435240.1 (GL: 982295518; SEQ ID NO: 901). Further information on MUC5B is provided, for example, in the NCBI Gene database at ncbi.nlm.nih.gov/gene/727897. The entire contents of each of the foregoing GenBank Accession numbers and the Gene database numbers are incorporated herein by reference as of the date of filing this application.

The terms “Mucin-5B” and “MUC5B,” as used herein, also refer to naturally occurring DNA sequence variations of the MUC5B gene. Numerous sequence variations within the MUC5B gene have been identified and may be found at, for example, NCBI dbSNP and UniProt (see, e.g., ncbi.nlm.nih.gov/snp/?term=muc5b), the entire contents of which is incorporated herein by reference as of the date of filing this application.

As used herein, the term “strand comprising a sequence” refers to an oligonucleotide comprising a chain of nucleotides that is described by the sequence referred to using the standard nucleotide nomenclature.

“G,” “C,” “A,” “T,” and “U” each generally stand for a nucleotide that contains guanine, cytosine, adenine, thymidine, and uracil as a base, respectively. However, it will be understood that the term “ribonucleotide” or “nucleotide” can also refer to a modified nucleotide, as further detailed below, or a surrogate replacement moiety. The skilled person is well aware that guanine, cytosine, adenine, and uracil can be replaced by other moieties without substantially altering the base pairing properties of an oligonucleotide comprising a nucleotide bearing such replacement moiety. It is understood that when a cDNA sequence is provided, the corresponding mRNA, RNAi agent, or ASO would include a U in place of a T. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the invention by a nucleotide containing, for example, inosine. In another example, adenine and cytosine anywhere in the oligonucleotide can be replaced with guanine and uracil, respectively, to form a G-U Wobble base pairing with the target mRNA. Sequences containing such replacement moieties are suitable for the compositions and methods featured in the invention.

The terms “antisense oligonucleotide” and “ASO,” as used herein, refer to a single-stranded oligonucleotide having a nucleobase sequence that permits hybridization to a corresponding segment of a target nucleic acid. ASOs are used for gene silencing by RNA cleavage and RNA editing. ASOs may also be used to modulate the splicing of a target gene by enhancing the retention or skipping of a specific exon.

The terms “iRNA”, “RNAi agent,” “iRNA agent,” and “RNA interference agent,” as used interchangeably herein, refer to an agent that contains RNA as that term is defined herein and which mediates the targeted cleavage of an RNA transcript via an RNA-induced silencing complex (RISC) pathway. RNA interference (RNAi) is a process that directs the sequence-specific degradation of mRNA. RNAi modulates, e.g., inhibits, the expression of a MUC5B gene in a cell, e.g., a cell within a subject, such as a mammalian subject.

In one embodiment, an RNAi agent of the disclosure includes a single-stranded RNAi that interacts with a target RNA sequence, e.g., a MUC5B mRNA sequence, to direct the cleavage of the target RNA. Without wishing to be bound by theory, it is believed that long double-stranded RNA introduced into cells is broken down into double-stranded short interfering RNAs (siRNAs) comprising a sense strand and an antisense strand by a Type HI endonuclease known as Dicer (Sharp et al. (2001) Genes Dev. 15:485). Dicer, a ribonuclease-III-like enzyme, processes these dsRNA into 19-23 base pair short interfering RNAs with characteristic two-base 3′ overhangs (Bernstein, et al., (2001) Nature 409:363). These siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect, the disclosure relates to a single stranded RNA (ssRNA) (the antisense strand of an siRNA duplex) generated within a cell and which promotes the formation of a RISC complex to effect silencing of the target gene. Accordingly, the term “siRNA” is also used herein to refer to an RNAi as described above.

In another embodiment, the RNAi agent may be a single-stranded RNA that is introduced into a cell or organism to inhibit a target mRNA. Single-stranded RNAi agents bind to the RISC endonuclease, Argonaute 2, which then cleaves the target mRNA. The single-stranded siRNAs are generally 15-30 nucleotides and are chemically modified. The design and testing of single-stranded RNAs are described in U.S. Pat. No. 8,101,348 and in Lima et al., (2012) Cell 150:883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150:883-894.

In another embodiment, an “RNAi agent” for use in the compositions and methods of the disclosure is a double-stranded RNA and is referred to herein as a “double-stranded RNAi agent,” “double-stranded RNA (dsRNA) molecule,” “dsRNA agent,” or “dsRNA”. The term “dsRNA” refers to a complex of ribonucleic acid molecules, having a duplex structure comprising two anti-parallel and substantially complementary nucleic acid strands, referred to as having “sense” and “antisense” orientations with respect to a target RNA, i.e., a MUC5B mRNA sequence. In some embodiments of the disclosure, a double-stranded RNA (dsRNA) triggers the degradation of a target RNA, e.g., an mRNA, through a post-transcriptional gene-silencing mechanism referred to herein as RNA interference or RNAi.

In general, a dsRNA molecule can include ribonucleotides, but as described in detail herein, each or both strands can also include one or more non-ribonucleotides, e.g., a deoxyribonucleotide, or a modified nucleotide. In addition, as used in this specification, an “RNAi agent” may include ribonucleotides with chemical modifications; an RNAi agent may include substantial modifications at multiple nucleotides.

As used herein, the term “modified nucleotide” refers to a nucleotide having, independently, a modified sugar moiety, a modified internucleotide linkage, or a modified nucleobase. Thus, the term modified nucleotide encompasses substitutions, additions, or removal of, e.g., a functional group or atom to internucleotide linkages, sugar moieties, or nucleobases. The modifications suitable for use in the agents of the disclosure include all types of modifications disclosed herein or known in the art. Any such modifications, as used in an siRNA type molecule or ASO, are encompassed by “RNAi agent” or “ASO” for the purposes of this specification and claims.

As used herein, the term “nucleotide overhang” refers to at least one unpaired nucleotide that protrudes from the duplex structure of an RNAi agent, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively, the overhang can comprise at least two nucleotides, at least three nucleotides, at least four nucleotides, at least five nucleotides, or more. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5′-end, 3′-end, or both ends of either an antisense or sense strand of a dsRNA.

In one embodiment of the dsRNA, at least one strand comprises a 3′ overhang of at least 1 nucleotide. In another embodiment, at least one strand comprises a 3′ overhang of at least 2 nucleotides, e.g., 2, 3, 4, 5, 6, 7, 9, 10, 11, 12, 13, 14, or 15 nucleotides. In other embodiments, at least one strand of the RNAi agent comprises a 5′ overhang of at least 1 nucleotide. In certain embodiments, at least one strand comprises a 5′ overhang of at least 2 nucleotides, e.g., 2, 3, 4, 5, 6, 7, 9, 10, 11, 12, 13, 14, or 15 nucleotides. In still other embodiments, both the 3′ and the 5′ end of one strand of the RNAi agent comprise an overhang of at least 1 nucleotide.

In one embodiment, the antisense strand of a dsRNA has a 1-10 nucleotide, e.g., 0-3, 1-3, 2-4, 2-5, 4-10, 5-10, e.g., a 1, 2, 3, 4, 5, 6, 7, 8, 9, or 10 nucleotide, overhang at the 3′-end or the 5-end. In one embodiment, the sense strand of a dsRNA has a 1-10 nucleotide, e.g., a 1, 2, 3, 4, 5, 6, 7, 8, 9, or 10 nucleotide, overhang at the 3′-end or the 5′-end. In another embodiment, one or more of the nucleotides in the overhang is replaced with a nucleoside thiophosphate.

In certain embodiments, the overhang on the sense strand or the antisense strand, or both, can include extended lengths longer than 10 nucleotides, e.g., 1-30 nucleotides, 2-30 nucleotides, 10-30 nucleotides, or 10-15 nucleotides in length. In certain embodiments, an extended overhang is on the sense strand of the duplex. In certain embodiments, an extended overhang is present on the 3′end of the sense strand of the duplex. In certain embodiments, an extended overhang is present on the 5′end of the sense strand of the duplex. In certain embodiments, an extended overhang is on the antisense strand of the duplex. In certain embodiments, an extended overhang is present on the 3′end of the antisense strand of the duplex. In certain embodiments, an extended overhang is present on the 5′end of the antisense strand of the duplex. In certain embodiments, one or more of the nucleotides in the overhang is replaced with a nucleoside thiophosphate. In certain embodiments, the overhang includes a self-complementary portion such that the overhang is capable of forming a hairpin structure that is stable under physiological conditions.

The terms “blunt” or “blunt ended,” as used herein in reference to a dsRNA, mean that there are no unpaired nucleotides or nucleotide analogs at a given terminal end of a dsRNA, i.e., no nucleotide overhang. One or both ends of a dsRNA can be blunt. Where both ends of a dsRNA are blunt, the dsRNA is said to be blunt ended. To be clear, a “blunt ended” dsRNA is a dsRNA that is blunt at both ends, i.e., no nucleotide overhang at either end of the molecule. Most often, such a molecule will be double stranded over its entire length.

The term “antisense strand” or “guide strand” refers to the strand of an iRNA, e.g., a dsRNA, which includes a region complementary to a target sequence, e.g., a MUC5B mRNA sequence. In some embodiments, the complementarity is full or partial. In some instances, “full” or “perfectly complementary” or “fully complementary” means that in a hybridized pair of nucleobase or nucleotide sequence molecules, all (100%) of the bases in a contiguous sequence of a first oligonucleotide will hybridize with the same number of bases in a contiguous sequence of a second oligonucleotide. The contiguous sequence may comprise all or a part of a first or second nucleotide sequence. In some instances, “partial” or “partially complementary” means that in a hybridized pair of nucleobase or nucleotide sequence molecules, at least 70%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

The term “sense strand” or “passenger strand” as used herein, refers to the strand of a RNAi agent that includes a region that is substantially complementary to a region of the antisense strand as that term is defined herein.

As used herein in reference to a nucleic acid, “substantially all of the nucleotides are modified” means that the majority but not all of the nucleotides in the nucleic acid are modified, wherein the nucleic acid can include not more than 5, 4, 3, 2, or 1 unmodified nucleotides.

The term “lipophile” or “lipophilic moiety” broadly refers to any compound or chemical moiety having an affinity for lipids. One way to characterize the lipophilicity of the lipophilic moiety is by the octanol-water partition coefficient, log K_{ow}, where K_{ow} is the ratio of a chemical's concentration in the octanol-phase to its concentration in the aqueous phase of a two-phase system at equilibrium. The octanol-water partition coefficient is a laboratory-measured property of a substance. However, it may also be predicted by using coefficients attributed to the structural components of a chemical, which are calculated using first-principle or empirical methods (see, for example, Tetko et al., J. Chem. Inf. Comput. Sci. 41:1407-21 (2001), which is incorporated herein by reference in its entirety). It provides a thermodynamic measure of the tendency of the substance to prefer a non-aqueous or oily milieu rather than water (i.e., its hydrophilic/lipophilic balance). In principle, a chemical substance is lipophilic when its log K_{ow} exceeds 0. Typically, the lipophilic moiety possesses a log K_{ow} exceeding 1, exceeding 1.5, exceeding 2, exceeding 3, exceeding 4, exceeding 5, or exceeding 10. For instance, the log K_{ow} of 6-amino hexanol is predicted to be approximately 0.7. Using the same method, the log K_{ow} of cholesteryl N-(hexan-6-ol) carbamate is expected to be 10.7.

The lipophilicity of a molecule can change with respect to the functional group it carries. For instance, adding a hydroxyl group or amine group to the end of a lipophilic moiety can increase or decrease the partition coefficient (e.g., log K_{ow}) value of the lipophilic moiety.

Accordingly, conjugating the lipophilic moieties to the internal position(s) of the double-stranded RNAi agent or ASO may provide optimal hydrophobicity for the enhanced in vivo siRNA or ASO delivery.

The term “lipid nanoparticle” or “LNP” is a vesicle comprising a lipid layer encapsulating a pharmaceutically active molecule, such as a nucleic acid molecule, e.g., an RNAi agent or ASO, or a plasmid from which an RNAi agent or ASO is transcribed. LNPs are described in, for example, U.S. Pat. Nos. 6,858,225, 6,815,432, 8,158,601, and 8,058,069, the entire contents of which are hereby incorporated herein by reference.

As used herein, a “subject” is an animal, such as a mammal, including a primate (such as a human, a non-human primate, e.g., a monkey, and a chimpanzee), or a non-primate (such as a cow, a pig, a horse, a goat, a rabbit, a sheep, a hamster, a guinea pig, a cat, a dog, a rat, or a mouse), or a bird that expresses the target gene, either endogenously or heterologously. In a preferred embodiment, the subject is a human, such as a human being treated or assessed for a disease, disorder, or condition that would benefit from reduction in MUC5B expression; a human at risk for a disease, disorder, or condition that would benefit from reduction in MUC5B expression; a human having a disease, disorder, or condition that would benefit from reduction in MUC5B expression; or human being treated for a disease, disorder, or condition that would benefit from reduction in MUC5B expression as described herein. In some embodiments, the subject is a female human. In other embodiments, the subject is a male human. In one embodiment, the subject is an adult subject. In another embodiment, the subject is a pediatric subject.

As used herein, the terms “treating” or “treatment” refer to a beneficial or desired result including, but not limited to, alleviation or amelioration of one or more signs or symptoms associated with abnormal or unwanted MUC5B expression or MUC5B protein production, e.g., a MUC5B-associated disease, e.g., a lung disease, e.g., cystic fibrosis, chronic obstructive pulmonary disease (COPD), and/or pulmonary fibrosis, e.g., idiopathic pulmonary fibrosis (IPF) or symptoms associated with unwanted MUC5B expression; diminishing the extent of unwanted MUC5B activation or stabilization; amelioration or palliation of unwanted MUC5B activation or stabilization. “Treatment” can also mean prolonging survival as compared to expected survival in the absence of treatment.

The term “lower” in the context of the level of MUC5B in a subject or a disease marker or symptom refers to a statistically significant decrease in such level. The decrease can be, for example, at least 10%, 15%, 20%, 25%, 30%, %, 40%, 45%, 50%, 55%, 60%, 65%, 70%, 75%, 80%, 85%, 90%, 95%, or more. In certain embodiments, a decrease is at least 20%. In certain embodiments, the decrease is at least 50% in a disease marker, e.g., protein or gene expression level. “Lower” in the context of the level of MUC5B in a subject is preferably down to a level accepted as within the range of normal for an individual without such disorder. In certain embodiments, the expression of the target is normalized, i.e., decreased towards or to a level accepted as within the range of normal for an individual without such disorder, e.g., blood oxygen level, white blood cell count, kidney function, liver function. As used here, “lower” in a subject can refer to lowering of gene expression or protein production in a cell in a subject but does not require lowering of expression in all cells or tissues of a subject. For example, as used herein, lowering in a subject can include lowering of gene expression or protein production in a subject.

The term “lower” can also be used in association with normalizing a symptom of a disease or condition, i.e., decreasing the difference between a level in a subject suffering from a MUC5B-associated disease towards or to a level in a normal subject not suffering from a MUC5B-associated disease. As used herein, if a disease is associated with an elevated value for a symptom, “normal” is considered to be the upper limit of normal. If a disease is associated with a decreased value for a symptom, “normal” is considered to be the lower limit of normal.

As used herein, “prevention” or “preventing,” when used in reference to a disease, disorder, or condition thereof, that would benefit from a reduction in expression of a MUC5B gene or production of a MUC5B protein, refers to a reduction in the likelihood that a subject will develop a symptom associated with such a disease, disorder, or condition, e.g., a symptom of a MUC5B-associated disease, e.g., cystic fibrosis, chronic obstructive pulmonary disease (COPD), and/or pulmonary fibrosis, e.g., IPF. The failure to develop a disease, disorder, or condition, or the reduction in the development of a symptom associated with such a disease, disorder, or condition (e.g., by at least about 10% on a clinically accepted scale for that disease or disorder), or the exhibition of delayed symptoms delayed (e.g., by days, weeks, months or years) is considered effective prevention.

As used herein, the term “MUC5B-associated disease” is a disease or disorder that would benefit from reduction in the expression or activity of MUC5B. Such MUC5B-associated diseases include a MUC5B-associated disease.

The term “MUC5B-associated disease” is a disease or disorder caused by or associated with MUC5B expression or MUC5B protein production. The term “MUC5B-associated disease” includes a disease, disorder, or condition that would benefit from a decrease in MUC5B expression or MUC5B protein activity. Non-limiting examples of MUC5B-associated diseases include, for example, lung diseases, e.g., cystic fibrosis, chronic obstructive pulmonary disease (COPD), and/or pulmonary fibrosis, e.g., idiopathic pulmonary fibrosis (IPF).

As used herein, the term “pulmonary fibrosis” refers to a condition of the lungs in which the tissue thickens and becomes scarred. This thickened, stiff tissue makes it more difficult for the lungs to work properly. As pulmonary fibrosis worsens, people become progressively more short of breath. In some embodiments, the cause of pulmonary fibrosis is unknown. In those instances, the pulmonary fibrosis is referred to as “idiopathic pulmonary fibrosis (IPF).”

As used herein, the term “chronic obstructive pulmonary disease (COPD)” refers to a lung disease characterized by chronic obstruction of airflow. In COPD, the lung damage over time leads to a loss in elasticity of the lung tissue responsible for proper exhalation. When this elasticity is lost, some waste carbon dioxide is left in the lungs at the end of exhalation, leading to carbon dioxide buildup in the body. COPD leads to emphysema, which is the destruction of the alveoli, and chronic bronchitis, which is inflammation of the airway tubes in the lungs.

As used herein, the term “cystic fibrosis” refers to a genetic disorder resulting in thickening tissue and mucus buildup in the lungs, pancreas, liver, kidneys, and intestines. Individuals with cystic fibrosis develop a thick mucus that can block the airways in the lungs. This mucus buildup results in troubled breathing and an increased susceptibility to respiratory infections, as mucus traps the bacteria and cannot be removed efficiently. This condition also has severely debilitating effects on the digestive system, resulting in stunted growth and weight.

The symptoms of a MUC5B-associated disease include, for example, exertional dyspnea, a nonproductive cough, weight loss, low-grade fevers, fatigue, arthralgias, fine bibasilar inspiratory crackles (Velcro crackles), digital clubbing, pulmonary hypertension at rest, loud P2 component of the second heart sound, a fixed split S2, a holosystolic tricuspid regurgitation murmur, pedal edema, histopathologic and/or radiologic pattern of usual interstitial pneumonia (UIP), mucus buildup in the airways, troubled breathing, increased susceptibility to respiratory infections, stunted growth and weight, a loss in elasticity of the lung tissue, carbon dioxide buildup in the body, emphysema, chronic bronchitis, shortness of breath, chronic cough and excessive mucus, wheezing, a tight feeling in the chest, blue lips and nail beds, and uncontrollable weight loss. Further details regarding signs and symptoms of the various diseases or conditions are provided herein and are well-known in the art.

“Therapeutically effective amount,” as used herein, is intended to include the amount of an RNAi agent or ASO that, when administered to a subject having a MUC5B-associated disease, is sufficient to effect treatment of the disease (e.g., by diminishing, ameliorating, or maintaining the existing disease or one or more symptoms of disease). The “therapeutically effective amount” may vary depending on the RNAi agent or ASO, how the agent is administered, the disease and its severity and the history, age, weight, family history, genetic makeup, the types of preceding or concomitant treatments, if any, and other individual characteristics of the subject to be treated.

The phrase “pharmaceutically acceptable” is employed herein to refer to those compounds, materials, compositions, or dosage forms that are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition, or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt, (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline: (18) Ringer's solution; (19) ethyl alcohol; (20) pH buffered solutions; (21) polyesters, polycarbonates or polyanhydrides; (22) bulking agents, such as polypeptides and amino acids (23) serum component, such as serum albumin, HDL and LDL; and (22) other non-toxic compatible substances employed in pharmaceutical formulations. Pharmaceutically acceptable carriers for pulmonary delivery are known in the art and will vary depending on the desired location for deposition of the agent, e.g., upper or lower respiratory system, and the type of device to be used for delivery, e.g., sprayer, nebulizer, dry powder inhaler.

As used herein, “respiratory system” is understood as the structures through which air moves from outside the body into the lungs and back out, e.g., the mouth, nose and nasal cavity, sinus, trachea, pharynyx, larynx, bronchial tubes/bronchi, bronchioles, alveoli, and vasculature, e.g., capillaries, hematopoietic cells, lymphatics, and lungs, and the cells, tissues, and fluids present therein.

As used herein, “delivery by inhalation” and the like include delivery by inhalation through the nose or mouth, including intratracheal administration. Delivery by inhalation typically is performed using a device, e.g., inhaler, sprayer, nebulizer, that is selected, in part, based on the location that the agent is to be delivered, e.g., nose, mouth, lungs, and the type of material to be delivered, e. g., drops, mist, dry powder.

The term “sample,” as used herein, includes a collection of similar fluids, cells, or tissues isolated from a subject and fluids, cells, or tissues present within a subject. Examples of biological fluids include blood, serum and serosal fluids, plasma, bronchial fluids, sputum, cerebrospinal fluid, ocular fluids, lymph, urine, saliva, sputum, and the like. Tissue samples may include samples from tissues, organs or localized regions. For example, samples may be derived from particular organs, parts of organs, or fluids or cells within those organs. In certain embodiments, samples may be derived from a nasal swab. In certain embodiments, samples may be derived from a throat swab. In certain embodiments, samples may be derived from the lung, or certain types of cells in the lung. In some embodiments, the samples may be derived from the bronchioles. In some embodiments, the samples may be derived from the bronchus. In some embodiments, the samples may be derived from the alveoli. In other embodiments, a “sample derived from a subject” refers to liver tissue (or subcomponents thereof) derived from the subject. In some embodiments, a “sample derived from a subject” refers to blood drawn from the subject or plasma or serum derived therefrom. In further embodiments, a “sample derived from a subject” refers to pulmonary tissue (or subcomponents thereof) derived from the subject.

### II. RNAi Agents of the Disclosure

Described herein are RNAi agents that inhibit the expression of a MUC5B gene. In one embodiment, the RNAi agent includes double-stranded ribonucleic acid (dsRNA) molecules for inhibiting the expression of a MUC5B gene in a subject, e.g., a mammal, such as a human, e.g., a subject having a MUC5B-associated disorder, e.g., a lung disease, e.g., cystic fibrosis, chronic obstructive pulmonary disease (COPD), and/or pulmonary fibrosis, e.g., IPF, or a subject at risk of a MUC5B-associated disease, such as LPF, e.g., a subject carrying an rs35705950 variant.

The dsRNA includes two RNA strands (a sense strand and an antisense strand) that are complementary and hybridized to form a duplex structure under conditions in which the dsRNA will be used. The antisense strand includes a region of complementarity that is substantially complementary, and in some embodiments, fully complementary, to a target sequence, for example, the sequence derived from the sequence of an mRNA formed during the expression of a MUC5B gene. The sense strand includes a region complementary to the antisense strand, such that the two strands hybridize and form a double-stranded region when combined under suitable conditions. As described elsewhere herein and as known in the art, the complementary sequences of a dsRNA can also be contained as self-complementary regions of a single nucleic acid molecule, as opposed to being on separate oligonucleotides.

In some embodiments, the antisense strand comprises a nucleotide sequence selected from SEQ ID NOs: 178-354. In some embodiments, the sense strand comprises a nucleotide sequence selected from SEQ ID NOs: 1-177. In some embodiments, the sense strand comprises a nucleotide sequence selected from SEQ ID NOs: 1-177 and the antisense strand comprises a nucleotide sequence selected from SEQ ID NOs: 178-354.

In some embodiments, the antisense strand comprises a nucleotide sequence comprising at least 22 contiguous nucleotides differing by no more than 1 nucleotide from any one of SEQ ID NOs: 179 and 180. In some of these embodiments, the sense strand comprises a nucleotide sequence comprising at least 20 contiguous nucleotides differing by no more than 1 nucleotide from any one of SEQ ID NOs: 2 and 3.

In some embodiments, the antisense strand comprises a nucleotide sequence comprising at least 19 contiguous nucleotides differing by no more than 5 nucleotides from any one of SEQ ID NOs: 178 and 181-187. In some embodiments, the antisense strand comprises a nucleotide sequence having at least 66% nucleotide sequence identity to any one of SEQ ID NOs: 178 and 181-187. In some of these embodiments, the sense strand comprises a nucleotide sequence comprising at least 17 contiguous nucleotides differing by no more than 3 nucleotides from any one of SEQ ID NOs: 1 and 4-10, or the sense strand comprises a nucleotide sequence having at least 62% nucleotide sequence identity to any one of SEQ ID NOs: 1 and 4-10.

In some embodiments, the sense strand comprises a nucleotide sequence comprising at least 17 contiguous nucleotides differing by no more than 4 nucleotides from SEQ ID NO. 8, and the antisense strand comprises a nucleotide sequence comprising at least 19 contiguous nucleotides differing by no more than 6 nucleotides from SEQ ID NO: 185. In some embodiments, wherein the sense strand comprises a nucleotide sequence comprising at least 17 contiguous nucleotides differing by no more than 5 nucleotides from SEQ ID NO: 9, and the antisense strand comprises a nucleotide sequence comprising at least 19 contiguous nucleotides differing by no more than 7 nucleotides from SEQ ID NO: 186. In some embodiments, the sense strand comprises a nucleotide sequence comprising at least 17 contiguous nucleotides differing by no more than 7 nucleotides from SEQ ID NO: 10, and the antisense strand comprises a nucleotide sequence comprising at least 19 contiguous nucleotides differing by no more than 9 nucleotides from SEQ ID NO: 187.

Generally, the duplex structure is 15 to 30 base pairs in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length.

In certain preferred embodiments, the duplex structure is 18 to 25 base pairs in length, e.g., 18-25, 18-24, 18-23, 18-22, 18-21, 18-20, 19-25, 19-24, 19-23, 19-22, 19-21, 19-20, 20-25, 20-24,20-23, 20-22, 20-21, 21-25, 21-24, 21-23, 21-22, 22-25, 22-24, 22-23, 23-25, 23-24 or 24-25 base pairs in length, for example, 19-21 base pairs in length. Ranges and lengths intermediate to the above-recited ranges and lengths are also considered part of the disclosure. Similarly, the region of complementarity to the target sequence is 15 to 30 nucleotides in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length, for example 19-23 nucleotides in length or 21-23 nucleotides in length. Ranges and lengths intermediate to the above-recited ranges and lengths are also considered part of the disclosure.

In some embodiments, the dsRNA is 15 to 23 nucleotides in length or 25 to 30 nucleotides in length. In general, the dsRNA is long enough to serve as a substrate for the Dicer enzyme. For example, it is well known in the art that dsRNAs longer than about 21-23 nucleotides can serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA, e.g., a duplex region of about 15 to 36 base pairs, e.g., 15-36, 15-35, 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs, for example, 19-21 base pairs. Thus, in one embodiment, to the extent that it becomes processed to a functional duplex, of, e.g., 15-30 base pairs, that targets a desired RNA for cleavage, an RNA molecule or complex of RNA molecules having a duplex region greater than 30 base pairs is a dsRNA. Thus, an ordinarily skilled artisan will recognize that in one embodiment, an miRNA is a dsRNA. In another embodiment, a dsRNA is not a naturally occurring miRNA. In another embodiment, an RNAi agent useful to target MUC5B expression is not generated in the target cell by cleavage of a larger dsRNA.

A dsRNA, as described herein, can further include one or more single-stranded nucleotide overhangs, e.g., 1, 2, 3, or 4 nucleotides. A nucleotide overhang can comprise or consist of a nucleotide/nucleoside analog, including a deoxynucleotide/nucleoside. The overhang(s) can be on the sense strand, the antisense strand, or any combination thereof. Furthermore, an overhang's nucleotide(s) can be present on the 5′-end, 3′-end, or both ends of either an antisense or sense strand of a dsRNA. In certain embodiments, longer, extended overhangs are possible.

A dsRNA can be synthesized by standard methods known in the art as further discussed below, e.g., by use of an automated DNA synthesizer, such as are commercially available from, for example, Biosearch, Applied Biosystems, Inc. iRNA compounds of the invention may be prepared using a two-step procedure. First, the individual strands of the double-stranded RNA molecule are prepared separately. Then, the component strands are annealed. The individual strands of the siRNA compound can be prepared using solution-phase or solid-phase organic synthesis or both. Organic synthesis offers the advantage that the oligonucleotide strands comprising unnatural or modified nucleotides can be easily prepared. Single-stranded oligonucleotides of the invention can be prepared using solution-phase or solid-phase organic synthesis or both.

An siRNA can be produced, e.g., in bulk, by a variety of methods. Exemplary methods include organic synthesis and RNA cleavage, e.g., in vitro cleavage.

In one embodiment, the RNA of the RNAi agent of the disclosure, e.g., a dsRNA, is un-modified and does not comprise, e.g., chemical modifications or conjugations known in the art and described herein. In some embodiments, the RNA of an RNAi agent of the disclosure, e.g., a dsRNA, is chemically modified to enhance stability or other beneficial characteristics. In some embodiments, the dsRNA comprises at least one modified nucleotide. In some embodiments, at least 1, 2, 3, 4, 5, 6, 7, 8, 9, 10, 11, 12, 13, 14, 15, 16, 17, 18, 19, or 20 nucleotides of the sense strand are modified. In some embodiments, at least 1, 2, 3, 4, 5, 6, 7, 8, 9, 10, 11, 12, 13, 14, 15, 16, 17, 18, 19, or 20 nucleotides of the antisense strand are modified. In some embodiments, at least 99, 98, 97, 96, 95, 94, 93, 92, 91, 90, 89, 88, 87, 86, 85, 84, 83, 82, 81, or 80% of the nucleotides of the sense strand are modified. In some embodiments, at least 99, 98, 97, 96, 95, 94, 93, 92, 91, 90, 89, 88, 87, 86, 85, 84, 83, 82, 81, or 80% of the nucleotides of the antisense strand are modified.

In certain embodiments of the disclosure, substantially all of the nucleotides of an RNAi agent of the disclosure are modified. In other embodiments of the disclosure, all of the nucleotides of an RNAi agent of the disclosure are modified. RNAi agents of the disclosure in which “substantially all of the nucleotides are modified” are largely but not wholly modified and can include not more than 5, 4, 3, 2, or 1 unmodified nucleotides. In still other embodiments of the disclosure, RNAi agents of the disclosure can include not more than 5, 4, 3, 2, or 1 modified nucleotides.

In one aspect, a dsRNA of the disclosure includes at least two nucleotide sequences, a sense sequence and an antisense sequence. The sense strand sequence for MUC5B may be selected from the group of sequences provided in any one of Tables 2-3, and the corresponding nucleotide sequence of the antisense strand of the sense strand may be selected from the group of sequences of any one of Tables 2-3. In this aspect, one of the two sequences is complementary to the other of the two sequences, with one of the sequences being substantially complementary to a sequence of an mRNA generated in the expression of a MUC5B gene. As such, in this aspect, a dsRNA will include two oligonucleotides, where one oligonucleotide is described as the sense strand (passenger strand) in any one of Tables 2-3, and the second oligonucleotide is described as the corresponding antisense strand (guide strand) of the sense strand in any one of Tables 2-3 for MUC5B.

In some embodiments, the sense strand comprises a nucleotide sequence selected from SEQ ID NOs: 355-531 and the antisense strand comprises a nucleotide sequence selected from SEQ ID NOs: 532-708. It will be understood that, although the sequences provided in Table 3 are described as modified or conjugated sequences, the RNA of the RNAi agent of the disclosure e.g., a dsRNA of the disclosure, may comprise any one of the sequences set forth in any one of Table 3 that is un-modified (for example, the sequences provided in Table 2), un-conjugated, or modified or conjugated differently than described therein. One or more lipophilic ligands can be included in any of the positions of the RNAi agents provided in the instant application.

The skilled person is well aware that dsRNAs having a duplex structure of about 20 to 23 base pairs, e.g., 21 base pairs, have been hailed as particularly effective in inducing RNA interference (Elbashir et al., (2001) EMBO J., 20:6877-6888). However, others have found that shorter or longer RNA duplex structures can also be effective (Chu and Rana (2007) RNA 14: 1714-1719; Kim et al. (2005) Nat Biotech 23:222-226). In the embodiments described above, by virtue of the nature of the oligonucleotide sequences provided herein, dsRNAs described herein can include at least one strand of a length of minimally 19 nucleotides. It can be reasonably expected that shorter duplexes minus only a few nucleotides on one or both ends can be similarly effective compared to the dsRNAs described above. Hence, dsRNAs having a sequence of at least 15, 16, 17, 18, 19, 20, or more contiguous nucleotides derived from one of the sequences provided herein are contemplated to be within the scope of the present disclosure.

In some embodiments, a double stranded ribonucleic acid (dsRNA) targeting Mucin-5B (MUC5B) is provided, wherein the dsRNA comprises a sense strand and an antisense strand forming a double stranded region, wherein the antisense strand comprises a region of complementarity to an mRNA encoding MUC5B, and wherein the antisense strand comprises a nucleotide sequence selected from any one of SEQ ID NOs: 18,793-36,683. In some embodiments, a double stranded ribonucleic acid (dsRNA) targeting Mucin-5B (MUC5B) is provided, wherein the dsRNA comprises a sense strand and an antisense strand forming a double stranded region, wherein the antisense strand comprises a region of complementarity to an mRNA encoding MUC5B, and wherein the antisense strand comprises a nucleotide sequence selected from any one of SEQ ID NOs: 18,793-36,683 and at least one modification. In some embodiments, the sense strand comprises a nucleotide sequence selected from any one of SEQ ID NOs: 902-18,792. In some embodiments, the sense and antisense strand are complementary, wherein the sense strand comprises a nucleotide sequence selected from any one of SEQ ID NOs: 902-18,792, and the antisense strand comprises a nucleotide sequence selected from any one of SEQ ID NOs: 18,793-36,683. In some embodiments, a double stranded ribonucleic acid (dsRNA) targeting Mucin-5B (MUC5B) is provided, wherein the dsRNA comprises a sense strand and an antisense strand forming a double stranded region, wherein the antisense strand comprises a region of complementarity to an mRNA encoding MUC5B, and wherein the antisense strand comprises a nucleotide sequence selected from any one of SEQ ID NOs: 54,575-72,465. In some embodiments, a double stranded ribonucleic acid (dsRNA) targeting Mucin-5B (MUC5B) is provided, wherein the dsRNA comprises a sense strand and an antisense strand forming a double stranded region, wherein the antisense strand comprises a region of complementarity to an mRNA encoding MUC5B, and wherein the antisense strand comprises a nucleotide sequence selected from any one of SEQ ID NOs: 54,575-72,465 and at least one modification. In some embodiments, the sense strand comprises a nucleotide sequence selected from any one of SEQ ID NOs: 36,684-54,574. In some embodiments, the sense and antisense strand are complementary, wherein the sense strand comprises a nucleotide sequence selected from any one of SEQ ID NOs: 36,684-54,574, and the antisense strand comprises a nucleotide sequence selected from any one of SEQ ID NOs: 54,575-72,465.

In some embodiments, a double stranded ribonucleic acid (dsRNA) targeting Mucin-5B (MUC5B) is provided, wherein the dsRNA comprises a sense strand and an antisense strand forming a double stranded region, wherein the antisense strand comprises a region of complementarity to an mRNA encoding MUC5B, and wherein the antisense strand comprises a nucleotide sequence selected from any one of SEQ ID NOs: 90,357-108,247. In some embodiments, a double stranded ribonucleic acid (dsRNA) targeting Mucin-5B (MUC5B) is provided, wherein the dsRNA comprises a sense strand and an antisense strand forming a double stranded region, wherein the antisense strand comprises a region of complementarity to an mRNA encoding MUC5B, and wherein the antisense strand comprises a nucleotide sequence selected from any one of SEQ ID NOs: 90,357-108,247 and at least one modification. In some embodiments, the sense strand comprises a nucleotide sequence selected from any one of SEQ ID NOs: 72,466-90,356. In some embodiments, the sense and antisense strand are complementary, wherein the sense strand comprises a nucleotide sequence selected from any one of SEQ ID NOs: 72,466-90,356, and the antisense strand comprises a nucleotide sequence selected from any one of SEQ ID NOs: 90,357-108,247.

In some embodiments, a double stranded ribonucleic acid (dsRNA) targeting Mucin-5B (MUC5B) is provided, wherein the dsRNA comprises a sense strand and an antisense strand forming a double stranded region, wherein the antisense strand comprises a region of complementarity to an mRNA encoding MUC5B, and wherein the antisense strand comprises a nucleotide sequence selected from SEQ ID NOs: 178-354. In some embodiments, a double stranded ribonucleic acid (dsRNA) targeting Mucin-5B (MUC5B) is provided, wherein the dsRNA comprises a sense strand and an antisense strand forming a double stranded region, wherein the antisense strand comprises a region of complementarity to an mRNA encoding MUC5B, and wherein the antisense strand comprises a nucleotide sequence selected from SEQ ID NOs: 178-354 and at least one modification.

In some embodiments, the sense strand comprises a nucleotide sequence selected from SEQ ID NOs: 1-177.

In some embodiments, the sense and antisense strands are selected from any one of the sense and antisense pairs in Table 2.

In some embodiments, the sense and antisense strands are selected from any one of the sense and antisense pairs in Table 3.

In some embodiments, a double stranded ribonucleic acid (dsRNA) targeting Mucin-5B (MUC5B) is provided, wherein the dsRNA comprises a sense strand and an antisense strand forming a double stranded region, wherein the antisense strand comprises a region of complementarity to an mRNA encoding MUC5B, and wherein
1) the sense strand comprises a nucleotide sequence comprising at least 15 contiguous nucleotides differing in sequence by no more than 3 nucleotides from SEQ ID NO: 1, and the antisense strand comprises a nucleotide sequence comprising at least 15 contiguous nucleotides differing in sequence by no more than 3 nucleotides from SEQ ID NO: 178;
2) the sense strand comprises a nucleotide sequence comprising at least 15 contiguous nucleotides differing in sequence by no more than 3 nucleotides from SEQ ID NO: 2, and the antisense strand comprises a nucleotide sequence comprising at least 15 contiguous nucleotides differing in sequence by no more than 3 nucleotides from SEQ ID NO: 179;
3) the sense strand comprises a nucleotide sequence comprising at least 15 contiguous nucleotides differing in sequence by no more than 3 nucleotides from SEQ ID NO: 3, and the antisense strand comprises a nucleotide sequence comprising at least 15 contiguous nucleotides differing in sequence by no more than 3 nucleotides from SEQ ID NO: 180;
4) the sense strand comprises a nucleotide sequence comprising at least 15 contiguous nucleotides differing in sequence by no more than 3 nucleotides from SEQ ID NO: 4, and the antisense strand comprises a nucleotide sequence comprising at least 15 contiguous nucleotides differing in sequence by no more than 3 nucleotides from SEQ ID NO: 181;
5) the sense strand comprises a nucleotide sequence comprising at least 15 contiguous nucleotides differing in sequence by no more than 3 nucleotides from SEQ ID NO: 5, and the antisense strand comprises a nucleotide sequence comprising at least 15 contiguous nucleotides differing in sequence by no more than 3 nucleotides from SEQ ID NO: 182;
6) the sense strand comprises a nucleotide sequence comprising at least 15 contiguous nucleotides differing in sequence by no more than 3 nucleotides from SEQ ID NO: 6, and the antisense strand comprises a nucleotide sequence comprising at least 15 contiguous nucleotides differing in sequence by no more than 3 nucleotides from SEQ ID NO: 183;
7) the sense strand comprises a nucleotide sequence comprising at least 15 contiguous nucleotides differing in sequence by no more than 3 nucleotides from SEQ ID NO: 7, and the antisense strand comprises a nucleotide sequence comprising at least 15 contiguous nucleotides differing in sequence by no more than 3 nucleotides from SEQ ID NO: 184;
8) the sense strand comprises a nucleotide sequence comprising at least 15 contiguous nucleotides differing in sequence by no more than 3 nucleotides from SEQ ID NO: 8, and the antisense strand comprises a nucleotide sequence comprising at least 15 contiguous nucleotides differing in sequence by no more than 3 nucleotides from SEQ ID NO: 185;
9) the sense strand comprises a nucleotide sequence comprising at least 15 contiguous nucleotides differing in sequence by no more than 3 nucleotides from SEQ ID NO: 9, and the antisense strand comprises a nucleotide sequence comprising at least 15 contiguous nucleotides differing in sequence by no more than 3 nucleotides from SEQ ID NO: 186; or
10) the sense strand comprises a nucleotide sequence comprising at least 15 contiguous nucleotides differing in sequence by no more than 3 nucleotides from SEQ ID NO: 10, and the antisense strand comprises a nucleotide sequence comprising at least 15 contiguous nucleotides differing in sequence by no more than 3 nucleotides from SEQ ID NO: 187.

In some embodiments, a double stranded ribonucleic acid (dsRNA) targeting Mucin-5B (MUC5B) is provided, wherein the dsRNA comprises a sense strand and an antisense strand forming a double stranded region, wherein the antisense strand comprises a region of complementarity to an mRNA encoding MUC5B, and wherein
1) the sense strand comprises a nucleotide sequence comprising at least 15 contiguous nucleotides differing in sequence by no more than 3 nucleotides from SEQ ID NO: 1 and at least one modification, and the antisense strand comprises a nucleotide sequence comprising at least 15 contiguous nucleotides differing in sequence by no more than 3 nucleotides from SEQ ID NO: 178;
2) the sense strand comprises a nucleotide sequence comprising at least 15 contiguous nucleotides differing in sequence by no more than 3 nucleotides from SEQ ID NO: 2 and at least one modification, and the antisense strand comprises a nucleotide sequence comprising at least 15 contiguous nucleotides differing in sequence by no more than 3 nucleotides from SEQ ID NO: 179;
3) the sense strand comprises a nucleotide sequence comprising at least 15 contiguous nucleotides differing in sequence by no more than 3 nucleotides from SEQ ID NO: 3 and at least one modification, and the antisense strand comprises a nucleotide sequence comprising at least 15 contiguous nucleotides differing in sequence by no more than 3 nucleotides from SEQ ID NO: 180;
4) the sense strand comprises a nucleotide sequence comprising at least 15 contiguous nucleotides differing in sequence by no more than 3 nucleotides from SEQ ID NO: 4 and at least one modification, and the antisense strand comprises a nucleotide sequence comprising at least 15 contiguous nucleotides differing in sequence by no more than 3 nucleotides from SEQ ID NO: 181;
5) the sense strand comprises a nucleotide sequence comprising at least 15 contiguous nucleotides differing in sequence by no more than 3 nucleotides from SEQ ID NO: 5 and at least one modification, and the antisense strand comprises a nucleotide sequence comprising at least 15 contiguous nucleotides differing in sequence by no more than 3 nucleotides from SEQ ID NO: 182,
6) the sense strand comprises a nucleotide sequence comprising at least 15 contiguous nucleotides differing in sequence by no more than 3 nucleotides from SEQ ID NO: 6 and at least one modification, and the antisense strand comprises a nucleotide sequence comprising at least 15 contiguous nucleotides differing in sequence by no more than 3 nucleotides from SEQ ID NO: 183;
7) the sense strand comprises a nucleotide sequence comprising at least 15 contiguous nucleotides differing in sequence by no more than 3 nucleotides from SEQ ID NO: 7 and at least one modification, and the antisense strand comprises a nucleotide sequence comprising at least 15 contiguous nucleotides differing in sequence by no more than 3 nucleotides from SEQ ID NO: 184;
8) the sense strand comprises a nucleotide sequence comprising at least 15 contiguous nucleotides differing in sequence by no more than 3 nucleotides from SEQ ID NO: 8 and at least one modification, and the antisense strand comprises a nucleotide sequence comprising at least 15 contiguous nucleotides differing in sequence by no more than 3 nucleotides from SEQ ID NO: 185;
9) the sense strand comprises a nucleotide sequence comprising at least 15 contiguous nucleotides differing in sequence by no more than 3 nucleotides from SEQ ID NO: 9 and at least one modification, and the antisense strand comprises a nucleotide sequence comprising at least 15 contiguous nucleotides differing in sequence by no more than 3 nucleotides from SEQ ID NO: 186; or
10) the sense strand comprises a nucleotide sequence comprising at least 15 contiguous nucleotides differing in sequence by no more than 3 nucleotides from SEQ ID NO. 10 and at least one modification, and the antisense strand comprises a nucleotide sequence comprising at least 15 contiguous nucleotides differing in sequence by no more than 3 nucleotides from SEQ ID NO: 187.

In some embodiments, the antisense strand differs in sequence from an antisense strand sequence recited in the immediately preceding numbered paragraphs (1)-(10) by no more than 2 nucleotides. In some embodiments, the antisense strand differs in sequence by no more than 1 nucleotide. In some embodiments, the sense strand differs in sequence from a sense strand sequence recited in the immediately preceding numbered paragraphs (1)-(10) by no more than 2 nucleotides. In some embodiments, the antisense strand comprises at least 16, at least 17, at least 18, at least 19, at least 20, at least 21, at least 22, or 23 contiguous nucleotides of an antisense strand sequence recited in the immediately preceding numbered paragraphs (1)-(10). In some embodiments, the sense strand comprises at least 16, at least 17, at least 18, at least 19, at least 20, at least 21, at least 22, or 23 contiguous nucleotides of a sense strand sequence recited in the immediately preceding numbered paragraphs (1)-(10). In some embodiments, the antisense strand comprises 23 contiguous nucleotides and the sense strand comprises 21 contiguous nucleotides of an antisense and sense strand sequence recited in the immediately preceding paragraphs (1)-(10), respectively.

In some embodiments, a double stranded ribonucleic acid (dsRNA) targeting Mucin-5B (MUC5B) is provided, wherein the dsRNA comprises a sense strand and an antisense strand forming a double stranded region, wherein the antisense strand comprises a region of complementarity to an mRNA encoding MUC5B, and wherein
1) the sense strand comprises a nucleotide sequence comprising at least 15 contiguous nucleotides differing in sequence by no more than 3 nucleotides from SEQ ID NO: 362, and the antisense strand comprises a nucleotide sequence comprising at least 15 contiguous nucleotides differing in sequence by no more than 3 nucleotides from SEQ ID NO: 539;
2) the sense strand comprises a nucleotide sequence comprising at least 15 contiguous nucleotides differing in sequence by no more than 3 nucleotides from SEQ ID NO: 363, and the antisense strand comprises a nucleotide sequence comprising at least 15 contiguous nucleotides differing in sequence by no more than 3 nucleotides from SEQ ID NO: 540;
3) the sense strand comprises a nucleotide sequence comprising at least 15 contiguous nucleotides differing in sequence by no more than 3 nucleotides from SEQ ID NO: 356, and the antisense strand comprises a nucleotide sequence comprising at least 15 contiguous nucleotides differing in sequence by no more than 3 nucleotides from SEQ ID NO: 533;
4) the sense strand comprises a nucleotide sequence comprising at least 15 contiguous nucleotides differing in sequence by no more than 3 nucleotides from SEQ ID NO: 357, and the antisense strand comprises a nucleotide sequence comprising at least 15 contiguous nucleotides differing in sequence by no more than 3 nucleotides from SEQ ID NO: 534;
5) the sense strand comprises a nucleotide sequence comprising at least 15 contiguous nucleotides differing in sequence by no more than 3 nucleotides from SEQ ID NO: 359, and the antisense strand comprises a nucleotide sequence comprising at least 15 contiguous nucleotides differing in sequence by no more than 3 nucleotides from SEQ ID NO: 536;
6) the sense strand comprises a nucleotide sequence comprising at least 15 contiguous nucleotides differing in sequence by no more than 3 nucleotides from SEQ ID NO: 355, and the antisense strand comprises a nucleotide sequence comprising at least 15 contiguous nucleotides differing in sequence by no more than 3 nucleotides from SEQ ID NO: 532;
7) the sense strand comprises a nucleotide sequence comprising at least 15 contiguous nucleotides differing in sequence by no more than 3 nucleotides from SEQ ID NO: 361, and the antisense strand comprises a nucleotide sequence comprising at least 15 contiguous nucleotides differing in sequence by no more than 3 nucleotides from SEQ ID NO: 538;
8) the sense strand comprises a nucleotide sequence comprising at least 15 contiguous nucleotides differing in sequence by no more than 3 nucleotides from SEQ ID NO: 364, and the antisense strand comprises a nucleotide sequence comprising at least 15 contiguous nucleotides differing in sequence by no more than 3 nucleotides from SEQ ID NO: 541;
9) the sense strand comprises a nucleotide sequence comprising at least 15 contiguous nucleotides differing in sequence by no more than 3 nucleotides from SEQ ID NO: 358, and the antisense strand comprises a nucleotide sequence comprising at least 15 contiguous nucleotides differing in sequence by no more than 3 nucleotides from SEQ ID NO: 535; or
10) the sense strand comprises a nucleotide sequence comprising at least 15 contiguous nucleotides differing in sequence by no more than 3 nucleotides from SEQ ID NO: 360, and the antisense strand comprises a nucleotide sequence comprising at least 15 contiguous nucleotides differing in sequence by no more than 3 nucleotides from SEQ ID NO: 537.

In some embodiments, the antisense strand differs in sequence from an antisense strand sequence recited in the immediately preceding numbered paragraphs (1)-(10) by no more than 2 nucleotides. In some embodiments, the antisense strand differs in sequence by no more than 1 nucleotide. In some embodiments, the sense strand differs in sequence from a sense strand sequence recited in the immediately preceding numbered paragraphs (1)-(10) by no more than 2 nucleotides. In some embodiments, the antisense strand comprises at least 16, at least 17, at least 18, at least 19, at least 20, at least 21, at least 22, or 23 contiguous nucleotides of an antisense strand sequence recited in the immediately preceding numbered paragraphs (1)-(10). In some embodiments, the sense strand comprises at least 16, at least 17, at least 18, at least 19, at least 20, at least 21, at least 22, or 23 contiguous nucleotides of a sense strand sequence recited in the immediately preceding numbered paragraphs (1)-(10). In some embodiments, the antisense strand comprises 23 contiguous nucleotides and the sense strand comprises 21 contiguous nucleotides of an antisense and sense strand sequence recited in the immediately preceding paragraphs (1)-(10), respectively.

In some embodiments, the sense and antisense strands are selected from any one of the sense and antisense pairs in Table 9. In some embodiments, in the antisense strand comprises a nucleotide sequence selected from SEQ ID NOs: 535, 538, 540, 541, 533, 536, or 186,538-186,624. In some embodiments, the sense strand comprises a nucleotide sequence selected from SEQ ID NOs: 356, 358, 359, 361, 363, 364, or 186,502-186,537.

In some embodiments, a double stranded ribonucleic acid (dsRNA) targeting Mucin-5B (MUC5B) is provided, wherein the dsRNA comprises a sense strand and an antisense strand forming a double stranded region, wherein the antisense strand comprises a region of complementarity to an mRNA encoding MUC5B, and wherein;
1) the sense strand comprises the nucleotide sequence of SEQ ID NO: 358, and the antisense strand comprises the nucleotide sequence of SEQ ID NO: 186,538;
2) the sense strand comprises the nucleotide sequence of SEQ ID NO: 358, and the antisense strand comprises the nucleotide sequence of SEQ ID NO: 186,539;
3) the sense strand comprises the nucleotide sequence of SEQ ID NO: 358, and the antisense strand comprises the nucleotide sequence of SEQ ID NO: 186,540;
4) the sense strand comprises the nucleotide sequence of SEQ ID NO: 186,502, and the antisense strand comprises the nucleotide sequence of SEQ ID NO: 186,541;
5) the sense strand comprises the nucleotide sequence of SEQ ID NO: 186,503, and the antisense strand comprises the nucleotide sequence of SEQ ID NO: 535;
6) the sense strand comprises the nucleotide sequence of SEQ ID NO: 186,504, and the antisense strand comprises the nucleotide sequence of SEQ ID NO: 535;
7) the sense strand comprises the nucleotide sequence of SEQ ID NO: 186,505, and the antisense strand comprises the nucleotide sequence of SEQ ID NO: 535;
8) the sense strand comprises the nucleotide sequence of SEQ ID NO: 186,506, and the antisense strand comprises the nucleotide sequence of SEQ ID NO: 186,542;
9) the sense strand comprises the nucleotide sequence of SEQ ID NO: 186,507, and the antisense strand comprises the nucleotide sequence of SEQ ID NO: 186,543;
10) the sense strand comprises the nucleotide sequence of SEQ ID NO: 358, and the antisense strand comprises the nucleotide sequence of SEQ ID NO: 186,544;
11) the sense strand comprises the nucleotide sequence of SEQ ID NO: 358, and the antisense strand comprises the nucleotide sequence of SEQ ID NO: 186,545;
12) the sense strand comprises the nucleotide sequence of SEQ ID NO: 358, and the antisense strand comprises the nucleotide sequence of SEQ ID NO: 186,546;
13) the sense strand comprises the nucleotide sequence of SEQ ID NO: 358, and the antisense strand comprises the nucleotide sequence of SEQ ID NO: 186,547;
14) the sense strand comprises the nucleotide sequence of SEQ ID NO: 186,507, and the antisense strand comprises the nucleotide sequence of SEQ ID NO: 186,548;
15) the sense strand comprises the nucleotide sequence of SEQ ID NO: 186,507, and the antisense strand comprises the nucleotide sequence of SEQ ID NO: 186,549;
16) the sense strand comprises the nucleotide sequence of SEQ ID NO: 186,508, and the antisense strand comprises the nucleotide sequence of SEQ ID NO: 186,550;
17) the sense strand comprises the nucleotide sequence of SEQ ID NO: 186,508, and the antisense strand comprises the nucleotide sequence of SEQ ID NO: 186,551;
18) the sense strand comprises the nucleotide sequence of SEQ ID NO: 358, and the antisense strand comprises the nucleotide sequence of SEQ ID NO: 186,552;
19) the sense strand comprises the nucleotide sequence of SEQ ID NO: 358, and the antisense strand comprises the nucleotide sequence of SEQ ID NO: 186,553;
20) the sense strand comprises the nucleotide sequence of SEQ ID NO: 358, and the antisense strand comprises the nucleotide sequence of SEQ ID NO: 186,554; or
21) the sense strand comprises the nucleotide sequence of SEQ ID NO: 358, and the antisense strand comprises the nucleotide sequence of SEQ ID NO: 186,555.

In some embodiments, a double stranded ribonucleic acid (dsRNA) targeting Mucin-5B (MUC5B) is provided, wherein the dsRNA comprises a sense strand and an antisense strand forming a double stranded region, wherein the antisense strand comprises a region of complementarity to an mRNA encoding MUC5B, and wherein;
1) the sense strand comprises the nucleotide sequence of SEQ ID NO: 361, and the antisense strand comprises the nucleotide sequence of SEQ ID NO: 186,556;
2) the sense strand comprises the nucleotide sequence of SEQ ID NO: 361, and the antisense strand comprises the nucleotide sequence of SEQ ID NO: 186,557;
3) the sense strand comprises the nucleotide sequence of SEQ ID NO: 361, and the antisense strand comprises the nucleotide sequence of SEQ ID NO: 186,558;
4) the sense strand comprises the nucleotide sequence of SEQ ID NO: 186,509, and the antisense strand comprises the nucleotide sequence of SEQ ID NO: 186,559;
5) the sense strand comprises the nucleotide sequence of SEQ ID NO: 186,510, and the antisense strand comprises the nucleotide sequence of SEQ ID NO: 538;
6) the sense strand comprises the nucleotide sequence of SEQ ID NO: 186,511, and the antisense strand comprises the nucleotide sequence of SEQ ID NO: 538;
7) the sense strand comprises the nucleotide sequence of SEQ ID NO: 186,512, and the antisense strand comprises the nucleotide sequence of SEQ ID NO: 538;
8) the sense strand comprises the nucleotide sequence of SEQ ID NO: 186,513, and the antisense strand comprises the nucleotide sequence of SEQ ID NO: 186,560;
9) the sense strand comprises the nucleotide sequence of SEQ ID NO: 186,514, and the antisense strand comprises the nucleotide sequence of SEQ ID NO: 186,561;
10) the sense strand comprises the nucleotide sequence of SEQ ID NO: 361, and the antisense strand comprises the nucleotide sequence of SEQ ID NO: 186,562;
11) the sense strand comprises the nucleotide sequence of SEQ ID NO: 361, and the antisense strand comprises the nucleotide sequence of SEQ ID NO: 186,563;
12) the sense strand comprises the nucleotide sequence of SEQ ID NO: 361, and the antisense strand comprises the nucleotide sequence of SEQ ID NO: 186,564;
13) the sense strand comprises the nucleotide sequence of SEQ ID NO: 361, and the antisense strand comprises the nucleotide sequence of SEQ ID NO: 186,565;
14) the sense strand comprises the nucleotide sequence of SEQ ID NO: 186,514, and the antisense strand comprises the nucleotide sequence of SEQ ID NO: 186,566;
15) the sense strand comprises the nucleotide sequence of SEQ ID NO: 186,514, and the antisense strand comprises the nucleotide sequence of SEQ ID NO: 186,567;
16) the sense strand comprises the nucleotide sequence of SEQ ID NO: 186,515, and the antisense strand comprises the nucleotide sequence of SEQ ID NO: 186,568;
17) the sense strand comprises the nucleotide sequence of SEQ ID NO: 186,515, and the antisense strand comprises the nucleotide sequence of SEQ ID NO: 186,569;
18) the sense strand comprises the nucleotide sequence of SEQ ID NO: 361, and the antisense strand comprises the nucleotide sequence of SEQ ID NO: 186,570;
19) the sense strand comprises the nucleotide sequence of SEQ ID NO: 361, and the antisense strand comprises the nucleotide sequence of SEQ ID NO: 186,571;
20) the sense strand comprises the nucleotide sequence of SEQ ID NO: 361, and the antisense strand comprises the nucleotide sequence of SEQ ID NO: 186,572; or
21) the sense strand comprises the nucleotide sequence of SEQ ID NO: 361, and the antisense strand comprises the nucleotide sequence of SEQ ID NO: 186,573.

In some embodiments, a double stranded ribonucleic acid (dsRNA) targeting Mucin-5B (MUC5B) is provided, wherein the dsRNA comprises a sense strand and an antisense strand forming a double stranded region, wherein the antisense strand comprises a region of complementarity to an mRNA encoding MUC5B, and wherein;
1) the sense strand comprises the nucleotide sequence of SEQ ID NO: 363, and the antisense strand comprises the nucleotide sequence of SEQ ID NO: 186,574;
2) the sense strand comprises the nucleotide sequence of SEQ ID NO: 363, and the antisense strand comprises the nucleotide sequence of SEQ ID NO: 186,575;
3) the sense strand comprises the nucleotide sequence of SEQ ID NO: 363, and the antisense strand comprises the nucleotide sequence of SEQ ID NO: 186,576;
4) the sense strand comprises the nucleotide sequence of SEQ ID NO: 186,516, and the antisense strand comprises the nucleotide sequence of SEQ ID NO: 540;
5) the sense strand comprises the nucleotide sequence of SEQ ID NO: 186,517, and the antisense strand comprises the nucleotide sequence of SEQ ID NO: 540;
6) the sense strand comprises the nucleotide sequence of SEQ ID NO: 186,518, and the antisense strand comprises the nucleotide sequence of SEQ ID NO: 540;
7) the sense strand comprises the nucleotide sequence of SEQ ID NO: 186,519, and the antisense strand comprises the nucleotide sequence of SEQ ID NO: 540;
8) the sense strand comprises the nucleotide sequence of SEQ ID NO: 186,520, and the antisense strand comprises the nucleotide sequence of SEQ ID NO: 186,577;
9) the sense strand comprises the nucleotide sequence of SEQ ID NO: 363, and the antisense strand comprises the nucleotide sequence of SEQ ID NO: 186,578;
10) the sense strand comprises the nucleotide sequence of SEQ ID NO: 363, and the antisense strand comprises the nucleotide sequence of SEQ ID NO: 186,579;
11) the sense strand comprises the nucleotide sequence of SEQ ID NO: 363, and the antisense strand comprises the nucleotide sequence of SEQ ID NO: 186,580;
12) the sense strand comprises the nucleotide sequence of SEQ ID NO: 363, and the antisense strand comprises the nucleotide sequence of SEQ ID NO: 186,581;
13) the sense strand comprises the nucleotide sequence of SEQ ID NO: 363, and the antisense strand comprises the nucleotide sequence of SEQ ID NO: 186,582 (usAfscga(C3)auccaugGfaCfuuguasgsu);
14) the sense strand comprises the nucleotide sequence of SEQ ID NO: 363, and the antisense strand comprises the nucleotide sequence of SEQ ID NO: 186,583; or
15) the sense strand comprises the nucleotide sequence of SEQ ID NO: 363, and the antisense strand comprises the nucleotide sequence of SEQ ID NO: 186,584.

In some embodiments, a double stranded ribonucleic acid (dsRNA) targeting Mucin-5B (MUC5B) is provided, wherein the dsRNA comprises a sense strand and an antisense strand forming a double stranded region, wherein the antisense strand comprises a region of complementarity to an mRNA encoding MUC5B, and wherein;
1) the sense strand comprises the nucleotide sequence of SEQ ID NO: 364, and the antisense strand comprises the nucleotide sequence of SEQ ID NO: 186,585;
2) the sense strand comprises the nucleotide sequence of SEQ ID NO: 364, and the antisense strand comprises the nucleotide sequence of SEQ ID NO: 186,586;
3) the sense strand comprises the nucleotide sequence of SEQ ID NO: 364, and the antisense strand comprises the nucleotide sequence of SEQ ID NO: 186,587;
4) the sense strand comprises the nucleotide sequence of SEQ ID NO: 186,521, and the antisense strand comprises the nucleotide sequence of SEQ ID NO: 186,588;
5) the sense strand comprises the nucleotide sequence of SEQ ID NO: 186,522, and the antisense strand comprises the nucleotide sequence of SEQ ID NO: 541;
6) the sense strand comprises the nucleotide sequence of SEQ ID NO: 186,523, and the antisense strand comprises the nucleotide sequence of SEQ ID NO: 541;
7) the sense strand comprises the nucleotide sequence of SEQ ID NO: 186,524, and the antisense strand comprises the nucleotide sequence of SEQ ID NO: 541;
8) the sense strand comprises the nucleotide sequence of SEQ ID NO: 186,525, and the antisense strand comprises the nucleotide sequence of SEQ ID NO: 186,589;
9) the sense strand comprises the nucleotide sequence of SEQ ID NO: 186,526, and the antisense strand comprises the nucleotide sequence of SEQ ID NO: 186,590;
10) the sense strand comprises the nucleotide sequence of SEQ ID NO: 364, and the antisense strand comprises the nucleotide sequence of SEQ ID NO: 186,591;
11) the sense strand comprises the nucleotide sequence of SEQ ID NO: 364, and the antisense strand comprises the nucleotide sequence of SEQ ID NO: 186,592;
12) the sense strand comprises the nucleotide sequence of SEQ ID NO: 364, and the antisense strand comprises the nucleotide sequence of SEQ ID NO: 186,593;
13) the sense strand comprises the nucleotide sequence of SEQ ID NO: 364, and the antisense strand comprises the nucleotide sequence of SEQ ID NO: 186,594;
14) the sense strand comprises the nucleotide sequence of SEQ ID NO: 186,526, and the antisense strand comprises the nucleotide sequence of SEQ ID NO: 186,595;
15) the sense strand comprises the nucleotide sequence of SEQ ID NO: 186,526, and the antisense strand comprises the nucleotide sequence of SEQ ID NO: 186,596;
16) the sense strand comprises the nucleotide sequence of SEQ ID NO: 186,527, and the antisense strand comprises the nucleotide sequence of SEQ ID NO: 186,597;
17) the sense strand comprises the nucleotide sequence of SEQ ID NO: 186,527, and the antisense strand comprises the nucleotide sequence of SEQ ID NO: 186,598;
18) the sense strand comprises the nucleotide sequence of SEQ ID NO: 364, and the antisense strand comprises the nucleotide sequence of SEQ ID NO: 186,599;
19) the sense strand comprises the nucleotide sequence of SEQ ID NO: 364, and the antisense strand comprises the nucleotide sequence of SEQ ID NO: 186,600;
20) the sense strand comprises the nucleotide sequence of SEQ ID NO: 364, and the antisense strand comprises the nucleotide sequence of SEQ ID NO: 186,601; or
21) the sense strand comprises the nucleotide sequence of SEQ ID NO: 364, and the antisense strand comprises the nucleotide sequence of SEQ ID NO: 186,602.

In some embodiments, a double stranded ribonucleic acid (dsRNA) targeting Mucin-5B (MUC5B) is provided, wherein the dsRNA comprises a sense strand and an antisense strand forming a double stranded region, wherein the antisense strand comprises a region of complementarity to an mRNA encoding MUC5B, and wherein;
1) the sense strand comprises the nucleotide sequence of SEQ ID NO: 356, and the antisense strand comprises the nucleotide sequence of SEQ ID NO: 186,603;
2) the sense strand comprises the nucleotide sequence of SEQ ID NO: 356, and the antisense strand comprises the nucleotide sequence of SEQ ID NO: 186,604;
3) the sense strand comprises the nucleotide sequence of SEQ ID NO: 356, and the antisense strand comprises the nucleotide sequence of SEQ ID NO: 186,605;
4) the sense strand comprises the nucleotide sequence of SEQ ID NO: 186,528, and the antisense strand comprises the nucleotide sequence of SEQ ID NO: 533;
5) the sense strand comprises the nucleotide sequence of SEQ ID NO: 186,529, and the antisense strand comprises the nucleotide sequence of SEQ ID NO: 533;
6) the sense strand comprises the nucleotide sequence of SEQ ID NO: 186,530, and the antisense strand comprises the nucleotide sequence of SEQ ID NO: 533;
7) the sense strand comprises the nucleotide sequence of SEQ ID NO: 186,531, and the antisense strand comprises the nucleotide sequence of SEQ ID NO: 533;
8) the sense strand comprises the nucleotide sequence of SEQ ID NO: 186,532, and the antisense strand comprises the nucleotide sequence of SEQ ID NO: 186,606;
9) the sense strand comprises the nucleotide sequence of SEQ ID NO: 356, and the antisense strand comprises the nucleotide sequence of SEQ ID NO: 186,607;
10) the sense strand comprises the nucleotide sequence of SEQ ID NO: 356, and the antisense strand comprises the nucleotide sequence of SEQ ID NO: 186,608;
11) the sense strand comprises the nucleotide sequence of SEQ ID NO: 356, and the antisense strand comprises the nucleotide sequence of SEQ ID NO: 186,609;
12) the sense strand comprises the nucleotide sequence of SEQ ID NO: 356, and the antisense strand comprises the nucleotide sequence of SEQ ID NO: 186,610;
13) the sense strand comprises the nucleotide sequence of SEQ ID NO: 356, and the antisense strand comprises the nucleotide sequence of SEQ ID NO: 186,611 (usAfsccg(C3)aauuuggUfcAfaacagsgsa);
14) the sense strand comprises the nucleotide sequence of SEQ ID NO: 356, and the antisense strand comprises the nucleotide sequence of SEQ ID NO: 186,612; or
15) the sense strand comprises the nucleotide sequence of SEQ ID NO: 356, and the antisense strand comprises the nucleotide sequence of SEQ ID NO: 186,613.

In some embodiments, a double stranded ribonucleic acid (dsRNA) targeting Mucin-5B (MUC5B) is provided, wherein the dsRNA comprises a sense strand and an antisense strand forming a double stranded region, wherein the antisense strand comprises a region of complementarity to an mRNA encoding MUC5B, and wherein;
1) the sense strand comprises the nucleotide sequence of SEQ ID NO: 359, and the antisense strand comprises the nucleotide sequence of SEQ ID NO: 186,614;
2) the sense strand comprises the nucleotide sequence of SEQ ID NO: 359, and the antisense strand comprises the nucleotide sequence of SEQ ID NO: 186,615;
3) the sense strand comprises the nucleotide sequence of SEQ ID NO: 359, and the antisense strand comprises the nucleotide sequence of SEQ ID NO: 186,616;
4) the sense strand comprises the nucleotide sequence of SEQ ID NO: 186,533, and the antisense strand comprises the nucleotide sequence of SEQ ID NO: 536;
5) the sense strand comprises the nucleotide sequence of SEQ ID NO: 186,534, and the antisense strand comprises the nucleotide sequence of SEQ ID NO: 536;
6) the sense strand comprises the nucleotide sequence of SEQ ID NO: 186,535, and the antisense strand comprises the nucleotide sequence of SEQ ID NO: 536;
7) the sense strand comprises the nucleotide sequence of SEQ ID NO: 186,536, and the antisense strand comprises the nucleotide sequence of SEQ ID NO: 536;
8) the sense strand comprises the nucleotide sequence of SEQ ID NO: 186,537, and the antisense strand comprises the nucleotide sequence of SEQ ID NO: 186,617;
9) the sense strand comprises the nucleotide sequence of SEQ ID NO: 359, and the antisense strand comprises the nucleotide sequence of SEQ ID NO: 186,618;
10) the sense strand comprises the nucleotide sequence of SEQ ID NO: 359, and the antisense strand comprises the nucleotide sequence of SEQ ID NO: 186,619;
11) the sense strand comprises the nucleotide sequence of SEQ ID NO: 359, and the antisense strand comprises the nucleotide sequence of SEQ ID NO: 186,620;
12) the sense strand comprises the nucleotide sequence of SEQ ID NO: 359, and the antisense strand comprises the nucleotide sequence of SEQ ID NO: 186,621;
13) the sense strand comprises the nucleotide sequence of SEQ ID NO: 359, and the antisense strand comprises the nucleotide sequence of SEQ ID NO: 186,622 (usAfsgug(C3)cggguggAfaCfaaagcsusc);
14) the sense strand comprises the nucleotide sequence of SEQ ID NO: 359, and the antisense strand comprises the nucleotide sequence of SEQ ID NO: 186,623; or
15) the sense strand comprises the nucleotide sequence of SEQ ID NO: 359, and the antisense strand comprises the nucleotide sequence of SEQ ID NO: 186,624.

In some embodiments, a double stranded ribonucleic acid (dsRNA) targeting Mucin-5B (MUC5B) is provided, wherein the dsRNA comprises a sense strand and an antisense strand forming a double stranded region, wherein the antisense strand comprises a region of complementarity to an mRNA encoding MUC5B, and wherein
1) the sense strand comprises the nucleotide sequence of SEQ ID NO: 186,507, and the antisense strand comprises the nucleotide sequence of SEQ ID NO: 186,543;
2) the sense strand comprises the nucleotide sequence of SEQ ID NO: 186,512, and the antisense strand comprises the nucleotide sequence of SEQ ID NO: 538;
3) the sense strand comprises the nucleotide sequence of SEQ ID NO: 186,514, and the antisense strand comprises the nucleotide sequence of SEQ ID NO: 186,561;
4) the sense strand comprises the nucleotide sequence of SEQ ID NO: 361, and the antisense strand comprises the nucleotide sequence of SEQ ID NO: 186,562;
5) the sense strand comprises the nucleotide sequence of SEQ ID NO: 361, and the antisense strand comprises the nucleotide sequence of SEQ ID NO: 186,563;
6) the sense strand comprises the nucleotide sequence of SEQ ID NO: 364, and the antisense strand comprises the nucleotide sequence of SEQ ID NO: 186,593;
7) the sense strand comprises the nucleotide sequence of SEQ ID NO: 364, and the antisense strand comprises the nucleotide sequence of SEQ ID NO: 186,600;
8) the sense strand comprises the nucleotide sequence of SEQ ID NO: 356, and the antisense strand comprises the nucleotide sequence of SEQ ID NO: 186,603;
9) the sense strand comprises the nucleotide sequence of SEQ ID NO: 356, and the antisense strand comprises the nucleotide sequence of SEQ ID NO: 186,605;
10) the sense strand comprises the nucleotide sequence of SEQ ID NO: 186,532, and the antisense strand comprises the nucleotide sequence of SEQ ID NO: 186,606;
11) the sense strand comprises the nucleotide sequence of SEQ ID NO: 356, and the antisense strand comprises the nucleotide sequence of SEQ ID NO: 186,607;
12) the sense strand comprises the nucleotide sequence of SEQ ID NO: 359, and the antisense strand comprises the nucleotide sequence of SEQ ID NO: 186,614;
13) the sense strand comprises the nucleotide sequence of SEQ ID NO: 186,536, and the antisense strand comprises the nucleotide sequence of SEQ ID NO: 536; or
14) the sense strand comprises the nucleotide sequence of SEQ ID NO: 186,537, and the antisense strand comprises the nucleotide sequence of SEQ ID NO: 186,617.

### III. Antisense Oligonucleotides (ASOs) of the Disclosure

In some embodiments, the present disclosure provides a synthetic antisense oligonucleotide that inhibits expression of MUC5B by targeting MUC5B RNA. In some embodiments, the ASO inhibits expression of a mucin that is secreted, which, in one embodiment, comprises MUC5B. In some embodiments, the ASO inhibits expression of a cell surface-associated mucin that comprises MUC5B.

In some embodiments, the present disclosure provides an oligonucleotide having 8 to 30 linked nucleosides having a nucleobase sequence comprising a complementary region, wherein the complementary region comprises at least 8 contiguous nucleobases complementary to an equal-length portion of a target region of a MUC5B mRNA or a MUC5B transcript. In other embodiments, the antisense oligonucleotide has a nucleobase sequence comprising at least 12, at least 13, at least 14, at least 15, at least 16, at least 17, at least 18, or at least 19 contiguous nucleobases. In some embodiments, the synthetic antisense oligonucleotide comprises 14-25 or 17-22 nucleotides.

In some embodiments, targeting includes the determination of at least one target segment to which an oligonucleotide hybridizes such that a desired effect occurs. A target region may contain one or more target segments. Multiple target segments within a target region may be overlapping. Alternatively, they may be non-overlapping. In some embodiments, target segments within a target region are separated by no more than about 300 nucleotides. In some embodiments, target segments within a target region are separated by a number of nucleotides that is no more than 250, 200, 150, 100, 90, 80, 70, 60, 50, 40, 30, 20, or 10 nucleotides on the target nucleic acid or is a range defined by any two of the preceding values. In some embodiments, target segments within a target region are separated by no more than five nucleotides on the target nucleic acid. In some embodiments, target segments are contiguous.

In some embodiments, a target region is a structurally defined region of the target nucleic acid. In some embodiments, a suitable target segment may be found within a 5′ UTR, a coding region, a 3′ UTR, an intron, an exon, or an exon/intron junction. The target segment is in an exon/intron junction in one embodiment. In some embodiments, the target segment is within an exon of MUC5B. In some embodiments, the target segment is an exonic splicing silencer. In other embodiments, the target segment is an exonic splicing enhancer. In other embodiments, the target region comprises a translation initiation region, translation termination region, or other defined nucleic acid region. The structurally defined regions for MUC5B can be obtained by accession number from sequence databases such as NCBI, and such information is incorporated herein by reference.

In some embodiments, the antisense oligonucleotide is complementary to and/or targeted to MUC5B RNA (for example, GenBank Accession No. NM_002458.3). In some embodiments, the antisense oligonucleotide is complementary to and/or targeted to a portion of MUC5B mRNA. In other embodiments, the antisense oligonucleotide is complementary to and/or targeted to MUC5B pre-mRNA.

In some embodiments, the antisense oligonucleotide as described herein comprises a nucleotide sequence having at least 80%, 85%, 90%, 91%, 92%, 93%, 94%, 95%, 96%, 97%, 98%, or 99% nucleotide sequence identity to any one of SEQ ID NOs: 709-802 of Table 4. In some embodiments, the antisense oligonucleotide comprises any one of SEQ ID NOs: 709-802.

In one embodiment, the antisense oligonucleotide as described herein, for example, in Table 4, is unmodified and does not comprise, e.g., chemical modifications or conjugations known in the art and described herein. In some embodiments, the antisense oligonucleotide, as described herein, is chemically modified to enhance stability or other beneficial characteristics. In some embodiments, the antisense oligonucleotide, as described herein, comprises a nucleotide sequence selected from any one of SEQ ID NOs: 803-896 of Table 5. It will be understood that, although the sequences provided in Table 5 are described as modified or conjugated sequences, the antisense oligonucleotide of the disclosure may comprise any one of the sequences set forth in any one of Table 5 that is unmodified (for example, the sequences provided in Table 4), unconjugated, or modified or conjugated differently than described therein. One or more lipophilic ligands can be included in any of the positions of the antisense oligonucleotide provided in the instant application.

In some embodiments, at least 1, 2, 3, 4, 5, 6, 7, 8, 9, 10, 11, 12, 13, 14, 15, 16, or 17 nucleotides of the antisense oligonucleotide are modified. In some embodiments, at least 99, 98, 97, 96, 95, 94, 93, 92, 91, 90, 89, 88, 87, 86, 85, 84, 83, 82, 81, or 80% of the nucleotides of antisense oligonucleotide are modified.

In some embodiments, the present disclosure provides a synthetic antisense oligonucleotide that inhibits expression of MUC5B by targeting MUC5B RNA, wherein the antisense oligonucleotide comprises a nucleotide sequence selected from any one of SEQ ID NO: 147,339-186,429. In some embodiments, the present disclosure provides a synthetic antisense oligonucleotide that inhibits expression of MUC5B by targeting MUC5B RNA, wherein the sense sequence corresponding to the antisense oligonucleotide sequence comprises a nucleotide sequence selected from any one of SEQ ID NO: 108,248-147,338.

In some embodiments, the present disclosure provides a synthetic antisense oligonucleotide that inhibits expression of MUC5B by targeting MUC5B RNA, wherein the antisense oligonucleotide comprises at least 15, 16, or 17 contiguous nucleotides differing by no more than 3, 2, or 1 nucleotide from a nucleotide sequence selected from any one of SEQ ID NO: 147,339-186,429. In some embodiments, the present disclosure provides a synthetic antisense oligonucleotide that inhibits expression of MUC5B by targeting MUC5B RNA, wherein the sense sequence corresponding to the sequence of the antisense oligonucleotide comprises at least 15, 16, or 17 contiguous nucleotides differing by no more than 3, 2, or 1 nucleotide from a nucleotide sequence selected from any one of SEQ ID NO: 108,248-147,338.

In certain embodiments of the disclosure, substantially all of the nucleotides of the antisense oligonucleotide of the disclosure are modified. In other embodiments of the disclosure, all of the nucleotides of the antisense oligonucleotide of the disclosure are modified. The antisense oligonucleotides of the disclosure in which “substantially all of the nucleotides are modified” are largely but not wholly modified and can include not more than 5, 4, 3, 2, or 1 unmodified nucleotides. In still other embodiments of the disclosure, the antisense oligonucleotide of the disclosure can include not more than 5, 4, 3, 2, or 1 modified nucleotides.

### IV. Modified RNAi Agents and ASOs of the Disclosure

The nucleic acids featured in the disclosure can be synthesized or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; or backbone modifications, including modification or replacement of the phosphodiester linkages. Modification or replacement of the phosphodiester linkages can include stereodefined phosphorothioates and neutral linkages. Stereodefined phosphorothioates can include, for example, right-handed (Rp) diastereomers at the 5′ end and left-handed (Sp) diastereomers at the 3′ end of the antisense strand of an RNAi agent or an ASO of the disclosure. A neutral phosphorothioate linkage may be, for example, an alkylphosphonate linkage, such as a methyl phosphonate (MP) or methoxypropylphosphonate (MOP) linkage. Specific examples of RNAi agents and ASOs useful in the embodiments described herein include but are not limited to, RNAs and ASOs containing modified backbones or no natural internucleoside linkages. RNAs and ASOs having modified backbones include, among others, those that do not have a phosphorus atom in the backbone. For the purposes of this specification, and as sometimes referenced in the art, modified RNAs and ASOs that do not have a phosphorus atom in their internucleoside backbone can also be considered to be oligonucleotides. In some embodiments, a modified RNAi agent or ASO will have a phosphorus atom in its internucleoside backbone.

Modified RNA and ASO backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, e.g., sodium salts, mixed salts and free acid forms are also included. In some embodiments, an ASO comprises at least one phosphorothioate linkage between nucleotides. In some embodiments, an ASO comprises a phosphorothioate linkage between each nucleotide. In some embodiments, the sense strand of a dsRNA molecule comprises 1-10 blocks of two to ten phosphorothioate or methylphosphonate internucleotide linkages separated by 1, 2, 3, 4, 5, 6, 7, 8, 9, 10, 11, 12, 13, 14, 15, or 16 phosphate internucleotide linkages, wherein one of the phosphorothioate or methylphosphonate internucleotide linkages is placed at any position in the oligonucleotide sequence and the said sense strand is paired with an antisense strand comprising any combination of phosphorothioate, methylphosphonate and phosphate internucleotide linkages or an antisense strand comprising either phosphorothioate or methylphosphonate or phosphate linkage. In some embodiments, the antisense strand of the dsRNA molecule comprises two blocks of two phosphorothioate or methylphosphonate internucleotide linkages separated by 1, 2, 3, 4, 5, 6, 7, 8, 9, 10, 11, 12, 13, 14, 15, 16, 17, or 18 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

Modified RNA and ASO backbones that do not include a phosphorus atom therein have backbones that are formed by short chain alkyl or cycloalkyl internucleotide linkages, mixed heteroatoms and alkyl or cycloalkyl internucleotide linkages, or one or more short chain heteroatomic or heterocyclic internucleotide linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,664,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, the entire contents of each of which are hereby incorporated herein by reference.

In other embodiments, suitable RNA and ASO mimetics are contemplated for use in RNAi agents and ASOs in which both the sugar and the internucleotide linkage, i.e., the backbone, of the nucleotide units are replaced with novel groups. The base units are maintained for hybridization with an appropriate nucleic acid target compound. One such oligomeric compound, an RNA and ASO mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the RNAi agents or ASOs of the disclosure are described in, for example, in Nielsen et al., Science, 1991, 254, 1497-1500.

Some embodiments featured in the disclosure include RNAs and ASOs with phosphorothioate backbones and oligonucleosides with heteroatom backbones, and in particular —CH₂—NH—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂— and —N(CH)—CH₂—CH₂— [wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs and ASOs can also contain one or more substituted sugar moieties. The RNAi agents, e.g., dsRNAs, and ASOs featured herein can include one of the following at the 2′-position: OH; F; O—, S—, or N-alkyl; O—, S—, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂).ₙOCH₃, O(CH₂)nNH2, O(CH₂)ₙCH₃, O(CH₂)nONH2, and O(CH₂)nON[(CH₂)nCH₃)]₂, where n and m are from 1 to about 10. In other embodiments, dsRNAs and ASOs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an RNAi agent or ASO, or a group for improving the pharmacodynamic properties of an RNAi agent or ASO, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂. Further exemplary modifications include: 5′-Me-2′-F nucleotides, 5′-Me-2′-OMe nucleotides, 5′-Me-2′-deoxynucleotides, (both R and S isomers in these three families); 2′-alkoxyalkyl; and 2′-NMA (N-methylacetamide).

Other modifications include 2′-methoxy (2′-OCH₃), 2′-aminopropoxy (2′-OCH₂CH₂CH₂NH₂), 2′-O-hexadecyl, and 2′-fluoro (2′-F). Similar modifications can also be made at other positions on an RNAi agent or ASO, particularly the 3′ position of the sugar on the 3′ terminal nucleotide or in 2′-5′ linked dsRNAs or ASO, and the 5′ position of 5′ terminal nucleotide. RNAi agents and ASOs can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference.

An RNAi agent or ASO of the disclosure can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008, those disclosed in The Concise Encyclopedia Of Polymer Science And Engineering, pages 858-859, Kroschwitz, J. L, ed. John Wiley & Sons, 1990, these disclosed by Englisch et al., (1991) Angewandte Chemie, International Edition, 30:613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the disclosure. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

In some embodiments, at least one of the modified nucleotides is selected from a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2-amino-modified nucleotide, a 2′-O-allyl-modified nucleotide, 2′-C-alkyl-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a 5′-phosphorothioate group, a phosphorothioate linkage, a nucleotide comprising a 5′-methylphosphonate group, a nucleotide comprising a 5′ phosphate or 5′ phosphate mimic, a nucleotide comprising vinyl phosphonate, a nucleotide comprising adenosine-glycol nucleic acid (GNA), a nucleotide comprising thymidine-glycol nucleic acid (GNA) S-Isomer, a nucleotide comprising 2-hydroxymethyl-tetrahydrofurane-5-phosphate, a nucleotide comprising 2′-deoxythymidine-3′ phosphate, a nucleotide comprising 2′-deoxyguanosine-3′-phosphate, a 2′-0 hexadecyl nucleotide, a nucleotide comprising a 2′-phosphate, a cytidine-2′-phosphate nucleotide, a guanosine-2′-phosphate nucleotide, a 2′-O-hexadecyl-cytidine-3′-phosphate nucleotide, a 2′-O-hexadecyl-adenosine-3′-phosphate nucleotide, a 2′-O-hexadecyl-guanosine-3′-phosphate nucleotide, a 2′-O-hexadecyl-uridine-3′-phosphate nucleotide, a 5′-vinyl phosphonate (VP or vin- (e.g., vin-u)), a 2′-deoxyadenosine-3′-phosphate nucleotide, a 2′-deoxycytidine-3′-phosphate nucleotide, a 2′-deoxyguanosine-3′-phosphate nucleotide, a 2′-deoxythymidine-3′-phosphate nucleotide, a 2′-deoxyuridine nucleotide, and a terminal nucleotide linked to a cholesteryl derivative and a dodecanoic acid bisdecylamide group; and combinations thereof.

An RNAi agent or ASO of the disclosure can also be modified to include one or more bicyclic sugar moieties. A “bicyclic sugar” is a furanosyl ring modified by the bridging of two atoms. A “bicyclic nucleoside” (“BNA”) is a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring. Thus, in some embodiments, an RNAi agent or ASO of the disclosure may include one or more locked nucleic acids (LNA). A locked nucleic acid is a nucleotide having a modified ribose moiety in which the ribose moiety comprises an extra bridge connecting the 2′ and 4′ carbons. In other words, an LNA is a nucleotide comprising a bicyclic sugar moiety comprising a 4′-CH2-O-2′ bridge. This structure effectively “locks” the ribose in the 3′-endo structural conformation. The addition of locked nucleic acids has been shown to increase stability in serum, and to reduce off-target effects (Elmen, J. et al., (2005) Nucleic Acids Research 33(1):439-447; Mook, OR. et al., (2007) Mol Cane Ther 6(3):833-843; Grunweller, A. et al., (2003) Nucleic Acids Research 31(12):3185-3193). The entire contents of each of the foregoing are hereby incorporated herein by reference. In some embodiments, ASOs of the disclosure can comprise at least one locked nucleotide at both the 5′ and 3′ end. In some embodiments, ASOs of the disclosure can comprise 17 nucleotides and two locked nucleotides at both the 5′ and 3′ end. In some embodiments, ASOs of the disclosure can comprise 17 nucleotides and three locked nucleotides at both the 5′ and 3′ end.

Examples of bicyclic nucleosides for use in the polynucleotides of the disclosure include, without limitation, nucleosides comprising a bridge between the 4′ and the 2′ ribosyl ring atoms. In certain embodiments, the antisense polynucleotide agents of the disclosure include one or more bicyclic nucleosides comprising a 4′ to 2′ bridge. Examples of such 4′ to 2′ bridged bicyclic nucleosides, include but are not limited to 4′-(CH2)-O-2′ (LNA); 4′-(CH2)-S-2′; 4′-(CH2)2-O-2′ (ENA); 4′-CH(CH3)-O-2′ (also referred to as “constrained ethyl” or “cEt”) and 4′-CH(CH2OCH3)-O-2′ (and analogs thereof; see, e.g., U.S. Pat. No. 7,399,845); 4′-C(CH3)(CH3)-O-2′ (and analogs thereof; see e.g., U.S. Pat. No. 8,278,283); 4′-CH2-N(OCH3)-2′ (and analogs thereof; see e.g., U.S. Pat. No. 8,278,425); 4′-CH2-O—N—(CH3)-2′ (see, e.g., U.S. Patent Publication No. 2004/0171570); 4′-CH2-N(R)—O-2′, wherein R is H, C1-C12 alkyl, or a protecting group (see, e.g., U.S. Pat. No. 7,427,672); 4′-CH2-C(H)(CH3)-2′ (see, e.g., Chattopadhyaya et al., J. Org. Chem., 2009, 74, 118-134); and 4′-CH2-C(═CH2)-2′ (and analogs thereof; see, e.g., U.S. Pat. No. 8,278,426).

Additional representative U.S. Patents and U.S. Patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034, 133;7, 084, 125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

A RNAi agent or ASO of the disclosure can also be modified to include one or more constrained ethyl nucleotides. As used herein, a “constrained ethyl nucleotide” or “cEt” is a locked nucleic acid comprising a bicyclic sugar moiety comprising a 4′-CH(CH3)-O-2′ bridge. In one embodiment, a constrained ethyl nucleotide is in the S conformation referred to herein as “S-cEt.”

In some embodiments, an RNAi agent or ASO of the disclosure comprises one or more monomers that are UNA (unlocked nucleic acid) nucleotides. A UNA is an unlocked acyclic nucleic acid, wherein any of the bonds of the sugar have been removed, forming an unlocked “sugar” residue. In one example, a UNA also encompasses monomer in which bonds between C1′-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

In some embodiments, an RNAi agent or ASO of the disclosure comprises one or more nucleotides with a C3 spacer modification. The C3 spacer is a 3-carbon chain attached to the 3′ end of the nucleotide and replaces a nucleotide base in the sense or antisense strand. SEQ ID NOs: 186,582, 186,611, and 186,622 provided herein in Table 9 have C3 spacers at position 6. The full sequence of each of these antisense strands is shown in Table 9 herein.

Representative U.S. publications that teach the preparation of UNAs include, but are not limited to, U.S. Pat. No. 8,314,227; and U.S. Patent Publication Nos. 2013/0096289; 2013/0011922; and 2011/0313020, the entire contents of each of which are hereby incorporated herein by reference.

Potentially stabilizing modifications to the ends of RNA molecules or ASOs can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others.

Other modifications of an RNAi agent or ASO of the disclosure include a 5′ phosphate or 5′ phosphate mimic, e.g., a 5′-terminal phosphate or phosphate mimic on the ASO or the antisense strand of an RNAi agent. Suitable phosphate mimics are disclosed in, for example US 2012/0157511, the entire contents of which are incorporated herein by reference.

Modifications of an RNAi agent or ASO of the disclosure include modifications described in International Patent Publication No. WO2021/142245, the contents of which are incorporated herein by reference.

In some embodiments, a double stranded ribonucleic acid (dsRNA) targeting Mucin-5B (MUC5B) is provided, wherein the dsRNA comprises a sense strand and an antisense strand forming a double stranded region, wherein the antisense strand comprises a region of complementarity to an mRNA encoding MUC5B. In some embodiments, the sense and antisense strands are selected from any one of the sense and antisense pairs in Table 2 or Table 3. In some embodiments, the sense strand comprises the nucleic acid sequence of any one of SEQ ID NOs: 1, 7, 10, 20, 23, 26, 29, 32, 37, 41, 44, 48, 53, 56, 60, 65, 71, 75, 81, 86, 89, 95, 98, 101, 104, 111, 114, 117, 120, 123, 126, 130, 133, 137, 140, 143, 148, 151, 153, 161, 166, 169, 172, and 177; and the antisense strand comprises the nucleic acid sequence of any one of SEQ ID NOs: 178, 184, 187, 197, 200, 203, 206, 209, 214, 218, 221, 225, 230, 233, 237, 242, 248, 252, 258, 263, 266, 272, 275, 278, 281, 288, 291, 294, 297, 300, 303, 307, 310, 314, 317, 320, 325, 328, 330, 338, 343, 346, 349, and 354.

In some embodiments, the sense strand is 21 nucleotides in length and the antisense strand is 23 nucleotides in length. In some embodiments, the sense strand further comprises the following modifications: 2′-OMe modified nucleotides at positions 1-4, 6-8, 12-18, and 20-21, and a phosphorothioate (PTO) modified nucleotide at position 20; and the antisense strand further comprises the following modifications: a 2′-deoxiuridine-3′-phosphate at position 1, 2′-OMe modified nucleotides at positions 13 and 22-23, and a phosphorothioate (PTO) modified nucleotide at position 22, wherein the numbering of nucleotides is in 5′ to 3′ order. In some embodiments, the sense and antisense strands further comprise any one or more of the following modifications (wherein the numbering of nucleotides is in 5′ to 3′ order);
1) 2′-OMe modified nucleotides at positions 2-4 of the antisense strand;
2) 2′-OMe modified nucleotides at positions 2-5 of the antisense strand;
3) 2′-OMe modified nucleotides at positions 3-5 of the antisense strand;
4) Replacement of adenine or cytosine at positions 17 or 18 of the sense strand and positions 5 or 4 of the antisense strand with guanine or uracil to form a G-U Wobble base pairing at either of the corresponding duplex positions;
5) A duplex internal mismatch at position 12 in the sense strand, wherein position 12 of the sense strand comprises the same nucleotide as the corresponding duplex position, position 10, of the antisense strand;
6) A duplex internal mismatch at position 11 in the sense strand, wherein position 11 of the sense strand comprises the same nucleotide as the corresponding duplex position, position 11, of the antisense strand;
7) A duplex internal mismatch at position 10 in the sense strand, wherein position 10 of the sense strand comprises the same nucleotide as the corresponding duplex position, position 12, of the antisense strand;
8) Replacement of any one of positions 1 or 2 of the sense strand and/or positions 21 or 20 of the antisense strand with a guanine or a cytosine to form a C-G stabilizing duplex corresponding to: position 1 of the sense strand duplexed with position 21 of the antisense strand, or position 2 of the sense strand duplexed with position 20 of the antisense strand;
9) Phosphorothioate (PTO) modified nucleotides at positions 1-2 and 19-20 of the sense strand and positions 1-2 and 21-22 of the antisense strand;
10) A 5′-vinyl phosphonate modified nucleotide at position 1 of the antisense strand;
11) A GNA modified nucleotide at position 6 of the antisense strand;
12) A GNA modified nucleotide at position 7 of the antisense strand;
13) 2′-methoxyethoxy (2′-O—CH2CH2OCH3) modified nucleotides (MOE) at positions 9 and 10 of the antisense strand;
14) Phosphorothioate (PTO) modified nucleotides at positions 1-2 and 19-20 of the sense strand, 2′-OMe modified nucleotides at positions 3-5, 7-13, 15, and 17-23 of the antisense strand, phosphorothioate (PTO) modified nucleotides at positions 1-2 and 21-22 of the antisense strand, and a 5′-vinyl phosphonate modified nucleotide at position 1 of the antisense strand;
15) Replacement of RNA nucleotides with DNA nucleotides at positions 5, and 9-11, and 19 of the sense strand, phosphorothioate (PTO) modified nucleotides at positions 1-2 and 19-20 of the sense strand, 2′-OMe modified nucleotides at positions 3-5, 8-13, 15, and 17-23 of the antisense strand, phosphorothioate (PTO) modified nucleotides at positions 1-2 and 21-22 of the antisense strand, replacement of RNA nucleotides with DNA nucleotides at positions 6, 14, and 16 of the antisense strand, and a GNA modified nucleotide at position 7 of the antisense strand;
16) A 2′OMe modified nucleotide at position 2 of the antisense strand and a 5′-vinyl phosphonate modified nucleotide at position 1 of the antisense strand;
17) Phosphorothioate (PTO) modified nucleotides at positions 1-2 and 19-20 of the sense strand and positions 1-2 and 21-22 of the antisense strand, and a 5′-vinyl phosphonate modified nucleotide at position 1 of the antisense strand;
18) Phosphorothioate (PTO) modified nucleotides at positions 1-2 and 19-20 of the sense strand, replacement of RNA nucleotides with DNA nucleotides at positions 5, 9-11, and 19 of the sense strand, phosphorothioate (PTO) modified nucleotides at positions 1-2 and 21-22 of the antisense strand, replacement of RNA nucleotides with DNA nucleotides at positions 2, 6, 14, and 16 of the antisense strand, and a 5′-vinyl phosphonate modified nucleotide at position 1 of the antisense strand;
19) A 5′-vinyl phosphonate modified nucleotide at position 1 of the antisense strand and a GNA modified nucleotide at position 7 of the antisense strand;
20) A 5′-vinyl phosphonate modified nucleotide at position 1 of the antisense strand, a GNA modified nucleotide at position 7 of the antisense strand, and 2′-OMe modified nucleotides at positions 3-5 of the antisense strand; or
21) A 5′-vinyl phosphonate modified nucleotide at position 1 of the antisense strand, a GNA modified nucleotide at position 7 of the antisense strand, 2′-OMe modified nucleotides at positions 3-5 of the antisense strand, and MOE modified nucleotides at positions 9 and 10 of the antisense strand.

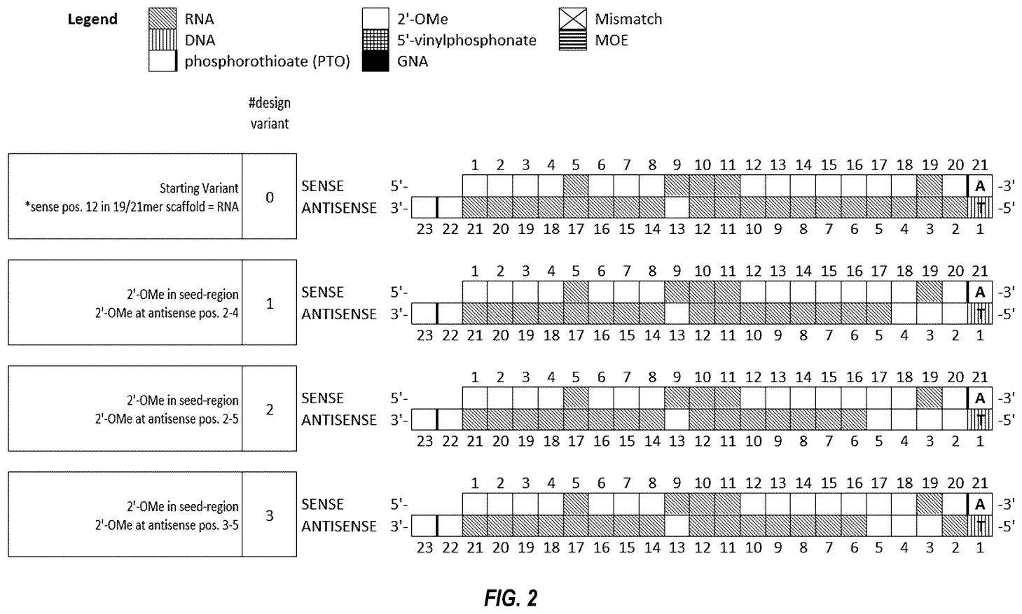

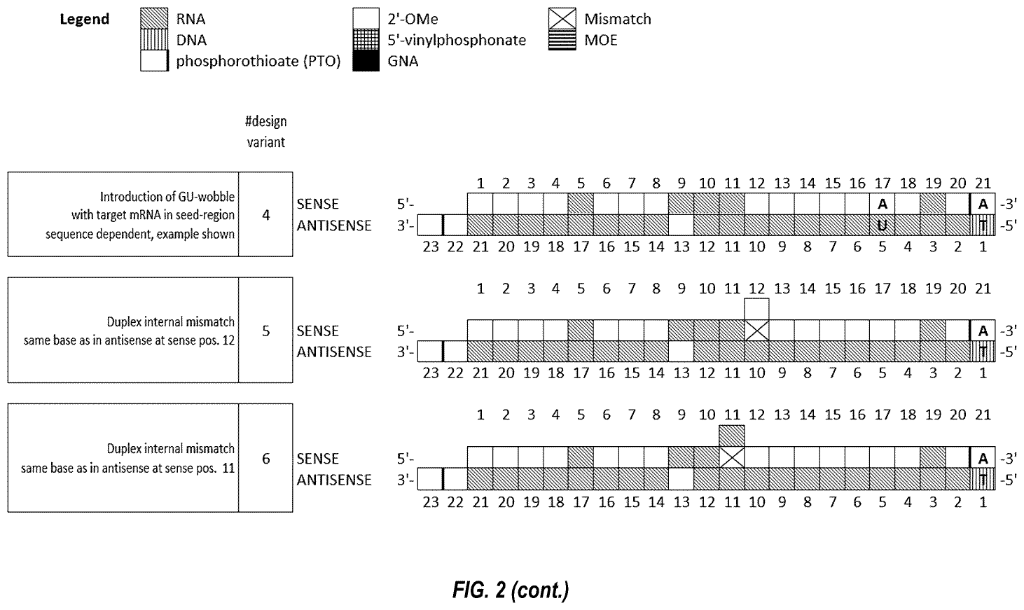

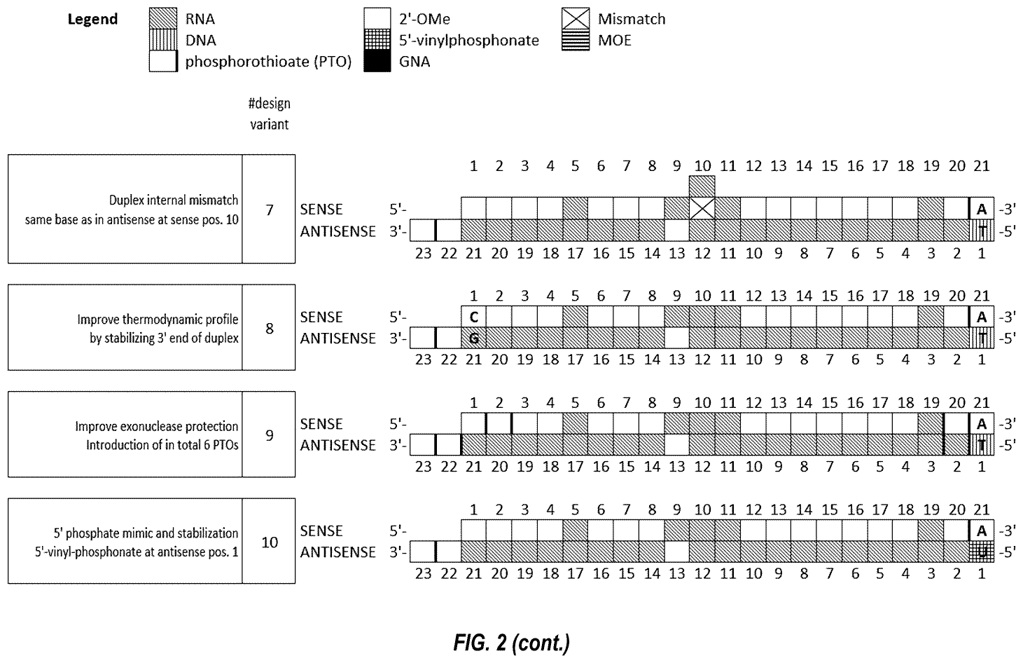

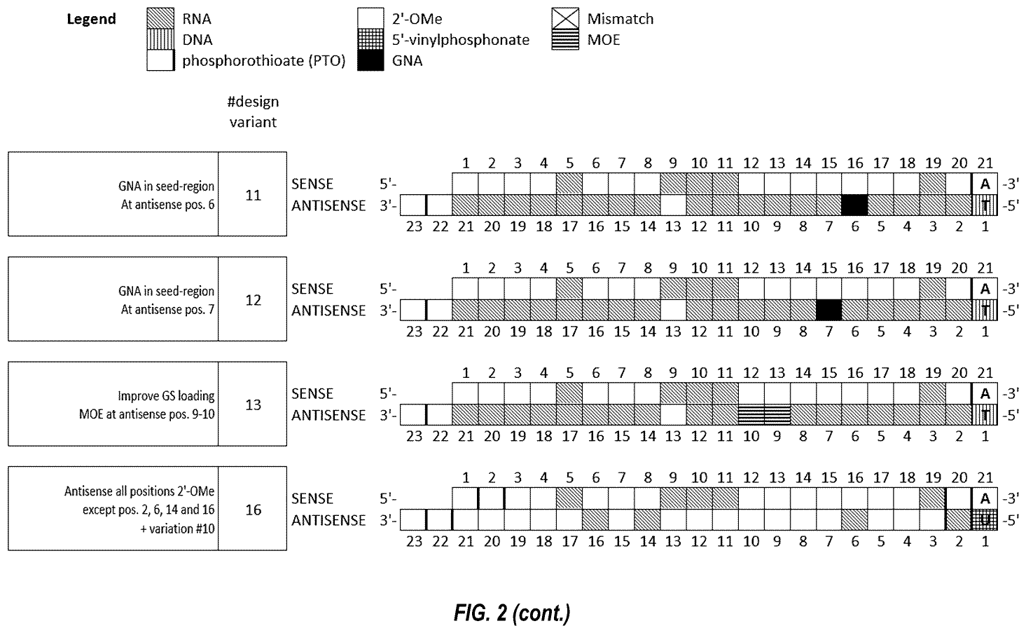

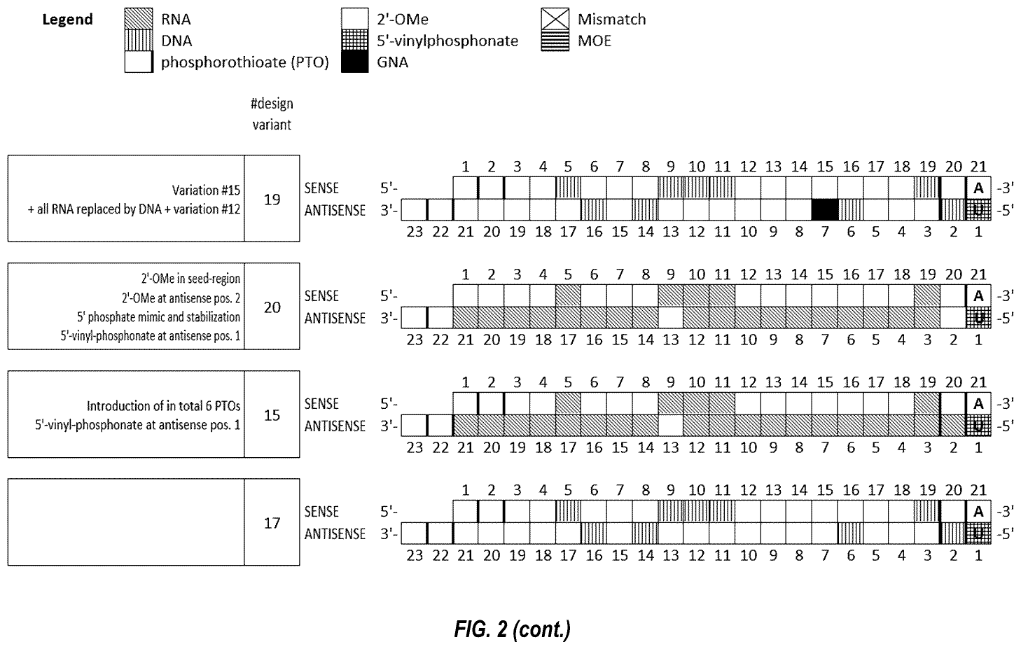

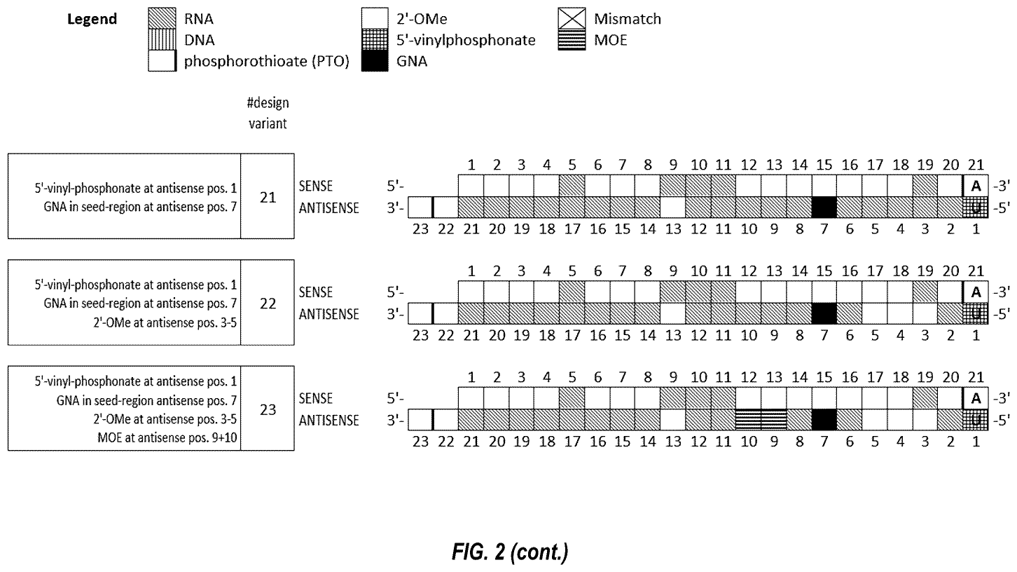

The modifications described in the previous paragraph are demonstrated, for example, in FIG. 2.

In some embodiments, the sense strand comprises the nucleic acid sequence of any one of SEQ ID NOs: 4, 6, 19, 22, 25, 31, 36, 40, 43, 47, 50, 52, 55, 59, 64, 70, 74, 80, 85, 88,94, 97, 100, 103, 110, 113, 116, 119, 122, 125, 129, 132, 136, 139, 142, 147, 150, 160, 165, 168, 171, and 176, and the antisense strand comprises the nucleic acid sequence of any one of SEQ ID NOs: 181, 183, 196, 199, 202, 208, 213, 217, 220, 224, 227, 229, 232, 236, 241, 247, 251, 257, 262, 265, 271, 274, 277, 280, 287, 290, 293, 296, 299, 302, 306, 309, 313, 316, 319, 324, 327, 337, 342, 345, 348, and 353. In some embodiments, the sense strand is 19 nucleotides in length and the antisense strand is 21 nucleotides in length. In some embodiments, the sense strand further comprises the following modifications: 2′-OMe modified nucleotides at positions 1-2, 4-6, 10-11, 13-16, and 18-19, and a phosphorothioate (PTO) modified nucleotide at position 18; and the antisense strand further comprises the following modifications: a 2′-deoxiuridine-3′-phosphate at position 1, 2′-OMe modified nucleotides at positions 13 and 20-21, and a phosphorothioate (PTO) modified nucleotide position 20, wherein the numbering of nucleotides is in 5′ to 3′ order. In some embodiments, the sense and antisense strands further comprise any one or more of the following modifications (wherein the numbering of nucleotides is in 5′ to 3′ order);
1) 2′-OMe modified nucleotides at positions 2-4 of the antisense strand;
2) 2′-OMe modified nucleotides at positions 2-5 of the antisense strand;
3) 2′-OMe modified nucleotides at positions 3-5 of the antisense strand;
4) Replacement of adenine or cytosine at positions 15 or 16 of the sense strand and positions 5 or 4 of the antisense strand with guanine or uracil to form a G-U Wobble base pairing at either of the corresponding duplex positions;
5) A duplex internal mismatch at position 10 in the sense strand, wherein position 10 of the sense strand comprises the same nucleotide as the corresponding duplex position, position 10, of the antisense strand;
6) A duplex internal mismatch at position 9 in the sense strand, wherein position 9 of the sense strand comprises the same nucleotide as the corresponding duplex position, position 11, of the antisense strand;
7) A duplex internal mismatch at position 8 in the sense strand, wherein position 8 of the sense strand comprises the same nucleotide as the corresponding duplex position, position 12, of the antisense strand;
8) Replacement of any one of positions 1 or 2 of the sense strand and/or positions 19 or 18 of the antisense strand with a guanine or a cytosine to form a C-G stabilizing duplex corresponding to: position 1 of the sense strand duplexed with position 19 of the antisense strand, or position 2 of the sense strand duplexed with position 18 of the antisense strand;
9) Phosphorothioate (PTO) modified nucleotides at positions 1-2 and 17-19 of the sense strand and positions 1-2 and 20-21 of the antisense strand;
10) A 5′-vinyl phosphonate modified nucleotide at position 1 of the antisense strand;
11) A GNA modified nucleotide at position 6 of the antisense strand;
12) A GNA modified nucleotide at position 7 of the antisense strand;
13) 2′-methoxyethoxy (2′-O—CH2CH2OCH3) modified nucleotides (MOE) at positions 7 and 9 of the antisense strand;
14) Phosphorothioate (PTO) modified nucleotides at positions 1-2 and 17-18 of the sense strand, 2′-OMe modified nucleotides at positions 3-5, 7-13, 15, and 17-21 of the antisense strand, phosphorothioate (PTO) modified nucleotides at positions 1-2 and 20-21 of the antisense strand, and a 5′-vinyl phosphonate modified nucleotide at position 1 of the antisense strand;
15) Replacement of RNA nucleotides with DNA nucleotides at positions 3, and 7-9, and 17 of the sense strand, phosphorothioate (PTO) modified nucleotides at positions 1-2 and 17-18 of the sense strand, 2′-OMe modified nucleotides at positions 3-5, 8-13, 15, and 17-21 of the antisense strand, phosphorothioate (PTO) modified nucleotides at positions 1-2 and 20-21 of the antisense strand, replacement of RNA nucleotides with DNA nucleotides at positions 6, 14, and 16 of the antisense strand, and a GNA modified nucleotide at position 7 of the antisense strand;
16) A 2′OMe modified nucleotide at position 2 of the antisense strand and a 5′-vinyl phosphonate modified nucleotide at position 1 of the antisense strand;
17) Phosphorothioate (PTO) modified nucleotides at positions 1-2 and 17-18 of the sense strand and positions 1-2 and 19-20 of the antisense strand, and a 5′-vinyl phosphonate modified nucleotide at position 1 of the antisense strand;
18) Phosphorothioate (PTO) modified nucleotides at positions 1-2 and 17-18 of the sense strand, replacement of RNA nucleotides with DNA nucleotides at positions 3, 7-9, and 17 of the sense strand, phosphorothioate (PTO) modified nucleotides at positions 1-2 and 19-20 of the antisense strand, replacement of RNA nucleotides with DNA nucleotides at positions 2, 6, 14, and 16 of the antisense strand, and a 5′-vinyl phosphonate modified nucleotide at position 1 of the antisense strand;
19) A 5′-vinyl phosphonate modified nucleotide at position 1 of the antisense strand and a GNA modified nucleotide at position 7 of the antisense strand;
20) A 5′-vinyl phosphonate modified nucleotide at position 1 of the antisense strand, a GNA modified nucleotide at position 7 of the antisense strand, and 2′-OMe modified nucleotides at positions 3-5 of the antisense strand; or
21) A 5′-vinyl phosphonate modified nucleotide at position 1 of the antisense strand, a GNA modified nucleotide at position 7 of the antisense strand, 2′-OMe modified nucleotides at positions 3-5 of the antisense strand, and MOE modified nucleotides at positions 9 and 10 of the antisense strand.

The modifications described in the previous paragraph are demonstrated, for example, in FIG. 2.

In some embodiments, the sense strand comprises the nucleic acid sequence of any one of SEQ ID NOs: 2, 3, 5, 8, 9, 11, 12, 13, 14, 15, 16, 17, 18, 21, 24, 27, 28, 30, 33, 34, 35, 38, 39, 42, 45, 46, 49, 51, 54, 57, 58, 61, 62, 63, 66, 67, 68, 69, 72, 73, 76, 77, 78, 79, 82, 83, 84, 87, 90, 91, 92, 93, 96, 99, 102, 105, 106, 107, 108, 109, 112, 115, 118, 121, 124, 127, 128, 131, 134, 135, 138, 141, 144, 145, 146, 149, 152, 154, 155, 156, 157, 158, 159, 162, 163, 164, 167, 170, 173, 174, and 175, and the antisense strand comprises the nucleic acid sequence of any one of SEQ ID NOs: 179, 180, 182, 185, 186, 188, 189, 190, 191, 192, 193, 194, 195, 198, 201, 204, 205, 207, 210, 211, 212, 215, 216, 219, 222, 223, 226, 228, 231, 234, 235, 238, 239, 240, 243, 244, 245, 246, 249, 250, 253, 254, 255, 256, 259, 260, 261, 264, 267, 268, 269, 270, 273, 276, 279, 282, 283, 284, 285, 286, 289, 292, 295, 298, 301, 304, 305, 308, 311, 312, 315, 318, 321, 322, 323, 326, 329, 331, 332, 333, 334, 335, 336, 339, 340, 341, 344, 347, 350, 351, and 352. In some embodiments, the sense strand is 21 nucleotides in length and the antisense strand is 23 nucleotides in length. In some embodiments, the sense strand further comprises the following modifications: 2′-OMe modified nucleotides at positions 1-6, 8, and 12-21, phosphorothioate (PTO) modified nucleotides at positions 1-2, and 2′-Fluoro modified nucleotides at positions 7, and 9-11; and the antisense strand further comprises the following modifications: 2′-OMe modified nucleotides at positions 1, 3-5, 7-13, 15, and 17-23, phosphorothioate (PTO) modified nucleotides at positions 1-2, and 21-22, and 2′-Fluoro modified nucleotides at positions 2, 14, and 16, wherein the numbering of nucleotides is in 5′ to 3′ order. In some embodiments, the sense and antisense strands further comprise any one or more of the following modifications (wherein the numbering of nucleotides is in 5′ to 3′ order);
1) A 5′-vinyl phosphonate modified nucleotide at position 1 of the antisense strand;
2) A GNA modified nucleotide at position 6 of the antisense strand;
3) A GNA modified nucleotide at position 7 of the antisense strand;
4) Replacement of the RNA nucleotide at position 11 of the sense strand with a DNA nucleotide;
5) A duplex internal mismatch at position 12 in the sense strand, wherein position 12 of the sense strand comprises the same nucleotide as the corresponding duplex position, position 10, of the antisense strand;
6) A duplex internal mismatch at position 11 in the sense strand, wherein position 11 of the sense strand comprises the same nucleotide as the corresponding duplex position, position 11, of the antisense strand;
7) A duplex internal mismatch at position 10 in the sense strand, wherein position 10 of the sense strand comprises the same nucleotide as the corresponding duplex position, position 12, of the antisense strand;
8) Replacement of adenine or cytosine at any one of positions 15-19 of the sense strand and the corresponding positions 3-7 of the antisense strand with guanine or uracil to form a G-U Wobble base pairing at the corresponding duplex positions;
9) MOE modified nucleotides at positions 22 and 23 of the antisense strand;
10) A 2′-OMe modified nucleotide at position 2 of the antisense strand and a 5′-vinyl phosphonate modified nucleotide at position 1 of the antisense strand.
11) An abasic RNA nucleotide at position 6 of the antisense strand;
12) An abasic DNA nucleotide at position 6 of the antisense strand;
13) A C3 spacer at position 6 of the antisense strand;
14) A 2′-OMe modified abasic RNA nucleotide at position 6 of the antisense strand; or
15) A 2′-Fluoro modified abasic RNA nucleotide at position 6 of the antisense strand.

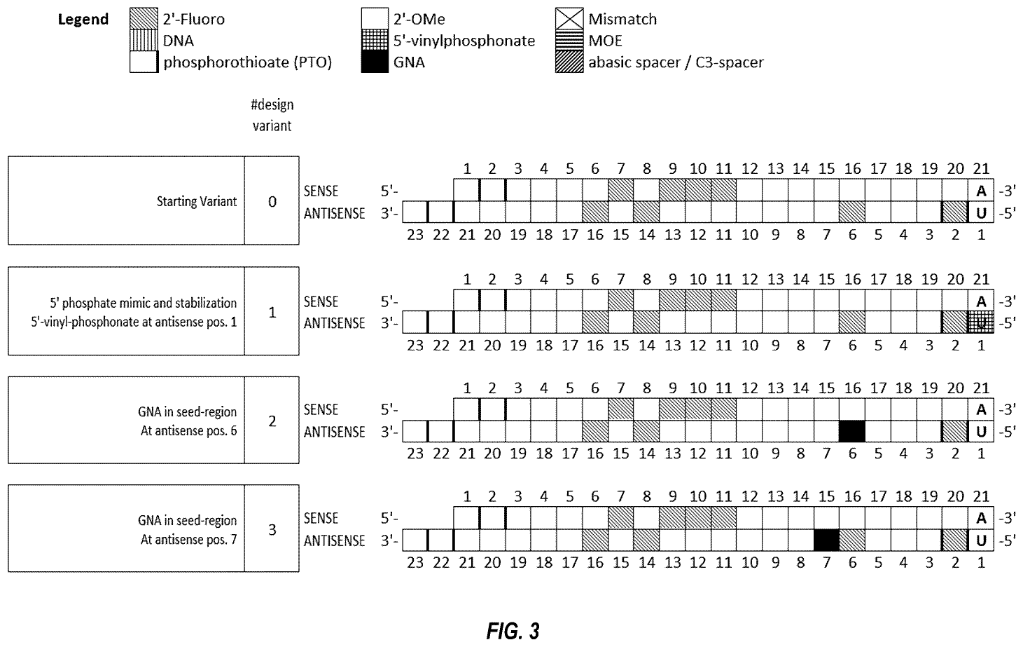

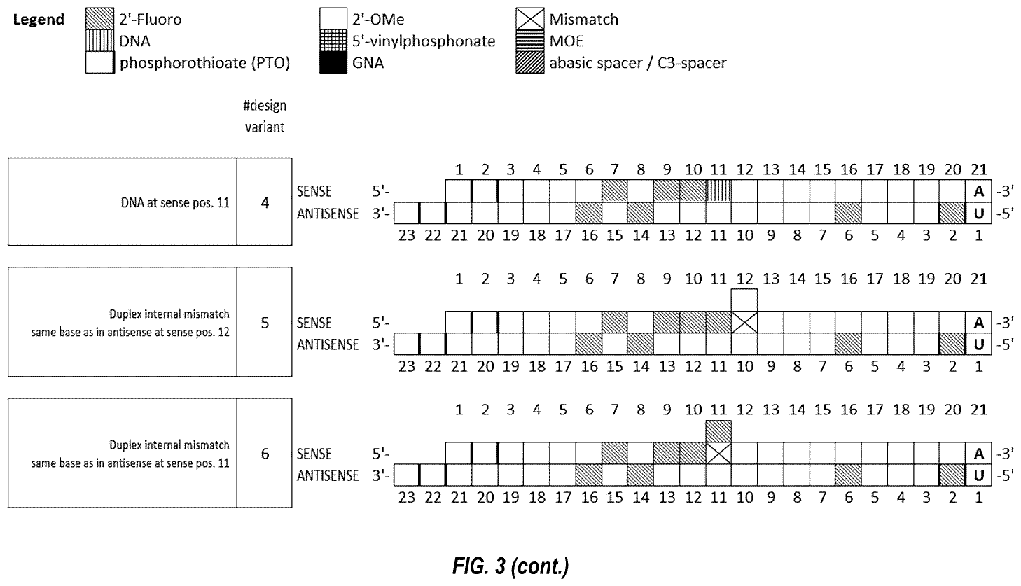

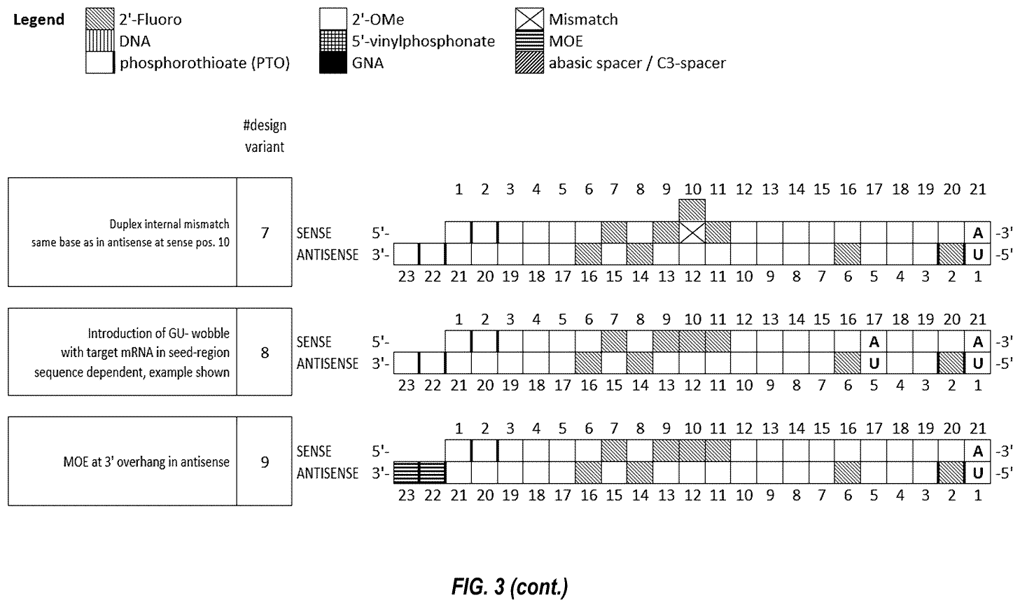

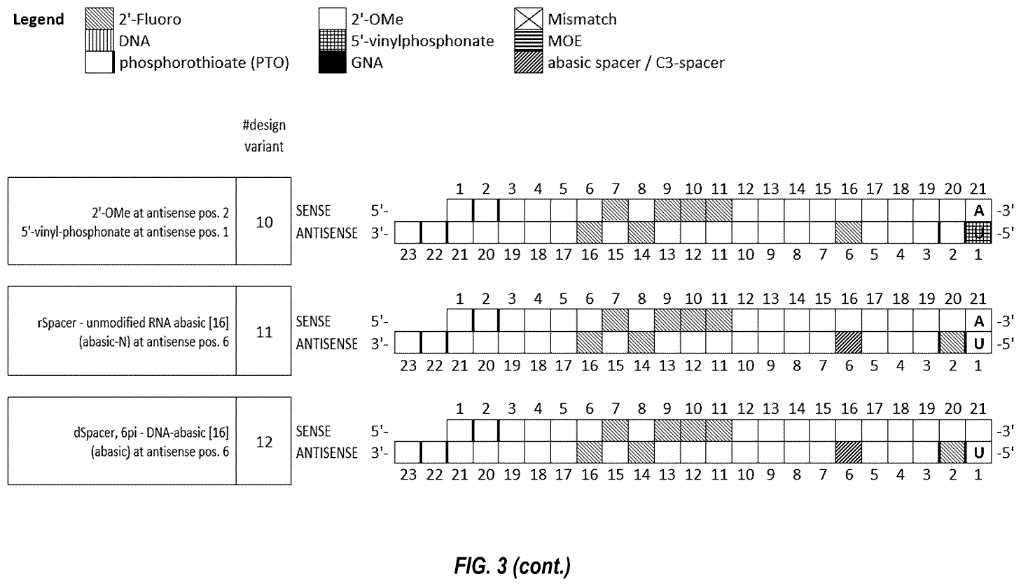

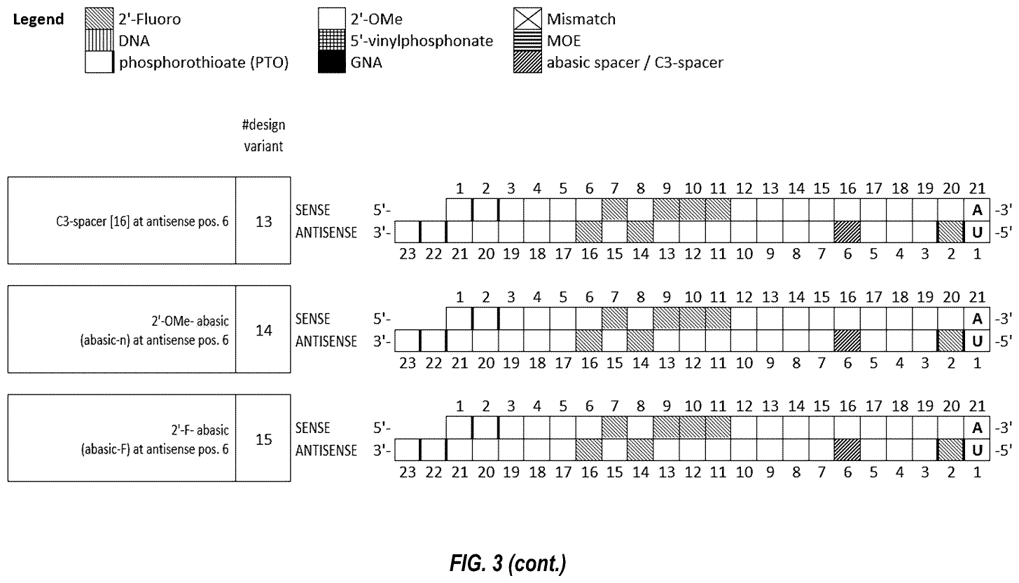

The modifications described in the previous paragraph are demonstrated, for example, in FIG. 3.

In some embodiments, the double stranded ribonucleic acid (dsRNA) targeting Mucin-5B (MUC5B), comprises any one or more modified nucleotides which confer any one or more of the following characteristics compared to the unmodified dsRNA sequence;
1) Improved efficacy;
2) Improved potency;
3) Improved stability; and/or
4) Reduced off-target effects.

In some embodiments, the double stranded ribonucleic acid (dsRNA) targeting Mucin-5B (MUC5B), comprises any one or more modified nucleotides which confer any one or more of the above characteristics 1-4 as compared to the unmodified dsRNA or to the starting V0 dsRNA described in Example 4, Table 9. For example, for the 12706_EEL_19/21mer, the starting V0 dsRNA sense strand comprises the nucleotide sequence of SEQ ID NO: 358, and the antisense strand comprises the nucleotide sequence of SEQ ID NO: 535. This starting V0 dsRNA was further modified as described in Example 4 to generate 21 new modified dsRNA variants, listed as V01-V23 in Table 9.

Efficacy, in the context of the characteristics above, is defined herein as the extent of MUC5B silencing by the dsRNA. Efficacy can be measured by various assays, including the on-target reporter assay described in Example 4 herein. In some embodiments, efficacy is defined as the on-target mean maximum score at 24 hours post transfection, as described in Example 4. Exemplary results for modified dsRNA efficacy are shown in Table 10. In some embodiments, modified dsRNA of the present disclosure generate a lower mean maximum on-target score at 24 hours post transfection than the corresponding starting V0 dsRNA, indicating improved efficacy of the modified dsRNA compared to the starting V0 dsRNA.

Potency, in the context of the characteristics above, is defined herein as the dose of dsRNA required for MUC5B silencing. In some embodiments, potency is assayed using a dose-response experiment, described in Example 4 herein. In some embodiments, potency is defined as the IC50, or concentration of dsRNA required to reach 50% of the maximum silencing effect. Exemplary results for modified dsRNA potency are shown in Table 10. In some embodiments, modified dsRNA of the present disclosure generate a lower IC50 than the corresponding starting V0 dsRNA, indicating improved potency of the modified dsRNA, as a lower dose of dsRNA is required to generate the same degree of silencing.

Stability, in the context of the characteristics above, is defined herein as reduced MUC5B expression more than 24 hours after transfection with dsRNA. In some embodiments, dsRNA stability is assayed using real time quantitative PCR (RT-PCR) 5 days after dsRNA transfection, as described in Example 4. Exemplary results for modified dsRNA stability are shown in Table 10. In some embodiments, modified dsRNA of the present disclosure generate a lower mean maximum on-target score at day 5 than the corresponding starting V0 dsRNA, indicating increased stability of the modified dsRNA.

Off-target effects, or off-target activity, in the context of the characteristics above, are defined herein as dsRNA binding to non-target sequences. Off-target effects can be assayed in various ways, including the off-target reporter assay described in Example 4 herein. In some embodiments, reduced off-target effects of modified dsRNA are defined as any mean maximum off-target score greater than that of the corresponding V0 starting variant. In some embodiments, a reduced off-target score is defined as an off-target mean maximum score greater than, for example, 90% as assayed by the off-target reporter assay described in Example 4. Exemplary results for modified dsRNA off-target effects are shown in Table 10.

In some embodiments, the double stranded ribonucleic acid (dsRNA) targeting Mucin-5B (MUC5B), comprises any one or more modified nucleotides which confer any one or more of the following characteristics compared to the corresponding starting dsRNA, V0 as described in Example 4 and Table 9:
1) Improved efficacy, wherein efficacy is defined as an on-target mean maximum score at 24 hours within the range of the on-target mean maximum score of the starting V0 dsRNA 2, 3, or 4 standard deviations. In some embodiments, efficacy is assayed using an on-target reporter system described in Example 4. In some embodiments, efficacy is defined as an on-target mean maximum score at 24 hours that is no greater than the mean maximum on-target score of starting V0 variant+10%;
2) Improved potency, wherein potency is defined as a reduced on-target IC50 compared to that of the starting V0 dsRNA. In some embodiments, potency is assayed using an on-target reporter system described in Example 4. In some embodiments, the IC50 is more than 2-fold, 5-fold, or 10-fold lower than that of the starting V0 dsRNA;
3) An off-target score greater than 80%, 85%, 90%, or 95%, as defined in Table 10. In some embodiments, the off-target activity is assayed using an off-target reporter system described in Example 4.
4) Improved stability, defined as an on-target mean maximum score at day 5 within the range of the mean on-target score at day 5 of the starting V0 dsRNA ±2, 3, or 4 standard deviations. In some embodiments, the on-target score at day 5 is assayed using real-time quantitative PCR for MUC5B mRNA expression as described in Example 4.

In a specific embodiment, for example, the modified dsRNA of the present disclosure displays improved off-target activity while retaining the efficacy, potency, and stability of the starting V0 dsRNA and/or the unmodified dsRNA.

Experimental results for exemplary dsRNA pairs shown in Table 9 are presented in Table 10.

In one embodiment, the modified dsRNA of the present disclosure display any one or more of the following characteristics as compared to the starting V0 dsRNA: improved efficacy, improved potency, improved stability, and/or reduced off-target effects. Exemplary dsRNA exhibiting one or more of these characteristics include those that are set forth below;
1) 12706_EEL_19/21mer_V09, wherein the sense strand comprises the nucleotide sequence of SEQ ID NO: 186,507, and the antisense strand comprises the nucleotide sequence of SEQ ID NO: 186,543;
2) 12706_EEL_21/23mer_V07, wherein the sense strand comprises the nucleotide sequence of SEQ ID NO: 186,512, and the antisense strand comprises the nucleotide sequence of SEQ ID NO: 538;
3) 12706_EEL_21/23mer_V09, wherein the sense strand comprises the nucleotide sequence of SEQ ID NO: 186,514, and the antisense strand comprises the nucleotide sequence of SEQ ID NO: 186,561;
4) 12706_EEL_21/23mer_V10, wherein the sense strand comprises the nucleotide sequence of SEQ ID NO: 361, and the antisense strand comprises the nucleotide sequence of SEQ ID NO: 186,562;
5) 12706_EEL_21/23mer_V11, wherein the sense strand comprises the nucleotide sequence of SEQ ID NO: 361, and the antisense strand comprises the nucleotide sequence of SEQ ID NO: 186,563;
6) 15478_EEL_21/23mer_V12, wherein the sense strand comprises the nucleotide sequence of SEQ ID NO: 364, and the antisense strand comprises the nucleotide sequence of SEQ ID NO: 186,593;
7) 15478_EEL_21/23mer_V21, wherein the sense strand comprises the nucleotide sequence of SEQ ID NO: 364, and the antisense strand comprises the nucleotide sequence of SEQ ID NO: 186,600;
8) 15539_DV22_21/23mer_V01, wherein the sense strand comprises the nucleotide sequence of SEQ ID NO: 356, and the antisense strand comprises the nucleotide sequence of SEQ ID NO: 186,603;
9) 15539_DV22_21/23mer_V03, wherein the sense strand comprises the nucleotide sequence of SEQ ID NO: 356, and the antisense strand comprises the nucleotide sequence of SEQ ID NO: 186,605;
10) 15539_DV22_21/23mer_V08, wherein the sense strand comprises the nucleotide sequence of SEQ ID NO: 186,532, and the antisense strand comprises the nucleotide sequence of SEQ ID NO: 186,606;
11) 15539_DV22_21/23mer_V09, wherein the sense strand comprises the nucleotide sequence of SEQ ID NO: 356, and the antisense strand comprises the nucleotide sequence of SEQ ID NO: 186,607;
12) 219_DV22_21/23mer_V01, wherein the sense strand comprises the nucleotide sequence of SEQ ID NO: 359, and the antisense strand comprises the nucleotide sequence of SEQ ID NO: 186,614;
13) 219_DV22_21/23mer_V07, wherein the sense strand comprises the nucleotide sequence of SEQ ID NO: 186,536, and the antisense strand comprises the nucleotide sequence of SEQ ID NO: 536; and
14) 219_DV22_21/23mer_V08, wherein the sense strand comprises the nucleotide sequence of SEQ ID NO: 186,537, and the antisense strand comprises the nucleotide sequence of SEQ ID NO: 186,617.

In another embodiment, the modified dsRNA of the present disclosure display improved efficacy, defined as a mean maximum on-target score at 24 hours within 3 standard deviations from the mean of the starting V0 dsRNA. The following modified dsRNA of the present disclosure, described further in Example 4 herein, for example, display improved efficacy, defined as a mean maximum on-target score at 24 hours within 3 standard deviations from the mean of the starting V0 dsRNA;
1) 12706_EEL_19/21mer_V09, wherein the sense strand comprises the nucleotide sequence of SEQ ID NO: 186,507, and the antisense strand comprises the nucleotide sequence of SEQ ID NO: 186,543;
2) 15478_EEL_21/23mer_V12, wherein the sense strand comprises the nucleotide sequence of SEQ ID NO: 364, and the antisense strand comprises the nucleotide sequence of SEQ ID NO: 186,593;
3) 15478_EEL_21/23mer_V21, wherein the sense strand comprises the nucleotide sequence of SEQ ID NO: 364, and the antisense strand comprises the nucleotide sequence of SEQ ID NO: 186,600;
4) 15539_DV22_21/23mer_V01, wherein the sense strand comprises the nucleotide sequence of SEQ ID NO: 356, and the antisense strand comprises the nucleotide sequence of SEQ ID NO: 186,603;
5) 15539_DV22_21/23mer_V03, wherein the sense strand comprises the nucleotide sequence of SEQ ID NO: 356, and the antisense strand comprises the nucleotide sequence of SEQ ID NO: 186,605;
6) 15539_DV22_21/23mer_V08, wherein the sense strand comprises the nucleotide sequence of SEQ ID NO: 186,532, and the antisense strand comprises the nucleotide sequence of SEQ ID NO: 186,606;
7) 15539_DV22_21/23mer_V09, wherein the sense strand comprises the nucleotide sequence of SEQ ID NO: 356, and the antisense strand comprises the nucleotide sequence of SEQ ID NO: 186,607;
8) 219_DV22_21/23mer_V01, wherein the sense strand comprises the nucleotide sequence of SEQ ID NO: 359, and the antisense strand comprises the nucleotide sequence of SEQ ID NO: 186,614; and
9) 219_DV22_21/23mer_V08, wherein the sense strand comprises the nucleotide sequence of SEQ ID NO: 186,537, and the antisense strand comprises the nucleotide sequence of SEQ ID NO: 186,617.

In a further embodiment, modified dsRNA of the present disclosure, described further in Example 4 herein, display improved efficacy, defined as a mean maximum on-target score at 24 hours that is no greater than the mean maximum on-target score of starting V0 variant+10%. For example, the following modified dsRNA of the present disclosure, described further in Example 4 herein, display improved efficacy, defined as a mean maximum on-target score at 24 hours that is no greater than the mean maximum on-target score of starting V0 variant+10%;
1) 12706_EEL_21/23mer_V07, wherein the sense strand comprises the nucleotide sequence of SEQ ID NO: 186,512, and the antisense strand comprises the nucleotide sequence of SEQ ID NO: 538;
2) 12706_EEL_21/23mer_V09, wherein the sense strand comprises the nucleotide sequence of SEQ ID NO: 186,514, and the antisense strand comprises the nucleotide sequence of SEQ ID NO: 186,561;
3) 12706_EEL_21/23mer_V10, wherein the sense strand comprises the nucleotide sequence of SEQ ID NO: 361, and the antisense strand comprises the nucleotide sequence of SEQ ID NO: 186,562; and
4) 12706_EEL_21/23mer_V11, wherein the sense strand comprises the nucleotide sequence of SEQ ID NO: 361, and the antisense strand comprises the nucleotide sequence of SEQ ID NO: 186,563.

In another embodiment, the modified dsRNA of the present disclosure, described further in Example 4 herein, display a potency as determined by an IC50 equivalent to or less than the IC50 of the staring V0 dsRNA For example, the following modified dsRNA of the present disclosure, described further in Example 4 herein, have IC50 of equal to or less than the IC50 of the staring V0 dsRNA;
1) 219_DV22_21/23mer_V01, wherein the sense strand comprises the nucleotide sequence of SEQ ID NO: 359, and the antisense strand comprises the nucleotide sequence of SEQ ID NO: 186,614
2) 219_DV22_21/23mer_V07, wherein the sense strand comprises the nucleotide sequence of SEQ ID NO: 186,536, and the antisense strand comprises the nucleotide sequence of SEQ ID NO: 536; and
3) 219_DV22_21/23mer_V08, wherein the sense strand comprises the nucleotide sequence of SEQ ID NO: 186,537, and the antisense strand comprises the nucleotide sequence of SEQ ID NO: 186,617.

In another embodiment, the modified dsRNA of the present disclosure, described further in Example 4 herein, display improved stability, defined as a mean maximum on-target score at day 5 within 3 standard deviations from the mean of the starting V0 dsRNA. For example, the following modified dsRNA of the present disclosure, described further in Example 4 herein, display improved stability, defined as a mean maximum on-target score at day 5 within 3 standard deviations from the mean of the starting V0 dsRNA;
1) 12706_EEL_19/21mer_V09, wherein the sense strand comprises the nucleotide sequence of SEQ ID NO: 186,507, and the antisense strand comprises the nucleotide sequence of SEQ ID NO: 186,543;
2) 12706_EEL_21/23mer_V07, wherein the sense strand comprises the nucleotide sequence of SEQ ID NO: 186,512, and the antisense strand comprises the nucleotide sequence of SEQ ID NO: 538;
3) 12706_EEL_21/23mer_V09, wherein the sense strand comprises the nucleotide sequence of SEQ ID NO: 186,514, and the antisense strand comprises the nucleotide sequence of SEQ ID NO: 186,561;
4) 12706_EEL_21/23mer_V10, wherein the sense strand comprises the nucleotide sequence of SEQ ID NO: 361, and the antisense strand comprises the nucleotide sequence of SEQ ID NO: 186,562;
5) 15478_EEL_21/23mer_V12, wherein the sense strand comprises the nucleotide sequence of SEQ ID NO: 364, and the antisense strand comprises the nucleotide sequence of SEQ ID NO: 186,593;
6) 15478_EEL_21/23mer_V21, wherein the sense strand comprises the nucleotide sequence of SEQ ID NO: 364, and the antisense strand comprises the nucleotide sequence of SEQ ID NO: 186,600;
7) 15539_DV22_21/23mer_V01, wherein the sense strand comprises the nucleotide sequence of SEQ ID NO: 356, and the antisense strand comprises the nucleotide sequence of SEQ ID NO: 186,603;
8) 219_DV22_21/23mer_V01, wherein the sense strand comprises the nucleotide sequence of SEQ ID NO: 359, and the antisense strand comprises the nucleotide sequence of SEQ ID NO: 186,614;
9) 219_DV22_21/23mer_V07, wherein the sense strand comprises the nucleotide sequence of SEQ ID NO: 186,536, and the antisense strand comprises the nucleotide sequence of SEQ ID NO: 536; or
10) 219_DV22_21/23mer_V08, wherein the sense strand comprises the nucleotide sequence of SEQ ID NO: 186,537, and the antisense strand comprises the nucleotide sequence of SEQ ID NO: 186,617.

In a further embodiment, modified dsRNA of the present disclosure, described further in Example 4 herein, display reduced off-target effects, defined as an off-target score greater than 95%. For example, the following modified dsRNA of the present disclosure, described further in Example 4 herein, display reduced off-target effects, defined as an off-target score greater than 95% as described in Example 4, Table 10;
1) 12706_EEL_19/21mer_V09
2) 12706_EEL_21/23mer_V07
3) 12706 EEL_21/23mer_V09
4) 12706_EEL_21/23mer_V10
5) 12706_EEL_21/23mer_V11
6) 15478_EEL_21/23mer_V12
7) 15478 EEL_21/23mer_V21
8) 15539_DV22_21/23mer_V03
9) 15539_DV22_21/23mer_V08
10) 219_DV22_21/23mer_V01
11) 219_DV22_21/23mer_V07
12) 219_DV22_21/23mer_V08

### V. RNAis and ASOs Conjugated to Ligands

The RNAi and ASO agents of the disclosure can contain conjugations that optimize one or more properties of the RNAi agent or ASO. In many cases, a carbohydrate moiety will be attached to a modified subunit of the RNAi agent or ASO. For example, the ribose sugar of one or more ribonucleotide subunits of a dsRNA agent or ASO can be replaced with another moiety, e.g., a non-carbohydrate (preferably cyclic) carrier to which is attached a carbohydrate ligand. A ribonucleotide subunit in which the ribose sugar of the subunit has been replaced is referred to herein as a ribose replacement modification subunit (RRMS). A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system or may contain two or more rings, e.g., fused rings. The cyclic carrier may be a fully saturated ring system, or it may contain one or more double bonds.

The ligand may be attached to the polynucleotide via a carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g. monosaccharide, disaccharide, trisaccharide, tetrasaccharide, oligosaccharide and polysaccharide. Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring. The cyclic group carrier can be selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and decalin. In some cases, the cyclic carrier is an acyclic moiety, for example an acyclic moiety, such as a moiety based on a serinol backbone or a diethanolamine backbone.

An RNAi agent or ASO of the disclosure can be chemically linked to one or more ligands, moieties, or conjugates that enhance the activity, cellular distribution, or cellular uptake of the iRNA or ASO, e.g., into a cell. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10: 1111-1118; Kabanov et al., FEBS Lett., 1990, 259:327-330: Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In certain embodiments, a ligand alters the distribution, targeting or lifetime of an iRNA agent or ASO into which it is incorporated. In some embodiments, a ligand provides an enhanced affinity for a selected target, e.g., molecule, cell or cell type, compartment, e.g., a cellular or organ compartment, tissue, organ or region of the body, as, e.g., compared to a species absent such a ligand. Typical ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or a helical peptide.

Ligands can also include targeting groups, e.g., a cell or tissue targeting agent, such as a targeting ligand that targets a lung tissue, e.g., a lectin, glycoprotein, lipid, or protein, e.g., an antibody, that binds to a specified cell type. A targeting group can be a thyrotropin, melanotropin, lectin, glycoprotein, surfactant protein A, mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic. In some embodiments, the targeting ligand is a lipophilic moiety, e.g., a C16, and/or a carbohydrate moiety, e.g., a GalNAc ligand, or any other ligand that directs the RNAi agent or ASO to a site of interest. In some embodiments, the lipophilic moiety contains a saturated or unsaturated C4-C30 hydrocarbon chain and an optional functional group selected from the group consisting of hydroxyl, amine, carboxylic acid, sulfonate, phosphate, thiol, azide, and alkyne. In some embodiments, the lipophilic moiety contains a saturated or unsaturated C6-C18 hydrocarbon chain. In certain embodiments, the lipophilic moiety targeting ligand is a 2′-O-hexadecyl (C16) conjugate. In some embodiments, the lipophilic moiety contains a saturated or unsaturated C16 hydrocarbon chain. In one embodiment, the saturated or unsaturated C16 hydrocarbon chain is conjugated to position 6, counting from the 5′-end of the strand.

Ligands that may be conjugated to an RNAi agent or ASO of the disclosure for the purpose of targeting include moieties disclosed in International Patent Publication No. WO2021/142245, the contents of which are incorporated herein by reference.

### VI. Lipid Particles

RNAi agents, e.g., dsRNAs, or ASOs of the present disclosure may be fully encapsulated in a lipid formulation, e.g., a lipid nanoparticle (LNP), or other nucleic acid-lipid particles. As used herein, the term “LNP” refers to a stable nucleic acid-lipid particle.

LNP formulations may include ionizable lipids, PEG-lipids, helper lipids, cholesterol and cholesterol-based lipids, amphiphilic block copolymers and/or polymers, and combinations thereof. Ionizable lipids mediate encapsulation of nucleic acids and self-assembly of LNPs during formulation and promote endosomal release of the nucleic acids once the LNP is taken up by a cell. The ionizable lipid may be a cationic lipid, which LNPs typically contain together with a non-cationic lipid and a lipid that prevents aggregation of the particle (e.g., a PEG-lipid conjugate). Cationic lipids have a head group with permanent positive charges. Ionizable lipids are protonated and thus positively charged at low pH, but they are generally neutral at physiological pH. This pH sensitivity can aid RNAi agent or ASO delivery, because neutral lipids may have fewer interactions with the anionic membranes of blood cells and, thus, improve the biocompatibility of lipid nanoparticles. Ionizable lipids are protonated in endosomes, where the pH is lower than outside the cell. Thus, in the endosomes, ionizable lipids will be positively charged, facilitating membrane destabilization and endosomal escape of the LNPs.

Cationic or ionizable lipids for LNPs may include, for example, 3,6-bis(4-(bis(2-hydroxydodecyl)amino)butyl)piperazine-2,5-dione (cKK-E12); 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol) (C12-200); ((4-hydroxybutyl)azanediyl)bis(hexane-6,1-diyl)bis(2-hexyldecanoate) (ALC-0315); Dimethyldioctadecylammonium bromide (DDAB); heptadecan-9-yl 8-((2-hydroxyethylx6-oxo-6-(undecyloxy)hexyl)amino) octanoate (Lipid H/SM-102); (6Z,9Z,28Z,31Z)-heptatriaconta-6,9,28,31-tetraen-19-yl 4-(dimethylamino) butanoate (DLin-MC3-DMA); DLin-KC2-DMA; 1,2-dilinoleyloxy-N,N-dimethyl-3-aminopropane (DLin-DMA); hexa(octan-3-yl) 9,9′,9″,9″,9″″,9′″″-((((benzene-1,3,5-tricarbonyl)yris(azanediyl)) tris (propane-3,1-diyl)) tris(azanetriyl))hexanonanoate (FTT5); LP01; (((3,6-dioxopiperazine-2,5-diyl)bis(butane-4, 1-diyl))bis(azanetriyl))tetrakis(ethane-2,1-diyl) (9Z,9′Z,9″Z,9″Z,12Z,12′Z,12″Z,12′″Z)-tetrakis (octadeca-9,12-dienoate) (OF-Deg-Lin); ethyl 5,5-di((Z)-heptadec-8-en-1-yl)-1-(3-(pyrrolidin-1-yl)propyl)-2,5-dihydro-1H-imidazole-2-carboxylate (A2-Iso5-2DC18); tetrakis(8-methylnonyl) 3,3′,3″,3′-(((methylazanediyl) bis(propane-3,1 diyl))bis (azanetriyl))tetrapropionate (306Oᵢ₁ₒ); bis(2-(dodecyldisulfanyl)ethyl) 3,3′-((3-methyl-9-oxo-10-oxa-13,14-dithia-3,6-diazahexacosyl)azanediyl)dipropionate (BAME-O16B); N¹,N³,N⁵-tris(3-(didodecylamino)propyl)benzene-1,3,5-tricarboxamide (TT3); or decyl (2-(dioctylammonio)ethyl) phosphate (9A1p9). Additional cationic lipids can include 1,2-di-O-octadecenyl-3-trimethylammonium-propane (DOTMA); 2,3-dioleyloxy-N-[2-(sperminecarboxamido)ethyl]-N,N-dimethyl-1-propanaminium trifluoroacetate (DOSPA); 1,2-dioleoyl-3-trimethylammonium-propane (DOTAP); or ethylphosphatidylcholine (ePC).

Cholesterol-based compounds can include cholesterol or a modified version or analogue thereof, such as oxidized cholesterols, esterified cholesterols, or phytosterols. Exemplary modified cholesterols include (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol (p-sitosterol); 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide (BHEM-Cholesterol); and 3β-[N—(N′,N′-dimethylaminoethane)-carbamoyl]cholesterol (DC-Cholesterol).

PEG-lipids can include compounds such as polyethylene glycol-2000-C-DMG (PEG-2000-C-DMG); 1,2-dimyristoyl-rac-glycero-3-methoxypolyethylene glycol-2000 (PEG-2000-DMG, a.k.a. DMG-PEG₂₀₀₀); and 2-[(polyethylene glycol)-2000]—N,N-ditetradecylacetamide (ALC-0159).

“Helper lipids” can include compounds such as 1,2-distearoyl-sn-glycero-3-phosphocholine (DSPC); and 1,2-dioleoyl-sn-glycero-3-phosphoethanolamine (DOPE); or other phospholipids.

In some embodiments, an LNP includes one of each of a cationic lipid, a cholesterol or cholesterol derivative, a PEG-lipid, and a helper lipid. Certain structures or types of LNP components or LNP charge ratio (lipid to RNA ratio) may be selected for specific delivery profiles. A delivery profile may be determined by the size and charge of the LNP, which is acquired through changes in the molar compositions of the four types of lipids typically used in an LNP formulation. Examples include the use of increasing amounts of DMG-PEG2000 from 0.004 μmol to 0.12 μmol to reduce the LNP size from 200 nm to 30 nm and the use of CHEMS at ˜20 mol % to obtain negatively charged LNP. (Nakamura T., et al., The Effect of Size and Charge of Lipid Nanoparticles Prepared by Microfluidic Mixing on Their Lymph Node Transitivity and Distribution. Mol. Pharm. 17:944-953 (2020)); replacing traditional linear PEG-lipids with 3% Tween 20, which contains three PEG chains and a single lipid chain (Zukancic D., et al., The Importance of Poly(ethylene glycol) and Lipid Structure in Targeted Gene Delivery to Lymph Nodes by Lipid Nanoparticles. Pharmaceutics. 12:1068 (2020), the disclosures of which are incorporated herein by reference. An additional lipid may be introduced to achieve passive targeting, where such lipids have been termed selective organ targeting (SORT) lipids (Dilliard S. A., Cheng Q., Siegwart D. J. On the mechanism of tissue-specific mRNA delivery by selective organ targeting nanoparticles. Proc. Natl. Acad. Sci. USA. 118:e2109256118 (2021); and Cheng Q., et al., Selective organ targeting (SORT) nanoparticles for tissue-specific mRNA delivery and CRISPR-Cas gene editing. Nat. Nanotechnol. 15:313-320 (2020), the disclosures of which are incorporated herein by reference. These SORT lipids may be introduced to the LNP formulation by dissolution in THF or ethanol at different molar ratios before mixing with the RNA to obtain targeting to specific tissues including the lung. (Cheng Q., et al., Nat. Nanotechnol. 15:313-320 (2020 (Id); Liu S., et al., Membrane-destabilizing ionizable phospholipids for organ-selective mRNA delivery and CRISPR-Cas gene editing. Nat. Mater. 20:701-710 (2021); Alvarez-Benedicto E., et al., Optimization of phospholipid chemistry for improved lipid nanoparticle (LNP) delivery of messenger RNA (mRNA) Biomater. Sci. 10:549-559 (2022); Lee S. M., et al., A Systematic Stud of Unsaturation in Lipid Nanoparticles Leads to Improved mRNA Transfection In Vivo. Angew. Chem. Int. Ed. Engl. 60:5848-5853 (2021), the disclosures of which are incorporated herein by reference.

Further examples of LNP formulation designs that facilitate targeting to specific tissues include those described in Dahlman et al. In vivo endothelial siRNA delivery using polymeric nanoparticles with low molecular weight. Nature Nanotechnology 9(8), 648-655 (2014); Khan, O. F. et al. Endothelial siRNA delivery in nonhuman primates using ionizable low-molecular weight polymeric nanoparticles. Sci. Adv. 4, eaar8409 (2018); Sago, C. D. et al. Nanoparticles that deliver RNA to bone marrow identified by in vivo directed evolution. J. Am. Chem. Soc. 140, 17095-17105 (2018); Paunovska, K. et al. Analyzing 2000 in vivo drug delivery data points reveals cholesterol structure impacts nanoparticle delivery. ACS Nano 12, 8341-8349 (2018); Lokugamage, M. P. et al. Optimization of lipid nanoparticles for the delivery of nebulized therapeutic mRNA to the lungs. Nat. Biomed. Eng. 5, 1059-1068 (2021); Mui, B. L. et al. Influence of polyethylene glycol lipid desorption rates on pharmacokinetics and pharmacodynamics of siRNA lipid nanoparticles. Mol. Ther. Nucleic acids 2, e139 (2013); and Ryals, R. C. et al. The effects of PEGylation on LNP based mRNA delivery to the eye. PLoS ONE 15, e0241006 (2020) (changing the cholesterol, PEG-lipid or ‘helper’ lipid can alter delivery profile), the disclosures of which are each incorporated herein by reference. In some embodiments, LNPs with increased zeta potential or increased positive charge, for example, may increase delivery to the lungs, as described in Kranz, L. M. et al. Systemic RNA delivery to dendritic cells exploits antiviral defence for cancer immunotherapy. Nature 534, 396-401 (2016); and Kauffman, K. J. et al. Rapid, single-cell analysis and discovery of vectored mRNA transfection in vivo with a loxP-flanked tdtomato reporter mouse. Mol. Ther. Nucleic Acids 10, 55-63 (2018), the disclosures of which are incorporated herein by reference. Additional exemplary LNP components may be one or more of those described in Kulkarni, J. A., Cullis, P. R. & van der Meel, R. Lipid nanoparticles enabling gene therapies: from concepts to clinical utility. Nucleic Acid. Ther. 28, 146-157 (2018); and Cheng, X. & Lee, R. J. The role of helper lipids in lipid nanoparticles (LNPs) designed for oligonucleotide delivery. Adv. Drug Deliv. Rev. 99, 129-137 (2016), the disclosures of which are incorporated herein by reference. In some embodiments, an RNAi agent or ASO may be formulated in an LNP that comprises each of a cationic lipid, a cholesterol or a modified version or analogue thereof, a PEG-lipid, and a helper lipid.

In some embodiments, an LNP may incorporate biodegradable lipids containing ester linkages in the lipid tails and/or linker group which may be eliminated more rapidly and may exhibit improved safety profiles compared with non-biodegradable lipids. For example, introducing ester bonds in the linker and lipidic tails of MC3, yielding the lipid di((Z)-non-2-en-1-yl) 9-((4-(dimethylamino)butanoyl)oxy)heptadecanedioate (L319), has been observed to improve delivery efficacy and faster elimination from the liver and plasma in vivo relative to MC3. Improved in vivo delivery efficacy and pharmacokinetics have also been observed using biodegradable lipids heptadecan-9-yl 8-((2-hydroxyethylx8-(nonyloxy)-8-oxooctyl)amino)octanoate (Lipid 5), heptadecan-9-yl 8-((2-hydroxyethyl)(6-oxo-6-(undecyloxy)hexyl)amino) octanoate (Lipid H (SM-102)) and ((4-hydroxybutyl)azanediyl)bis(hexane-6,1-diyl)bis(2-hexyldecanoate) (ALC-0315) compared to MC3. Two of these biodegradable lipids, SM-102 and ALC-0315, are the ionizable lipids in the mRNA-1273 and BNT162b COVID-19 vaccines, respectively. Biodegradable lipids can also include ester and/or disulfide linkages. Cleavage of the disulfide bonds can promote intraparticle nucleophilic attack on an ester linkage, thereby facilitating degradation of the lipids.

Zwitterionic ionizable lipids can also be employed in an LNP for delivery of an RNAi agent or ASO of the disclosure. For example, lipids composed of a pH-switchable zwitterion and three hydrophobic tails can assemble into a cone in the pH environment of the endosome, whereby membrane hexagonal transformation can occur and allow endosomal escape, allowing delivery of the RNA cargo to the cytoplasm.

Libraries of chemically distinct lipid molecules that have been synthesized using chemistries such as Michael addition-based, epoxide-based and alcohol-based reactions (See, e.g., Altinoglu, S., Wang, M. & Xu, Q. Combinatorial library strategies for synthesis of cationic lipid-like nanoparticles and their potential medical applications. Nanomedicine 10, 643-657 (2015); and Zhang, Y., Sun, C., Wang, C., Jankovic, K. E. & Dong, Y. Lipids and lipid derivatives for RNA delivery. Chem. Rev. 121, 12181-12277 (2021), incorporated herein by reference) may be screened to identify lipids, lipid derivatives and lipid-derived molecules that may be used for delivery of RNAi agents or ASOs of the disclosure.

LNPs useful for formulation of the RNAi agents or ASOs of the disclosure also include tissue-targeted LNPs obtained by introducing targeting ligands e.g., directly to the formulation in ethanol, chemically conjugating to the LNP surface, or by modifying the composition of the lipids in the formulation. Such “active targeting” of LNPs may be via attachment of antibodies, whereby, for example, a functionalized DSPE-PEG may be introduced during LNP formulation (e.g. at 12.5-25% of total PEG) followed by chemical grafting of an antibody specific for target tissue or cells, as described in Li Q., et al. Engineering Caveolae-Targeted Lipid Nanoparticles To Deliver mRNA to the Lungs. ACS Chem. Biol. 15:830-836 (2020); Kedmi R., et al. A modular platform for targeted RNAi therapeutics. Nat. Nanotechnol. 13:214-219 (2018); and Ramishetti S., et al., Systemic Gene Silencing in Primary T Lymphocytes Using Targeted Lipid Nanoparticles. ACS Nano. 9:6706-6716 (2015), the disclosures of which are each incorporated herein by reference. In some embodiments, the targeting ligand is specific for the lungs. For example, an Fab-C4 may be conjugated to DSPE-PEG-maleimide via a Diels-Alder reaction, where the Fab-C4 contains a cyclopentadiene lysine derivative to allow the Diels-Alder transformation, as described in Li Q, et al, ACS Chem. Biol. 15:830-836 (2020).

Exemplary LNPs also include pre-condensed stable plasmid lipid particles (pSPLPs), which include an encapsulated condensing agent-nucleic acid complex as set forth in WO 00/03683. The particles of the present disclosure typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present disclosure are resistant in aqueous solution to degradation with a nuclease. Further nucleic acid-lipid particles and their method of preparation are disclosed in, e.g., U.S. Pat. Nos. 5,976,567; 5,981,501; 6,534,484; 6,586,410; 6,815,432; United States Patent publication No. 2010/0324120 and WO 96/40964.

In one embodiment, the lipid to drug ratio (mass/mass ratio) (e.g., lipid to dsRNA or ASO ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part of the disclosure. Certain specific LNP formulations have been described in the art, including, e.g., “LNP01” formulations as described in, e.g., WO 2008/042973, which is hereby incorporated by reference. Lipids and LNP formulations that may be employed with an RNAi agent or ASO of the disclosure include those described in International Patent Publication No. WO2021/142245, the contents of which are incorporated herein by reference.

### VII. Vectors, Cells, Pharmaceutical Compositions, Devices

In some embodiments, the present invention provides vectors, cells, pharmaceutical compositions, devices, and methods as described herein, which comprise an RNAi agent or ASO of the present disclosure.

### Vectors, Cells

RNAi agents or ASOs targeting the MUC5B gene can be expressed from transcription units inserted into DNA or RNA vectors (see, e.g., Couture, A, et al., TIG. (1996), 12:5-10; WO 00/22113, WO 00/22114, and U.S. Pat. No. 6,054,299). Expression is preferably sustained (months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., (1995) Proc. Natl. Acad. Sci. USA 92:1292).

RNAi agent and ASO expression vectors are generally DNA plasmids or viral vectors. Expression vectors compatible with eukaryotic cells, preferably those compatible with vertebrate cells, can be used to produce recombinant constructs for the expression of an RNAi agent or ASO as described herein.

Viral vector systems which can be utilized with the methods and compositions described herein include, but are not limited to, (a) adenovirus vectors; (b) retrovirus vectors, including but not limited to lentiviral vectors, moloney murine leukemia virus, etc., (c) adeno-associated virus vectors; (d) herpes simplex virus vectors; (e) SV40 vectors; (f) polyoma virus vectors; (g) papilloma virus vectors; (h) picomavirus vectors; (i) pox virus vectors such as an orthopox, e.g., vaccinia virus vectors or avipox, e.g. canary pox or fowl pox; and (j) a helper-dependent or gutless adenovirus. Other aspects to consider for vectors and constructs are known in the art.

### Pharmaceutical Compositions

The present disclosure also includes pharmaceutical compositions and formulations which include an RNAi agent or ASO of the disclosure. In one embodiment, provided herein are pharmaceutical compositions containing an RNAi agent or ASO, as described herein, and a pharmaceutically acceptable carrier. The pharmaceutical compositions containing the RNAi agent or ASO are useful for treating a subject who would benefit from inhibiting or reducing the expression of a MUC5B gene, e.g., a subject having a MUC5B-associated disorder, e.g., a subject having or at risk of having or at risk of developing a lung disease, e.g., cystic fibrosis, chronic obstructive pulmonary disease (COPD), and/or pulmonary fibrosis, e.g., idiopathic pulmonary fibrosis (IPF). Such pharmaceutical compositions are formulated based on the mode of delivery. One example is compositions that are formulated for direct delivery into the pulmonary system by intrapulmonary administration, intranasal administration, or oral inhalation administration, e.g., by inhalation or insufflation of powders or aerosols, including by nebulizer or intratracheal instillation.

The pharmaceutical compositions of the disclosure may be administered in dosages sufficient to inhibit the expression of a MUC5B gene. In general, a suitable dose of an RNAi agent or ASO of the disclosure will be a flat dose in the range of about 0.001 to about 200.0 mg about once per month to about once per year, typically about once per quarter (i.e., about once every three months) to about once per year, generally a flat dose in the range of about 1 to 50 mg about once per month to about once per year, typically about once per quarter to about once per year. In certain embodiments, the dose will be a fixed dose, e.g., a fixed dose of about 25 μg to about 5 mg.

A repeat-dose regimen may include administration of a therapeutic amount of an RNAi agent or ASO on a regular basis, such as monthly to once every six months. In certain embodiments, the RNAi agent or ASO is administered about once per quarter (i.e., about once every three months) to about twice per year, particularly for the treatment of a chronic disease.

After an initial treatment regimen (e.g., loading dose) of once per day, twice per week, or once per week, the treatments can be administered less frequently.

In other embodiments, a single dose of a pharmaceutical composition disclosed herein can be long-lasting, such that subsequent doses are administered at intervals of not more than 1, 2, 3, or 4 months or more. In some embodiments of the disclosure, a single dose of a pharmaceutical composition of the disclosure is administered once per month. In other embodiments of the disclosure, a single dose of a pharmaceutical composition of the disclosure is administered once per quarter to twice per year.

The skilled artisan will appreciate that certain factors can influence the dosage and timing required to effectively treat a subject, including but not limited to the severity of the disease or disorder, previous treatments, the general health or age of the subject, and other diseases present. Moreover, the treatment of a subject with a therapeutically effective amount of a composition can include a single treatment or a series of treatments.

The RNAi agents or ASOs can be delivered in a manner that targets a particular tissue, such as the lung (e.g., bronchioles, alveoli, or bronchus of the lung). Pharmaceutical compositions that may include and may be used to provide an RNAi agent or ASO of the disclosure include compositions described in International Patent Publication No. WO2021/142245, the contents of which are incorporated herein by reference.

### Devices

The present disclosure also includes devices for inhalation administration that include an RNAi agent or ASO of the disclosure. Nasal delivery devices include, but are not limited to, vapor inhalers, nasal droppers, spray bottles, metered dose spray pumps, gas driven spray atomizers, nebulizers, mechanical powder sprayers, breath actuated inhalers, and insufflators. Devices for delivery deeper into the respiratory system, e.g., into the lung, include nebulizers, pressured metered-dose inhalers, dry powder inhalers, and thermal vaporization aerosol devices. Devices for delivery by inhalation are available from commercial suppliers. Devices can be fixed- or variable-dose, single or multidose, disposable or reusable depending on, for example, the disease or disorder to be prevented or treated, the volume of the agent to be delivered, the frequency of delivery of the agent, and other considerations in the art.

Oral inhalative administration may include use of device, e.g., a passive breath driven or active power driven single/-multiple dose dry powder inhaler (DPI), to deliver a double stranded RNAi agent or ASO of the disclosure to the pulmonary system. Suitable dosage forms for oral inhalative administration include powders and solutions. Suitable devices for oral inhalative administration include nebulizers, metered-dose inhalers, and dry powder inhalers. The amount of RNAi agent or ASO for pulmonary system administration may vary from one target gene to another target gene, and the appropriate amount that has to be applied may have to be determined individually for each target gene. Typically, this amount ranges from 10 μg to 2 mg, preferably 50 μg to 1500 μg, more preferably 100 μg to 1000 μg.

Appropriate devices that may be used to administer an RNAi agent or ASO of the disclosure include devices described in International Patent Publication No. WO2021/142245, the contents of which are incorporated herein by reference.

### Viii. Methods of Treating or Preventing MUC5B-Associated Diseases

The present disclosure also provides methods of inhibiting the expression of a MUC5B gene in a cell, specifically by inhibiting the levels of MUC5B RNA in a cell. The methods include contacting a cell with an RNAi agent, e.g., a double stranded RNAi agent or an ASO, in an amount effective to inhibit expression of a MUC5B gene in the cell, thereby inhibiting expression of MUC5B in the cell. In certain embodiments of the disclosure, expression of a MUC5B gene is inhibited preferentially in the pulmonary system (e.g., lung, bronchial, alveoli) cells. In certain embodiments of the disclosure, expression of a MUC5B gene is inhibited in the pulmonary system (e.g., lung, bronchial, alveoli) cells and in liver cells (e.g., hepatocytes).

Contacting a cell with an RNAi agent, e.g., a double stranded RNAi agent or ASO, may be done in vitro or in vivo. Contacting a cell in vivo with an RNAi agent or ASO includes contacting a cell or group of cells within a subject, e.g., a human subject, with the RNAi agent or ASO. Combinations of in vitro and in vivo methods of contacting a cell are also possible.

In some embodiments of the methods of the disclosure, expression of a MUC5B gene is inhibited by at least 20%, 30%, 40%, preferably at least 50%, 60%, 70%, 80%, 85%, 90%, or 95%, or to below the level of detection of the assay.

The present disclosure also provides methods for treating a subject having a lung disease or a subject at risk of developing a lung disease, comprising administering to the subject in need thereof a therapeutically effective amount of the RNAi agent, ASO, or the pharmaceutical composition thereof. In some embodiments, the subject is a human. The lung disease is associated with overexpression of MUC5B, optionally wherein overexpression of MUC5B is associated with one or more of reduced mucociliary function, reduced alveolar repair, and increased lung fibrosis, for example, one or more of pulmonary fibrosis, cystic fibrosis, chronic obstructive pulmonary disease (COPD), asthma, Primary Ciliary Dyskinesia, or bronchiectasis. In some embodiments, pulmonary fibrosis is idiopathic pulmonary fibrosis (IPF). In some embodiments, treating comprises amelioration of at least symptom of the disease.

The dsRNA agent, ASO, or the pharmaceutical composition thereof can be administered to the subject subcutaneously, intravenously, orotracheally, via oral inhalation, or via intranasal administration. In some embodiments, an additional agent or a therapy suitable for treatment or prevention of a lung disease can be administered to the subject. Exemplary additional therapeutics and treatments include, for example, an anti-inflammatory agent (e.g., a systemic corticosteroid (e.g., prednisone), an immune modulator (e.g., an immunosuppressant agent (e.g., azathioprine, cyclophosphamide), a phosphodiesterase-5 inhibitor, a tyrosine kinase inhibitor (e.g., nintedanib), an antifibrotic agent (e.g., pirfenidone), and a combination of any of the foregoing.

An RNAi agent or ASO of the disclosure may be used to treat a lung disease or at risk of having a lung disease, such as a condition that may benefit from inhibition of MUC5B expression, according to methods described for such diseases or conditions in International Patent Publication No. WO2021/142245, the contents of which are incorporated herein by reference in their entirety.

### IX. EXAMPLES

The following examples are presented in order to more fully illustrate some embodiments of the invention. They should in no way be construed, however, as limiting the broad scope of the invention. Those of ordinary skill in the art can readily adopt the underlying principles of this discovery to design various compounds without departing from the spirit of the current invention.

### Example 1. RNAi Agent and ASO Synthesis

### Source of Reagents

Where the source of a reagent is not specifically given herein, such reagent can be obtained from any supplier of reagents for molecular biology at a quality/purity standard for application in molecular biology.

The selection of RNAi agent and ASO designs targeting human MUC5B gene (human NCBI refseqID: NM_002458.3; NCBI GeneID: 727897) was performed using an integrated bioinformatics approach.

siRNAs: In silico, all possible siRNAs targeting human MUC5B mRNA (NM_002458.3), having a length of 17,911 bases, were generated. The siRNAs' MUC5B mRNA knockdown activity and off-target binding were predicted for humans, rhesus monkeys, cynomolgus monkeys, mice, rats, and ferrets. A specificity score was assigned to each RNAi agent strand. Also, RNAi agent strands were analyzed for the presence of human, rhesus monkey, dog, pig, mouse, rat, and rabbit miRNA seed regions. Specificity categories were assigned to RNAi agents (combined specificity score+miRNA seed analysis). Human SNPs were mapped to RNAi agent target sites in MUC5B transcript NM_002458.3. Based on in-silico overall predictive performance, top RNAi agents were synthesized according to three possible RNA designs using particular chemical compositions, each of which has a specific chemical siRNA pattern with sense and antisense strand lengths, respectively, of 19 and 21 nucleotides, or of 21 and 23 nucleotides, and comprising 2′-O-Me and 2′Fluoro RNA. RNAi agents were synthesized and annealed using standard methods.

A set of unmodified RNAi agent sense and antisense strand sequences targeting MUC5B is shown in Table 2.

Chemical modifications to siRNAs may, in some embodiments, improve their efficacy, potency, duration, and off-target profiles. (See e.g., references 1-17). A set of modified RNAi agent sense and antisense strand sequences targeting MUC5B is shown in Table 3. A list of abbreviations of chemical modifications is shown in Table 1.

ASOs: All possible single-stranded ASO sequences were designed by targeting the human MUC5B gene, Gene ID: 727897; genomic locus NC_000011.10(1223066 . . . 1262172). ASOs were designed to mediate RNaseH-dependent cleavage of the human MUC5B mRNA. ASO sequences were designed to be 17 nucleotides long and have 3 LNA (Locked Nucleic Acids) modifications at the 5′ and 3′ ends and an LNA-free central gap with 11 DNA nucleotides. The LNA-containing flanking regions confer nuclease resistance while increasing target binding affinity, regardless of the GC content. The central DNA “gap” activates RNase H cleavage of the target RNA upon binding. ASOs have fully modified phosphorothioate (PS) backbones, which ensure exceptional resistance to enzymatic degradation. ASOs were scored based on predicted on- and off-target activity, runs of A, C, G, and T, continuous runs of CG, GC content, hepatotoxic and non-toxic motifs, acute neurotoxic potential, potential immune-stimulatory CpG motifs, SNPs, G tetraplexes and motifs effecting activity.

A detailed list of unmodified ASO sequences targeting MUC5B is shown in Table 4. A detailed list of modified ASO sequences targeting MUC5B is shown in Table 5.

siRNAs and ASOs were purchased from Axolabs. RNAi agents and ASOs were synthesized on a 0.2 μmol scale followed by HPLC purification using a Mermade 192 synthesizer (BioAutomation) with phosphoramidite chemistry on solid supports. The solid support was controlled pore glass (500-1000 A) loaded with a custom GalNAc ligand (3′-GalNAc conjugates), universal solid support (AM Chemicals), or the first nucleotide of interest. siRNA quality control criteria were as follows. single strand identity: (+/−0.05% of calculated masse (by ESI/MS analysis); single strand purity: >85.0% intact oligonucleotide by HPLC analysis (UV260 nm); and duplex purity: >90% duplex (by non-denaturing HPLC analysis). ASO quality control criteria were as follows: Identity: +/−0.05% of calculated mass by ESI/MS analysis and purity; >85% intact oligonucleotide by HPLC analysis (UV260).

TABLE 1
Abbreviations of nucleotide monomers used in nucleic
acid sequence representation. It will be understood
that these monomers, when present in an oligonucleotide,
are mutually linked by 5′-3′-phosphodiester bonds.

| Abbreviation | Nucleotide(s) |
|---|---|
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| N | any nucleotide, modified or unmodified |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methyguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| S | phosphorothioate linkage (PTO) |
| L1O | N-(cholesterylcarboxamidocaproyl)-4- |
| | hydroxyprolinol (Hvo-C6-Chol) |
| Y34 | 2-hydroxymethyl-tetrahydrofurane-4-methoxy-3- |
| | phosphate(abasic 2′-OMe furanose) |
| Y44 | inverted abasic DNA (2-hydroxymethyl- |
| | tetrahydrofurane-5-phosphate) |
| (Aim) or GNA-A | Adenosine-glycol nucleic acid (GNA) |
| (Cim) or GNA-C | Cytidine-glycol nucleic acid (GNA) |
| (Gim) or GNA-G | Guanosine-glycol nucleic acid (GNA) |
| (Tim) or GNA-T | Thymidine-glycol nucleic acid (GNA) S-Isomer |
| p | Phosphate |
| VP or (vin-) | 5′Vinyl-phosphonate |
| (e.g., vin-u) | |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxiuridine-3′-phosphate |
| dTs | 2′-deoxythymidine-3′-phosphorothioate |
| dU | 2′-deoxyuridine |
| dUs | 2′-deoxyuridine-3′-phosphorothioate |
| (C2p) | cytidine-2′-phosphate |
| (G2p) | guanosine-2′-phosphate |
| (U2p) | uridine-2′-phosphate |
| (A2p) | adenosine-2′-phosphate |
| (vin-u) | 5′vinyl-phosphonate modified 2′-O- |
| | methyluridine-3′-phosphate |
| Nm | MOE RNA |
| abasic-N | rSpacer - unmodified RNA abasic |
| abasic | dSpacer, 6pi - DNA-abasic |
| C3 | C3-spacer |
| abasic-n | 2′-OMe-abasic |
| abasic-F | 2′-F-abasic |

TABLE 2
Unmodified Sense and Antisense MUCSB siRNA Sequences

| | | | SEQ | | SEQ |
|---|---|---|---|---|---|
| | | Sense Sequence | ID | Antisense Sequence | ID |
| SIRNA | SIRNA_ID | 5′ to 3′ | NO: | 5′ to 3′ | NO: |
| XD-60355 | 1399_EEL_21/23mer | cagcuacguucuguccaagaa | 1 | tucuuggacagaacguagcugca | 178 |
| XD-60339 | 15539_DV22_21/23mer | cuguuugaccaaauuccggua | 2 | uaccggaauuuggucaaacagga | 179 |
| XD-61278 | 214_DV22_21/23mer | cgugagcuuuguuccacccga | 3 | ucggguggaacaaagcucacgcg | 180 |
| XD-60426 | 12706_EEL_19/21mer | aaauccguguguucugcua | 4 | tagcagaacacacggauuuca | 181 |
| XD-61279 | 219_DV22_21/23mer | gcuuuguuccacccgucacua | 5 | uagugacggguggaacaaagcuc | 182 |
| XD-60430 | 15258_EEL_19/21mer | cacuaugagugcgagugca | 6 | tgcacucgcacucauagugga | 183 |
| XD-60380 | 12706_EEL_21/23mer | ugaaauccguguguucugcua | 7 | tagcagaacacacggauuucaua | 184 |
| XD-60331 | 15311_DV22_21/23mer | accuuugacggcaccucuuaa | 8 | uuaagaggugccgucaaaggugg | 185 |
| XD-60336 | 15476_DV22_21/23mer | uacaaguccauggauaucgua | 9 | uacgauauccauggacuuguagu | 186 |
| XD-60390 | 15478_EEL_21/23mer | caaguccauggauaucgucca | 10 | tggacgauauccauggacuugua | 187 |
| XD-60316 | 12616_DV22_21/23mer | guugggccaggucguggaaua | 11 | uauuccacgaccuggcccaacuc | 188 |
| XD-60317 | 12621_DV22_21/23mer | gccaggucguggaaugcagca | 12 | ugcugcauuccacgaccuggccc | 189 |
| XD-60318 | 12706_DV22_21/23mer | ugaaauccguguguucugcua | 13 | uagcagaacacacggauuucaua | 190 |
| XD-60320 | 12717_DV22_21/23mer | uguucugcugcaacuacggca | 14 | ugccguaguugcagcagaacaca | 191 |
| XD-60319 | 13206_DV22_21/23mer | ccuccacuccggggacgacca | 15 | uggucguccccggaguggaggag | 192 |
| XD-60273 | 1399_DV22_21/23mer | cagcuacguucuguccaagaa | 16 | uucuuggacagaacguagcugca | 193 |
| XD-60321 | 15132_DV22_21/23mer | gcacgguggccaggugcguga | 17 | ucacgcaccuggccaccgugcag | 194 |
| XD-60322 | 15140_DV22_21/23mer | gccaggugcgugggugacaaa | 18 | uuugucacccacgcaccuggcca | 195 |
| XD-60427 | 15140_EEL_19/21mer | caggugcgugggugacaaa | 19 | tuugucacccacgcaccuggc | 196 |
| XD-60381 | 15140_EEL_21/23mer | gccaggugcgugggugacaaa | 20 | tuugucacccacgcaccuggcca | 197 |
| XD-60323 | 15197_DV22_21/23mer | gucaccugcgugaacaagcaa | 21 | uugcuuguucacgcaggugacgu | 198 |
| XD-60428 | 15197_EEL_19/21mer | caccugcgugaacaagcaa | 22 | tugcuuguucacgcaggugac | 199 |
| XD-60382 | 15197_EEL_21/23mer | gucaccugcgugaacaagcaa | 23 | tugcuuguucacgcaggugacgu | 200 |
| XD-60324 | 15252_DV22_21/23mer | gugacuuccacuaugagugca | 24 | ugcacucauaguggaagucacag | 201 |
| XD-60429 | 15252_EEL_19/21mer | gacuuccacuaugagugca | 25 | tgcacucauaguggaagucac | 202 |
| XD-60383 | 15252_EEL_21/23mer | gugacuuccacuaugagugca | 26 | tgcacucauaguggaagucacag | 203 |
| XD-60325 | 15254_DV22_21/23mer | gacuuccacuaugagugcgaa | 27 | uucgcacucauaguggaagucac | 204 |
| XD-60326 | 15258_DV22_21/23mer | uccacuaugagugcgagugca | 28 | ugcacucgcacucauaguggaag | 205 |
| XD-60384 | 15258_EEL_21/23mer | uccacuaugagugcgagugca | 29 | tgcacucgcacucauaguggaag | 206 |
| XD-60327 | 15261_DV22_21/23mer | acuaugagugcgagugcauca | 30 | ugaugcacucgcacucauagugg | 207 |
| XD-60431 | 15261_EEL_19/21mer | uaugagugcgagugcauca | 31 | tgaugcacucgcacucauagu | 208 |
| XD-60385 | 15261_EEL_21/23mer | acuaugagugcgagugcauca | 32 | tgaugcacucgcacucauagugg | 209 |
| XD-61286 | 15262_DV22_21/23mer | cuaugagugcgagugcaucua | 33 | uagaugcacucgcacucauagug | 210 |
| XD-60328 | 15304_DV22_21/23mer | cuauuccaccuuugacggcaa | 34 | uugccgucaaagguggaauagug | 211 |
| XD-60329 | 15305_DV22_21/23mer | uauuccaccuuugacggcaca | 35 | ugugccgucaaagguggaauagu | 212 |
| XD-60432 | 15305_EEL_19/21mer | uuccaccuuugacggcaca | 36 | tgugccgucaaagguggaaua | 213 |
| XD-60386 | 15305_EEL_21/23mer | uauuccaccuuugacggcaca | 37 | tgugccgucaaagguggaauagu | 214 |
| XD-60330 | 15310_DV22_21/23mer | caccuuugacggcaccucuua | 38 | uaagaggugccgucaaaggugga | 215 |
| XD-60332 | 15312_DV22_21/23mer | ccuuugacggcaccucuuaca | 39 | uguaagaggugccgucaaaggug | 216 |
| XD-60433 | 15312_EEL_19/21mer | uuugacggcaccucuuaca | 40 | tguaagaggugccgucaaagg | 217 |
| XD-60387 | 15312_EEL_21/23mer | ccuuugacggcaccucuuaca | 41 | tguaagaggugccgucaaaggug | 218 |
| XD-60333 | 15314_DV22_21/23mer | uuugacggcaccucuuacaca | 42 | uguguaagaggugccgucaaagg | 219 |
| XD-60434 | 15314_EEL_19/21mer | ugacggcaccucuuacaca | 43 | tguguaagaggugccgucaaa | 220 |
| XD-60388 | 15314_EEL_21/23mer | uuugacggcaccucuuacaca | 44 | tguguaagaggugccgucaaagg | 221 |
| XD-60334 | 15315_DV22_21/23mer | uugacggcaccucuuacacca | 45 | ugguguaagaggugccgucaaag | 222 |
| XD-60335 | 15473_DV22_21/23mer | cacuacaaguccauggauaua | 46 | uauauccauggacuuguagugga | 223 |
| XD-60435 | 15473_EEL_19/21mer | cuacaaguccauggauaua | 47 | tauauccauggacuuguagug | 224 |
| XD-60389 | 15473_EEL_21/23mer | cacuacaaguccauggauaua | 48 | tauauccauggacuuguagugga | 225 |
| XD-60337 | 15478_DV22_21/23mer | caaguccauggauaucgucca | 49 | uggacgauauccauggacuugua | 226 |
| XD-60436 | 15478_EEL_19/21mer | aguccauggauaucgucca | 50 | tggacgauauccauggacuug | 227 |
| XD-60338 | 15479_DV22_21/23mer | aaguccauggauaucguccua | 51 | uaggacgauauccauggacuugu | 228 |
| XD-60437 | 15539_EEL_19/21mer | guuugaccaaauuccggua | 52 | taccggaauuuggucaaacag | 229 |
| XD-60391 | 15539_EEL_21/23mer | cuguuugaccaaauuccggua | 53 | taccggaauuuggucaaacagga | 230 |
| XD-60340 | 15543_DV22_21/23mer | uugaccaaauuccggugagca | 54 | ugcucaccggaauuuggucaaac | 231 |
| XD-60438 | 15543_EEL_19/21mer | gaccaaauuccggugagca | 55 | tgcucaccggaauuuggucaa | 232 |
| XD-60392 | 15543_EEL_21/23mer | uugaccaaauuccggugagca | 56 | tgcucaccggaauuuggucaaac | 233 |
| XD-61288 | 15546_DV22_21/23mer | accaaauuccggugagcagca | 57 | ugcugcucaccggaauuugguca | 234 |
| XD-60341 | 15547_DV22_21/23mer | ccaaauuccggugagcagcga | 58 | ucgcugcucaccggaauuugguc | 235 |
| XD-60439 | 15547_EEL_19/21mer | aaauuccggugagcagcga | 59 | tcgcugcucaccggaauuugg | 236 |
| XD-60393 | 15547_EEL_21/23mer | ccaaauuccggugagcagcga | 60 | tcgcugcucaccggaauuugguc | 237 |
| XD-60344 | 15568_DV22_21/23mer | uuucagcaagaacggcgugca | 61 | ugcacgccguucuugcugaaacc | 238 |
| XD-60345 | 15572_DV22_21/23mer | agcaagaacggcgugcuugua | 62 | uacaagcacgccguucuugcuga | 239 |
| XD-60346 | 15576_DV22_21/23mer | agaacggcgugcuugugucua | 63 | uagacacaagcacgccguucuug | 240 |
| XD-60442 | 15576_EEL_19/21mer | aacggcgugcuugugucua | 64 | tagacacaagcacgccguucu | 241 |
| XD-60396 | 15576_EEL_21/23mer | agaacggcgugcuugugucua | 65 | tagacacaagcacgccguucuug | 242 |
| XD-61281 | 1564_DV22_21/23mer | caacuccaucuacacgcagca | 66 | ugcugcguguagauggaguugag | 243 |
| XD-60347 | 15692_DV22_21/23mer | cucuuccacaacaacaccgaa | 67 | uucgguguuguuguggaagaggc | 244 |
| XD-60348 | 15731_DV22_21/23mer | accaacaaccagagggacgaa | 68 | uucgucccucugguuguuggugc | 245 |
| XD-60275 | 1617_DV22_21/23mer | cacccucgagcuucuucauca | 69 | ugaugaagaagcucgagggugug | 246 |
| XD-61291 | 1617_EEL_19/21mer | cccucgagcuucuucauca | 70 | tgaugaagaagcucgagggug | 247 |
| XD-61289 | 1617_EEL_21/23mer | cacccucgagcuucuucauca | 71 | tgaugaagaagcucgagggugug | 248 |
| XD-60276 | 1618_DV22_21/23mer | acccucgagcuucuucaucga | 72 | ucgaugaagaagcucgagggugu | 249 |
| XD-60277 | 1619_DV22_21/23mer | cccucgagcuucuucaucgua | 73 | uacgaugaagaagcucgagggug | 250 |
| XD-60403 | 1619_EEL_19/21mer | cucgagcuucuucaucgua | 74 | tacgaugaagaagcucgaggg | 251 |
| XD-60357 | 1619_EEL_21/23mer | cccucgagcuucuucaucgua | 75 | tacgaugaagaagcucgagggug | 252 |
| XD-60349 | 16985_DV22_21/23mer | agccucaggaaaaccggcuga | 76 | ucagccgguuuuccugaggcucc | 253 |
| XD-60350 | 17255_DV22_21/23mer | gaugaguguggcugcacgcca | 77 | uggcgugcagccacacucaucca | 254 |
| XD-60278 | 1756_DV22_21/23mer | ccagaaccaggcugacgacua | 78 | uagucgucagccugguucugguu | 255 |
| XD-60279 | 1808_DV22_21/23mer | ggcgcagccuucgccaacaca | 79 | uguguuggcgaaggcugcgcccg | 256 |
| XD-60404 | 1808_EEL_19/21mer | cgcagccuucgccaacaca | 80 | tguguuggcgaaggcugcgcc | 257 |
| XD-60358 | 1808_EEL_21/23mer | ggcgcagccuucgccaacaca | 81 | tguguuggcgaaggcugcgcccg | 258 |
| XD-60280 | 1896_DV22_21/23mer | agaaugagaacuacgcccgga | 82 | uccgggcguaguucucauucucc | 259 |
| XD-60281 | 1897_DV22_21/23mer | gaaugagaacuacgcccggca | 83 | ugccgggguaguucucauucuc | 260 |
| XD-60282 | 2112_DV22_21/23mer | acggcgucugcaccaaguaca | 84 | uguacuuggugcagacgccgucc | 261 |
| XD-60405 | 2112_EEL_19/21mer | ggcgucugcaccaaguaca | 85 | tguacuuggugcagacgccgu | 262 |
| XD-60359 | 2112_EEL_21/23mer | acggcgucugcaccaaguaca | 86 | tguacuuggugcagacgccgucc | 263 |
| XD-60283 | 2113_DV22_21/23mer | cggcgucugcaccaaguacaa | 87 | uuguacuuggugcagacgccguc | 264 |
| XD-60406 | 2113_EEL_19/21mer | gcgucugcaccaaguacaa | 88 | tuguacuuggugcagacgccg | 265 |
| XD-60360 | 2113_EEL_21/23mer | cggcgucugcaccaaguacaa | 89 | tuguacuuggugcagacgccguc | 266 |
| XD-60266 | 215_DV22_21/23mer | gugagcuuuguuccacccgua | 90 | uacggguggaacaaagcucacgc | 267 |
| XD-61282 | 2198_DV22_21/23mer | ggccugagugaggccgacgua | 91 | uacgucggccucacucaggccgc | 268 |
| XD-60284 | 2199_DV22_21/23mer | gccugagugaggccgacguca | 92 | ugacgucggccucacucaggccg | 269 |
| XD-60285 | 2273_DV22_21/23mer | uuccucaaugacgcgggcgca | 93 | ugcgcccgcgucauugaggaagg | 270 |
| XD-60407 | 2273_EEL_19/21mer | ccucaaugacgcggggca | 94 | tgcgcccgcgucauugaggaa | 271 |
| XD-60361 | 2273_EEL_21/23mer | uuccucaaugacgcgggcgca | 95 | tgcgcccgcgucauugaggaagg | 272 |
| XD-60286 | 2275_DV22_21/23mer | ccucaaugacgcggggccua | 96 | uaggcgcccgcgucauugaggaa | 273 |
| XD-60408 | 2275_EEL_19/21mer | ucaaugacgcgggcgccua | 97 | taggcgcccgcgucauugagg | 274 |
| XD-60362 | 2275_EEL_21/23mer | ccucaaugacgcgggcgccua | 98 | taggcgcccgcgucauugaggaa | 275 |
| XD-60287 | 2326_DV22_21/23mer | ucacggcaccgugcuggcuca | 99 | ugagccagcacggugccgugagc | 276 |
| XD-60409 | 2326_EEL_19/21mer | acggcaccgugcuggcuca | 100 | tgagccagcacggugccguga | 277 |
| XD-60363 | 2326_EEL_21/23mer | ucacggcaccgugcuggcuca | 101 | tgagccagcacggugccgugagc | 278 |
| XD-60288 | 2525_DV22_21/23mer | gacgugggcuguuucagcaca | 102 | ugugcugaaacagcccacgucca | 279 |
| XD-60410 | 2525_EEL_19/21mer | cgugggcuguuucagcaca | 103 | tgugcugaaacagcccacguc | 280 |
| XD-60364 | 2525_EEL_21/23mer | gacgugggcuguuucagcaca | 104 | tgugcugaaacagcccacgucca | 281 |
| XD-60289 | 2752_DV22_21/23mer | cggggauggccacuucaucaa | 105 | uugaugaaguggccauccccgua | 282 |
| XD-60290 | 2854_DV22_21/23mer | cgggaccuuccgcaucgucaa | 106 | uugacgaugcggaaggucccgug | 283 |
| XD-60291 | 2874_DV22_21/23mer | ccgagaacauccccuguggga | 107 | ucccacaggggauguucucggug | 284 |
| XD-61283 | 3058_DV22_21/23mer | ggccguguccugggaccggaa | 108 | uuccggucccaggacacggccau | 285 |
| XD-60292 | 3086_DV22_21/23mer | guguucauccgacugcaccaa | 109 | uuggugcagucggaugaacacgc | 286 |
| XD-60411 | 3086_EEL_19/21mer | guucauccgacugcaccaa | 110 | tuggugcagucggaugaacac | 287 |
| XD-60365 | 3086_EEL_21/23mer | guguucauccgacugcaccaa | 111 | tuggugcagucggaugaacacgc | 288 |
| XD-60293 | 3088_DV22_21/23mer | guucauccgacugcaccagga | 112 | uccuggugcagucggaugaacac | 289 |
| XD-60412 | 3088_EEL_19/21mer | ucauccgacugcaccagga | 113 | tccuggugcagucggaugaac | 290 |
| XD-60366 | 3088_EEL_21/23mer | guucauccgacugcaccagga | 114 | tccuggugcagucggaugaacac | 291 |
| XD-60294 | 3090_DV22_21/23mer | ucauccgacugcaccaggaca | 115 | uguccuggugcagucggaugaac | 292 |
| XD-60413 | 3090_EEL_19/21mer | auccgacugcaccaggaca | 116 | tguccuggugcagucggauga | 293 |
| XD-60367 | 3090_EEL_21/23mer | ucauccgacugcaccaggaca | 117 | tguccuggugcagucggaugaac | 294 |
| XD-60295 | 3356_DV22_21/23mer | guugacuccaccaaguacuaa | 118 | uuaguacuugguggagucaaccu | 295 |
| XD-60414 | 3356_EEL_19/21mer | ugacuccaccaaguacuaa | 119 | tuaguacuugguggagucaac | 296 |
| XD-60368 | 3356_EEL_21/23mer | guugacuccaccaaguacuaa | 120 | tuaguacuugguggagucaaccu | 297 |
| XD-60296 | 3360_DV22_21/23mer | acuccaccaaguacuacgaga | 121 | ucucguaguacuugguggaguca | 298 |
| XD-60415 | 3360_EEL_19/21mer | uccaccaaguacuacgaga | 122 | tcucguaguacuugguggagu | 299 |
| XD-60369 | 3360_EEL_21/23mer | acuccaccaaguacuacgaga | 123 | tcucguaguacuugguggaguca | 300 |
| XD-60297 | 3594_DV22_21/23mer | gccggaaccccagugggcaca | 124 | ugugcccacugggguuccggcag | 301 |
| XD-60416 | 3594_EEL_19/21mer | cggaaccccagugggcaca | 125 | tgugcccacugggguuccggc | 302 |
| XD-60370 | 3594_EEL_21/23mer | gccggaaccccagugggcaca | 126 | tgugcccacugggguuccggcag | 303 |
| XD-60298 | 3691_DV22_21/23mer | gaugaagugcguggcccagua | 127 | uacugggccacgcacuucaucug | 304 |
| XD-60299 | 3770_DV22_21/23mer | gagaacugccagagcuguaaa | 128 | uuuacagcucuggcaguucuccg | 305 |
| XD-61292 | 3770_EEL_19/21mer | gaacugccagagcuguaaa | 129 | tuuacagcucuggcaguucuc | 306 |
| XD-61290 | 3770_EEL_21/23mer | gagaacugccagagcuguaaa | 130 | tuuacagcucuggcaguucuccg | 307 |
| XD-60300 | 3771_DV22_21/23mer | agaacugccagagcuguaaca | 131 | uguuacagcucuggcaguucucc | 308 |
| XD-60417 | 3771_EEL_19/21mer | aacugccagagcuguaaca | 132 | tguuacagcucuggcaguucu | 309 |
| XD-60371 | 3771_EEL_21/23mer | agaacugccagagcuguaaca | 133 | tguuacagcucuggcaguucucc | 310 |
| XD-60301 | 3842_DV22_21/23mer | accuaugaggacaggaccuaa | 134 | uuagguccuguccucauaggugc | 311 |
| XD-60302 | 3871_DV22_21/23mer | ggacgucaucuacaacaccaa | 135 | uugguguuguagaugacguccug | 312 |
| XD-60418 | 3871_EEL_19/21mer | acgucaucuacaacaccaa | 136 | tugguguuguagaugacgucc | 313 |
| XD-60372 | 3871_EEL_21/23mer | ggacgucaucuacaacaccaa | 137 | tugguguuguagaugacguccug | 314 |
| XD-60303 | 4057_DV22_21/23mer | uguccgcgaggucugccgcua | 138 | uagcggcagaccucgcggacaca | 315 |
| XD-60419 | 4057_EEL_19/21mer | uccgcgaggucugccgcua | 139 | tagcggcagaccucgcggaca | 316 |
| XD-60373 | 4057_EEL_21/23mer | uguccgcgaggucugccgcua | 140 | tagcggcagaccucgcggacaca | 317 |
| XD-60304 | 4339_DV22_21/23mer | cgaauacgugcccuguggcca | 141 | uggccacagggcacguauucgca | 318 |
| XD-60420 | 4339_EEL_19/21mer | aauacgugcccuguggcca | 142 | tggccacagggcacguauucg | 319 |
| XD-60374 | 4339_EEL_21/23mer | cgaauacgugcccuguggcca | 143 | tggccacagggcacguauucgca | 320 |
| XD-60305 | 4601_DV22_21/23mer | gaggacuaccccaagucugaa | 144 | uucagacuugggguaguccucau | 321 |
| XD-60306 | 4604_DV22_21/23mer | gacuaccccaagucugaacaa | 145 | uuguucagacuugggguaguccu | 322 |
| XD-60307 | 4618_DV22_21/23mer | ugaacaacuuggaggggacga | 146 | ucguccccuccaaguuguucaga | 323 |
| XD-60421 | 4618_EEL_19/21mer | aacaacuuggaggggacga | 147 | tcguccccuccaaguuguuca | 324 |
| XD-60375 | 4618_EEL_21/23mer | ugaacaacuuggaggggacga | 148 | tcguccccuccaaguuguucaga | 325 |
| XD-60308 | 4620_DV22_21/23mer | aacaacuuggaggggacguua | 149 | uaacguccccuccaaguuguuca | 326 |
| XD-60422 | 4620_EEL_19/21mer | caacuuggaggggacguua | 150 | taacguccccuccaaguuguu | 327 |
| XD-60376 | 4620_EEL_21/23mer | aacaacuuggaggggacguua | 151 | taacguccccuccaaguuguuca | 328 |
| XD-60309 | 4633_DV22_21/23mer | ggacguugaguccuacgauaa | 152 | uuaucguaggacucaacgucccc | 329 |
| XD-60377 | 4633_EEL_21/23mer | ggacguugaguccuacgauaa | 153 | tuaucguaggacucaacgucccc | 330 |
| XD-60311 | 4635_DV22_21/23mer | acguugaguccuacgauaaga | 154 | ucuuaucguaggacucaacgucc | 331 |
| XD-60312 | 4641_DV22_21/23mer | aguccuacgauaagaucagga | 155 | uccugaucuuaucguaggacuca | 332 |
| XD-61284 | 4818_DV22_21/23mer | ugugcuacaacuacaggauca | 156 | ugauccuguaguuguagcacauc | 333 |
| XD-60313 | 4826_DV22_21/23mer | aacuacaggauccggguccua | 157 | uaggacccggauccuguaguugu | 334 |
| XD-60267 | 525_DV22_21/23mer | agcugccuuacagccgcacua | 158 | uagugcggcuguaaggcagcucc | 335 |
| XD-60314 | 5277_DV22_21/23mer | cgcugaccacgagccuggcga | 159 | ucgccaggcucguggucagcggc | 336 |
| XD-60425 | 5277_EEL_19/21mer | cugaccacgagccuggcga | 160 | tcgccaggcucguggucagcg | 337 |
| XD-60379 | 5277_EEL_21/23mer | cgcugaccacgagccuggcga | 161 | tcgccaggcucguggucagcggc | 338 |
| XD-60315 | 5425_DV22_21/23mer | guggacagagugguuugacga | 162 | ucgucaaaccacucuguccacuc | 339 |
| XD-61285 | 7169_DV22_21/23mer | cgggaguugggccaggucgua | 163 | uacgaccuggcccaacucccgca | 340 |
| XD-60268 | 734_DV22_21/23mer | cugaccccgcuccaguuugga | 164 | uccaaacuggagggggucagcc | 341 |
| XD-60397 | 734_EEL_19/21mer | gaccccgcuccaguuugga | 165 | tccaaacuggagcggggucag | 342 |
| XD-60351 | 734_EEL_21/23mer | cugaccccgcuccaguuugga | 166 | tccaaacuggagcggggucagcc | 343 |
| XD-60269 | 737_DV22_21/23mer | accccgcuccaguuugggaaa | 167 | uuucccaaacuggagcgggguca | 344 |
| XD-60398 | 737_EEL_19/21mer | cccgcuccaguuugggaaa | 168 | tuucccaaacuggagcggggu | 345 |
| XD-60352 | 737_EEL_21/23mer | accccgcuccaguuugggaaa | 169 | tuucccaaacuggagcgggguca | 346 |
| XD-60270 | 960_DV22_21/23mer | ccuuuguggaauacucacgca | 170 | ugcgugaguauuccacaaaggug | 347 |
| XD-60399 | 960_EEL_19/21mer | uuuguggaauacucacgca | 171 | tgcgugaguauuccacaaagg | 348 |
| XD-60353 | 960_EEL_21/23mer | ccuuuguggaauacucacgca | 172 | tgcgugaguauuccacaaaggug | 349 |
| XD-60271 | 962_DV22_21/23mer | uuuguggaauacucacgccaa | 173 | uuggcgugaguauuccacaaagg | 350 |
| XD-61280 | 966_DV22_21/23mer | uggaauacucacgccagugca | 174 | ugcacuggcgugaguauuccaca | 351 |
| XD-60272 | 972_DV22_21/23mer | acucacgccagugcgcccaca | 175 | ugugggcgcacuggcgugaguau | 352 |
| XD-60400 | 972_EEL_19/21mer | ucacgccagugcgcccaca | 176 | tgugggcgcacuggcgugagu | 353 |
| XD-60354 | 972_EEL_21/23mer | acucacgccagugcgcccaca | 177 | tgugggcgcacuggcgugaguau | 354 |

TABLE 3
Modified Sense and Antisense MUCSB siRNA Sequences

| | | | SEQ | | SEQ |
|---|---|---|---|---|---|
| | | Sense Sequence | ID | Antisense Sequence | ID |
| SIRNA | SIRNA_ID | 5′ to 3′ | NO: | 5′ to 3′ | NO: |
| XD-60355 | 1399_EEL_21/23mer | cagcUacgUUCuguccaaGasa | 355 | dTUCUUGGACAGAaCGUAGCUGcsa | 532 |
| XD-60339 | 15539_DV22_21/23mer | csusguuuGfaCfCfAfaauuccggua | 356 | usAfsccgGfaauuuggUfcAfaacagsgsa | 533 |
| XD-61278 | 214_DV22_21/23mer | csgsugagCfuUfUfGfuuccacccga | 357 | usCfsgggUfggaacaaAfgCfucacgscsg | 534 |
| XD-60426 | 12706_EEL_19/21mer | aaAuccGUGugUucugCusa | 358 | dTAGCAGAACACAcGGAUUUcsa | 535 |
| XD-61279 | 219_DV22_21/23mer | gscsuuugUfuCfCfAfcccgucacua | 359 | usAfsgugAfcggguggAfaCfaaagcsusc | 536 |
| XD-60430 | 15258_EEL_19/21mer | caCuauGAGugCgaguGcsa | 360 | dTGCACUCGCACUCAUAGUGgsa | 537 |
| XD-60380 | 12706_EEL_21/23mer | ugaaAuccGUGuguucugCusa | 361 | dTAGCAGAACACACGGAUUUCAusa | 538 |
| XD-60331 | 15311_DV22_21/23mer | ascscuuuGfaCfGfGfcaccucuuaa | 362 | usUfsaagAfggugccgUfcAfaaggusgsg | 539 |
| XD-60336 | 15476_DV22_21/23mer | usascaagUfcCfAfUfggauaucgua | 363 | usAfscgaUfauccaugGfaCfuuguasgsu | 540 |
| XD-60390 | 15478_EEL_21/23mer | caagUccaUGGauaucguCcsa | 364 | dTGGACGAUAUCCaUGGACUUGusa | 541 |
| XD-60316 | 12616_DV22_21/23mer | gsusugggCfcAfGfGfucguggaaua | 365 | usAfsuucCfacgaccuGfgCfccaacsusc | 542 |
| XD-60317 | 12621_DV22_21/23mer | gscscaggUfcGfUfGfgaaugcagca | 366 | usGfscugCfauuccacGfaCfcuggcscsc | 543 |
| XD-60318 | 12706_DV22_21/23mer | usgsaaauCfcGfUfGfuguucugcua | 367 | usAfsgcaGfaacacacGfgAfuuucasusa | 544 |
| XD-60320 | 12717_DV22_21/23mer | usgsuucuGfcUfGfCfaacuacggca | 368 | usGfsccgUfaguugcaGfcAfgaacascsa | 545 |
| XD-60319 | 13206_DV22_21/23mer | cscsuccaCfuCfCfGfgggacgacca | 369 | usGfsgucGfuccccggAfgUfggaggsasg | 546 |
| XD-60273 | 1399_DV22_21/23mer | csasgcuaCfgUfUfCfuguccaagaa | 370 | usUfscuuGfgacagaaCfgUfagcugscsa | 547 |
| XD-60321 | 15132_DV22_21/23mer | gscsacggUfgGfCfCfaggugcguga | 371 | usCfsacgCfaccuggcCfaCfcgugcsasg | 548 |
| XD-60322 | 15140_DV22_21/23mer | gscscaggUfgCfGfUfgggugacaaa | 372 | usUfsuguCfacccacgCfaCfcuggcscsa | 549 |
| XD-60427 | 15140_EEL_19/21mer | caGgugCGUggGugacAasa | 373 | dTUUGUCACCCACgCACCUGgsc | 550 |
| XD-60381 | 15140_EEL_21/23mer | gccaGgugCGUgggugacAasa | 374 | dTUUGUCACCCACgCACCUGGCcsa | 551 |
| XD-60323 | 15197_DV22_21/23mer | gsuscaccUfgCfGfUfgaacaagcaa | 375 | usUfsgcuUfguucacgCfaGfgugacsgsu | 552 |
| XD-60428 | 15197_EEL_19/21mer | caCcugCGUgaAcaagCasa | 376 | dTUGCUUGUUCACgCAGGUGasc | 553 |
| XD-60382 | 15197_EEL_21/23mer | gucaCcugCGUgaacaagCasa | 377 | dTUGCUUGUUCACgCAGGUGACgsu | 554 |
| XD-60324 | 15252_DV22_21/23mer | gsusgacuUfcCfAfCfuaugagugca | 378 | usGfscacUfcauagugGfaAfgucacsasg | 555 |
| XD-60429 | 15252_EEL_19/21mer | gaCuucCACuaUgaguGcsa | 379 | dTGCACUCAUAGUgGAAGUCasc | 556 |
| XD-60383 | 15252_EEL_21/23mer | gugaCuucCACuaugaguGcsa | 380 | dTGCACUCAUAGUgGAAGUCACasg | 557 |
| XD-60325 | 15254_DV22_21/23mer | gsascuucCfaCfUfAfugagugcgaa | 381 | usUfscgcAfcucauagUfgGfaagucsasc | 558 |
| XD-60326 | 15258_DV22_21/23mer | uscscacuAfuGfAfGfugcgagugca | 382 | usGfscacUfcgcacucAfuAfguggasasg | 559 |
| XD-60384 | 15258_EEL_21/23mer | uccaCuauGAGugcgaguGcsa | 383 | dTGCACUCGCACUCAUAGUGGAasg | 560 |
| XD-60327 | 15261_DV22_21/23mer | ascsuaugAfgUfGfCfgagugcauca | 384 | usGfsaugCfacucgcaCfuCfauagusgsg | 561 |
| XD-60431 | 15261_EEL_19/21mer | uaUgagUGCgaGugcaUcsa | 385 | dTGAUGCACUCGCaCUCAUAgsu | 562 |
| XD-60385 | 15261_EEL_21/23mer | acuaUgagUGCgagugcaUcsa | 386 | dTGAUGCACUCGCaCUCAUAGUgsg | 563 |
| XD-61286 | 15262_DV22_21/23mer | csusaugaGfuGfCfGfagugcaucua | 387 | usAfsgauGfcacucgcAfcUfcauagsusg | 564 |
| XD-60328 | 15304_DV22_21/23mer | csusauucCfaCfCfUfuugacggcaa | 388 | usUfsgccGfucaaaggUfgGfaauagsusg | 565 |
| XD-60329 | 15305_DV22_21/23mer | usasuuccAfcCfUfUfugacggcaca | 389 | usGfsugcCfgucaaagGfuGfgaauasgsu | 566 |
| XD-60432 | 15305_EEL_19/21mer | uuCcacCUUugAcggcAcsa | 390 | dTGUGCCGUCAAAgGUGGAAusa | 567 |
| XD-60386 | 15305_EEL_21/23mer | uauuCcacCUUugacggcAcsa | 391 | dTGUGCCGUCAAAgGUGGAAUAgsu | 568 |
| XD-60330 | 15310_DV22_21/23mer | csasccuuUfgAfCfGfgcaccucuua | 392 | usAfsagaGfgugccguCfaAfaggugsgsa | 569 |
| XD-60332 | 15312_DV22_21/23mer | cscsuuugAfcGfGfCfaccucuuaca | 393 | usGfsuaaGfaggugccGfuCfaaaggsusg | 570 |
| XD-60433 | 15312_EEL_19/21mer | uuUgacGGCacCucuuAcsa | 394 | dTGUAAGAGGUGCcGUCAAAgsg | 571 |
| XD-60387 | 15312_EEL_21/23mer | ccuuUgacGGCaccucuuAcsa | 395 | dTGUAAGAGGUGCCGUCAAAGGusg | 572 |
| XD-60333 | 15314_DV22_21/23mer | ususugacGfgCfAfCfcucuuacaca | 396 | usGfsuguAfagaggugCfcGfucaaasgsg | 573 |
| XD-60434 | 15314_EEL_19/21mer | ugAcggCACcuCuuacAcsa | 397 | dTGUGUAAGAGGUgCCGUCAasa | 574 |
| XD-60388 | 15314_EEL_21/23mer | uuugAcggCACcucuuacAcsa | 398 | dTGUGUAAGAGGUgCCGUCAAAgsg | 575 |
| XD-60334 | 15315_DV22_21/23mer | ususgacgGfcAfCfCfucuuacacca | 399 | usGfsgugUfaagagguGfcCfgucaasasg | 576 |
| XD-60335 | 15473_DV22_21/23mer | csascuacAfaGfUfCfcauggauaua | 400 | usAfsuauCfcauggacUfuGfuagugsgsa | 577 |
| XD-60435 | 15473_EEL_19/21mer | cuAcaaGUCcaUggauAusa | 401 | dTAUAUCCAUGGACUUGUAGusg | 578 |
| XD-60389 | 15473_EEL_21/23mer | cacuAcaaGUCcauggauAusa | 402 | dTAUAUCCAUGGACUUGUAGUGgsa | 579 |
| XD-60337 | 15478_DV22_21/23mer | csasagucCfaUfGfGfauaucgucca | 403 | usGfsgacGfauauccaUfgGfacuugsusa | 580 |
| XD-60436 | 15478_EEL_19/21mer | agUccaUGGauAucguCcsa | 404 | dTGGACGAUAUCCaUGGACUusg | 581 |
| XD-60338 | 15479_DV22_21/23mer | asasguccAfuGfGfAfuaucguccua | 405 | usAfsggaCfgauauccAfuGfgacuusgsu | 582 |
| XD-60437 | 15539_EEL_19/21mer | guUugaCCAaaUuccgGusa | 406 | dTACCGGAAUUUGgUCAAACasg | 583 |
| XD-60391 | 15539_EEL_21/23mer | cuguUugaCCAaauuccgGusa | 407 | dTACCGGAAUUUGgUCAAACAGgsa | 584 |
| XD-60340 | 15543_DV22_21/23mer | ususgaccAfaAfUfUfccggugagca | 408 | usGfscucAfccggaauUfuGfgucaasasc | 585 |
| XD-60438 | 15543_EEL_19/21mer | gaCcaaAUUccGgugaGcsa | 409 | dTGCUCACCGGAAuUUGGUCasa | 586 |
| XD-60392 | 15543_EEL_21/23mer | uugaCcaaAUUccggugaGcsa | 410 | dTGCUCACCGGAAuUUGGUCAAasc | 587 |
| XD-61288 | 15546_DV22_21/23mer | ascscaaaUfuCfCfGfgugagcagca | 411 | usGfscugCfucaccggAfaUfuugguscsa | 588 |
| XD-60341 | 15547_DV22_21/23mer | cscsaaauUfcCfGfGfugagcagcga | 412 | usCfsgcuGfcucaccgGfaAfuuuggsusc | 589 |
| XD-60439 | 15547_EEL_19/21mer | aaAuucCGGugAgcagCgsa | 413 | dTCGCUGCUCACCgGAAUUUgsg | 590 |
| XD-60393 | 15547_EEL_21/23mer | ccaaAuucCGGugagcagCgsa | 414 | dTCGCUGCUCACCgGAAUUUGGusc | 591 |
| XD-60344 | 15568_DV22_21/23mer | ususucagCfaAfGfAfacggcgugca | 415 | usGfscacGfccguucuUfgCfugaaascsc | 592 |
| XD-60345 | 15572_DV22_21/23mer | asgscaagAfaCfGfGfcgugcuugua | 416 | usAfscaaGfcacgccgUfuCfuugcusgsa | 593 |
| XD-60346 | 15576_DV22_21/23mer | asgsaacgGfcGfUfGfcuugugucua | 417 | usAfsgacAfcaagcacGfcCfguucususg | 594 |
| XD-60442 | 15576_EEL_19/21mer | aaCggcGUGcuUguguCusa | 418 | dTAGACACAAGCAcGCCGUUcsu | 595 |
| XD-60396 | 15576_EEL_21/23mer | agaaCggcGUGcuuguguCusa | 419 | dTAGACACAAGCAcGCCGUUCUusg | 59€ |
| XD-61281 | 1564_DV22_21/23mer | csasacucCfaUfCfUfacacgcagca | 420 | usGfscugCfguguagaUfgGfaguugsasg | 597 |
| XD-60347 | 15692_DV22_21/23mer | csuscuucCfaCfAfAfcaacaccgaa | 421 | usUfscggUfguuguugUfgGfaagagsgsc | 598 |
| XD-60348 | 15731_DV22_21/23mer | ascscaacAfaCfCfAfgagggacgaa | 422 | usUfscguCfccucuggUfuGfuuggusgsc | 599 |
| XD-60275 | 1617_DV22_21/23mer | csascccuCfgAfGfCfuucuucauca | 423 | usGfsaugAfagaagcuCfgAfgggugsusg | 600 |
| XD-61291 | 1617_EEL_19/21mer | ccCucgAGCuuCuucaUcsa | 424 | dTGAUGAAGAAGCuCGAGGGusg | 601 |
| XD-61289 | 1617_EEL_21/23mer | caccCucgAGCuucuucaUcsa | 425 | dTGAUGAAGAAGCuCGAGGGUGusg | 602 |
| XD-60276 | 1618_DV22_21/23mer | ascsccucGfaGfCfUfucuucaucga | 426 | usCfsgauGfaagaagcUfcGfagggusgsu | 603 |
| XD-60277 | 1619_DV22_21/23mer | cscscucgAfgCfUfUfcuucaucgua | 427 | usAfscgaUfgaagaagCfuCfgagggsusg | 604 |
| XD-60403 | 1619_EEL_19/21mer | cuCgagCUUcuUcaucGusa | 428 | dTACGAUGAAGAAgCUCGAGgsg | 605 |
| XD-60357 | 1619_EEL_21/23mer | cccuCgagCUUcuucaucGusa | 429 | dTACGAUGAAGAAgCUCGAGGGusg | 606 |
| XD-60349 | 16985_DV22_21/23mer | asgsccucAfgGfAfAfaaccggcuga | 430 | usCfsagcCfgguuuucCfuGfaggcuscsc | 607 |
| XD-60350 | 17255_DV22_21/23mer | gsasugagUfgUfGfGfcugcacgcca | 431 | usGfsgcgUfgcagccaCfaCfucaucscsa | 608 |
| XD-60278 | 1756_DV22_21/23mer | cscsagaaCfcAfGfGfcugacgacua | 432 | usAfsgucGfucagccuGfgUfucuggsusu | 609 |
| XD-60279 | 1808_DV22_21/23mer | gsgscgcaGfcCfUfUfcgccaacaca | 433 | usGfsuguUfggcgaagGfcUfgcgccscsg | 610 |
| XD-60404 | 1808_EEL_19/21mer | cgCagcCUUcgCcaacAcsa | 434 | dTGUGUUGGCGAAgGCUGCGcsc | 611 |
| XD-60358 | 1808_EEL_21/23mer | ggcgCagcCUUcgccaacAcsa | 435 | dTGUGUUGGCGAAgGCUGCGCCcsg | 612 |
| XD-60280 | 1896_DV22_21/23mer | asgsaaugAfgAfAfCfuacgcccgga | 436 | usCfscggGfcguaguuCfuCfauucuscsc | 613 |
| XD-60281 | 1897_DV22_21/23mer | gsasaugaGfaAfCfUfacgcccggca | 437 | usGfsccgGfgcguaguUfcUfcauucsusc | 614 |
| XD-60282 | 2112_DV22_21/23mer | ascsggcgUfcUfGfCfaccaaguaca | 438 | usGfsuacUfuggugcaGfaCfgccguscsc | 615 |
| XD-60405 | 2112_EEL_19/21mer | ggCgucUGCacCaaguAcsa | 439 | dTGUACUUGGUGCaGACGCCgsu | 616 |
| XD-60359 | 2112_EEL_21/23mer | acggCgucUGCaccaaguAcsa | 440 | dTGUACUUGGUGCaGACGCCGUcsc | 617 |
| XD-60283 | 2113_DV22_21/23mer | csgsgcguCfuGfCfAfccaaguacaa | 441 | usUfsguaCfuuggugcAfgAfcgccgsusc | 618 |
| XD-60406 | 2113_EEL_19/21mer | gcGucuGCAccAaguaCasa | 442 | dTUGUACUUGGUGcAGACGCcsg | 619 |
| XD-60360 | 2113_EEL_21/23mer | cggcGucuGCAccaaguaCasa | 443 | dTUGUACUUGGUGcAGACGCCGusc | 620 |
| XD-60266 | 215_DV22_21/23mer | gsusgagcUfuUfGfUfuccacccgua | 444 | usAfscggGfuggaacaAfaGfcucacsgsc | 621 |
| XD-61282 | 2198_DV22_21/23mer | gsgsccugAfgUfGfAfggccgacgua | 445 | usAfscguCfggccucaCfuCfaggccsgsc | 622 |
| XD-60284 | 2199_DV22_21/23mer | gscscugaGfuGfAfGfgccgacguca | 446 | usGfsacgUfcggccucAfcUfcaggcscsg | 623 |
| XD-60285 | 2273_DV22_21/23mer | ususccucAfaUfGfAfcgcgggcgca | 447 | usGfscgcCfcgcgucaUfuGfaggaasgsg | 624 |
| XD-60407 | 2273_EEL_19/21mer | ccUcaaUGAcgCgggcGcsa | 448 | dTGCGCCCGCGUCaUUGAGGasa | 625 |
| XD-60361 | 2273_EEL_21/23mer | uuccUcaaUGAcgcgggcGcsa | 449 | dTGCGCCCGCGUCaUUGAGGAAgsg | 626 |
| XD-60286 | 2275_DV22_21/23mer | cscsucaaUfgAfCfGfcgggcgccua | 450 | usAfsggcGfcccgcguCfaUfugaggsasa | 627 |
| XD-60408 | 2275_EEL_19/21mer | ucAaugACGcgGgcgcCusa | 451 | dTAGGCGCCCGCGuCAUUGAgsg | 628 |
| XD-60362 | 2275_EEL_21/23mer | ccucAaugACGcgggcgcCusa | 452 | dTAGGCGCCCGCGuCAUUGAGGasa | 629 |
| XD-60287 | 2326_DV22_21/23mer | uscsacggCfaCfCfGfugcuggcuca | 453 | usGfsagcCfagcacggUfgCfcgugasgsc | 630 |
| XD-60409 | 2326_EEL_19/21mer | acGgcaCCGugCuggcUcsa | 454 | dTGAGCCAGCACGgUGCCGUgsa | 631 |
| XD-60363 | 2326_EEL_21/23mer | ucacGgcaCCGugcuggcUcsa | 455 | dTGAGCCAGCACGgUGCCGUGAgsc | 632 |
| XD-60288 | 2525_DV22_21/23mer | gsascgugGfgCfUfGfuuucagcaca | 456 | usGfsugcUfgaaacagCfcCfacgucscsa | 633 |
| XD-60410 | 2525_EEL_19/21mer | cgUgggCUGuuUcagcAcsa | 457 | dTGUGCUGAAACAgCCCACGusc | 634 |
| XD-60364 | 2525_EEL_21/23mer | gacgUgggCUGuuucagcÅcsa | 458 | dTGUGCUGAAACAgCCCACGUCcsa | 635 |
| XD-60289 | 2752_DV22_21/23mer | csgsgggaUfgGfCfCfacuucaucaa | 459 | usUfsgauGfaaguggcCfaUfccccgsusa | 636 |
| XD-60290 | 2854_DV22_21/23mer | csgsggacCfuUfCfCfgcaucgucaa | 460 | usUfsgacGfaugcggaAfgGfucccgsusg | 637 |
| XD-60291 | 2874_DV22_21/23mer | cscsgagaAfcAfUfCfcccuguggga | 461 | usCfsccaCfaggggauGfuUfcucggsusg | 638 |
| XD-61283 | 3058_DV22_21/23mer | gsgsccguGfuCfCfUfgggaccggaa | 462 | usUfsccgGfucccaggAfcAfcggccsasu | 639 |
| XD-60292 | 3086_DV22_21/23mer | gsusguucAfuCfCfGfacugcaccaa | 463 | usUfsgguGfcagucggAfuGfaacacsgsc | 640 |
| XD-60411 | 3086_EEL_19/21mer | guUcauCCGacUgcacCasa | 464 | dTUGGUGCAGUCGgAUGAACasc | 641 |
| XD-60365 | 3086_EEL_21/23mer | guguUcauCCGacugcacCasa | 465 | dTUGGUGCAGUCGgAUGAACACgsc | 642 |
| XD-60293 | 3088_DV22_21/23mer | gsusucauCfcGfAfCfugcaccagga | 466 | usCfscugGfugcagucGfgAfugaacsasc | 643 |
| XD-60412 | 3088_EEL_19/21mer | ucAuccGACugCaccaGgsa | 467 | dTCCUGGUGCAGUcGGAUGAasc | 644 |
| XD-60366 | 3088_EEL_21/23mer | guucAuccGACugcaccaGgsa | 468 | dTCCUGGUGCAGUCGGAUGAACasc | 645 |
| XD-60294 | 3090_DV22_21/23mer | uscsauccGfaCfUfGfcaccaggaca | 469 | usGfsuccUfggugcagUfcGfgaugasasc | 646 |
| XD-60413 | 3090_EEL_19/21mer | auCcgaCUGcaCcaggAcsa | 470 | dTGUCCUGGUGCAgUCGGAUgsa | 647 |
| XD-60367 | 3090_EEL_21/23mer | ucauCcgaCUGcaccaggAcsa | 471 | dTGUCCUGGUGCAgUCGGAUGAasc | 648 |
| XD-60295 | 3356_DV22_21/23mer | gsusugacUfcCfAfCfcaaguacuaa | 472 | usUfsaguAfcuuggugGfaGfucaacscsu | 649 |
| XD-60414 | 3356_EEL_19/21mer | ugAcucCACcaAguacUasa | 473 | dTUAGUACUUGGUgGAGUCAasc | 650 |
| XD-60368 | 3356_EEL_21/23mer | guugAcucCACcaaguacUasa | 474 | dTUAGUACUUGGUgGAGUCAACcsu | 651 |
| XD-60296 | 3360_DV22_21/23mer | ascsuccaCfcAfAfGfuacuacgaga | 475 | usCfsucgUfaguacuuGfgUfggaguscsa | 652 |
| XD-60415 | 3360_EEL_19/21mer | ucCaccAAGuaCuacgAgsa | 476 | dTCUCGUAGUACUuGGUGGAgsu | 653 |
| XD-60369 | 3360_EEL_21/23mer | acucCaccAAGuacuacgAgsa | 477 | dTCUCGUAGUACUUGGUGGAGUcsa | 654 |
| XD-60297 | 3594_DV22_21/23mer | gscscggaAfcCfCfCfagugggcaca | 478 | usGfsugcCfcacugggGfuUfccggcsasg | 655 |
| XD-60416 | 3594_EEL_19/21mer | cgGaacCCCagUgggcAcsa | 479 | dTGUGCCCACUGGgGUUCCGgsc | 656 |
| XD-60370 | 3594_EEL_21/23mer | gccgGaacCCCagugggcAcsa | 480 | dTGUGCCCACUGGgGUUCCGGCasg | 657 |
| XD-60298 | 3691_DV22_21/23mer | gsasugaaGfuGfCfGfuggcccagua | 481 | usAfscugGfgccacgcAfcUfucaucsusg | 658 |
| XD-60299 | 3770_DV22_21/23mer | gsasgaacUfgCfCfAfgagcuguaaa | 482 | usUfsuacAfgcucuggCfaGfuucucscsg | 659 |
| XD-61292 | 3770_EEL_19/21mer | gaAcugCCAgaGcuguAasa | 483 | dTUUACAGCUCUGgCAGUUCusc | 660 |
| XD-61290 | 3770_EEL_21/23mer | gagaAcugCCAgagcuguAasa | 484 | dTUUACAGCUCUGgCAGUUCUCcsg | 661 |
| XD-60300 | 3771_DV22_21/23mer | asgsaacuGfcCfAfGfagcuguaaca | 485 | usGfsuuaCfagcucugGfcAfguucuscsc | 662 |
| XD-60417 | 3771_EEL_19/21mer | aaCugcCAGagCuguaAcsa | 486 | dTGUUACAGCUCUgGCAGUUcsu | 663 |
| XD-60371 | 3771_EEL_21/23mer | agaaCugcCAGagcuguaAcsa | 487 | dTGUUACAGCUCUgGCAGUUCUcsc | 664 |
| XD-60301 | 3842_DV22_21/23mer | ascscuauGfaGfGfAfcaggaccuaa | 488 | usUfsaggUfccuguccUfcAfuaggusgsc | 665 |
| XD-60302 | 3871_DV22_21/23mer | gsgsacguCfaUfCfUfacaacaccaa | 489 | usUfsgguGfuuguagaUfgAfcguccsusg | 666 |
| XD-60418 | 3871_EEL_19/21mer | acGucaUCUacAacacCasa | 490 | dTUGGUGUUGUAGaUGACGUcsc | 667 |
| XD-60372 | 3871_EEL_21/23mer | ggacGucaUCUacaacacCasa | 491 | dTUGGUGUUGUAGaUGACGUCCusg | 668 |
| XD-60303 | 4057_DV22_21/23mer | usgsuccgCfgAfGfGfucugccgcua | 492 | usAfsgcgGfcagaccuCfgCfggacascsa | 669 |
| XD-60419 | 4057_EEL_19/21mer | ucCgcgAGGucUgccgCusa | 493 | dTAGCGGCAGACCUCGCGGAcsa | 670 |
| XD-60373 | 4057_EEL_21/23mer | ugucCgcgAGGucugccgCusa | 494 | dTAGCGGCAGACCUCGCGGACAcsa | 671 |
| XD-60304 | 4339_DV22_21/23mer | csgsaauaCfgUfGfCfccuguggcca | 495 | usGfsgccAfcagggcaCfgUfauucgscsa | 672 |
| XD-60420 | 4339_EEL_19/21mer | aaUacgUGCccUguggCcsa | 496 | dTGGCCACAGGGCaCGUAUUcsg | 673 |
| XD-60374 | 4339_EEL_21/23mer | cgaaUacgUGCccuguggCcsa | 497 | dTGGCCACAGGGCaCGUAUUCGcsa | 674 |
| XD-60305 | 4601_DV22_21/23mer | gsasggacUfaCfCfCfcaagucugaa | 498 | usUfscagAfcuuggggUfaGfuccucsasu | 675 |
| XD-60306 | 4604_DV22_21/23mer | gsascuacCfcCfAfAfgucugaacaa | 499 | usUfsguuCfagacuugGfgGfuagucscsu | 676 |
| XD-60307 | 4618_DV22_21/23mer | usgsaacaAfcUfUfGfgaggggacga | 500 | usCfsgucCfccuccaaGfuUfguucasgsa | 677 |
| XD-60421 | 4618_EEL_19/21mer | aaCaacUUGgaGgggaCgsa | 501 | dTCGUCCCCUCCAaGUUGUUcsa | 678 |
| XD-60375 | 4618_EEL_21/23mer | ugaaCaacUUGgaggggaCgsa | 502 | dTCGUCCCCUCCAaGUUGUUCAgsa | 679 |
| XD-60308 | 4620_DV22_21/23mer | asascaacUfuGfGfAfggggacguua | 503 | usAfsacgUfccccuccAfaGfuuguuscsa | 680 |
| XD-60422 | 4620_EEL_19/21mer | caAcuuGGAggGgacgUusa | 504 | dTAACGUCCCCUCCAAGUUGusu | 681 |
| XD-60376 | 4620_EEL_21/23mer | aacaAcuuGGAggggacgUusa | 505 | dTAACGUCCCCUCCAAGUUGUUcsa | 682 |
| XD-60309 | 4633_DV22_21/23mer | gsgsacguUfgAfGfUfccuacgauaa | 506 | usUfsaucGfuaggacuCfaAfcguccscsc | 683 |
| XD-60377 | 4633_EEL_21/23mer | ggacGuugAGUccuacgaUasa | 507 | dTUAUCGUAGGACUCAACGUCCCSC | 684 |
| XD-60311 | 4635_DV22_21/23mer | ascsguugAfgUfCfCfuacgauaaga | 508 | usCfsuuaUfcguaggaCfuCfaacguscsc | 685 |
| XD-60312 | 4641_DV22_21/23mer | asgsuccuAfcGfAfUfaagaucagga | 509 | usCfscugAfucuuaucGfuAfggacuscsa | 686 |
| XD-61284 | 4818_DV22_21/23mer | usgsugcuAfcAfAfCfuacaggauca | 510 | usGfsaucCfuguaguuGfuAfgcacasusc | 687 |
| XD-60313 | 4826_DV22_21/23mer | asascuacAfgGfAfUfccggguccua | 511 | usAfsggaCfccggaucCfuGfuaguusgsu | 688 |
| XD-60267 | 525_DV22_21/23mer | asgscugcCfuUfAfCfagccgcacua | 512 | usAfsgugCfggcuguaAfgGfcagcuscsc | 689 |
| XD-60314 | 5277_DV22_21/23mer | csgscugaCfcAfCfGfagccuggcga | 513 | usCfsgccAfggcucguGfgUfcagcgsgsc | 690 |
| XD-60425 | 5277_EEL_19/21mer | cuGaccACGagCcuggCgsa | 514 | dTCGCCAGGCUCGuGGUCAGcsg | 691 |
| XD-60379 | 5277_EEL_21/23mer | cgcuGaccACGagccuggOgsa | 515 | dTCGCCAGGCUCGuGGUCAGCGgsc | 692 |
| XD-60315 | 5425_DV22_21/23mer | gsusggacAfgAfGfUfgguuugacga | 516 | usCfsgucAfaaccacuCfuGfuccacsusc | 693 |
| XD-61285 | 7169_DV22_21/23mer | csgsggagUfuGfGfGfccaggucgua | 517 | usAfscgaCfcuggcccAfaCfucccgscsa | 694 |
| XD-60268 | 734_DV22_21/23mer | csusgaccCfcGfCfUfccaguuugga | 518 | usCfscaaAfcuggagcGfgGfgucagscsc | 695 |
| XD-60397 | 734_EEL_19/21mer | gaCcccGCUccAguuuGgsa | 519 | dTCCAAACUGGAGcGGGGUCasg | 696 |
| XD-60351 | 734_EEL_21/23mer | cugaCcccGCUccaguuuGgsa | 520 | dTCCAAACUGGAGGGGGGUCAGcsc | 697 |
| XD-60269 | 737_DV22_21/23mer | ascscccgCfuCfCfAfguuugggaaa | 521 | usUfsuccCfaaacuggAfgCfgggguscsa | 698 |
| XD-60398 | 737_EEL_19/21mer | ccCgcuCCAguUugggAasa | 522 | dTUUCCCAAACUGgAGCGGGgsu | 699 |
| XD-60352 | 737_EEL_21/23mer | acccCgcuCCAguuugggAasa | 523 | dTUUCCCAAACUGgAGCGGGGUcsa | 700 |
| XD-60270 | 960_DV22_21/23mer | cscsuuugUfgGfAfAfuacucacgca | 524 | usGfscguGfaguauucCfaCfaaaggsusg | 701 |
| XD-60399 | 960_EEL_19/21mer | uuUgugGAAuaCucacGcsa | 525 | dTGCGUGAGUAUUcCACAAAgsg | 702 |
| XD-60353 | 960_EEL_21/23mer | ccuuUgugGAAuacucacGcsa | 526 | dTGCGUGAGUAUUcCACAAAGGusg | 703 |
| XD-60271 | 962_DV22_21/23mer | ususugugGfaAfUfAfcucacgccaa | 527 | usUfsggcGfugaguauUfcCfacaaasgsg | 704 |
| XD-61280 | 966_DV22_21/23mer | usgsgaauAfcUfCfAfcgccagugca | 528 | usGfscacUfggcgugaGfuAfuuccascsa | 705 |
| XD-60272 | 972_DV22_21/23mer | ascsucacGfcCfAfGfugcgcccaca | 529 | usGfsuggGfcgcacugGfcGfugagusasu | 706 |
| XD-60400 | 972_EEL_19/21mer | ucAcgcCAGugCgcccAcsa | 530 | dTGUGGGCGCACUgGCGUGAgsu | 707 |
| XD-60354 | 972_EEL_21/23mer | acucAcgcCAGugcgcccAcsa | 531 | dTGUGGGCGCACUgGCGUGAGUasu | 708 |

TABLE 4
Unmodified MUCSB ASO Sequences

| ASO ID | Sequence | SEQ ID NO: |
|---|---|---|
| X164897 | GTTGTAGATGACGTCCT | 709 |
| X164902 | TAGTCGTGACAGAGCGG | 710 |
| X164915 | CCGTAGTTGCAGCAGAA | 711 |
| X164916 | GCCGTAGTTGCAGCAGA | 712 |
| X164924 | GGTGTAAGAGGTGCCGT | 713 |
| X164926 | GGACGATATCCATGGAC | 714 |
| X164845 | AAAGCCCATGGTCGGCT | 715 |
| X164848 | GCGTGAGTATTCCACAA | 716 |
| X164849 | CGCACTGGCGTGAGTAT | 717 |
| X164850 | GCGCACTGGCGTGAGTA | 718 |
| X164851 | TTGGACAGAACGTAGCT | 719 |
| X164852 | CCTTGGACAGAACGTAG | 720 |
| X164853 | ACCTTGGACAGAACGTA | 721 |
| X164854 | GTTCTCGTTGTCCGTCA | 722 |
| X164855 | AGGCAGTTCTCGTTGTC | 723 |
| X164856 | AGCTCGAGGGTGTGAAC | 724 |
| X164857 | TGAAGAAGCTCGAGGGT | 725 |
| X164858 | CGATGAAGAAGCTCGAG | 726 |
| X164859 | ACGATGAAGAAGCTCGA | 727 |
| X164860 | TGGCGCTAGTCCAGGCC | 728 |
| X164861 | AGCCTGGCGCTAGTCCA | 729 |
| X164862 | GCAGCCTGGCGCTAGTC | 730 |
| X164863 | TGTAGGTGGCCTCGTTG | 731 |
| X164864 | GCAGTCGACCCTGATGG | 732 |
| X164865 | GTGTTGCAGTCGACCCT | 733 |
| X164866 | GGTGTTGCAGTCGACCC | 734 |
| X164867 | CAGGTGTTGCAGTCGAC | 735 |
| X164868 | CCGTAGGCCACGCAGGT | 736 |
| X164869 | CCCGTAGGCCACGCAGG | 737 |
| X164870 | CCCCGTAGGCCACGCAG | 738 |
| X164871 | TCCCCGTAGGCCACGCA | 739 |
| X164872 | ATCCCCGTAGGCCACGC | 740 |
| X164873 | CATCCCCGTAGGCCACG | 741 |
| X164874 | ATGCGGAAGGTCCCGTG | 742 |
| X164875 | GÅCGATGCGGAAGGTCC | 743 |
| X164876 | CTCGGTGACGATGCGGA | 744 |
| X164877 | GGATGTTCTCGGTGACG | 745 |
| X164878 | GGTGCGCACGGGCTCTT | 746 |
| X164879 | AAGATCCCCATGTAGCG | 747 |
| X164880 | TCCGGTCCCAGGACACG | 748 |
| X164881 | GGTGCAGTOGGATGAAC | 749 |
| X164882 | TGGTGCAGTCGGATGAA | 750 |
| X164883 | GTGTGCCGTGGGACATG | 751 |
| X164884 | CTCGTAGTACTTGGTGG | 752 |
| X164885 | GTCCGGAGTCCGCCAGG | 753 |
| X164886 | TGTCCGGAGTCCGCCAG | 754 |
| X164887 | GTGTCCGGAGTCCGCCA | 755 |
| X164888 | ACGGCAGGTGTCCGGAG | 756 |
| X164889 | CACGGCAGGTGTCCGGA | 757 |
| X164890 | CCCGACTCACGGCAGGT | 758 |
| X164891 | ACGGACAGAGCCCGACT | 759 |
| X164892 | ATGCGTGTTGGATGTCT | 760 |
| X164893 | ACATGCGTGTTGGATGT | 761 |
| X164894 | GGCACATGCGTGTTGGA | 762 |
| X164895 | TCGGGTAGCAGCCTAGG | 763 |
| X164896 | TTGTAGATGACGTCCTG | 764 |
| X164898 | GACCTCGCGGACACACA | 765 |
| X164899 | AGACCTCGCGGACACAC | 766 |
| X164900 | CGGCAGACCTCGCGGAC | 767 |
| X164901 | TTGGCGCACATCAGCCC | 768 |
| X164903 | GGCCACAGGGCACGTAT | 769 |
| X164904 | TATCGTAGGACTCAACG | 770 |
| X164905 | TTATCGTAGGACTCAAC | 771 |
| X164906 | CTGATCTTATCGTAGGA | 772 |
| X164907 | CCTGATCTTATCGTAGG | 773 |
| X164908 | GCCCTGATCTTATCGTA | 774 |
| X164909 | CGGATCCTGTAGTTGTA | 775 |
| X164910 | GACCCGGATCCTGTAGT | 776 |
| X164911 | GGACCCGGATCCTGTAG | 777 |
| X164912 | CGGGGTAGTTCTCCGCC | 778 |
| X164913 | TCGGGGTAGTTCTCCGC | 779 |
| X164914 | ACCTCGGGGTAGTTCTC | 780 |
| X164917 | TTGTTCACGCAGGTGAC | 781 |
| X164918 | CTTGTTCACGCAGGTGA | 782 |
| X164919 | GGTGCTTGTTCACGCAG | 783 |
| X164920 | CACTCGCACTCATAGTG | 784 |
| X164921 | GAAGCCGCTGTTAGGAC | 785 |
| X164922 | GTAAGAGGTGCCGTCAA | 786 |
| X164923 | GTGTAAGAGGTGCCGTC | 787 |
| X164925 | GACGATATCCATGGACT | 788 |
| X164927 | GTGAGGACGATATCCAT | 789 |
| X164928 | GCTCACCGGAATTTGGT | 790 |
| X164929 | CGCTGCTCACCGGAATT | 791 |
| X164930 | CCGCTGCTCACCGGAAT | 792 |
| X164931 | CACGCCGTTCTTGCTGA | 793 |
| X164932 | GCACGCCGTTCTTGCTG | 794 |
| X164933 | GTTGCACCTCGCCAGTC | 795 |
| X164934 | TTACCGCACACCGTCTC | 796 |
| X164935 | CTTACCGCACACCGTCT | 797 |
| X164936 | TCTTACCGCACACCGTC | 798 |
| X164937 | GTCTTACCGCACACCGT | 799 |
| X164938 | CCAATGGACGTGGAGAG | 800 |
| X165856 | CAACACCAGCGTCCGGC | 801 |
| X165857 | AGGGCGTCCCAGTGACA | 802 |

TABLE 5
Modified MUCSB ASO Sequences

| | | SEQ |
|---|---|---|
| | | ID |
| ASO ID | Sequence | NO: |
| X164897 | GbsTbsTbsdGsdTsdAsdGsdAsdTsdGsdAs(5MdC)sdGsdTsCbsCbsTb | 803 |
| X164902 | TbsAbsGbsdTs(5MdC)sdGsdTsdGsdAs(5MdC)sdAsdGsdAsdGsCbsGbsGb | 804 |
| X164915 | CbsCbsGbsdTsdAsdGsdTsdTsdGs(5MdC)sdAsdGs(5MdC)sdAsGbsAbsAb | 805 |
| X164916 | GbsCbsCbsdGsdTsdAsdGsdTsdTsdGs(5MdC)sdAsdGs(5MdC)sAbsGbsAb | 806 |
| X164924 | GbsGbsTbsdGsdTsdAsdAsdGsdAsdGsdGsdTsdGs(5MdC)sCbsGbsTb | 807 |
| X164926 | GbsGbsAbs(5MdC)sdGsdAsdTsdAsdTs(5MdC)s(5MdC)sdAsdTsdGsGbsAbsCb | 808 |
| X164845 | AbsAbsAbsdGs(5MdC)s(5MdC)s(5MdC)sdAsdTsdGsdGsdTs(5MdC)sdGsGbsCbsTb | 809 |
| X164848 | GbsCbsGbsdTsdGsdAsdGsdTsdAsdTsdTs(5MdC)s(5MdC)sdAsCbsAbsAb | 810 |
| X164849 | CbsGbsCbsdAs(5MdC)sdTsdGsdGs(5MdC)sdGsdTsdGsdAsdGsTbsAbsTb | 811 |
| X164850 | GbsCbsGbs(5MdC)sdAs(5MdC)sdTsdGsdGs(5MdC)sdGsdTsdGsdAsGbsTbsAb | 812 |
| X164851 | TbsTbsGbsdGsdAs(5MdC)sdAsdGsdAsdAs(5MdC)sdGsdTsdAsGbsCbsTb | 813 |
| X164852 | CbsCbsTbsdTsdGsdGsdAs(5MdC)sdAsdGsdAsdAs(5MdC)sdGsTbsAbsGb | 814 |
| X164853 | AbsCbsCbsdTsdTsdGsdGsdAs(5MdC)sdAsdGsdAsdAs(5MdC)sGbsTbsAb | 815 |
| X164854 | GbsTbsTbs(5MdC)sdTs(5MdC)sdGsdTsdTsdGsdTs(5MdC)s(5MdC)sdGsTbsCbsAb | 816 |
| X164855 | AbsGbsGbs(5MdC)sdAsdGsdTsdTs(5MdC)sdTs(5MdC)sdGsdTsdTsGbsTbsCb | 817 |
| X164856 | AbsGbsCbsdTs(5MdC)sdGsdAsdGsdGsdGsdTsdGsdTsdGsÅbsAbsCb | 818 |
| X164857 | TbsGbsAbsdAsdGsdAsdAsdGs(5MdC)sdTs(5MdC)sdGsdAsdGsGbsGbsTb | 819 |
| X164858 | CbsGbsAbsdTsdGsdAsdAsdGsdAsdAsdGs(5MdC)sdTs(5MdC)sGbsAbsGb | 820 |
| X164859 | AbsCbsGbsdAsdTsdGsdAsdAsdGsdAsdAsdGs(5MdC)sdTsCbsGbsAb | 821 |
| X164860 | TbsGbsGbs(5MdC)sdGs(5MdC)sdTsdAsdGsdTs(5MdC)s(5MdC)sdAsdGsGbsCbsCb | 822 |
| X164861 | AbsGbsCbs(5MdC)sdTsdGsdGs(5MdC)sdGs(5MdC)sdTsdAsdGsdTsCbsCbsAb | 823 |
| X164862 | GbsCbsAbsdGs(5MdC)s(5MdC)sdTsdGsdGs(5MdC)sdGs(5MdC)sdTsdAsGbsTbsCb | 824 |
| X164863 | TbsGbsTbsdAsdGsdGsdTsdGsdGs(5MdC)s(5MdC)sdTs(5MdC)sdGsTbsTbsGb | 825 |
| X164864 | GbsCbsAbsdGsdTs(5MdC)sdGsdAs(5MdC)s(5MdC)s(5MdC)sdTsdGsdAsTbsGbsGb | 826 |
| X164865 | GbsTbsGbsdTsdTsdGs(5MdC)sdAsdGsdTs(5MdC)sdGsdAs(5MdC)sCbsCbsTb | 827 |
| X164866 | GbsGbsTbsdGsdTsdTsdGs(5MdC)sdAsdGsdTs(5MdC)sdGsdAsCbsCbsCb | 828 |
| X164867 | CbsAbsGbsdGsdTsdGsdTsdTsdGs(5MdC)sdAsdGsdTs(5MdC)sGbsAbsCb | 829 |
| X164868 | CbsCbsGbsdTsdAsdGsdGs(5MdC)s(5MdC)sdAs(5MdC)sdGs(5MdC)sdAsGbsGbsTb | 830 |
| X164869 | CbsCbsCbsdGsdTsdAsdGsdGs(5MdC)s(5MdC)sdAs(5MdC)sdGs(5MdC)sAbsGbsGb | 831 |
| X164870 | CbsCbsCbs(5MdC)sdGsdTsdAsdGsdGs(5MdC)s(5MdC)sdAs(5MdC)sdGsCbsAbsGb | 832 |
| X164871 | TbsCbsCbs(5MdC)s(5MdC)sdGsdTsdAsdGsdGs(5MdC)s(5MdC)sdAs(5MdC)sGbsCbsAb | 833 |
| X164872 | AbsTbsCbs(5MdC)s(5MdC)s(5MdC)sdGsdTsdAsdGsdGs(5MdC)s(5MdC)sdAsCbsGbsCb | 834 |
| X164873 | CbsAbsTbs(5MdC)s(5MdC)s(5MdC)s(5MdC)sdGsdTsdAsdGsdGs(5MdC)s(5MdC)sÅbsCbsGb | 835 |
| X164874 | AbsTbsGbs(5MdC)sdGsdGsdAsdAsdGsdGsdTs(5MdC)s(5MdC)s(5MdC)sGbsTbsGb | 836 |
| X164875 | GbsAbsCbsdGsdAsdTsdGs(5MdC)sdGsdGsdAsdAsdGsdGsTbsCbsCb | 837 |
| X164876 | CbsTbsCbsdGsdGsdTsdGsdAs(5MdC)sdGsdAsdTsdGs(5MdC)sGbsGbsAb | 838 |
| X164877 | GbsGbsAbsdTsdGsdTsdTs(5MdC)sdTs(5MdC)sdGsdGsdTsdGsAbsCbsGb | 839 |
| X164878 | GbsGbsTbsdGs(5MdC)sdGs(5MdC)sdAs(5MdC)sdGsdGsdGs(5MdC)sdTsCbsTbsTb | 840 |
| X164879 | AbsAbsGbsdAsdTs(5MdC)s(5MdC)s(5MdC)s(5MdC)sdAsdTsdGsdTsdAsGbsCbsGb | 841 |
| X164880 | TbsCbsCbsdGsdGsdTs(5MdC)s(5MdC)s(5MdC)sdAsdGsdGsdAs(5MdC)sAbsCbsGb | 842 |
| X164881 | GbsGbsTbsdGs(5MdC)sdAsdGsdTs(5MdC)sdGsdGsdAsdTsdGsAbsAbsCb | 843 |
| X164882 | TbsGbsGbsdTsdGs(5MdC)sdAsdGsdTs(5MdC)sdGsdGsdAsdTsGbsAbsAb | 844 |
| X164883 | GbsTbsGbsdTsdGs(5MdC)s(5MdC)sdGsdTsdGsdGsdGsdAs(5MdC)sAbsTbsGb | 845 |
| X164884 | CbsTbsCbsdGsdTsdAsdGsdTsdAs(5MdC)sdTsdTsdGsdGsTbsGbsGb | 846 |
| X164885 | GbsTbsCbs(5MdC)sdGsdGsdAsdGsdTs(5MdC)s(5MdC)sdGs(5MdC)s(5MdC)sAbsGbsGb | 847 |
| X164886 | TbsGbsTbs(5MdC)s(5MdC)sdGsdGsdAsdGsdTs(5MdC)s(5MdC)sdGs(5MdC)sCbsAbsGb | 848 |
| X164887 | GbsTbsGbsdTs(5MdC)s(5MdC)sdGsdGsdAsdGsdTs(5MdC)s(5MdC)sdGsCbsCbsAb | 849 |
| X164888 | AbsCbsGbsdGs(5MdC)sdAsdGsdGsdTsdGsdTs(5MdC)s(5MdC)sdGsGbsAbsGb | 850 |
| X164889 | CbsAbsCbsdGsdGs(5MdC)sdAsdGsdGsdTsdGsdTs(5MdC)s(5MdC)sGbsGbsAb | 851 |
| X164890 | CbsCbsCbsdGsdAs(5MdC)sdTs(5MdC)sdAs(5MdC)sdGsdGs(5MdC)sdAsGbsGbsTb | 852 |
| X164891 | AbsCbsGbsdGsdAs(5MdC)sdAsdGsdAsdGs(5MdC)s(5MdC)s(5MdC)sdGsAbsCbsTb | 853 |
| X164892 | AbsTbsGbs(5MdC)sdGsdTsdGsdTsdTsdGsdGsdAsdTsdGsTbsCbsTb | 854 |
| X164893 | AbsCbsAbsdTsdGs(5MdC)sdGsdTsdGsdTsdTsdGsdGsdAsTbsGbsTb | 855 |
| X164894 | GbsGbsCbsdAs(5MdC)sdAsdTsdGs(5MdC)sdGsdTsdGsdTsdTsGbsGbsAb | 856 |
| X164895 | TbsCbsGbsdGsdGsdTsdAsdGs(5MdC)sdAsdGs(5MdC)s(5MdC)sdTsAbsGbsGb | 857 |
| X164896 | TbsTbsGbsdTsdAsdGsdAsdTsdGsdAs(5MdC)sdGsdTs(5MdC)sCbsTbsGb | 858 |
| X164898 | GbsAbsCbs(5MdC)sdTs(5MdC)sdGs(5MdC)sdGsdGsdAs(5MdC)sdAs(5MdC)sAbsCbsAb | 859 |
| X164899 | AbsGbsAbs(5MdC)s(5MdC)sdTs(5MdC)sdGs(5MdC)sdGsdGsdAs(5MdC)sdAsCbsAbsCb | 860 |
| X164900 | CbsGbsGbs(5MdC)sdAsdGsdAs(5MdC)s(5MdC)sdTs(5MdC)sdGs(5MdC)sdGsGbsAbsCb | 861 |
| X164901 | TbsTbsGbsdGs(5MdC)sdGs(5MdC)sdAs(5MdC)sdAsdTs(5MdC)sdAsdGsCbsCbsCb | 862 |
| X164903 | GbsGbsCbs(5MdC)sdAs(5MdC)sdAsdGsdGsdGs(5MdC)sdAs(5MdC)sdGsTbsAbsTb | 863 |
| X164904 | TbsAbsTbs(5MdC)sdGsdTsdAsdGsdGsdAs(5MdC)sdTs(5MdC)sdAsAbsCbsGb | 864 |
| X164905 | TbsTbsAbsdTs(5MdC)sdGsdTsdAsdGsdGsdAs(5MdC)sdTs(5MdC)sAbsAbsCb | 865 |
| X164906 | CbsTbsGbsdAsdTs(5MdC)sdTsdTsdAsdTs(5MdC)sdGsdTsdAsGbsGbsAb | 866 |
| X164907 | CbsCbsTbsdGsdAsdTs(5MdC)sdTsdTsdAsdTs(5MdC)sdGsdTsAbsGbsGb | 867 |
| X164908 | GbsCbsCbs(5MdC)sdTsdGsdAsdTs(5MdC)sdTsdTsdAsdTs(5MdC)sGbsTbsAb | 868 |
| X164909 | CbsGbsGbsdAsdTs(5MdC)s(5MdC)sdTsdGsdTsdÅsdGsdTsdTsGbsTbsAb | 869 |
| X164910 | GbsAbsCbs(5MdC)s(5MdC)sdGsdGsdAsdTs(5MdC)s(5MdC)sdTsdGsdTsAbsGbsTb | 870 |
| X164911 | GbsGbsAbs(5MdC)s(5MdC)s(5MdC)sdGsdGsdAsdTs(5MdC)s(5MdC)sdTsdGsTbsAbsGb | 871 |
| X164912 | CbsGbsGbsdGsdGsdTsdAsdGsdTsdTs(5MdC)sdTs(5MdC)s(5MdC)sGbsCbsCb | 872 |
| X164913 | TbsCbsGbsdGsdGsdGsdTsdAsdGsdTsdTs(5MdC)sdTs(5MdC)sCbsGbsCb | 873 |
| X164914 | AbsCbsCbsdTs(5MdC)sdGsdGsdGsdGsdTsdAsdGsdTsdTsCbsTbsCb | 874 |
| X164917 | TbsTbsGbsdTsdTs(5MdC)sdAs(5MdC)sdGs(5MdC)sdAsdGsdGsdTsGbsAbsCb | 875 |
| X164918 | CbsTbsTbsdGsdTsdTs(5MdC)sdAs(5MdC)sdGs(5MdC)sdAsdGsdGsTbsGbsAb | 876 |
| X164919 | GbsGbsTbsdGs(5MdC)sdTsdTsdGsdTsdTs(5MdC)sdAs(5MdC)sdGsCbsAbsGb | 877 |
| X164920 | CbsAbsCbsdTs(5MdC)sdGs(5MdC)sdAs(5MdC)sdTs(5MdC)sdAsdTsdAsGbsTbsGb | 878 |
| X164921 | GbsAbsAbsdGs(5MdC)s(5MdC)sdGs(5MdC)sdTsdGsdTsdTsdAsdGsGbsAbsCb | 879 |
| X164922 | GbsTbsAbsdAsdGsdAsdGsdGsdTsdGs(5MdC)s(5MdC)sdGsdTsCbsAbsAb | 880 |
| X164923 | GbsTbsGbsdTsdAsdAsdGsdAsdGsdGsdTsdGs(5MdC)s(5MdC)sGbsTbsCb | 881 |
| X164925 | GbsAbsCbsdGsdAsdTsdAsdTs(5MdC)s(5MdC)sdAsdTsdGsdGsAbsCbsTb | 882 |
| X164927 | GbsTbsGbsdAsdGsdGsdAs(5MdC)sdGsdAsdTsdAsdTs(5MdC)sCbsAbsTb | 883 |
| X164928 | GbsCbsTbs(5MdC)sdAs(5MdC)s(5MdC)sdGsdGsdAsdAsdTsdTsdTsGbsGbsTb | 884 |
| X164929 | CbsGbsCbsdTsdGs(5MdC)sdTs(5MdC)sdAs(5MdC)s(5MdC)sdGsdGsdAsAbsTbsTb | 885 |
| X164930 | CbsCbsGbs(5MdC)sdTsdGs(5MdC)sdTs(5MdC)sdAs(5MdC)s(5MdC)sdGsdGsAbsAbsTb | 886 |
| X164931 | CbsAbsCbsdGs(5MdC)s(5MdC)sdGsdTsdTs(5MdC)sdTsdTsdGs(5MdC)sTbsGbsAb | 887 |
| X164932 | GbsCbsAbs(5MdC)sdGs(5MdC)s(5MdC)sdGsdTsdTs(5MdC)sdTsdTsdGsCbsTbsGb | 888 |
| X164933 | GbsTbsTbsdGs(5MdC)sdAs(5MdC)s(5MdC)sdTs(5MdC)sdGs(5MdC)s(5MdC)sdAsGbsTbsCb | 889 |
| X164934 | TbsTbsAbs(5MdC)s(5MdC)sdGs(5MdC)sdAs(5MdC)sdAs(5MdC)s(5MdC)sdGsdTsCbsTbsCb | 890 |
| X164935 | CbsTbsTbsdAs(5MdC)s(5MdC)sdGs(5MdC)sdAs(5MdC)sdAs(5MdC)s(5MdC)sdGsTbsCbsTb | 891 |
| X164936 | TbsCbsTbsdTsdAs(5MdC)s(5MdC)sdGs(5MdC)sdAs(5MdC)sdAs(5MdC)s(5MdC)sGbsTbsCb | 892 |
| X164937 | GbsTbsCbsdTsdTsdAs(5MdC)s(5MdC)sdGs(5MdC)sdAs(5MdC)sdAs(5MdC)sCbsGbsTb | 893 |
| X164938 | CbsCbsAbsdAsdTsdGsdGsdAs(5MdC)sdGsdTsdGsdGsdAsGbsAbsGb | 894 |
| X165856 | CbsAbsAbs(5MdC)sdAs(5MdC)s(5MdC)sdAsdGs(5MdC)sdGsdTs(5MdC)s(5MdC)sGbsGbsCb | 895 |
| X165857 | AbsGbsGbsdGs(5MdC)sdGsdTs(5MdC)s(5MdC)s(5MdC)sdAsdGsdTsdGsAbsCbsAb | 896 |

### Example 2. In Vitro Gene Silencing Screening of RNAi Agents

### Cell Culture and Transfections

Pulmonary A549 cells (adenocarcinoma human alveolar basal epithelial cells) were grown at 37° C. in Dulbecco's modified Eagle's medium supplemented with 10% FBS, 100 units ml⁻¹ penicillin, and 100 ug ml⁻¹ streptomycin (ThermoFisher Scientific). Cells were regularly passaged to maintain exponential growth. Briefly, cells were transfected by adding 25 μL of Opti-MEM plus 0.5 μL of Lipofectamine 2000 (Thermo Scientific, 2599226) to 25 ul of Opti-MEM with siRNA or ASO to an individual well in a 96-well plate. Then, 100 μL of culture media containing ˜2.0×10⁵ A549 cells were added to the siRNA/ASO-Lipofectamine mixture. Cells were incubated for 24 hours prior to measuring hsMUC5B mRNA levels. hsMUC5B RNAi agents and ASOs were tested at 20 nM and 0.20 nM doses. GAPDH mRNA levels measured by a GAPDH bDNA assay were used as Normalization Control. Negative controls were cells treated with FVII RNAi agent, Luciferase (Luc), AHSA1 RNAi agent/ASO, and left untreated (mock). Positive controls cells were treated with AHSA1 RNAi agent/ASO, then measuring AHSA1 mRNA levels using a bDNA assay. All transfections were done with n=4.

mRNA Quantification

hsMUC5B, GAPDH, and AHSA mRNA levels were measured using QuantiGene Singleplex Assays (Thermo Scientific, Cat QS0013). Briefly, the cells were lysed using the lysis mixture in a 2:1 ratio (2 parts medium and cells:1 part lysis mixture). A lysate volume equivalent to 1,000 cells was transferred to a separate capture plate well for each gene tested. A diluted lysis mixture (2 parts distilled water and part lysis mixture) was added to bring the capture well volumes to 90 μL/well. Then 10 μL of the appropriate hsMUC5B, GAPDH, AHSA working probe set was added, and the plate was sealed and incubated at 53° C. overnight (18 hours). Following the overnight probe hybridization, the capture wells were washed and sequentially hybridized with bDNA amplifiers and label probes at 46° C. for 1 hour. After a final wash, dioxitane, a luminescent alkaline phosphatase substrate, was added, and the cells were incubated at 46° C. for 30 minutes. The luminescence signal was detected using an LMax™ Luminometer (Molecular Devices). MUC5B or ASHSA mRNA levels were normalized against GAPDH mRNA levels of the same treated cell sample using the following formula: MUC5B or ASHA mRNA level/GAPDH mRNA level. Then, the percent hsMUC5B mRNA expression rate was determined as follows: (Normalized MUC5B mRNA level in MUC5B RNAi agent/ASO treated cells/Average of normalized MUC5B mRNA level of control-treated cells)*100. Controls were cells treated with FVII, ASHA, and LUC RNAi agents/ASOs and left untreated (mock). Percent hsMUC5B mRNA expression rates after exposure to the siRNAs and ASOs are shown in Tables 6 and 7, respectively.

TABLE 6
Performance of MUC5B siRNAs in A549 cells

| | Normalized hsMUC5B/hsGAPDH (%) | | | |
|---|---|---|---|---|
| | Mean | SD | Mean | SD |
| siRNA ID | (20 nM) | (20 nM) | (0.2 nM) | (0.2 nM) |
| 12616_DV22_21/23mer | 82.2 | 7.1 | 90.4 | 0.6 |
| 12621_DV22_21/23mer | 34.1 | 3.7 | 68.1 | 9.0 |
| 12706_DV22_21/23mer | 78.5 | 6.6 | 81.2 | 5.0 |
| 12706_EEL_19/21mer | 23.7 | 2.5 | 28.8 | 1.5 |
| 12706_EEL_21/23mer | 24.7 | 2.0 | 28.2 | 4.4 |
| 12717_DV22_21/23mer | 67.5 | 4.1 | 77.4 | 2.6 |
| 13206_DV22_21/23mer | 37.5 | 4.6 | 72.1 | 4.7 |
| 1399_DV22_21/23mer | 33.5 | 5.9 | 78.8 | 9.7 |
| 1399_EEL_19/21mer | 19.9 | 2.8 | 35.4 | 7.1 |
| 1399_EEL_21/23mer | 22.5 | 2.5 | 41.1 | 5.3 |
| 1401_DV22_21/23mer | 37.9 | 6.1 | 60.1 | 1.0 |
| 1401_EEL_19/21mer | 35.9 | 3.0 | 47.4 | 6.9 |
| 1401_EEL_21/23mer | 35.2 | 1.9 | 47.5 | 6.9 |
| 15132_DV22_21/23mer | 59.9 | 5.4 | 73.4 | 4.8 |
| 15140_DV22_21/23mer | 46.6 | 3.3 | 68.4 | 4.5 |
| 15140_EEL_19/21mer | 27.4 | 2.8 | 44.0 | 2.6 |
| 15140_EEL_21/23mer | 38.7 | 4.3 | 55.9 | 3.7 |
| 15197_DV22_21/23mer | 55.4 | 7.7 | 75.4 | 6.6 |
| 15197_EEL_19/21mer | 27.5 | 3.1 | 42.2 | 2.1 |
| 15197_EEL_21/23mer | 23.9 | 4.7 | 44.0 | 7.7 |
| 15252_DV22_21/23mer | 38.3 | 5.2 | 60.2 | 6.2 |
| 15252_EEL_19/21mer | 34.3 | 3.0 | 60.3 | 5.0 |
| 15252_EEL_21/23mer | 31.6 | 2.0 | 51.1 | 7.5 |
| 15254_DV22_21/23mer | 37.1 | 2.5 | 63.4 | 6.4 |
| 15258_DV22_21/23mer | 69.1 | 9.4 | 77.0 | 3.5 |
| 15258_EEL_19/21mer | 25.6 | 2.3 | 40.4 | 8.3 |
| 15258_EEL_21/23mer | 31.6 | 3.9 | 42.5 | 6.1 |
| 15261_DV22_21/23mer | 47.3 | 8.5 | 62.5 | 4.8 |
| 15261_EEL_19/21mer | 26.6 | 1.4 | 37.1 | 4.9 |
| 15261_EEL_21/23mer | 26.9 | 1.6 | 35.9 | 3.3 |
| 15262_DV22_21/23mer | 41.1 | 1.4 | 64.4 | 7.0 |
| 15301_DV22_21/23mer | 203.8 | 6.3 | 90.6 | 8.3 |
| 15304_DV22_21/23mer | 121.5 | 2.7 | 68.9 | 4.0 |
| 15305_DV22_21/23mer | 77.6 | 6.5 | 77.4 | 6.9 |
| 15305_EEL_19/21mer | 55.0 | 1.2 | 56.1 | 4.2 |
| 15305_EEL_21/23mer | 33.6 | 2.3 | 46.6 | 2.8 |
| 15310_DV22_21/23mer | 63.2 | 6.1 | 75.5 | 4.7 |
| 15311_DV22_21/23mer | 40.8 | 5.9 | 41.7 | 1.2 |
| 15312_DV22_21/23mer | 61.9 | 6.8 | 75.0 | 4.6 |
| 15312_EEL_19/21mer | 44.0 | 1.4 | 41.5 | 1.9 |
| 15312_EEL_21/23mer | 19.6 | 1.1 | 27.9 | 4.6 |
| 15314_DV22_21/23mer | 32.4 | 4.6 | 55.4 | 2.2 |
| 15314_EEL_19/21mer | 20.2 | 1.0 | 32.3 | 2.1 |
| 15314_EEL_21/23mer | 60.6 | 7.7 | 46.2 | 4.2 |
| 15315_DV22_21/23mer | 44.2 | 5.3 | 79.7 | 1.9 |
| 15473_DV22_21/23mer | 35.1 | 3.5 | 58.0 | 3.1 |
| 15473_EEL_19/21mer | 37.8 | 7.3 | 48.4 | 8.9 |
| 15473_EEL_21/23mer | 31.4 | 1.4 | 38.7 | 1.8 |
| 15476_DV22_21/23mer | 34.6 | 3.9 | 63.9 | 6.1 |
| 15478_DV22_21/23mer | 52.3 | 4.1 | 51.2 | 4.8 |
| 15478_EEL_19/21mer | 32.0 | 6.4 | 35.7 | 5.5 |
| 15478_EEL_21/23mer | 29.1 | 4.4 | 24.7 | 2.2 |
| 15479_DV22_21/23mer | 32.8 | 2.5 | 59.2 | 4.4 |
| 15539_DV22_21/23mer | 30.2 | 3.4 | 62.1 | 5.2 |
| 15539_EEL_19/21mer | 25.4 | 3.4 | 43.5 | 9.8 |
| 15539_EEL_21/23mer | 40.8 | 1.6 | 35.6 | 4.7 |
| 15543_DV22_21/23mer | 57.6 | 3.1 | 88.2 | 6.5 |
| 15543_EEL_19/21mer | 42.5 | 6.4 | 54.5 | 5.7 |
| 15543_EEL_21/23mer | 36.4 | 1.7 | 43.4 | 3.5 |
| 15546_DV22_21/23mer | 55.3 | 7.3 | 82.2 | 8.7 |
| 15547_DV22_21/23mer | 47.6 | 3.2 | 85.4 | 3.5 |
| 15547_EEL_19/21mer | 29.5 | 4.6 | 51.2 | 10.2 |
| 15547_EEL_21/23mer | 28.8 | 1.8 | 46.1 | 2.2 |
| 15563_DV22_21/23mer | 45.4 | 0.6 | 83.6 | 6.0 |
| 15563_EEL_19/21mer | 35.0 | 5.4 | 42.1 | 5.0 |
| 15563_EEL_21/23mer | 33.7 | 1.3 | 42.9 | 0.8 |
| 15564_DV22_21/23mer | 43.7 | 4.9 | 85.0 | 8.7 |
| 15564_EEL_19/21mer | 31.1 | 4.9 | 49.4 | 4.3 |
| 15564_EEL_21/23mer | 29.0 | 2.4 | 39.4 | 4.1 |
| 15568_DV22_21/23mer | 63.7 | 1.6 | 88.2 | 9.8 |
| 15572_DV22_21/23mer | 38.2 | 1.1 | 70.0 | 3.2 |
| 15576_DV22_21/23mer | 65.9 | 3.6 | 76.8 | 2.6 |
| 15576_EEL_19/21mer | 55.1 | 7.5 | 73.9 | 8.0 |
| 15576_EEL_21/23mer | 60.9 | 9.8 | 65.5 | 3.9 |
| 1564_DV22_21/23mer | 58.7 | 8.0 | 85.6 | 9.3 |
| 15692_DV22_21/23mer | 42.0 | 5.8 | 57.1 | 1.4 |
| 15731_DV22_21/23mer | 58.9 | 9.4 | 69.2 | 4.0 |
| 1617_DV22_21/23mer | 37.6 | 3.7 | 70.7 | 6.4 |
| 1617_EEL_19/21mer | 32.7 | 2.7 | 35.4 | 1.4 |
| 1617_EEL_21/23mer | 31.8 | 3.4 | 37.8 | 4.8 |
| 1618_DV22_21/23mer | 41.3 | 2.4 | 68.0 | 5.6 |
| 1619_DV22_21/23mer | 54.1 | 6.1 | 82.9 | 5.1 |
| 1619_EEL_19/21mer | 53.4 | 5.4 | 55.2 | 9.5 |
| 1619_EEL_21/23mer | 44.7 | 4.3 | 59.1 | 9.4 |
| 16985_DV22_21/23mer | 64.3 | 5.2 | 69.3 | 3.0 |
| 17255_DV22_21/23mer | 39.1 | 4.8 | 73.1 | 3.5 |
| 1756_DV22_21/23mer | 49.3 | 2.8 | 73.8 | 8.1 |
| 1808_DV22_21/23mer | 68.5 | 3.2 | 77.8 | 6.3 |
| 1808_EEL_19/21mer | 70.9 | 7.3 | 84.8 | 8.9 |
| 1808_EEL_21/23mer | 46.4 | 4.8 | 75.9 | 10.5 |
| 1896_DV22_21/23mer | 47.1 | 4.3 | 84.1 | 8.4 |
| 1897_DV22_21/23mer | 60.0 | 4.4 | 86.2 | 5.8 |
| 2112_DV22_21/23mer | 51.8 | 5.4 | 80.9 | 6.2 |
| 2112_EEL_19/21mer | 36.2 | 2.1 | 50.9 | 1.9 |
| 2112_EEL_21/23mer | 40.3 | 2.5 | 55.8 | 8.7 |
| 2113_DV22_21/23mer | 48.1 | 3.1 | 84.1 | 2.9 |
| 2113_EEL_19/21mer | 35.7 | 0.4 | 52.7 | 3.4 |
| 2113_EEL_21/23mer | 44.6 | 3.5 | 60.2 | 1.2 |
| 214_DV22_21/23mer | 35.0 | 4.5 | 71.3 | 10.0 |
| 215_DV22_21/23mer | 36.2 | 4.9 | 70.6 | 10.5 |
| 219_DV22_21/23mer | 33.1 | 3.4 | 57.9 | 9.2 |
| 2198_DV22_21/23mer | 89.8 | 3.5 | 99.0 | 8.3 |
| 2199_DV22_21/23mer | 60.4 | 3.4 | 84.3 | 3.6 |
| 2273_DV22_21/23mer | 75.5 | 4.8 | 93.1 | 1.2 |
| 2273_EEL_19/21mer | 76.2 | 3.6 | 77.5 | 2.4 |
| 2273_EEL_21/23mer | 86.1 | 1.1 | 83.0 | 2.6 |
| 2275_DV22_21/23mer | 79.0 | 6.6 | 73.1 | 3.0 |
| 2275_EEL_19/21mer | 79.8 | 3.4 | 77.4 | 3.4 |
| 2275_EEL_21/23mer | 84.0 | 7.2 | 88.8 | 7.1 |
| 2326_DV22_21/23mer | 76.4 | 6.7 | 74.9 | 5.7 |
| 2326_EEL_19/21mer | 70.0 | 4.4 | 63.5 | 3.5 |
| 2326_EEL_21/23mer | 82.6 | 2.1 | 74.2 | 5.9 |
| 2525_DV22_21/23mer | 44.8 | 6.9 | 72.7 | 4.1 |
| 2525_EEL_19/21mer | 25.7 | 2.7 | 43.4 | 3.1 |
| 2525_EEL_21/23mer | 23.8 | 2.9 | 53.4 | 5.6 |
| 2752_DV22_21/23mer | 76.9 | 8.9 | 73.6 | 5.3 |
| 2854_DV22_21/23mer | 55.2 | 4.9 | 76.5 | 18.1 |
| 2874_DV22_21/23mer | 82.1 | 6.3 | 78.4 | 12.4 |
| 3058_DV22_21/23mer | 56.1 | 4.7 | 80.1 | 7.1 |
| 3086_DV22_21/23mer | 51.0 | 2.8 | 84.4 | 9.6 |
| 3086_EEL_19/21mer | 22.6 | 2.0 | 52.3 | 0.9 |
| 3086_EEL_21/23mer | 27.0 | 1.9 | 58.1 | 1.5 |
| 3088_DV22_21/23mer | 67.2 | 8.9 | 84.4 | 10.1 |
| 3088_EEL_19/21mer | 25.4 | 1.5 | 49.5 | 9.5 |
| 3088_EEL_21/23mer | 32.2 | 3.5 | 52.3 | 4.1 |
| 3090_DV22_21/23mer | 77.2 | 4.4 | 82.8 | 3.4 |
| 3090_EEL_19/21mer | 77.7 | 2.8 | 66.3 | 2.4 |
| 3090_EEL_21/23mer | 46.3 | 5.4 | 63.6 | 4.7 |
| 3356_DV22_21/23mer | 55.9 | 7.1 | 61.3 | 5.2 |
| 3356_EEL_19/21mer | 59.6 | 3.0 | 53.7 | 5.0 |
| 3356_EEL_21/23mer | 51.8 | 7.1 | 45.9 | 4.1 |
| 3360_DV22_21/23mer | 61.9 | 4.9 | 84.7 | 6.2 |
| 3360_EEL_19/21mer | 31.6 | 3.1 | 52.8 | 6.7 |
| 3360_EEL_21/23mer | 36.8 | 6.0 | 40.3 | 3.3 |
| 3594_DV22_21/23mer | 78.1 | 7.7 | 80.5 | 4.5 |
| 3594_EEL_19/21mer | 82.2 | 7.4 | 85.0 | 6.5 |
| 3594_EEL_21/23mer | 83.4 | 7.9 | 77.3 | 4.2 |
| 3691_DV22_21/23mer | 48.7 | 2.8 | 77.5 | 5.5 |
| 3770_DV22_21/23mer | 40.4 | 3.4 | 60.7 | 4.2 |
| 3770_EEL_19/21mer | 40.1 | 3.2 | 52.7 | 2.2 |
| 3770_EEL_21/23mer | 38.1 | 4.1 | 53.0 | 7.2 |
| 3771_DV22_21/23mer | 50.7 | 4.0 | 79.9 | 4.9 |
| 3771_EEL_19/21mer | 35.0 | 6.7 | 54.8 | 9.6 |
| 3771_EEL_21/23mer | 47.2 | 2.9 | 41.3 | 3.3 |
| 3842_DV22_21/23mer | 41.0 | 4.6 | 56.8 | 2.7 |
| 3871_DV22_21/23mer | 45.6 | 3.4 | 74.8 | 5.1 |
| 3871_EEL_19/21mer | 28.1 | 2.7 | 48.9 | 12.0 |
| 3871_EEL_21/23mer | 33.7 | 2.3 | 42.1 | 5.4 |
| 4057_DV22_21/23mer | 91.9 | 4.8 | 78.0 | 1.2 |
| 4057_EEL_19/21mer | 66.3 | 7.6 | 77.9 | 8.2 |
| 4057_EEL_21/23mer | 77.8 | 9.0 | 70.9 | 4.6 |
| 4339_DV22_21/23mer | 73.8 | 2.8 | 81.3 | 5.9 |
| 4339_EEL_19/21mer | 77.4 | 2.7 | 87.8 | 11.7 |
| 4339_EEL_21/23mer | 84.1 | 8.2 | 81.9 | 3.5 |
| 4601_DV22_21/23mer | 44.5 | 2.8 | 73.8 | 2.0 |
| 4604_DV22_21/23mer | 54.2 | 6.3 | 93.3 | 8.5 |
| 4618_DV22_21/23mer | 85.5 | 4.5 | 27.0 | 2.2 |
| 4618_EEL_19/21mer | 51.3 | 6.6 | 69.7 | 5.6 |
| 4618_EEL_21/23mer | 61.9 | 6.5 | 70.7 | 6.2 |
| 4620_DV22_21/23mer | 56.3 | 3.5 | 94.7 | 8.8 |
| 4620_EEL_19/21mer | 46.5 | 4.4 | 61.1 | 10.7 |
| 4620_EEL_21/23mer | 70.8 | 5.8 | 65.3 | 9.8 |
| 4633_DV22_21/23mer | 40.7 | 7.1 | 69.0 | 10.1 |
| 4633_EEL_19/21mer | 38.4 | 3.1 | 40.3 | 5.0 |
| 4633_EEL_21/23mer | 32.1 | 3.6 | 39.3 | 5.6 |
| 4634_DV22_21/23mer | 34.5 | 4.5 | 49.2 | 6.6 |
| 4634_EEL_19/21mer | 40.5 | 6.1 | 39.2 | 6.6 |
| 4634_EEL_21/23mer | 32.4 | 4.5 | 34.5 | 3.2 |
| 4635_DV22_21/23mer | 37.0 | 4.2 | 71.4 | 10.2 |
| 4641_DV22_21/23mer | 83.6 | 9.8 | 86.7 | 7.6 |
| 4818_DV22_21/23mer | 54.3 | 2.0 | 74.0 | 6.0 |
| 4826_DV22_21/23mer | 80.2 | 8.7 | 89.4 | 16.9 |
| 525_DV22_21/23mer | 68.1 | 8.6 | 81.4 | 5.5 |
| 5277_DV22_21/23mer | 94.2 | 9.4 | 83.8 | 10.8 |
| 5277_EEL_19/21mer | 65.1 | 2.6 | 75.7 | 6.9 |
| 5277_EEL_21/23mer | 66.8 | 3.0 | 74.0 | 5.1 |
| 5425_DV22_21/23mer | 48.4 | 6.8 | 87.1 | 8.3 |
| 7169_DV22_21/23mer | 38.7 | 3.4 | 68.3 | 5.1 |
| 734_DV22_21/23mer | 76.9 | 5.6 | 78.5 | 7.7 |
| 734_EEL_19/21mer | 56.9 | 5.4 | 71.5 | 3.4 |
| 734_EEL_21/23mer | 74.0 | 4.8 | 83.8 | 7.0 |
| 737_DV22_21/23mer | 54.0 | 6.8 | 80.6 | 4.1 |
| 737_EEL_19/21mer | 41.3 | 5.7 | 69.3 | 7.6 |
| 737_EEL_21/23mer | 46.5 | 2.4 | 69.7 | 5.5 |
| 960_DV22_21/23mer | 47.9 | 5.3 | 80.9 | 4.6 |
| 960_EEL_19/21mer | 41.0 | 1.4 | 55.2 | 10.1 |
| 960_EEL_21/23mer | 31.4 | 1.4 | 52.1 | 3.7 |
| 962_DV22_21/23mer | 36.0 | 4.3 | 69.0 | 7.4 |
| 966_DV22_21/23mer | 52.5 | 4.6 | 83.3 | 7.5 |
| 972_DV22_21/23mer | 69.5 | 5.3 | 82.3 | 2.8 |
| 972_EEL_19/21mer | 21.9 | 7.3 | 51.1 | 8.6 |
| 972_EEL_21/23mer | 26.4 | 1.8 | 57.3 | 3.6 |

TABLE 7
Performance of MUC5B ASOs in A549 cells

Norm. hsMUC5B/hsGAPDH (%)

| | Mean | SD | Mean | SD |
|---|---|---|---|---|
| ASO ID | (20 nM) | (20 nM) | (0.2 nM) | (0.2 nM) |
| X164902 | 16.8 | 2.6 | 73.8 | 9.8 |
| X164897 | 19.6 | 2.2 | 57.6 | 6.7 |
| X164894 | 20.2 | 3.9 | 67.4 | 5.3 |
| X164898 | 20.2 | 1.9 | 76.6 | 10.1 |
| X164916 | 21.0 | 2.3 | 54.8 | 7.0 |
| X164931 | 21.2 | 2.2 | 83.7 | 1.7 |
| X164915 | 21.6 | 4.6 | 54.5 | 4.4 |
| X165857 | 22.0 | 2.7 | 80.7 | 5.0 |
| X164911 | 22.7 | 1.4 | 65.8 | 2.7 |
| X164926 | 22.9 | 2.6 | 61.7 | 3.9 |
| X164899 | 22.9 | 1.6 | 81.3 | 5.8 |
| X164906 | 24.1 | 2.5 | 59.4 | 4.8 |
| X164924 | 24.6 | 3.8 | 68.3 | 1.7 |
| X164933 | 24.7 | 2.8 | 89.7 | 3.3 |
| X164886 | 24.9 | 4.8 | 84.2 | 5.6 |
| X164918 | 25.1 | 2.6 | 77.1 | 6.7 |
| X164884 | 25.3 | 2.4 | 72.2 | 4.7 |
| X164932 | 25.3 | 3.1 | 89.7 | 4.3 |
| X164896 | 25.6 | 2.3 | 74.0 | 5.3 |
| X164909 | 26.5 | 2.6 | 66.8 | 3.6 |
| X164887 | 26.9 | 2.3 | 78.9 | 2.8 |
| X164919 | 27.1 | 2.5 | 85.3 | 12.8 |
| X164925 | 27.1 | 6.0 | 69.5 | 1.4 |
| X164922 | 27.7 | 9.5 | 84.5 | 6.0 |
| X164912 | 27.7 | 1.7 | 84.1 | 3.4 |
| X164927 | 27.8 | 4.9 | 67.2 | 3.2 |
| X164917 | 27.9 | 2.2 | 84.9 | 8.3 |
| X164930 | 28.3 | 2.1 | 85.3 | 3.2 |
| X164914 | 28.4 | 5.1 | 68.4 | 5.5 |
| X164848 | 28.5 | 5.9 | 90.1 | 8.4 |
| X164910 | 28.9 | 3.5 | 82.9 | 5.0 |
| X164865 | 29.2 | 1.9 | 68.1 | 4.3 |
| X164877 | 29.2 | 5.5 | 64.9 | 6.3 |
| X164923 | 29.2 | 9.0 | 79.7 | 3.0 |
| X164908 | 29.4 | 3.8 | 85.2 | 4.8 |
| X164913 | 29.6 | 3.2 | 92.6 | 6.6 |
| X164885 | 30.2 | 5.4 | 81.4 | 4.0 |
| X164864 | 30.3 | 2.0 | 68.7 | 1.4 |
| X164857 | 30.3 | 5.0 | 77.2 | 1.9 |
| X164929 | 31.2 | 4.0 | 82.1 | 7.5 |
| X164895 | 31.8 | 3.8 | 75.6 | 8.0 |
| X164928 | 32.2 | 1.1 | 77.7 | 1.4 |
| X164904 | 32.8 | 8.1 | 82.0 | 6.7 |
| X164901 | 32.9 | 3.6 | 89.0 | 5.4 |
| X164905 | 33.3 | 2.6 | 85.2 | 9.2 |
| X164874 | 34.1 | 4.6 | 81.5 | 4.0 |
| X164849 | 34.9 | 8.1 | 95.7 | 7.6 |
| X164883 | 34.9 | 2.5 | 85.6 | 2.3 |
| X164854 | 35.3 | 2.5 | 90.1 | 4.3 |
| X164900 | 36.0 | 3.9 | 86.2 | 9.1 |
| X164880 | 36.1 | 2.7 | 78.9 | 5.4 |
| X164873 | 36.6 | 7.6 | 88.7 | 1.2 |
| X164851 | 36.7 | 5.3 | 93.5 | 7.0 |
| X164859 | 36.7 | 4.6 | 93.3 | 6.8 |
| X164863 | 36.8 | 3.6 | 88.1 | 2.9 |
| X165856 | 37.4 | 5.1 | 97.5 | 8.0 |
| X164870 | 37.5 | 5.3 | 83.0 | 2.9 |
| X164879 | 37.7 | 3.2 | 76.9 | 4.3 |
| X164876 | 37.9 | 3.5 | 69.8 | 4.2 |
| X164882 | 38.0 | 7.0 | 86.1 | 4.4 |
| X164867 | 38.0 | 2.8 | 67.6 | 5.8 |
| X164858 | 38.2 | 6.5 | 81.2 | 9.1 |
| X164892 | 38.3 | 6.3 | 86.5 | 5.7 |
| X164856 | 39.2 | 5.9 | 90.0 | 8.7 |
| X164888 | 39.3 | 3.8 | 86.6 | 6.6 |
| X164852 | 39.6 | 9.8 | 91.6 | 6.6 |
| X164855 | 40.5 | 4.8 | 89.8 | 3.9 |
| X164871 | 40.5 | 4.7 | 85.0 | 5.4 |
| X164872 | 41.0 | 7.6 | 88.3 | 0.5 |
| X164881 | 41.1 | 4.3 | 84.3 | 1.5 |
| X164878 | 41.8 | 5.4 | 76.3 | 4.5 |
| X164920 | 41.9 | 2.9 | 95.7 | 13.4 |
| X164934 | 42.3 | 1.9 | 93.3 | 6.6 |
| X164889 | 42.3 | 4.6 | 87.4 | 4.6 |
| X164869 | 43.3 | 3.2 | 79.8 | 5.7 |
| X164853 | 43.3 | 6.5 | 96.7 | 6.5 |
| X164850 | 43.6 | 9.3 | 96.3 | 6.4 |
| X164868 | 45.2 | 3.9 | 77.5 | 6.0 |
| X164935 | 46.1 | 5.6 | 97.0 | 10.7 |
| X164907 | 46.2 | 1.6 | 82.9 | 5.6 |
| X164846 | 46.3 | 6.1 | 104.4 | 9.9 |
| X164891 | 46.4 | 3.8 | 91.4 | 6.0 |
| X164847 | 46.4 | 9.5 | 98.1 | 9.5 |
| X164861 | 46.5 | 5.7 | 93.6 | 6.6 |
| X164937 | 47.3 | 2.0 | 90.9 | 10.6 |
| X164938 | 49.9 | 2.6 | 83.6 | 4.2 |
| X164862 | 50.2 | 4.0 | 104.7 | 7.0 |
| X164921 | 50.2 | 4.2 | 94.3 | 3.7 |
| X164866 | 50.2 | 3.9 | 80.0 | 6.4 |
| X164845 | 51.6 | 5.9 | 103.0 | 5.5 |
| X164936 | 53.1 | 5.3 | 94.6 | 7.3 |
| X164875 | 55.0 | 2.8 | 83.0 | 1.3 |
| X164893 | 57.2 | 5.9 | 95.0 | 2.7 |
| X164890 | 61.4 | 3.1 | 94.5 | 6.4 |
| X164903 | 66.6 | 5.7 | 100.1 | 4.6 |
| X164860 | 69.3 | 10.2 | 95.6 | 2.2 |

### Example 3: Dose Response of siRNA Transfection in A549 Cells to Measure Efficacy and Potency

Selected siRNAs with chemical modifications (from Table 3) were tested in a dose response following 24 h of siRNA transfection in A549 cells.

Cell Culture and Transfection for qPCR Measurement of Endogenous Silencing of MUC5B

Diluted siRNA was prepared in a range from 1.21E-07 nM to 520.81 nM nM through 16-fold serial dilutions, and 5 μL of each dilution was transferred to a 384-well plate (Greiner, 781091) using a Biomek Fx liquid handling system (Beckman Coulter). Lipofectamine 2000 (Thermo Fisher Scientific, 11668500) was diluted by adding 0.4 μL to 20 μL of Opti-MEM™ I Reduced-Serum Medium (Thermo Fisher Scientific, 11058021) and mixed gently, followed by a 20-minute incubation at room temperature. Subsequently, 5 μL of the diluted Lipofectamine 2000 was dispensed into each well using a WellJet dispenser (Integra Biosciences), followed by shaking for 5 seconds. The mixture was incubated for 20 minutes at room temperature to allow siRNA-Lipofectamine 2000 complex formation. A549 cells were cultured in Dulbecco's Modified Eagle Medium F12 (DMEM/F12) supplemented with GlutaMax (Thermo Fisher Scientific, 10565042) and 2.5% fetal bovine serum (Sigma; F4135), and 5000 cells in 20 μL per well were added to the lipoplex and gently shaken for 5 seconds by using the WellJet dispenser. After 24 hours post-transfection, the medium was replaced with fresh cell culture medium containing 1× penicillin-streptomycin (Thermo Fisher Scientific, 15140122) every two days until day 5 post-transfection.

qPCR and Data Collection

MUC5B mRNA levels were quantified using real-time quantitative PCR (RT-qPCR) according to standard protocols. Briefly, 5 μL of cDNA was mixed with TaqMan Fast Advanced Master Mix (Thermo Fisher Scientific, 4444558) and 0.5 μM of pre-designed human MUC5B specific primers and fluorescein probes (Integrated DNA Technologies, Hs.PT.58.18743480). Human Glyceraldehyde 3-phosphate dehydrogenase (GAPDH) as internal reference was included using probes labeled with Hexachlorofluorescein (Integrated DNA Technologies, Hs.PT.39a.22214836). The reactions were performed on a qPCR machine (CFX384, Bio-Rad) with the following thermal cycling conditions: initial denaturation at 90° C. for 2 minutes, followed by 39 cycles of 95° C. for 15 seconds and 60° C. for 45 seconds. Data were exported, and the relative quantity of mRNA was calculated using the 2^{-(ΔΔCT)} method. Normalized percent hsMUC5B mRNA expression rates at 24 hours after exposure to the modified siRNAs at a maximum dose of 520 nM are shown as the efficacy in Table 8. Potency is expressed in Table 8 as IC₅₀ (Inhibitory concentration 50) in Molar (M) concentration for the dose response.

TABLE 8
Efficacy and potency of selected modified siRNAs

| | | | | | | | Normalized | |
|---|---|---|---|---|---|---|---|---|
| | | | | | | | hsMUCSB/hsGAPDH | |
| | | | | | | | (%) | |
| | | Sense | SEQ | Anti- | Antisense | SEQ | Potency | Efficacy |
| SIRNA | Sense | sequence | ID | sense | sequence | ID | (IC 50, | (Max |
| duplex ID | ID | 5′->3′ | NO: | ID | 5′->3′ | NO: | M) | Dose) |
| 12706_EEL_21/ | X158354 | ugaaAuccGU | 361 | X158355 | dTAGCAGAAC | 538 | 7.1E−12 | 80.3 |
| 23mer | | GuguucugCu | | | ACACGGADUU | | | |
| | | sa | | | CAusa | | | |
| 214_DV22_21/ | X160350 | csgsugagCf | 357 | X160351 | usCfsgggUf | 534 | 1.2E−11 | 88.8 |
| 23mer | | uUfUfGfuuc | | | ggaacaaAfg | | | |
| | | cacccga | | | Cfucacgscs | | | |
| | | | | | g | | | |
| 12706_EEL_19/ | X158446 | asAuccGUGu | 358 | X158447 | dTAGCAGAAC | 535 | 1.46−11 | 80.4 |
| 21mer | | gUucugCusa | | | ACACGGAUUU | | | |
| | | | | | csa | | | |
| 15478_EEL_21/ | X158374 | caagUccaUG | 364 | X158375 | dTGGACGAUA | 541 | 1.7E−11 | 76.0 |
| 23mer | | GauaucguCc | | | UCCaUGGACU | | | |
| | | sa | | | UGusa | | | |
| 219_DV22_21/ | X160352 | gscsuuugUf | 359 | X160353 | usAfsgugAf | 536 | 2.2E−11 | 87.5 |
| 23mer | | uCfCfAfccc | | | cggguggAfa | | | |
| | | gucacua | | | Cfaaagcsus | | | |
| | | | | | c | | | |
| 15258_EEL_19/ | X158454 | caCusuGAGu | 360 | X158455 | dTGCACUCGC | 537 | 2.4E−11 | 76.9 |
| 21mer | | gCgaguGcsa | | | ACUCAUAGUG | | | |
| | | | | | gsa | | | |
| 1399_EEL_21/ | X158304 | cagcUacgUU | 355 | X158305 | dTUCUUGGAC | 532 | 4.1E−11 | 80.5 |
| 23mer | | CuguccaaGa | | | AGABCGUAGC | | | |
| | | sa | | | UGcsa | | | |
| 4634_DV22_21/ | X158214 | gsascguuGf | 186,500 | X158215 | usUfsuauCf | 186,501 | 1.6E−10 | 76.5 |
| 23mer | | aGfUfCfcua | | | guaggacUfc | | | |
| | | cgauaaa | | | Afacgucscs | | | |
| | | | | | c | | | |
| 15476_DV22_21/ | X158266 | usascaagUf | 363 | X158267 | usAfscgaUf | 540 | 2.0E−10 | 79.5 |
| 23mer | | cCfAfUfgga | | | auccaugGfa | | | |
| | | uaucgua | | | Cfuuguasgs | | | |
| | | | | | u | | | |
| 15539_DV22_21/ | X158272 | csusguuuGf | 356 | X158273 | usAfsccgGf | 533 | 3.1E−10 | 80.8 |
| 23mer | | aCfCfAfaau | | | aauuuggUfc | | | |
| | | uccggua | | | Afaacagsgs | | | |
| | | | | | a | | | |
| 15539_EEL_19/ | X158468 | guUugaCCAa | 406 | X158469 | dTACCGGAAU | 583 | 4.1E−11 | 73.4 |
| 21mer | | aUuccgGusa | | | UUGgUCAAAC | | | |
| | | | | | asg | | | |
| 2525_EEL_19/ | X158414 | cgUgggCUGu | 457 | X158415 | dTGUGCUGAA | 634 | 7.8E−11 | 76.5 |
| 21mer | | uUcagcAcsa | | | ACAgCCCACG | | | |
| | | | | | usc | | | |
| 15311_DV22_21/ | X158256 | ascscuuuGf | 362 | X158257 | usUfsaagAf | 539 | 8.9E−11 | 71.2 |
| 23mer | | aCfGfGfcac | | | ggugccgUfc | | | |
| | | cucuuaa | | | Afaaggusgs | | | |
| | | | | | g | | | |
| 2525_EEL_21/ | X158322 | gacgUgggCU | 458 | X158323 | dTGUGCUGAA | 635 | 9.9E−11 | 74.5 |
| 23mer | | GuuucagcAc | | | ACAgCCCACG | | | |
| | | sa | | | UCcsa | | | |
| 15197_EEL_21/ | X158358 | gucaCcugCG | 377 | X158359 | dTUGCUUGUU | 554 | 1.25−10 | 74.7 |
| 23mer | | UgaacaagCa | | | CACgCAGGUG | | | |
| | | sa | | | ACgsu | | | |
| 972_EEL_19/ | X158394 | ucAcgcCAGu | 530 | X158395 | dTGUGGGCGC | 707 | 1.3E−10 | 73.5 |
| 21mer | | gCgcccAcsa | | | ACUgGCGUGA | | | |
| | | | | | gsu | | | |
| 15473_DV22_21/ | X158264 | csascuacAf | 400 | X158265 | usAfsuauCf | 577 | 1.9E−10 | 68.1 |
| 23mer | | aGfUfCfcau | | | cauggacUfu | | | |
| | | ggauaua | | | Gfuagugsgs | | | |
| | | | | | a | | | |
| 3088_EEL_19/ | X158418 | ucAuccGACu | 467 | X158419 | dTCCUGGUGC | 644 | 3.7E−10 | 76.5 |
| 21mer | | gCaccaGgsa | | | AGUcGGAUGA | | | |
| | | | | | asc | | | |
| 15314_DV22_21/ | X158260 | ususugacGf | 396 | X158261 | usGfsuguAf | 573 | 4.05−10 | 76.9 |
| 23mer | | gCfAfCfcuc | | | agaggugCfc | | | |
| | | uuacaca | | | Gfucaaasgs | | | |
| | | | | | g | | | |
| 3086_EEL_19/ | X158416 | guUcauCCGa | 464 | X158417 | dTUGGUGCAG | 641 | 4.5E−10 | 69.1 |
| 21mer | | cUgcacCasa | | | UCGgAUGAAC | | | |
| | | | | | asc | | | |
| 15479_DV22_21/ | X158270 | asasguccAf | 405 | X158271 | usAfsggaCf | 582 | 8.3E−10 | 72.4 |
| 23mer | | uGfGfAfuau | | | gauauccAfu | | | |
| | | cguccua | | | Gfgacuusgs | | | |
| | | | | | u | | | |
| 962_DV22_21/ | X158136 | ususugugGf | 527 | X158137 | usUfsggcGf | 704 | 2.1E−09 | 75.2 |
| 23mer | | aAfUfAfcuc | | | ugaguauUfc | | | |
| | | acgccaa | | | Cfacaaasgs | | | |
| | | | | | g | | | |
| 1399_DV22_21/ | X158140 | csasgcuaCf | 370 | X158141 | usUfscuuGf | 547 | 4.3E−09 | 73.9 |
| 23mer | | gUfUfCfugu | | | gacagaaCfg | | | |
| | | ccaagaa | | | Ufagcugscs | | | |
| | | | | | a | | | |
| 12621_DV22_21/ | X158228 | gscscaggUf | 366 | X158229 | usGfscugCf | 543 | — | 38.4 |
| 23mer | | cGfUfGfgaa | | | auuccacGfa | | | |
| | | ugcagca | | | Cfcuggcscs | | | |
| | | | | | c | | | |

### Example 4. In Vitro on and Off-Target Screening of Chemically Modified siRNA

In this study, additional chemical modifications were made to 6 modified dsRNA (MUC5B siRNA) sequences from Example 3, Table 8, to enhance their on-target efficacy while significantly reducing off-target effects. The 6 modified siRNA selected for additional chemical modification were: 12706_EEL_19/21mer (SEQ ID NOs: 358 and 535), 12706_EEL_21/23mer (SEQ ID NOs: 361 and 538), 15476_DV22_21/23mer (SEQ ID NOs: 363 and 540), 15478_EEL_21/23mer (SEQ ID NOs: 364 and 541), 15539_DV22_21/23mer (SEQ ID NOs: 356 and 533); and 219_DV22_21/23mer (SEQ ID NOs: 359 and 536). These starting siRNA duplexes are labeled as “V0” in Table 9, and are herein referred to as starting V0 dsRNA. 108 novel chemically modified dsRNAs were designed and evaluated for their performance in lung epithelial cells A549 (Table 9). The modifications were designed to increase efficacy, potency, and stability, and decrease off-target effects compared to the starting V0 dsRNA. All dsRNA were modified according to the backbone of the unmodified parent dsRNA, described in Table 2 (e.g., EEL or DV22 backbone as indicated in Table 2). FIG. 2 shows a schematic of the modifications for dsRNAs with the EEL backbone. The exemplary dsRNA in FIG. 2 is an EEL21/23mer, however the modifications also apply to the EEL19/21mers. FIG. 3 shows a schematic of the modifications for dsRNAs with the DV22 backbone.

### On- and Off-Target Activity

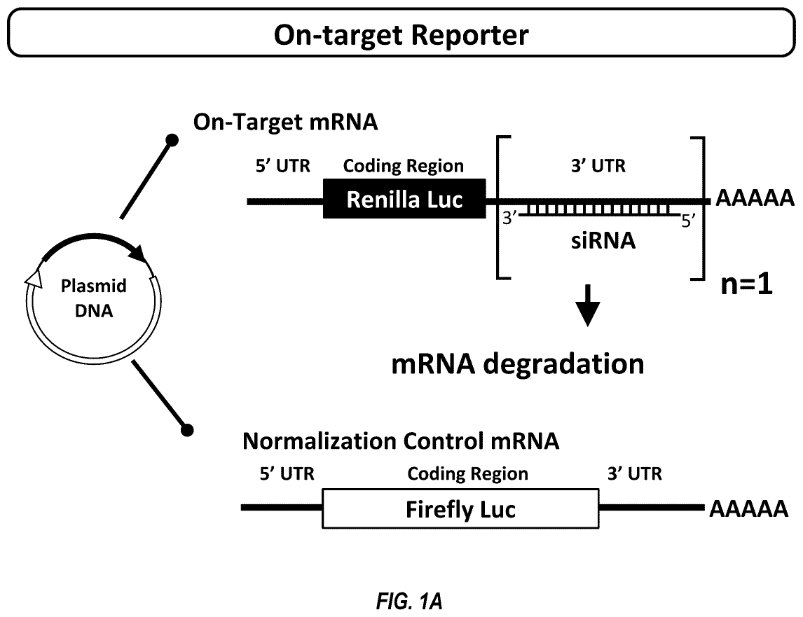

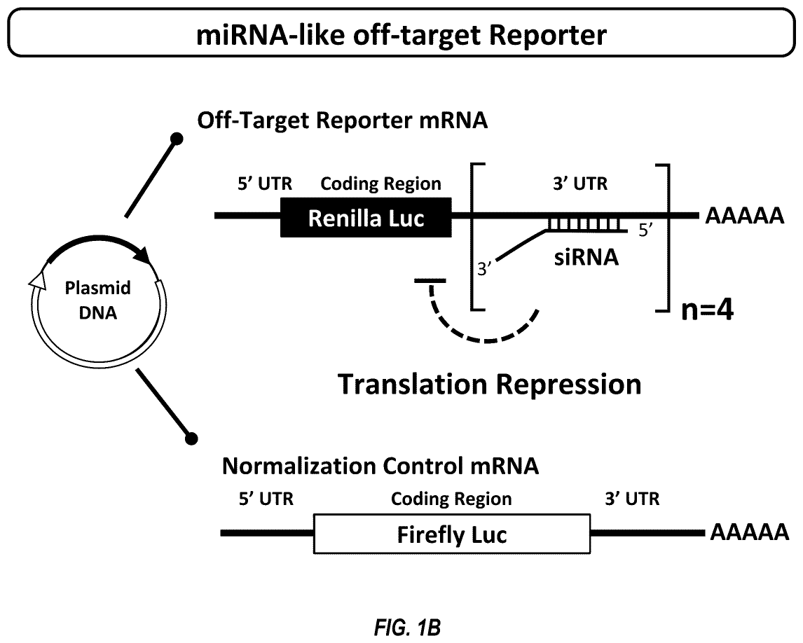

One of the main off-target effects observed in clinically designed siRNAs is miRNA-like off-target activity. Luciferase-based reporter systems were used to assess both the on-target and off-target efficacy of the siRNAs (see, FIGS. 1A-1B). This method enabled effective measurement of the activity of the siRNAs. These effects were quantified using an off-target reporter system, which demonstrated a reduction in luciferase expression when siRNAs partially bind to the 3′ UTR region of the mRNA, mimicking the function of miRNAs. To separately confirm on-target effectiveness and confirm that the silencing is durable after 5 days, the reduction in endogenous MUC5B mRNA levels following 5 days of siRNA treatment was also measured using RT-QPCR. On-target and off-target rates are shown in Table 10.

### Construction of On-Target and Off-Target Luciferase Reporter

The on/off target reporter plasmids (FIGS. 1A and 1B) were generated by cloning target sequences into the psiCHECK2 vector (Promega, C8021) between the XhoI and NotI restriction sites. The on-target reporter plasmid contained five target sites. Each complementary site is separated by a 19-nucleotide spacer (5′-TAATATTACATAAATAAAA-3′) and merged in the 3′-UTR of Renilla luciferase, matching the guide strand sequence perfectly to measure the on-target activity. The off-target reporter plasmid incorporated four tandem seed-complementary sites separated by the same 19-nucleotide spacer within the 3′-UTR of Renilla luciferase, and was used to evaluate the seed-dependent miRNA-like off-target effects. Both reporter constructs co-expressed firefly luciferase as a transfection loading control. The specific sequences of the inserted target regions are as follows:

On-target:
5′-GAGCTTTGTTCCACCCGTCACTGTAATATTACATAAATAAAATA
TGAAATCCGTGTGTTCTGCTGTAATATTACATAAATAAAAACTACA
AGTCCATGGATATCGTCCTTAATATTACATAAATAAAATCCTGTT
TGACCAAATTCCGGTGTAATATTACATAAATAAAAGAAACAGTGT
TCTTGCTCTATAA-3′.
Off-target: XD-61279
(5′-CCGTCACTGTAATATTACATAAATAAAACCGTCACTGTAATAT
TACATAAATAAAACCGTCACTGTAATATTACATAAATAAAACCGTCA
CTG-3′),
XD-60380 and XD-60426
(5′-GTTCTGCTGTAATATTACATAAATAAAAGTTCTGCTGTAATAT
TACATAAATAAAAGTTCTGCTGTAATATTACATAAATAAAAGTTCTG
CTG-3′),
XD-60336
(5′-GATATCGTCTAATATTACATAAATAAAAGATATCGTCTAATAT
TACATAAATAAAAGATATCGTCTAATATTACATAAATAAAAGATATC
GTC-3′),
XD-60390
(5′-TTATCGTCCTTAATATTACATAAATAAAATATCGTCCTTAATA
TTACATAAATAAAATATCGTCCTTAATATTACATAAATAAAATATCG
TCCT-3′),
and
XD-60339
(5′-ATTCCGGTGTAATATTACATAAATAAAAATTCCGGTGTAATAT
TACATAAATAAAAATTCCGGTGTAATATTACATAAATAAAAATTCCG
GTG-3′).

For both on- and off-target assays the underlined italic sequences represent inserted target sequences.

### Cell Culture and Transfection for On- and Off-Target Reporter Assay

A549 cells were cultured at 37° C. in a 5% CO2 incubator, using Dulbecco's Modified Eagle Medium F12 (DMEM/F12) supplemented with GlutaMax (Thermo Fisher Scientific, 10565042) and 10% fetal bovine serum (Sigma; F4135). For co-transfection, 5000 cells (20 μL per well) were suspended in a 384-well white plate (Thermo Fisher Scientific, 164610) and transfected with 25 ng of on- or off-target luciferase reporter plasmid with siRNA ranging from 7.579E-09 nM to 32.55 nM through 16-fold serial dilutions. Briefly, 0.4 μL of Lipofectamine 2000 (Thermo Fisher Scientific, 11668500) was diluted in 20 μL of Opti-MEM™ I Reduced-Serum Medium (Thermo Fisher Scientific, 11058021), mixed gently, and incubated for 5 minutes at room temperature. After 5 minutes, 20 μL of diluted Lipofectamine 2000 was transferred to an equal volume of 100 ng reporter plasmid and diluted siRNA mixture then shaken for 5 seconds. The mixture was further incubated for 20 minutes at room temperature to allow siRNA-reporter and Lipofectamine 2000 complex (lipoplex) formation, then 10 μL of the lipoplex was transferred to A549 cells and gently shaken for 5 seconds.

### Sirna on and Off Target Activity Assay

Transfected cells were harvested 24 hours post-transfection for dual-Glo Luciferase assay (Promega, E2980). 20 μL of Dual-Glo® luciferase reagent was dispensed to each well by WellJet dispenser (Integra Biosciences) and gently mixed on the plate shaker at room temperature. After 10 minutes of incubation, the firefly luciferase activity was measured on an EnVision 2105 reader (PerkinElmer). The renilla luciferase activity was measured by adding 20 μL per well of Dual-Glo® Stop & Glo® reagent to the original culture plate and incubating for 10 minutes. On- and off-target siRNA efficacy was calculated as the normalized Renilla/Firefly luminescence ratio (%) relative to the no siRNA treatment control (100% indicates no change compared to no siRNA control). Table 10 shows the mean and standard deviation of the on- and off-target siRNA efficiency at day 1 for the maximum concentration tested of 33 nM for 3 independent experiments (columns under heading “Normalized Renilla/Firefly (%) at Day 1”). Table 10 also shows the on-target siRNA potency IC50 (Inhibitory concentration 50) at day 1 for each siRNA tested. Lower values for on-target score indicate greater efficacy, as indicated by increased MUC5B silencing activity. Higher values for off-target score indicate reduced off-target activity. For example, an off-target mean maximum score of 100% indicates very low off-target activity.

Cell Culture and Transfection for qPCR Measurement of Silencing of Endogenous MUC5B mRNA

To confirm on-target effectiveness for endogenous MUC5B mRNA silencing in A549 cells, diluted siRNA was prepared in a range from 1.21E-07 nM to 520.81 nM nM through 16-fold serial dilutions, and 5 μL of each dilution was transferred to a 384-well plate (Greiner, 781091) using a Biomek Fx liquid handling system (Beckman Coulter). Lipofectamine 2000 (Thermo Fisher Scientific, 11668500) was diluted by adding 0.4 μL to 20 μL of Opti-MEM™ I Reduced-Serum Medium (Thermo Fisher Scientific, 11058021) and mixed gently, followed by a 20-minute incubation at room temperature. Subsequently, 5 μL of the diluted Lipofectamine 2000 was dispensed into each well using a WellJet dispenser (Integra Biosciences), followed by shaking for 5 seconds. The mixture was incubated for 20 minutes at room temperature to allow siRNA-Lipofectamine 2000 complex formation. A549 cells were cultured in Dulbecco's Modified Eagle Medium F12 (DMEM/F12) supplemented with GlutaMax (Thermo Fisher Scientific, 10565042) and 2.5% fetal bovine serum (Sigma; F4135), and 5000 cells in 20 μL per well were added to the lipoplex and gently shaken for 5 seconds by using the WellJet dispenser. After 24 hours post-transfection, the medium was replaced with fresh cell culture medium containing 1× penicillin-streptomycin (Thermo Fisher Scientific, 15140122) every two days until day 5 post-transfection.

Cell Lysis and cDNA Synthesis

Cell lysis buffer and cDNA synthesis reagents were prepared using the TaqMan™ Fast Advanced Cells-to-CT™ Kit (Thermo Fisher Scientific, A35378). The remaining cell culture medium was gently removed. 20 μL of cell lysis buffer was added to each well by WellJet dispenser, followed by a 10-minute incubation at room temperature with shaking at 1000 rpm. Subsequently, 10 μL of stop reagent was added to each well and incubated at room temperature for 2 minutes. After inactivating, 5 μL of the lysate was transferred to 10 μL of cDNA reverse transcription (RT) reagent and incubated at 37° C. for 30 minutes for cDNA synthesis. The reaction was inactivated by heating at 95° C. for 5 minutes. Synthesized cDNA was stored at −20° C. until further use.

qPCR and Data Collection

MUC5B mRNA levels were quantified using real-time quantitative PCR (RT-qPCR) according to standard protocols. Briefly, 5 μL of cDNA was mixed with TaqMan Fast Advanced Master Mix (Thermo Fisher Scientific, 4444558) and 0.5 μM of pre-designed human MUC5B specific primers and fluorescein probes (Integrated DNA Technologies, Hs.PT.58.18743480). Human Glyceraldehyde 3-phosphate dehydrogenase (GAPDH) as internal reference was included using probes labeled with Hexachlorofluorescein (Integrated DNA Technologies, Hs.PT.39a.22214836). The reactions were performed on a qPCR machine (CFX384, Bio-Rad) with the following thermal cycling conditions: initial denaturation at 90° C. for 2 minutes, followed by 39 cycles of 95° C. for 15 seconds and 60° C. for 45 seconds. Data were exported, and the relative quantity of mRNA was calculated using the 2^{-(ΔΔCT)} method. Normalized percent hsMUC5B mRNA expression rates at day 5 after exposure to the modified siRNAs at a maximum dose of 520 nM are shown in Table 10 (columns under heading “Normalized hsMUC5B/hsGAPDH (%) at Day 5”). Lower values for mean maximum on-target score at day 5 indicate improved siRNA stability as the effect of MUC5B silencing is maintained until day 5.

TABLE 9
Modified Sense and Antisense MUCSB siRNA Sequences

| siRNA | Sense | SEQ | Antisense | SEQ |
|---|---|---|---|---|
| duplex | sequence | ID | sequence | ID |
| ID | (5′-3′) | NO: | (5′-3′) | NO: |
| 12706_EEL_19/ | aaAuccGUGugUucu | 358 | dTAGCAGAACACAcG | 535 |
| 21mer_V0 | gCusa | | GAUUUcsa | |
| 12706_EEL_19/ | aaAuccGUGugUucu | 358 | dTagcAGAACACAc | 186,538 |
| 2lmer_V01 | gCusa | | GGAUUUcsa | |
| 12706_EEL_19/ | aaAuccGUGugUucu | 358 | dTagcaGAACACAc | 186,539 |
| 2lmer_V02 | gCusa | | GGAUUUcsa | |
| 12706_EEL_19/ | aaAuccGUGugUucu | 358 | dTAgcaGAACACAcG | 186,540 |
| 21mer_V03 | gCusa | | GAUUUcsa | |
| 12706_EEL_19/ | aaAuccGUGugUucu | 186,502 | dTAGUAGAACACAcG | 186,541 |
| 21mer_V04 | aCusa | | GAUUUcsa | |
| 12706_EEL_19/ | aaAuccGUGagUucu | 186,503 | dTAGCAGAACACAcG | 535 |
| 21mer_V05 | gCusa | | GAUUUcsa | |
| 12706_EEL_19/ | aaAuccGUCugUucu | 186,504 | dTAGCAGAACACAcG | 535 |
| 21mer_V06 | gCusa | | GAUUUcsa | |
| 12706_EEL_19/ | aaAuccGAGugUucu | 186,505 | dTAGCAGAACACAcG | 535 |
| 21mer_V07 | gCusa | | GAUUUcsa | |
| 12706_EEL_19/ | caAuccGUGugUucu | 186,506 | dTAGCAGAACACAcG | 186,542 |
| 21mer_V08 | gCusa | | GAUUGcsa | |
| 12706_EEL_19/ | asasAuccGUGugUu | 186,507 | dTsAsGCAGAACACA | 186,543 |
| 21mer_V09 | cugCsusa | | cGGAUUUscsa | |
| 12706_EEL_19/ | aaAuccGUGugUucu | 358 | (vin-u)AGCAGAAC | 186,544 |
| 21mer_V10 | gCusa | | ACAcGGAUUUcsa | |
| 12706_EEL_19/ | aaAuccGUGugUucu | 358 | dTAGCA(GNA-G)AA | 186,545 |
| 21mer_VII | gCusa | | CACAcGGAUUUcsa | |
| 12706_EEL_19/ | aaAuccGUGugUucu | 358 | dTAGCAG(GNA-A)A | 186,546 |
| 21mer_V12 | gCusa | | CACAcGGAUUUcsa | |
| 12706_EEL_19/ | aaAuccGUGugUucu | 358 | dTAGCAGAACmAmCA | 186,547 |
| 21mer_V13 | gCusa | | cGGAUUUcsa | |
| 12706_EEL_19/ | asasAuccGUGugUu | 186,507 | (vin-u)sAsGCAGA | 186,548 |
| 21mer_V15 | cugCsusa | | ACACAGGAUUUscsa | |
| 12706_EEL_19/ | asasAuccGUGugUu | 186,507 | (vin-u)sAsgcaGa | 186,549 |
| 21mer_V16 | cugCsusa | | acacacGgAuuuscs | |
| | | | a | |
| 12706_EEL_19/ | asasdAuccdGdTdG | 186,508 | (vin-u)sdAsgcad | 186,550 |
| 21mer_V17 | ugdTucugdCsusa | | GaacacacdGgdAuu | |
| | | | uscsa | |
| 12706_EEL_19/ | asasdAuccdGdTdG | 186,508 | (vin-u)sdAsgcad | 186,551 |
| 21mer_V19 | ugdTucugdCsusa | | G(GNA-A)acacacd | |
| | | | GgdAuuuscsa | |
| 12706_EEL_19/ | aaAuccGUGugUucu | 358 | (vin-u)aGCAGAAC | 186,552 |
| 21mer_V20 | gCusa | | ACAcGGAUUUcsa | |
| 12706_EEL_19/ | aaAuccGUGugUucu | 358 | (vin-u)AGCAG(GN | 186,553 |
| 21mer_V21 | gCusa | | A-A)ACACAcGGAUU | |
| | | | Ucsa | |
| 12706_EEL_19/ | aaAuccGUGugUucu | 358 | (vin-u)AgcaG(GN | 186,554 |
| 21mer_V22 | gCusa | | A-A)ACACAcGGAUU | |
| | | | Ucsa | |
| 12706_EEL_19/ | aaAuccGUGugUucu | 358 | (vin-u)AgcaG(GN | 186,555 |
| 21mer_V23 | gCusa | | A-A)ACmAmCAcGGA | |
| | | | UUUcsa | |
| 12706_EEL_21/ | ugaaAuccGUGuguu | 361 | dTAGCAGAACACAcG | 538 |
| 23mer_VO | cugCusa | | GAUUUCAusa | |
| 12706_EEL_21/ | ugaaAuccGUGuguu | 361 | dTagcAGAACACAcG | 186,556 |
| 23mer_V01 | cugCusa | | GAUUUCAusa | |
| 12706_EEL_21/ | ugaaAuccGUGuguu | 361 | dTagcaGAACACAcG | 186,557 |
| 23mer_V02 | cugCusa | | GAUUUCAusa | |
| 12706_EEL_21/ | ugaaAuccGUGuguu | 361 | dTAgcaGAACACAcG | 186,558 |
| 23mer_V03 | cugCusa | | GAUUUCAusa | |
| 12706_EEL_21/ | ugaaAuccGUGuguu | 186,509 | dTAGUAGAACACAcG | 186,559 |
| 23mer_V04 | cuaCusa | | GAUUUCAusa | |
| 12706_EEL_21/ | ugaaAuccGUGaguu | 186,510 | dTAGCAGAACACAcG | 538 |
| 23mer_V05 | cugCusa | | GAUUUCAusa | |
| 12706_EEL_21/ | ugaaAuccGUCuguu | 186,511 | dTAGCAGAACACAcG | 538 |
| 23mer_V06 | cugCusa | | GAUUUCAusa | |
| 12706_EEL_21/ | ugaaAuccGAGuguu | 186,512 | dTAGCAGAACACAcG | 538 |
| 23mer_V07 | cugCusa | | GAUUUCAusa | |
| 12706_EEL_21/ | cgaaAuccGUGuguu | 186,513 | dTAGCAGAACACAcG | 186,560 |
| 23mer_V08 | cugCusa | | GAUUUCGusa | |
| 12706_EEL_21/ | usgsaaAuccGUGug | 186,514 | dTsAsGCAGAACACA | 186,561 |
| 23mer_V09 | uucugCsusa | | cGGAUUUCAsusa | |
| 12706_EEL_21/ | ugaaAuccGUGuguu | 361 | (vin-u)AGCAGAAC | 186,562 |
| 23mer_V10 | cugCusa | | ACAcGGAUUUCAusa | |
| 12706_EEL_21/ | ugaaAuccGUGuguu | 361 | dTAGCA(GNA-G)AA | 186,563 |
| 23mer_VII | cugCusa | | CACAcGGAUUUCAus | |
| | | | a | |
| 12706_EEL_21/ | ugaaAuccGUGuguu | 361 | dTAGCAG(GNA-A)A | 186,564 |
| 23mer_V12 | cugCusa | | CACAcGGAUUUCAus | |
| | | | a | |
| 12706_EEL_21/ | ugaaAuccGUGuguu | 361 | dTAGCAGAACmAmCA | 186,565 |
| 23mer_V13 | cugCusa | | cGGAUUUCAusa | |
| 12706_EEL_21/ | usgsaaAuccGUGug | 186,514 | (vin-u)sAsGCAGA | 186,566 |
| 23mer_V15 | uucugCsusa | | ACACAcGGAUUUCAs | |
| | | | usa | |
| 12706_EEL_21/ | usgsaaAuccGUGug | 186,514 | (vin-u)sAsgcaGa | 186,567 |
| 23mer_V16 | uucugCsusa | | acacacGgAuuucas | |
| | | | usa | |
| 12706_EEL_21/ | usgsaadAuccdGdT | 186,515 | (vin-u)sdAsgcad | 186,568 |
| 23mer_V17 | dGuguucugdCsusa | | GaacacacdGgdAuu | |
| | | | ucasusa | |
| 12706_EEL_21/ | usgsaadAuccdGdT | 186,515 | (vin-u)sdAsgcad | 186,569 |
| 23mer_V19 | dGuguucugdCsusa | | G(GNA-A)acacacd | |
| | | | GgdAuuucasusa | |
| 12706_EEL_21/ | ugaaAuccGUGuguu | 361 | (vin-u)aGCAGAAC | 186,570 |
| 23mer_V20 | cugCusa | | ACAcGGAUUUCAusa | |
| 12706_EEL_21/ | ugaaAuccGUGuguu | 361 | (vin-u)AGCAG(GN | 186,571 |
| 23mer_V21 | cugCusa | | A-A)ACACAcGGAUU | |
| | | | UCAusa | |
| 12706_EEL_21/ | ugaaAuccGUGuguu | 361 | (vin-u)AgcaG(GN | 186,572 |
| 23mer_V22 | cugCusa | | A-A)ACACAcGGAUU | |
| | | | UCAusa | |
| 12706_EEL_21/ | ugaaAuccGUGuguu | 361 | (vin-u)AgcaG(GN | 186,573 |
| 23mer_V23 | cugCusa | | A-A)ACmAmCAcGGA | |
| | | | UUUCAusa | |
| 15476_DV22_21/ | usascaagUfcCfAf | 363 | usAfscgaUfaucca | 540 |
| 23mer_VO | Ufggauaucgua | | ugGfaCfuuguasgs | |
| | | | u | |
| 15476_DV22_21/ | usascaagUfcCfAf | 363 | (vin-u)sAfscgaU | 186,574 |
| 23mer_V01 | Ufggauaucgua | | fauccaugGfaCfuu | |
| | | | guasgsu | |
| 15476_DV22_21/ | usascaagUfcCfAf | 363 | usAfscga(GNA-T) | 186,575 |
| 23mer_V02 | Ufggauaucgua | | auccaugGfaCfuug | |
| | | | uasgsu | |
| 15476_DV22_21/ | usascaagUfcCfAf | 363 | usAfscgaUf(GNA- | 186,576 |
| 23mer_V03 | Ufggauaucgua | | A)uccaugGfaCfuu | |
| | | | guasgsu | |
| 15476_DV22_21/ | usascaagUfcCfAf | 186,516 | usAfscgaUfaucca | 540 |
| 23mer_V04 | dTggauaucgua | | ugGfaCfuuguasgs | |
| | | | u | |
| 15476_DV22_21/ | usascaagUfcCfAf | 186,517 | usAfscgaUfaucca | 540 |
| 23mer_V05 | Ufcgauaucgua | | ugGfaCfuuguasgs | |
| | | | u | |
| 15476_DV22_21/ | usascaagUfcCfAf | 186,518 | usAfscgaUfaucca | 540 |
| 23mer_V06 | Afggauaucgua | | ugGfaCfuuguasgs | |
| | | | u | |
| 15476_DV22_21/ | usascaagUfcCfUf | 186,519 | usAfscgaUfaucca | 540 |
| 23mer_V07 | Ufggauaucgua | | ugGfaCfuuguasgs | |
| | | | u | |
| 15476_DV22_21/ | usascaagUfcCfAf | 186,520 | usAfsuga Ufaucc | 186,577 |
| 23mer_V08 | Ufggauaucaua | | augGfaCfuuguasg | |
| | | | su | |
| 15476_DV22_21/ | usascaagUfcCfAf | 363 | usAfscgaUfaucca | 186,578 |
| 23mer_V09 | Ufggauaucgua | | ugGfaCfuuguasGm | |
| | | | sTm | |
| 15476_DV22_21/ | usascaagUfcCfAf | 363 | (vin-u)sascga U | 186,579 |
| 23mer_V10 | Ufggauaucgua | | fauccaug GfaCfu | |
| | | | uguasgsu | |
| 15476_DV22_21/ | usascaagUfcCfAf | 363 | usAfscga(abasic | 186,580 |
| 23mer_V11 | Ufggauaucgua | | -N)auccaugGfaCf | |
| | | | uuguasgsu | |
| 15476_DV22_21/ | usascaagUfcCfAf | 363 | usAfscga(abasic | 186,581 |
| 23mer_V12 | Ufggauaucgua | | )auccaugGfaCfuu | |
| | | | guasgsu | |
| 15476_DV22_21/ | usascaagUfcCfAf | 363 | usAfscga(C3)auc | 186,582 |
| 23mer_V13 | Ufggauaucgua | | caugGfaCfuuguas | |
| | | | gsu | |
| 15476_DV22_21/ | usascaagUfcCfAf | 363 | usAfscga(abasic | 186,583 |
| 23mer_V14 | Ufggauaucgua | | -n)auccaugGfaCf | |
| | | | uuguasgsu | |
| 15476_DV22_21/ | usascaagUfcCfAf | 363 | usAfscga(abasic | 186,584 |
| 23mer_V15 | Ufggauaucgua | | -F)auccaugGfaCf | |
| | | | uuguasgsu | |
| 15478_EEL_21/ | caagUccaUGGauau | 364 | dTGGACGAUAUCCaU | 541 |
| 23mer_VO | cguCosa | | GGACUUGusa | |
| 15478_EEL_21/ | caagUccaUGGanau | 364 | d′TggaCGAUAUCCa | 186.585 |
| 23mer_V01 | cguCcsa | | UGGACUUGusa | |
| 15478_EEL_21/ | caagUccaUGGauau | 364 | dTggacGAUAUCCaU | 186,586 |
| 23mer_V02 | cguCosa | | GGACUUGusa | |
| 15478_EEL_21/ | caagUccaUGGauau | 364 | dTGgacGAUAUCCaU | 186,587 |
| 23mer_V03 | cguCcsa | | GGACUUGusa | |
| 15478_EEL_21/ | caagUccaUGGauau | 186,521 | dTGGAUGAUAUCCaU | 186,588 |
| 23mer_V04 | cauCcsa | | GGACUUGusa | |
| 15478_EEL_21/ | caagUccaUGGuuau | 186,522 | dTGGACGAUAUCCaU | 541 |
| 23mer_V05 | cguCcsa | | GGACUUGusa | |
| 15478_EEL_21/ | caagUccaUGCauau | 186,523 | dTGGACGAUAUCCaU | 541 |
| 23mer_V06 | cguCosa | | GGACUUGusa | |
| 15478_EEL_21/ | caagUccaUCGauau | 186,524 | dTGGACGAUAUCCaU | 541 |
| 23mer_V07 | cguCosa | | GGACUUGusa | |
| 15478_EEL_21/ | cgagUccaUGGauau | 186,525 | dTGGACGAUAUCCaU | 186,589 |
| 23mer_V08 | cguCcsa | | GGACUCGusa | |
| 15478_EEL_21/ | csasagUccaUGGau | 186,526 | dTsGsGACGAUAUCC | 186,590 |
| 23mer_V09 | aucguCscsa | | aUGGACUUGsusa | |
| 15478_EEL_21/ | caagUccaUGGauau | 364 | (vin-u)GGACGAUA | 186,591 |
| 23mer_V10 | cguCcsa | | UCCaUGGACUUGusa | |
| 15478_EEL_21/ | caagUccaUGGauau | 364 | dTGGAC(GNA-G)AU | 186,592 |
| 23mer_V11 | cguCcsa | | AUCCaUGGACUUGus | |
| | | | a | |
| 15478_EEL_21/ | caagUccaUGGauau | 364 | dTGGACG(GNA-A)U | 186,593 |
| 23mer_V12 | cguCcsa | | AUCCaUGGACUUGus | |
| | | | a | |
| 15478_EEL_21/ | caagUccaUGGanau | 364 | dTGGACGAUAmTmCC | 186,594 |
| 23mer_V13 | cguCcsa | | aUGGACUUGusa | |
| 15478_EEL_21/ | csasagUccaUGGau | 186,526 | (vin-u)sGsGACGA | 186.595 |
| 23mer_V15 | aucguCscsa | | UAUCCaUGGACUUGs | |
| | | | usa | |
| 15478_EEL_21/ | csasagUccaUGGau | 186,526 | (vin-u)sGsgacGa | 186,596 |
| 23mer_V16 | aucguCscsa | | uauccaUgGacuugs | |
| | | | usa | |
| 15478_EEL_21/ | csasagdTccadTdG | 186,527 | (vin-u)sdGsgacd | 186,597 |
| 23mer_V17 | dGauaucgudCscsa | | GauauccadTgdGac | |
| | | | uugsusa | |
| 15478_EEL_21/ | csasagdTccadTdG | 186,527 | (vin-u)sdGsgacd | 186,598 |
| 23mer_V19 | dGauaucgudCscsa | | G(GNA-A)uauccad | |
| | | | TgdGacuugsusa | |
| 15478_EEL_21/ | caagUccaUGGauau | 364 | (vin-u)gGACGAUA | 186,599 |
| 23mer_V20 | cguCcsa | | UCCaUGGACUUGusa | |
| 15478_EEL_21/ | caagUccaUGGauau | 364 | (vin-u)GGACG(GN | 186,600 |
| 23mer_V21 | cguCcsa | | A-A)UAUCCaUGGAC | |
| | | | UUGusa | |
| 15478_EEL_21/ | caagUccaUGGauau | 364 | (vin-u)GgacG(GN | 186,601 |
| 23mer_V22 | cguCcsa | | A-A)UAUCCaUGGAC | |
| | | | UUGusa | |
| 15478_EEL_21/ | caagUccaUGGauau | 364 | (vin-u)GgacG(GN | 186,602 |
| 23mer_V23 | cguCcsa | | A-A)UAmTmCCaUGG | |
| | | | ACUUGusa | |
| 15539_DV22_21/ | csusguuuGfaCfCf | 356 | usAfsccgGfaauuu | 533 |
| 23mer_VO | Afaauuccggua | | ggUfcAfaacagsgs | |
| | | | a | |
| 15539_DV22_21/ | csusguuuGfaCfCf | 356 | (vin-u)sAfsccgG | 186,603 |
| 23mer_V01 | Afaauuccggua | | faauuugg UfcAfa | |
| | | | acagsgsa | |
| 15539_DV22_21/ | csusguuuGfaCfCf | 356 | usAfsccg(GNA-G) | 186,604 |
| 23mer_V02 | Afaauuccggua | | aauuugg UfcAfaa | |
| | | | cagsgsa | |
| 15539_DV22_21/ | csusguuuGfaCfCf | 356 | usAfsccgGf(GNA- | 186,605 |
| 23mer_V03 | Afaauuccggua | | A)auuugg UfcAfa | |
| | | | acagsgsa | |
| 15539_DV22_21/ | csusguuuGfaCfCf | 186,528 | usAfscogGfaauuu | 533 |
| 23mer_V04 | dAaauuccggua | | gg UfcAfaacagsg | |
| | | | sa | |
| 15539_DV22_21/ | csusguuuGfaCfCf | 186,529 | usAfsccgGfaauuu | 533 |
| 23mer_V05 | Afuauuccggua | | gg UfcAfaacagsg | |
| | | | sa | |
| 15539_DV22_21/ | csusguuuGfaCfCf | 186,530 | usAfsccgGfaauuu | 533 |
| 23mer_V06 | Ufaauuccggua | | gg UfcAfaacagsg | |
| | | | sa | |
| 15539_DV22_21/ | csusguuuGfaCfGf | 186,531 | usAfsccgGfaauuu | 533 |
| 23mer_V07 | Afaauuccggua | | gg UfcAfaacagsg | |
| | | | sa | |
| 15539_DV22_21/ | csusguuuGfaCfCf | 186,532 | usAfsucgGfaauuu | 186,606 |
| 23mer_V08 | Afaauuccgaua | | gg UfcAfaacagsg | |
| | | | sa | |
| 15539_DV22_21/ | csusguuuGfaCfCf | 356 | usAfsccgGfaauuu | 186,607 |
| 23mer_V09 | Afaauuccggua | | gg UfcAfaacagsG | |
| | | | msAm | |
| 15539_DV22_21/ | csusguuuGfaCfCf | 356 | (vin-u)sasccgGf | 186,608 |
| 23mer_V10 | Afaauuccggua | | aauuugg UfcAfaa | |
| | | | cagsgsa | |
| 15539_DV22_21/ | csusguuuGfaCfCf | 356 | usAfsccg(abasic | 186,609 |
| 23mer_VII | Afaauuccggua | | -N)aauuugg UfcA | |
| | | | faacagsgsa | |
| 15539_DV22_21/ | csusguuuGfaCfCf | 356 | usAfsccg(abasic | 186,610 |
| 23mer_V12 | Afaauuccggua | | )aauuugg UfcAfa | |
| | | | acagsgsa | |
| 15539_DV22_21/ | csusguuuGfaCfCf | 356 | usAfsccg(C3)aau | 186,611 |
| 23mer_V13 | Afaauuccggua | | uugg UfcAfaacag | |
| | | | sgsa | |
| 15539_DV22_21/ | csusguuuGfaCfCf | 356 | usAfsccg(abasic | 186,612 |
| 23mer_V14 | Afaauuccggua | | -n)aauuugg UfcA | |
| | | | faacagsgsa | |
| 15539_DV22_21/ | csusguuuGfaCfCf | 356 | usAfsccg(abasic | 186,613 |
| 23mer_V15 | Afaauuccggua | | -F)aauuugg UfcA | |
| | | | faacagsgsa | |
| 219_DV22_21/ | gscsuuugUfuCfCf | 359 | usAfsgugAfcgggu | 536 |
| 23mer_VO | Afcccgucacua | | ggAfaCfaaagcsus | |
| | | | c | |
| 219_DV22_21/ | gscsuuugUfuCfCf | 359 | (vin-u)sAfsgugA | 186,614 |
| 23mer_V01 | Afccogucacua | | fcggguggAfaCfaa | |
| | | | agcsusc | |
| 219_DV22_21/ | gscsuuugUfuCfCf | 359 | usAfsgug(GNA-A) | 186,615 |
| 23mer_V02 | Afcccgucacua | | cggguggAfaCfaaa | |
| | | | gcsusc | |
| 219_DV22_21/ | gscsuuugUfuCfCf | 359 | usAfsgugAf(GNA- | 186,616 |
| 23mer_V03 | Afccogucacua | | C)ggguggAfaCfaa | |
| | | | agcsusc | |
| 219_DV22_21/ | gscsuuugUfuCfCf | 186,533 | usAfsgugAfcgggu | 536 |
| 23mer_V04 | dAcccgucacua | | ggAfaCfaaagcsus | |
| | | | c | |
| 219_DV22_21/ | gscsuuugUfuCfCf | 186,534 | usAfsgug Afcggg | 536 |
| 23mer_V05 | Afgccgucacua | | ugg AfaCfaaagcs | |
| | | | usc | |
| 219_DV22_21/ | gscsuuugUfuCfCf | 186,535 | usAfsgugAfcgggu | 536 |
| 23mer_V06 | Ufcccgucacua | | ggAfaCfaaagcsus | |
| | | | c | |
| 219_DV22_21/ | gscsuuugUfuCfGf | 186,536 | usAfsgugAfcgggu | 536 |
| 23mer_V07 | Afcccgucacua | | ggAfaCfaaagcsus | |
| | | | c | |
| 219_DV22_21/ | gscsuuugUfuCfCf | 186,537 | usAfsgugAfugggu | 186,617 |
| 23mer_V08 | Afcccaucacua | | ggAfaCfaaagcsus | |
| | | | c | |
| 219_DV22_21/ | gscsuuugUfuCfCf | 359 | usAfsgugAfcgggu | 186,618 |
| 23mer_V09 | Afcccgucacua | | ggAfaCfaaagcsTm | |
| | | | sCm | |
| 219_DV22_21/ | gscsuuugUfuCfCf | 359 | (vin-u)sasgugAf | 186,619 |
| 23mer_V10 | Afcccgucacua | | cgggugg AfaCfaa | |
| | | | agcsusc | |
| 219_DV22_21/ | gscsuuugUfuCfCf | 359 | usAfsgug(abasic | 186,620 |
| 23mer_VII | Afcccgucacua | | -N)cggguggAfaCf | |
| | | | aaagcsusc | |
| 219_DV22_21/ | gscsuuugUfuCfCf | 359 | usAfsgug(abasic | 186,621 |
| 23mer_V12 | Afcccgucacua | | )cggguggAfaCfaa | |
| | | | agcsusc | |
| 219_DV22_21/ | gscsuuugUfuCfCf | 359 | usAfsgug(C3)cgg | 186,622 |
| 23mer_V13 | Afcccgucacua | | guggAfaCfaaagcs | |
| | | | usc | |
| 219_DV22_21/ | gscsuuugUfuCfCf | 359 | usAfsgug(abasic | 186,623 |
| 23mer_V14 | Afcccgucacua | | -n)cggguggAfaCf | |
| | | | aaagcsusc | |
| 219_DV22_21/ | gscsuuugUfuCfCf | 359 | usAfsgug(abasic | 186,624 |
| 23mer_V15 | Afcccgucacua | | -F)cggguggAfaCf | |
| | | | aaagcsusc | |

SIRNA ID: Location at the human MUC5B chemistry_backbone type_Length sense/Antisense in mer_Variant number

TABLE 10
ON-Target and OFF-Target siRNA performance in A549 cells.

| | | | | | | Normalized hsMUC5B/ | |
|---|---|---|---|---|---|---|---|
| | Normalized Renilla/Firefly (%) at Day 1 | | | | | hsGAPDH (%) at Day 5 | |
| | ON-TARGET | | | OFF-TARGET | | ON-TARGET | |
| | Mean | | | Mean | | Mean | |
| | Max | SD Max | IC50 | Max | Max SD | Max | SD Max |
| siRNA ID | (33 nM) | (33 nM) | (M) | (33 nM) | (33 nM) | (520 nM) | (520 nM) |
| 12706_EEL_19/21mer_V0 | 22.2 | 3.4 | 4.0E−12 | 65.9 | 13.0 | 3.3 | 0.7 |
| 12706_EEL_19/21mer_V01 | 20.2 | 2.7 | 9.3E−12 | 84.0 | 16.7 | 6.4 | 0.8 |
| 12706_EEL_19/21mer_V02 | 18.8 | 3.0 | 1.3E−11 | 76.2 | 19.1 | 8.9 | 0.8 |
| 12706_EEL_19/21mer_V03 | 22.0 | 5.3 | 5.6E−12 | 90.1 | 10.8 | 6.8 | 1.4 |
| 12706_EEL_19/21mer_V04 | 31.1 | 11.0 | 1.4E−11 | 81.9 | 18.7 | 7.3 | 1.7 |
| 12706_EEL_19/21mer_V05 | 27.1 | 3.0 | 2.9E−12 | 83.6 | 27.5 | 2.0 | 1.2 |
| 12706_EEL_19/21mer_V06 | 27.6 | 4.5 | 3.5E−12 | 84.8 | 11.8 | 7.6 | 5.1 |
| 12706_EEL_19/21mer_V07 | 28.7 | 6.0 | 4.2E−12 | 80.6 | 16.7 | 8.5 | 6.2 |
| 12706_EEL_19/21mer_V08 | 20.2 | 1.8 | 6.8E−12 | 65.3 | 15.2 | 0.8 | 0.5 |
| 12706_EEL_19/21mer_V09 | 22.6 | 3.6 | 4.7E−12 | 103.2 | 10.2 | 2.5 | 0.7 |
| 12706_EEL_19/21mer_V10 | 25.8 | 3.7 | 9.2E−12 | 85.5 | 20.4 | 1.3 | 0.3 |
| 12706_EEL_19/21mer_V11 | 62.5 | 6.8 | 2.2E−11 | 95.2 | 9.0 | 17.8 | 6.2 |
| 12706_EEL_19/2lmer_V12 | 51.1 | 9.9 | 1.5E−11 | 97.8 | 12.2 | 3.5 | 1.8 |
| 12706_EEL_19/21mer_V13 | 27.3 | 12.5 | 4.7E−12 | 68.5 | 9.1 | 2.6 | 1.1 |
| 12706_EEL_19/21mer_V15 | 31.8 | 8.1 | 6.3E−12 | 139.8 | 26.5 | 9.4 | 4.1 |
| 12706_EEL_19/21mer_V16 | 55.3 | 6.7 | 8.1E−12 | 122.0 | 22.6 | 2.1 | 1.8 |
| 12706_EEL_19/21mer_V17 | 94.1 | 12.5 | | 101.4 | 21.6 | 4.9 | 3.4 |
| 12706_EEL_19/21mer_V19 | 120.6 | 8.6 | | 138.7 | 9.6 | 30.6 | 6.3 |
| 12706_EEL_19/2lmer_V20 | 21.8 | 5.6 | 2.6E−11 | 77.9 | 14.2 | 15.7 | 6.9 |
| 12706_EEL_19/21mer_V21 | 78.1 | 5.0 | | 112.4 | 9.3 | 3.4 | 1.2 |
| 12706_EEL_19/21mer_V22 | 84.2 | 12.4 | | 101.0 | 15.5 | 10.7 | 6.7 |
| 12706_EEL_19/21mer_V23 | 69.7 | 11.7 | | 119.9 | 27.2 | 8.1 | 4.2 |
| 12706_EEL_21/23mer_V0 | 22.4 | 0.0 | 3.1E−12 | 65.6 | 11.4 | 2.1 | 0.7 |
| 12706_EEL_21/23mer_V01 | 20.5 | 2.6 | 2.4E−11 | 88.3 | 17.2 | 11.3 | 1.5 |
| 12706_EEL_21/23mer_V02 | 36.9 | 8.4 | 4.8E−11 | 97.7 | 1.2 | 15.0 | 4.4 |
| 12706_EEL_21/23mer_V03 | 24.3 | 2.8 | 4.3E−12 | 91.0 | 9.2 | 5.9 | 1.7 |
| 12706_EEL_21/23mer_V04 | 20.3 | 3.0 | 1.5E−11 | 88.6 | 21.9 | 4.0 | 0.2 |
| 12706_EEL_21/23mer_V05 | 28.1 | 4.7 | 2.1E−12 | 86.1 | 13.2 | 2.0 | 1.4 |
| 12706_EEL_21/23mer_V06 | 29.6 | 1.4 | 1.8E−12 | 88.6 | 5.9 | 2.0 | 0.9 |
| 12706_EEL_21/23mer_V07 | 29.8 | 2.3 | 3.7E−12 | 99.4 | 1.2 | 1.3 | 0.8 |
| 12706_EEL_21/23mer_V08 | 25.8 | 3.6 | 3.8E−12 | 87.9 | 16.5 | 1.1 | 0.8 |
| 12706_EEL_21/23mer_V09 | 24.5 | 2.5 | 4.9E−12 | 119.5 | 21.9 | 1.0 | 0.3 |
| 12706_EEL_21/23mer_V10 | 25.1 | 5.2 | 1.5E−11 | 96.7 | 7.6 | 1.8 | 1.0 |
| 12706_EEL_21/23mer_V11 | 26.8 | 3.6 | 1.4E−11 | 110.5 | 10.4 | 6.1 | 0.6 |
| 12706_EEL_21/23mer_V12 | 21.2 | 4.0 | 6.8E−12 | 85.6 | 6.3 | 1.3 | 0.2 |
| 12706_EEL_21/23mer_V13 | 20.0 | 3.2 | 2.8E−12 | 79.1 | 2.9 | 1.2 | 0.6 |
| 12706_EEL_21/23mer_V15 | 35.1 | 5.3 | 2.2E−11 | 126.2 | 14.2 | 7.1 | 3.6 |
| 12706_EEL_21/23mer_V16 | 70.8 | 9.2 | | 118.8 | 23.1 | 25.3 | 4.4 |
| 12706_EEL_21/23mer_V17 | 54.8 | 13.8 | | 134.4 | 39.1 | 1.2 | 0.8 |
| 12706_EEL_21/23mer_V19 | 96.9 | 14.5 | | 139.5 | 25.5 | 15.1 | 2.1 |
| 12706_EEL_21/23mer_V20 | 33.6 | 5.5 | 6.3E−11 | 69.6 | 5.0 | 36.2 | 12.3 |
| 12706_EEL_21/23mer_V21 | 30.8 | 5.6 | 5.1E−11 | 103.6 | 7.3 | 5.9 | 1.3 |
| 12706_EEL_21/23mer_V22 | 40.8 | 6.7 | 6.6E−11 | 105.4 | 12.1 | 19.9 | 3.7 |
| 12706_EEL_21/23mer_V23 | 41.4 | 8.2 | 2.9E−11 | 101.4 | 28.9 | 10.0 | 4.5 |
| 15476_DV22_21/23mer_V0 | 38.7 | 0.6 | 1.6E−11 | 112.1 | 3.7 | 9.1 | 0.9 |
| 15476_DV22_21/23mer_V01 | 53.1 | 4.4 | 7.5E−12 | 119.3 | 34.9 | 4.0 | 0.7 |
| 15476_DV22_21/23mer_V02 | 44.2 | 4.0 | 2.7E−11 | 102.4 | 16.5 | 8.9 | 1.5 |
| 15476_DV22_21/23mer_V03 | 72.7 | 6.7 | 5.2E−11 | 123.9 | 15.3 | 5.1 | 3.0 |
| 15476_DV22_21/23mer_V04 | 56.9 | 7.4 | 6.9E−11 | 102.6 | 4.5 | 6.9 | 1.6 |
| 15476_DV22_21/23mer_V05 | 73.6 | 4.0 | 1.1E−11 | 156.6 | 13.1 | 12.8 | 7.8 |
| 15476_DV22_21/23mer_V06 | 58.1 | 7.7 | 1.3E−11 | 130.7 | 16.3 | 12.9 | 4.9 |
| 15476_DV22_21/23mer_V07 | 67.6 | 2.8 | | 141.5 | 14.9 | 8.9 | 6.5 |
| 15476_DV22_21/23mer_V08 | 88.2 | 16.2 | | 123.7 | 18.6 | 1.7 | 1.2 |
| 15476_DV22_21/23mer_V09 | 56.2 | 12.0 | 7.2E−12 | 123.5 | 15.7 | 3.2 | 1.7 |
| 15476_DV22_21/23mer_V10 | 113.2 | 10.4 | | 147.5 | 25.9 | 3.6 | 0.6 |
| 15476_DV22_21/23mer_V11 | 70.6 | 2.4 | 2.8E−11 | 131.2 | 18.2 | 6.4 | 3.2 |
| 15476_DV22_21/23mer_V12 | 63.5 | 12.8 | 1.2E−11 | 143.5 | 28.2 | 9.3 | 1.3 |
| 15476_DV22_21/23mer_V13 | 77.3 | 8.7 | 1.5E−11 | 141.9 | 34.4 | 13.7 | 4.9 |
| 15476_DV22_21/23mer_V14 | 51.5 | 5.8 | 1.1E−11 | 124.8 | 16.5 | 9.4 | 0.6 |
| 15476_DV22_21/23mer_V15 | 58.9 | 14.0 | 1.7E−11 | 137.5 | 26.6 | 23.9 | 8.3 |
| 15478_EEL_21/23mer_V0 | 39.0 | 7.1 | 4.3E−12 | 42.0 | 17.1 | 2.6 | 1.4 |
| 15478_EEL_21/23mer_V01 | 33.4 | 7.2 | 3.3E−11 | 64.0 | 22.7 | 32.7 | 6.3 |
| 15478_EEL_21/23mer_V02 | 56.5 | 6.5 | | 73.1 | 7.4 | 27.6 | 6.3 |
| 15478_EEL_21/23mer_V03 | 36.5 | 13.7 | 6.9E−12 | 52.3 | 20.3 | 7.6 | 1.1 |
| 15478_EEL_21/23mer_V04 | 38.4 | 8.4 | 8.2E−12 | 96.5 | 18.1 | 18.3 | 4.0 |
| 15478_EEL_21/23mer_V05 | 51.0 | 14.5 | 6.5E−12 | 77.8 | 5.2 | 7.6 | 2.0 |
| 15478_EEL_21/23mer_V06 | 52.4 | 21.6 | 4.7E−12 | 72.3 | 17.8 | 7.7 | 0.6 |
| 15478_EEL_21/23mer_V07 | 47.4 | 9.4 | 6.2E−12 | 61.4 | 7.3 | 6.1 | 1.2 |
| 15478_EEL_21/23mer_V08 | 34.7 | 2.7 | 4.8E−12 | 75.3 | 12.6 | 3.1 | 0.8 |
| 15478_EEL_21/23mer_V09 | 80.3 | 9.4 | 7.3E−12 | 131.0 | 25.8 | 4.8 | 0.4 |
| 15478_EEL_21/23mer_V10 | 34.5 | 8.7 | 1.3E−11 | 56.5 | 6.1 | 4.1 | 1.5 |
| 15478_EEL_21/23mer_V11 | 41.4 | 3.5 | 1.2E−11 | 111.3 | 5.3 | 1.2 | 1.2 |
| 15478_EEL_21/23mer_V12 | 37.9 | 10.9 | 7.0E−12 | 98.8 | 17.6 | 1.8 | 0.9 |
| 15478_EEL_21/23mer_V13 | 30.5 | 11.5 | 5.1E−12 | 49.6 | 18.0 | 3.0 | 1.7 |
| 15478_EEL_21/23mer_V15 | 117.6 | 20.4 | 1.1E−11 | 133.9 | 14.3 | 34.1 | 4.1 |
| 15478_EEL_21/23mer_V16 | 99.5 | 14.6 | | 131.3 | 35.4 | 80.7 | 20.3 |
| 15478_EEL_21/23mer_V17 | 119.5 | 0.2 | | 106.5 | 24.7 | 113.8 | 16.5 |
| 15478_EEL_21/23mer_V19 | 103.0 | 17.8 | | 136.0 | 11.4 | 110.0 | 10.9 |
| 15478_EEL_21/23mer_V20 | 36.8 | 5.7 | 7.9E−11 | 70.0 | 6.7 | 45.3 | 5.8 |
| 15478_EEL_21/23mer_V21 | 46.3 | 9.2 | 3.7E−11 | 100.9 | 15.5 | 2.4 | 0.4 |
| 15478_EEL_21/23mer_V22 | 87.6 | 9.9 | | 103.7 | 4.5 | 57.5 | 24.3 |
| 15478_EEL_21/23mer_V23 | 83.9 | 8.2 | | 113.7 | 6.0 | 66.8 | 33.0 |
| 15539_DV22_21/23mer_V0 | 59.4 | 6.8 | | 62.1 | 5.5 | 0.9 | 0.4 |
| 15539_DV22_21/23mer_V01 | 58.3 | 14.1 | 8.8E−12 | 94.3 | 7.8 | 2.0 | 1.6 |
| 15539_DV22_21/23mer_V02 | 64.0 | 3.8 | | 117.0 | 12.0 | 7.5 | 2.7 |
| 15539_DV22_21/23mer_V03 | 57.2 | 2.1 | | 100.8 | 12.4 | 4.0 | 0.4 |
| 15539_DV22_21/23mer_V04 | 63.3 | 6.9 | | 77.8 | 19.5 | 1.7 | 1.2 |
| 15539_DV22_21/23mer_V05 | 74.0 | 14.8 | | 108.0 | 15.5 | 1.0 | 0.1 |
| 15539_DV22_21/23mer_V06 | 85.4 | 11.3 | | 109.4 | 23.8 | 2.1 | 0.2 |
| 15539_DV22_21/23mer_V07 | 76.6 | 3.7 | | 98.6 | 25.9 | 1.2 | 0.3 |
| 15539_DV22_21/23mer_V08 | 54.4 | 8.0 | | 113.1 | 13.1 | 4.7 | 0.7 |
| 15539_DV22_21/23mer_V09 | 45.6 | 0.6 | 2.0E−11 | 85.5 | 13.6 | 2.2 | 1.2 |
| 15539_DV22_21/23mer_V10 | 76.3 | 2.5 | | 119.9 | 20.7 | 17.2 | 10.3 |
| 15539_DV22_21/23mer_V11 | 98.7 | 20.5 | | 156.9 | 17.7 | 11.7 | 2.3 |
| 15539_DV22_21/23mer_V12 | 71.4 | 0.6 | | 135.3 | 40.6 | 10.8 | 1.9 |
| 15539_DV22_21/23mer_V13 | 90.0 | 12.7 | | 125.0 | 26.5 | 14.9 | 1.9 |
| 15539_DV22_21/23mer_V14 | 79.4 | 11.8 | | 133.5 | 16.6 | 11.6 | 3.0 |
| 15539_DV22_21/23mer_V15 | 92.7 | 12.3 | | 139.5 | 21.0 | 19.7 | 2.5 |
| 219_DV22_21/23mer_V0 | 26.3 | 5.6 | 9.7E−11 | 85.7 | 17.2 | 3.8 | 2.3 |
| 219_DV22_21/23mer_V01 | 22.7 | 2.4 | 1.6E−11 | 116.1 | 11.8 | 2.1 | 0.9 |
| 219_DV22_21/23mer_V02 | 46.2 | 18.4 | 7.1E−11 | 103.8 | 12.2 | 10.0 | 9.9 |
| 219_DV22_21/23mer_V03 | 48.2 | 14.8 | 8.8E−11 | 103.8 | 16.7 | 13.5 | 3.0 |
| 219_DV22_21/23mer_V04 | 42.0 | 16.5 | 8.2E−11 | 83.7 | 5.3 | 3.9 | 2.0 |
| 219_DV22_21/23mer_V05 | 27.6 | 7.6 | 8.9E−11 | 71.0 | 3.2 | 2.8 | 2.0 |
| 219_DV22_21/23mer_V06 | 67.8 | 25.9 | 2.1E−11 | 80.3 | 13.9 | 2.8 | 1.3 |
| 219_DV22_21/23mer_V07 | 44.0 | 14.1 | 7.3E−11 | 98.3 | 12.7 | 1.6 | 0.4 |
| 219_DV22_21/23mer_V08 | 15.4 | 9.6 | 7.0E−11 | 100.5 | 7.2 | 3.5 | 3.9 |
| 219_DV22_21/23mer_V09 | 57.5 | 30.5 | 4.4E−10 | 94.5 | 14.7 | 7.2 | 2.8 |
| 219_DV22_21/23mer_V10 | 117.5 | 17.4 | | 121.0 | 11.2 | 42.1 | 7.8 |
| 219_DV22_21/23mer_V11 | 75.8 | 24.6 | 4.1E−10 | 142.4 | 46.8 | 26.8 | 2.3 |
| 219_DV22_21/23mer_V12 | 46.4 | 5.3 | 1.3E−10 | 156.3 | 40.7 | 31.7 | 1.0 |
| 219_DV22_21/23mer_V13 | 56.9 | 5.3 | 4.9E−11 | 158.7 | 16.9 | 31.1 | 2.1 |
| 219_DV22_21/23mer_V14 | 53.9 | 11.3 | 1.3E−10 | 156.3 | 14.8 | 41.2 | 5.6 |
| 219_DV22_21/23mer_V15 | 48.6 | 2.7 | 1.4E−10 | 170.8 | 33.7 | 29.0 | 5.5 |

On-Target siRNA efficacy is expressed as normalized Renilla/Firefly (%) and Normalized hsMUC5B/hsGAPDH (%) at max concentration of 33 nM and 520 nM, respectively. On-Target siRNA potency is expressed as IC50 (Inhibitory concentration 50) at Molar (M) concentration. Off-Target siRNA efficacy is expressed as normalized Renilla/Firefly (%) at maximum concentration of 33 nM. Normalized Renilla/Firefly (%) and Normalized hsMUC5B/hsGAPDH (%) values were measured following 1 and 5 days of siRNA treatment, respectively. Values represent the mean +/− SD of 3 independent experiment. Blank rows in the IC50 column indicate that an IC50 could not be calculated due to low inhibitory activity of the siRNA.

In some embodiments, the additional modifications shown in Table 9 increased siRNA efficacy at 24 hours, as defined by a reduced maximum on-target score in Table 10. In some cases, the additional modifications increased potency, as defined by reduced 1C50 in Table 10. In some embodiments, the additional modifications decreased off-target activity at 24 hours, as defined by a higher 24 hour off-target score in Table 10. In some embodiments, the additional modifications increased stability, as defined by a low score for maximum on-target activity at day 5. For example, for the 12706_EEL_19/21mer and 12706_EEL_21/23mer siRNA, the V09 modifications (12706_EEL_19/21mer_V09 sense strand SEQ ID NO:186,507; antisense strand SEQ ID NO. 186,543; 12706_EEL_21/23mer_V09 sense strand SEQ ID NO: 186,514; antisense SEQ ID NO: 186,561, Table 9) demonstrated improved off-target activity while maintaining efficacy, potency and stability compared to the starting V0 siRNAs (12706_EEL_19/21mer_V0 sense strand SEQ ID NO: 358; antisense strand SEQ ID NO: 535; 12706_EEL_21/23mer_V0 sense strand SEQ ID NO: 361; antisense strand SEQ ID NO: 538). The V09 modification also led to a stable reduction in MUC5B mRNA expression at day 5 for these two sequences (Table 10). The 12706_EEL_19/21mer and 12706_EEL_21/23mer share the same starting sequence with the exception that the 21/23mer is 2 nucleotides longer in both the sense and antisense strand. These results indicate that for the 12706 EEL siRNA sequences, the V09 modification may lead to better overall therapeutic profile.

For the 15478_EEL_21/23mer, which does not have the same sequence, the V09 modified siRNA (sense strand SEQ ID NO: 186,526; antisense strand SEQ ID NO: 186,590) did not have improved on-target activity compared to the starting V0 siRNA (sense strand SEQ ID NO: 364; antisense strand SEQ ID NO: 541) at 24 hours but did have significantly improved off-target activity at 24 hours. This suggests that some modifications may have sequence-specific functional effects.

In some embodiments, the modifications improved on- and/or off-target effects at 24 hours but were not as stable as the starting V0 siRNA after 5 days in culture. For example, the 12706_EEL_21/23mer_V01 (sense strand SEQ ID NO: 361; antisense strand SEQ ID NO: 186,556) and 12706_EEL_21/23mer_V02 (sense strand SEQ ID NO: 361; antisense strand SEQ ID NO: 186,557) displayed similar on- and off-target activity as the starting 12706_EEL_21/23mer_V0 siRNA (sense strand SEQ ID NO: 361; antisense strand SEQ ID NO: 538) at 24 hours, but the on-target performance at day 5 was reduced.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### REFERENCES

1. Kobayashi, Y., et al. (2022) ACS Omega 7(2): 2398-2410.
2. Wu, H et al., (2011) PLoS ONE 6(12)e 28580.
3. Addepalli H et al., (2010) Nucleic Acids Res 38(20):7320-7331.
4. Parmar, R., et al., (2016) ChemBioChem 17(11): 985-989.
5. Elkayam, E., et al., (2017) Nucleic Acids Res 45(6): 3528-3536.
6. Haraszti, R. A., et al., (2017) Nucleic Acids Res 45(13): 7581-7592.
7. Prakash, T. P., et al., (2016) Bioorg Med Chem Lett 26(12): 2817-2820.
8. Lima, W. F., et al., (2012) Cell 150(5): 883-894.
9. Khvorova A et al., (2003) Cell 115(2):209-216.
10. Schwarz, D S et al., (2003) Cell 115(2):199-208.
11. Janas, M. M., et al., (2018) Nature Communications 9(1):723.
12. Song, X., et al., (2017) Mol Ther Nucleic Acids 9:242-250.
13. Jackson, A. L., et al., (2006) RNA 12(7): 1197-1205.
14. Jackson, A. L. and P. S. Linsley (2010) Nat Rev Drug Discov 9(1): 57-67.
15. Lima, W. F., et al., (2012) Cell 150(5):883-894.
16. Lee, H. S., et al., (2015) Nat Commun 6: 10154.
17. Hu B, Zhong L, Weng Y, Peng L, Huang Y, Zhao Y, Liang X J. Signal Transduct Target Ther (2020) June 19; 5(1):101.

1. A double stranded ribonucleic acid (dsRNA) targeting Mucin-5B (MUC5B), wherein the dsRNA comprises a sense strand and an antisense strand forming a double stranded region, wherein the antisense strand comprises a region of complementarity to an mRNA encoding MUC5B, and wherein the antisense strand comprises a nucleotide sequence selected from SEQ ID NOs: 178-354, wherein the sense and/or antisense strand comprises at least one modification.

2. The dsRNA of claim 1, wherein the sense strand comprises a nucleotide sequence selected from SEQ ID NOs: 1-177.

3. The dsRNA of claim 1, wherein the sense and antisense strands are selected from any one of the sense and antisense pairs in Table 2 or Table 3.

4. A double stranded ribonucleic acid (dsRNA) targeting Mucin-5B (MUC5B), wherein the dsRNA comprises a sense strand and an antisense strand forming a double stranded region, wherein the sense and/or antisense strand comprises at least one modification, and wherein:
a. the antisense strand comprises a region of complementarity to an mRNA encoding MUC5B, and wherein the antisense strand comprises a nucleotide sequence differing in sequence by no more than 1 nucleotide from any one of SEQ ID NOs: 179-192, 194-328, and 332-354;
b. the antisense strand comprises a nucleotide sequence comprising at least 22 contiguous nucleotides differing in sequence by no more than 1 nucleotide from any one of SEO ID NOs: 179-187, 189, 190, 192, 194-200, 202, 204-311, 313, 315-328, 332-334, and 336-35;
c. the antisense strand comprises a nucleotide sequence comprising at least 21 contiguous nucleotides differing in sequence by no more than 1 nucleotide from any one of SEO ID NOs: 181-187, 189, 190, 192, 194-200, 202, 204-229, 231-243, 246-310, 313, 315-328, 333-334, and 336-354;
d. the antisense strand comprises a nucleotide sequence comprising at least 20 contiguous nucleotides differing in sequence by no more than 1 nucleotide from any one of SEO ID NOs: 181-187, 190, 192, 194-200, 205-210, 212-229.231-242, 246-249, 253-258, 260-266, 268-285, 287, 289-294, 296, 298-310, 315-328, 333-334, 336-346, 348, and 350-354;
e. the antisense strand comprises a nucleotide sequence comprising at least 19 contiguous nucleotides differing in sequence by no more than 1 nucleotide from any one of SEO ID NOs: 181-187, 190, 192, 194-200, 205-210, 213, 215-228, 231-237, 239-242, 246-248, 253-258, 261-266, 268-269, 271, 273-285, 287, 289-294, 296, 298-303, 305-310, 315-328, 334, 336-338, 340-346, 348, and 350-354;
f. the antisense strand comprises a nucleotide sequence comprising at least 18 contiguous nucleotides differing in sequence by no more than 1 nucleotide from any one of SEO ID NOs: 181-187, 190, 192, 194-197, 205-210, 213, 215-228, 231-237, 239-242, 253-254, 261-266, 268-269, 271, 273-281, 283-285, 290, 292-294, 298-303, 305-310, 315-322, 324, 326-328, 334, 336-338, 340-346, and 351-354;
g. the antisense strand comprises a nucleotide sequence comprising at least 17 contiguous nucleotides differing in sequence by no more than 1 nucleotide from any one of SEO ID NOs: 181-187, 190, 192, 194-197, 205-210, 215-228, 232, 234-237, 239-242, 253-254, 261-266, 268-269, 274, 276-278, 284-285, 290, 292-294, 298-303, 305-310, 315-322, 324, 326-328, 334, 336-338, 341-346, and 351-354;
h. the antisense strand comprises a nucleotide sequence comprising at least 16 contiguous nucleotides differing in sequence by no more than 1 nucleotide from any one of SEO ID NOs: 181, 183-187, 190, 192, 194-197, 207-210, 215-228, 232, 234-237, 239-242, 253-254, 261-266, 268-269, 274, 276-278, 284-285, 293, 299, 301-303, 305-307, 315-321, 327, 334, 336-338, 341-346, and 351-354
i. the antisense strand comprises a nucleotide sequence comprising at least 15 contiguous nucleotides differing in sequence by no more than 1 nucleotide from any one of SEO ID NOs: 181, 183-187, 190, 192, 194-197, 207-210, 215-222, 224, 226-228, 234-237, 240-242, 253-254, 261-266, 268-269, 276-278, 284-285, 293, 299, 301-303, 315-317, 319, 321, 327, 334, 336-338, 341-346, and 351-354;
j. the antisense strand comprises a nucleotide sequence comprising at least 15 contiguous nucleotides differing in sequence by no more than 2 nucleotides from any one of SEO ID NOs: 185-187, 192, 194, 207-210, 216-222, 224, 226-228, 235-237, 240-242, 253-254, 261-266, 268-269, 276-278, 284-285, 301-303, 315-317, 319, 321, 334, 336-338, 341-346, and 352-354; or
k. the antisense strand comprises a nucleotide sequence comprising at least 15 contiguous nucleotides differing in sequence by no more than 3 nucleotides from any one of SEO ID NOs: 186-187, 192, 194, 208, 216-222, 226-228, 236, 240-242, 253-254, 268-269, 276-278, 284-285, 301-303, 316, 334, 336-338, 341-346, and 352-354.

5.-27. (canceled)

28. A double stranded ribonucleic acid (dsRNA) targeting Mucin-5B (MUC5B), wherein the dsRNA comprises a sense strand and an antisense strand forming a double stranded region, wherein the antisense strand comprises a region of complementarity to an mRNA encoding MUC5B, wherein
a. the sense strand comprises the nucleotide sequence of SEQ ID NO: 1, and the antisense strand comprises the nucleotide sequence of SEQ ID NO: 178;
b. the sense strand comprises a nucleotide sequence comprising at least 20 contiguous nucleotides differing in sequence by no more than 1 nucleotide from SEQ ID NO: 2, and the antisense strand comprises a nucleotide sequence comprising at least 22 contiguous nucleotides differing in sequence by no more than 1 nucleotide from SEQ ID NO: 179;
c. the sense strand comprises a nucleotide sequence comprising at least 20 contiguous nucleotides differing in sequence by no more than 1 nucleotide from any one of SEQ ID NO: 3, and the antisense strand comprises a nucleotide sequence comprising at least 22 contiguous nucleotides differing in sequence by no more than 1 nucleotide from SEQ ID NO: 180;
d. the sense strand comprises a nucleotide sequence comprising at least 15 contiguous nucleotides differing in sequence by no more than 1 nucleotide from SEQ ID NO: 5, and the antisense strand comprises a nucleotide sequence comprising at least 17 contiguous nucleotides differing in sequence by no more than 1 nucleotide from SEQ ID NO: 182;
e. the sense strand comprises a nucleotide sequence comprising at least 15 contiguous nucleotides differing in sequence by no more than 3 nucleotides from SEQ ID NO: 4, and the antisense strand comprises a nucleotide sequence comprising at least 15 contiguous nucleotides differing in sequence by no more than 1 nucleotide from SEQ ID NO: 181;
f. the sense strand comprises a nucleotide sequence comprising at least 15 contiguous nucleotides differing in sequence by no more than 3 nucleotides from SEQ ID NO: 6, and the antisense strand comprises a nucleotide sequence comprising at least 15 contiguous nucleotides differing in sequence by no more than 1 nucleotide from SEQ ID NO: 183;
g. the sense strand comprises a nucleotide sequence comprising at least 15 contiguous nucleotides differing in sequence by no more than 3 nucleotides from SEQ ID NO: 7, and the antisense strand comprises a nucleotide sequence comprising at least 15 contiguous nucleotides differing in sequence by no more than 1 nucleotide from SEQ ID NO: 184;
h. the sense strand comprises a nucleotide sequence comprising at least 15 contiguous nucleotides differing in sequence by no more than 3 nucleotides from SEQ ID NO: 8, and the antisense strand comprises a nucleotide sequence comprising at least 15 contiguous nucleotides differing in sequence by no more than 2 nucleotides from SEQ ID NOs: 185;
i. the sense strand comprises a nucleotide sequence comprising at least 15 contiguous nucleotides differing in sequence by no more than 3 nucleotides from SEQ ID NO: 9, and the antisense strand comprises a nucleotide sequence comprising at least 15 contiguous nucleotides differing in sequence by no more than 3 nucleotides from SEQ ID NO: 186;
j. the sense strand comprises a nucleotide sequence comprising at least 15 contiguous nucleotides differing in sequence by no more than 3 nucleotides from SEQ ID NO: 10, and the antisense strand comprises a nucleotide sequence comprising at least 15 contiguous nucleotides differing in sequence by no more than 3 nucleotides from SEQ ID NO: 187;
k. the antisense strand comprises a nucleotide sequence selected from any one of SEQ ID NOs: 18,793-36,683, and the sense strand comprises a nucleotide sequence selected from any one of SEQ ID NOs: 902-18,792;
l. the antisense strand comprises a nucleotide sequence selected from any one of SEQ ID NOs: 54,575-72,465, and the sense strand comprises a nucleotide sequence selected from any one of SEQ ID NOs: 36,684-54,574; or
m. the antisense strand comprises a nucleotide sequence selected from any one of SEQ ID NOs: 90,357-108,247, and the sense strand comprises a nucleotide sequence selected from any one of SEQ ID NOs: 72,466-90,356.

29. (canceled)

30. The dsRNA of claim 1, wherein each of the sense strand and/or the antisense strand:
a. is conjugated to one or more lipophilic moieties; and/or
b. is packaged in a lipid nanoparticle (LNP).

31.-32. (canceled)

33. The dsRNA of claim 1, wherein
a. at least 1, 2, 3, 4, 5, 6, 7, 8, 9, 10, 11, 12, 13, 14, 15, 16, 17, 18, 19, or 20 nucleotides of the sense strand are modified; and/or
b. at least 1, 2, 3, 4, 5, 6, 7, 8, 9, 10, 11, 12, 13, 14, 15, 16, 17, 18, 19, or 20 nucleotides of the antisense strand are modified; or
c. each nucleotide in the sense strand is modified; and/or
d. each nucleotide in the antisense strand is modified.

34.-38. (canceled)

39. The dsRNA of claim 1, wherein the dsRNA comprises at least one modification, wherein the modification comprises a 2′O-methyl, a 2′fluoro, or a phosphorothioate linkage, or a combination of a 2′O-methyl or 2′fluoro and a phosphorothioate linkage.

40.-44. (canceled)

45. The dsRNA of claim 1, wherein the dsRNA is a small-interfering RNA (siRNA).

46. A double stranded ribonucleic acid (dsRNA) targeting Mucin-5B (MUC5B), wherein the dsRNA comprises a sense strand and an antisense strand forming a double stranded region, wherein the antisense strand comprises a region of complementarity to an mRNA encoding MUC5B, and wherein
a. the antisense strand comprises a nucleotide sequence selected from SEQ ID NOs: 535, 538, 540, 541, 533, 536, or 186,538-186,624;
b. the sense strand comprises a nucleotide sequence selected from SEO ID NOs: 356, 358, 359, 361, 363, 364, or 186.502-186.537; and/or
c. the sense and antisense strands are selected from any one of the sense and antisense pairs in Table 9.

47.-48. (canceled)

49. The dsRNA of claim 46, wherein:
a. the sense strand comprises the nucleotide sequence of SEQ ID NO: 358, and the antisense strand comprises the nucleotide sequence of SEQ ID NO: 186,538;
b. the sense strand comprises the nucleotide sequence of SEQ ID NO: 358, and the antisense strand comprises the nucleotide sequence of SEQ ID NO: 186,539;
c. the sense strand comprises the nucleotide sequence of SEQ ID NO: 358, and the antisense strand comprises the nucleotide sequence of SEQ ID NO: 186,540;
d. the sense strand comprises the nucleotide sequence of SEQ ID NO: 186,502, and the antisense strand comprises the nucleotide sequence of SEQ ID NO: 186,541;
e. the sense strand comprises the nucleotide sequence of SEQ ID NO: 186,503, and the antisense strand comprises the nucleotide sequence of SEQ ID NO: 535;
f. the sense strand comprises the nucleotide sequence of SEQ ID NO: 186,504, and the antisense strand comprises the nucleotide sequence of SEQ ID NO: 535;
g. the sense strand comprises the nucleotide sequence of SEQ ID NO: 186,505, and the antisense strand comprises the nucleotide sequence of SEQ ID NO: 535;
h. the sense strand comprises the nucleotide sequence of SEQ ID NO: 186,506, and the antisense strand comprises the nucleotide sequence of SEQ ID NO: 186,542;
i. the sense strand comprises the nucleotide sequence of SEQ ID NO: 186,507, and the antisense strand comprises the nucleotide sequence of SEQ ID NO: 186,543;
j. the sense strand comprises the nucleotide sequence of SEQ ID NO: 358, and the antisense strand comprises the nucleotide sequence of SEQ ID NO: 186,544;
k. the sense strand comprises the nucleotide sequence of SEQ ID NO: 358, and the antisense strand comprises the nucleotide sequence of SEQ ID NO: 186,545;
l. the sense strand comprises the nucleotide sequence of SEQ ID NO: 358, and the antisense strand comprises the nucleotide sequence of SEQ ID NO: 186,546;
m. the sense strand comprises the nucleotide sequence of SEQ ID NO: 358, and the antisense strand comprises the nucleotide sequence of SEQ ID NO: 186,547;
n. the sense strand comprises the nucleotide sequence of SEQ ID NO: 186,507, and the antisense strand comprises the nucleotide sequence of SEQ ID NO: 186,548;
o. the sense strand comprises the nucleotide sequence of SEQ ID NO: 186,507, and the antisense strand comprises the nucleotide sequence of SEQ ID NO: 186,549;
p. the sense strand comprises the nucleotide sequence of SEQ ID NO: 186,508, and the antisense strand comprises the nucleotide sequence of SEQ ID NO: 186,550;
q. the sense strand comprises the nucleotide sequence of SEQ ID NO: 186,508, and the antisense strand comprises the nucleotide sequence of SEQ ID NO: 186,551;
r. the sense strand comprises the nucleotide sequence of SEQ ID NO: 358, and the antisense strand comprises the nucleotide sequence of SEQ ID NO: 186,552;
s. the sense strand comprises the nucleotide sequence of SEQ ID NO: 358, and the antisense strand comprises the nucleotide sequence of SEQ ID NO: 186,553;
t. the sense strand comprises the nucleotide sequence of SEQ ID NO: 358, and the antisense strand comprises the nucleotide sequence of SEQ ID NO: 186,554;
u. the sense strand comprises the nucleotide sequence of SEQ ID NO: 358, and the antisense strand comprises the nucleotide sequence of SEQ ID NO: 186,555;
v. the sense strand comprises the nucleotide sequence of SEQ ID NO: 361, and the antisense strand comprises the nucleotide sequence of SEQ ID NO: 186,556;
w. the sense strand comprises the nucleotide sequence of SEQ ID NO: 361, and the antisense strand comprises the nucleotide sequence of SEQ ID NO: 186,557;
x. the sense strand comprises the nucleotide sequence of SEQ ID NO: 361, and the antisense strand comprises the nucleotide sequence of SEQ ID NO: 186,558;
y. the sense strand comprises the nucleotide sequence of SEQ ID NO: 186,509, and the antisense strand comprises the nucleotide sequence of SEQ ID NO: 186,559;
z. the sense strand comprises the nucleotide sequence of SEQ ID NO: 186,510, and the antisense strand comprises the nucleotide sequence of SEQ ID NO: 538;
aa. the sense strand comprises the nucleotide sequence of SEQ ID NO: 186,511, and the antisense strand comprises the nucleotide sequence of SEQ ID NO: 538;
bb. the sense strand comprises the nucleotide sequence of SEQ ID NO: 186,512, and the antisense strand comprises the nucleotide sequence of SEQ ID NO: 538;
cc. the sense strand comprises the nucleotide sequence of SEQ ID NO: 186,513, and the antisense strand comprises the nucleotide sequence of SEQ ID NO: 186,560;
dd. the sense strand comprises the nucleotide sequence of SEQ ID NO: 186,514, and the antisense strand comprises the nucleotide sequence of SEQ ID NO: 186,561;
ee. the sense strand comprises the nucleotide sequence of SEQ ID NO: 361, and the antisense strand comprises the nucleotide sequence of SEQ ID NO: 186,562;
ff. the sense strand comprises the nucleotide sequence of SEQ ID NO: 361, and the antisense strand comprises the nucleotide sequence of SEQ ID NO: 186,563;
gg. the sense strand comprises the nucleotide sequence of SEQ ID NO: 361, and the antisense strand comprises the nucleotide sequence of SEQ ID NO: 186,564;
hh. the sense strand comprises the nucleotide sequence of SEQ ID NO: 361, and the antisense strand comprises the nucleotide sequence of SEQ ID NO: 186,565;
ii. the sense strand comprises the nucleotide sequence of SEQ ID NO: 186,514, and the antisense strand comprises the nucleotide sequence of SEQ ID NO: 186,566;
jj. the sense strand comprises the nucleotide sequence of SEQ ID NO: 186,514, and the antisense strand comprises the nucleotide sequence of SEQ ID NO: 186,567;
kk. the sense strand comprises the nucleotide sequence of SEQ ID NO: 186,515, and the antisense strand comprises the nucleotide sequence of SEQ ID NO: 186,568;
ll. the sense strand comprises the nucleotide sequence of SEQ ID NO: 186,515, and the antisense strand comprises the nucleotide sequence of SEQ ID NO: 186,569;
mm. the sense strand comprises the nucleotide sequence of SEQ ID NO: 361, and the antisense strand comprises the nucleotide sequence of SEQ ID NO: 186,570;
nn. the sense strand comprises the nucleotide sequence of SEQ ID NO: 361, and the antisense strand comprises the nucleotide sequence of SEQ ID NO: 186,571;
oo. the sense strand comprises the nucleotide sequence of SEQ ID NO: 361, and the antisense strand comprises the nucleotide sequence of SEQ ID NO: 186,572;
pp. the sense strand comprises the nucleotide sequence of SEQ ID NO: 361, and the antisense strand comprises the nucleotide sequence of SEQ ID NO: 186,573;
qq. the sense strand comprises the nucleotide sequence of SEQ ID NO: 363, and the antisense strand comprises the nucleotide sequence of SEQ ID NO: 186,574;
rr. the sense strand comprises the nucleotide sequence of SEQ ID NO: 363, and the antisense strand comprises the nucleotide sequence of SEQ ID NO: 186,575;
ss. the sense strand comprises the nucleotide sequence of SEQ ID NO: 363, and the antisense strand comprises the nucleotide sequence of SEQ ID NO: 186,576;
tt. the sense strand comprises the nucleotide sequence of SEQ ID NO: 186,516, and the antisense strand comprises the nucleotide sequence of SEQ ID NO: 540;
uu. the sense strand comprises the nucleotide sequence of SEQ ID NO: 186,517, and the antisense strand comprises the nucleotide sequence of SEQ ID NO: 540;
vv. the sense strand comprises the nucleotide sequence of SEQ ID NO: 186,518, and the antisense strand comprises the nucleotide sequence of SEQ ID NO: 540;
ww. the sense strand comprises the nucleotide sequence of SEQ ID NO: 186,519, and the antisense strand comprises the nucleotide sequence of SEQ ID NO: 540;
xx. the sense strand comprises the nucleotide sequence of SEQ ID NO: 186,520, and the antisense strand comprises the nucleotide sequence of SEQ ID NO: 186,577;
yy. the sense strand comprises the nucleotide sequence of SEQ ID NO: 363, and the antisense strand comprises the nucleotide sequence of SEQ ID NO: 186,578;
zz. the sense strand comprises the nucleotide sequence of SEQ ID NO: 363, and the antisense strand comprises the nucleotide sequence of SEQ ID NO: 186,579;
aaa. the sense strand comprises the nucleotide sequence of SEQ ID NO: 363, and the antisense strand comprises the nucleotide sequence of SEQ ID NO: 186,580;
bbb. the sense strand comprises the nucleotide sequence of SEQ ID NO: 363, and the antisense strand comprises the nucleotide sequence of SEQ ID NO: 186,581;
ccc. the sense strand comprises the nucleotide sequence of SEQ ID NO: 363, and the antisense strand comprises the nucleotide sequence of SEQ ID NO: 186,582 (usAfscga(C3)auccaugGfaCfuuguasgsu);
ddd. the sense strand comprises the nucleotide sequence of SEQ ID NO: 363, and the antisense strand comprises the nucleotide sequence of SEQ ID NO: 186,583;
eee. the sense strand comprises the nucleotide sequence of SEQ ID NO: 363, and the antisense strand comprises the nucleotide sequence of SEQ ID NO: 186,584;
fff. the sense strand comprises the nucleotide sequence of SEQ ID NO: 364, and the antisense strand comprises the nucleotide sequence of SEQ ID NO: 186,585;
ggg. the sense strand comprises the nucleotide sequence of SEQ ID NO: 364, and the antisense strand comprises the nucleotide sequence of SEQ ID NO: 186,586;
hhh. the sense strand comprises the nucleotide sequence of SEQ ID NO: 364, and the antisense strand comprises the nucleotide sequence of SEQ ID NO: 186,587;
iii. the sense strand comprises the nucleotide sequence of SEQ ID NO: 186,521, and the antisense strand comprises the nucleotide sequence of SEQ ID NO: 186,588;
jjj. the sense strand comprises the nucleotide sequence of SEQ ID NO: 186,522, and the antisense strand comprises the nucleotide sequence of SEQ ID NO: 541;
kkk. the sense strand comprises the nucleotide sequence of SEQ ID NO: 186,523, and the antisense strand comprises the nucleotide sequence of SEQ ID NO: 541;
lll. the sense strand comprises the nucleotide sequence of SEQ ID NO: 186,524, and the antisense strand comprises the nucleotide sequence of SEQ ID NO: 541;
mmm. the sense strand comprises the nucleotide sequence of SEQ ID NO: 186,525, and the antisense strand comprises the nucleotide sequence of SEQ ID NO: 186,589;
nnn. the sense strand comprises the nucleotide sequence of SEQ ID NO: 186,526, and the antisense strand comprises the nucleotide sequence of SEQ ID NO: 186,590;
ooo. the sense strand comprises the nucleotide sequence of SEQ ID NO: 364, and the antisense strand comprises the nucleotide sequence of SEQ ID NO: 186,591;
ppp. the sense strand comprises the nucleotide sequence of SEQ ID NO: 364, and the antisense strand comprises the nucleotide sequence of SEQ ID NO: 186,592;
qqq. the sense strand comprises the nucleotide sequence of SEQ ID NO: 364, and the antisense strand comprises the nucleotide sequence of SEQ ID NO: 186,593;
rrr. the sense strand comprises the nucleotide sequence of SEQ ID NO: 364, and the antisense strand comprises the nucleotide sequence of SEQ ID NO: 186,594;
sss. the sense strand comprises the nucleotide sequence of SEQ ID NO: 186,526, and the antisense strand comprises the nucleotide sequence of SEQ ID NO: 186,595;
ttt. the sense strand comprises the nucleotide sequence of SEQ ID NO: 186,526, and the antisense strand comprises the nucleotide sequence of SEQ ID NO: 186,596;
uuu. the sense strand comprises the nucleotide sequence of SEQ ID NO: 186,527, and the antisense strand comprises the nucleotide sequence of SEQ ID NO: 186,597;
vvv. the sense strand comprises the nucleotide sequence of SEQ ID NO: 186,527, and the antisense strand comprises the nucleotide sequence of SEQ ID NO: 186,598;
www. the sense strand comprises the nucleotide sequence of SEQ ID NO: 364, and the antisense strand comprises the nucleotide sequence of SEQ ID NO: 186,599;
xxx. the sense strand comprises the nucleotide sequence of SEQ ID NO: 364, and the antisense strand comprises the nucleotide sequence of SEQ ID NO: 186,600;
yyy. the sense strand comprises the nucleotide sequence of SEQ ID NO: 364, and the antisense strand comprises the nucleotide sequence of SEQ ID NO: 186,601;
zzz. the sense strand comprises the nucleotide sequence of SEQ ID NO: 364, and the antisense strand comprises the nucleotide sequence of SEQ ID NO: 186,602;
aaaa. the sense strand comprises the nucleotide sequence of SEQ ID NO: 356, and the antisense strand comprises the nucleotide sequence of SEQ ID NO: 186,603;
bbbb. the sense strand comprises the nucleotide sequence of SEQ ID NO: 356, and the antisense strand comprises the nucleotide sequence of SEQ ID NO: 186,604;
cccc. the sense strand comprises the nucleotide sequence of SEQ ID NO: 356, and the antisense strand comprises the nucleotide sequence of SEQ ID NO: 186,605;
dddd. the sense strand comprises the nucleotide sequence of SEQ ID NO: 186,528, and the antisense strand comprises the nucleotide sequence of SEQ ID NO: 533;
eeee. the sense strand comprises the nucleotide sequence of SEQ ID NO: 186,529, and the antisense strand comprises the nucleotide sequence of SEQ ID NO: 533;
ffff. the sense strand comprises the nucleotide sequence of SEQ ID NO: 186,530, and the antisense strand comprises the nucleotide sequence of SEQ ID NO: 533;
gggg. the sense strand comprises the nucleotide sequence of SEQ ID NO: 186,531, and the antisense strand comprises the nucleotide sequence of SEQ ID NO: 533;
hhhh. the sense strand comprises the nucleotide sequence of SEQ ID NO: 186,532, and the antisense strand comprises the nucleotide sequence of SEQ ID NO: 186,606;
iiii. the sense strand comprises the nucleotide sequence of SEQ ID NO: 356, and the antisense strand comprises the nucleotide sequence of SEQ ID NO: 186,607;
jjjj. the sense strand comprises the nucleotide sequence of SEQ ID NO: 356, and the antisense strand comprises the nucleotide sequence of SEQ ID NO: 186,608;
kkkk. the sense strand comprises the nucleotide sequence of SEQ ID NO: 356, and the antisense strand comprises the nucleotide sequence of SEQ ID NO: 186,609;
llll. the sense strand comprises the nucleotide sequence of SEQ ID NO: 356, and the antisense strand comprises the nucleotide sequence of SEQ ID NO: 186,610;
mmmm. the sense strand comprises the nucleotide sequence of SEQ ID NO: 356, and the antisense strand comprises the nucleotide sequence of SEQ ID NO: 186,611 (usAfsccg(C3)aauuuggUfcAfaacagsgsa);
nnnn. the sense strand comprises the nucleotide sequence of SEQ ID NO: 356, and the antisense strand comprises the nucleotide sequence of SEQ ID NO: 186,612;
oooo. the sense strand comprises the nucleotide sequence of SEQ ID NO: 356, and the antisense strand comprises the nucleotide sequence of SEQ ID NO: 186,613;
pppp. the sense strand comprises the nucleotide sequence of SEQ ID NO: 359, and the antisense strand comprises the nucleotide sequence of SEQ ID NO: 186,614;
qqqq. the sense strand comprises the nucleotide sequence of SEQ ID NO: 359, and the antisense strand comprises the nucleotide sequence of SEQ ID NO: 186,615;
rrrr. the sense strand comprises the nucleotide sequence of SEQ ID NO: 359, and the antisense strand comprises the nucleotide sequence of SEQ ID NO: 186,616;
ssss. the sense strand comprises the nucleotide sequence of SEQ ID NO: 186,533, and the antisense strand comprises the nucleotide sequence of SEQ ID NO: 536;
tttt. the sense strand comprises the nucleotide sequence of SEQ ID NO: 186,534, and the antisense strand comprises the nucleotide sequence of SEQ ID NO: 536;
uuuu. the sense strand comprises the nucleotide sequence of SEQ ID NO: 186,535, and the antisense strand comprises the nucleotide sequence of SEQ ID NO: 536;
vvvv. the sense strand comprises the nucleotide sequence of SEQ ID NO: 186,536, and the antisense strand comprises the nucleotide sequence of SEQ ID NO: 536;
wwww. the sense strand comprises the nucleotide sequence of SEQ ID NO: 186,537, and the antisense strand comprises the nucleotide sequence of SEQ ID NO: 186,617;
xxxx. the sense strand comprises the nucleotide sequence of SEQ ID NO: 359, and the antisense strand comprises the nucleotide sequence of SEQ ID NO: 186,618;
yyyy. the sense strand comprises the nucleotide sequence of SEQ ID NO: 359, and the antisense strand comprises the nucleotide sequence of SEQ ID NO: 186,619;
zzzz. the sense strand comprises the nucleotide sequence of SEQ ID NO: 359, and the antisense strand comprises the nucleotide sequence of SEQ ID NO: 186,620;
aaaaa. the sense strand comprises the nucleotide sequence of SEQ ID NO: 359, and the antisense strand comprises the nucleotide sequence of SEQ ID NO: 186,621;
bbbbb. the sense strand comprises the nucleotide sequence of SEQ ID NO: 359, and the antisense strand comprises the nucleotide sequence of SEQ ID NO: 186,622 (usAfsgug(C3)cggguggAfaCfaaagcsusc);
ccccc. the sense strand comprises the nucleotide sequence of SEQ ID NO: 359, and the antisense strand comprises the nucleotide sequence of SEQ ID NO: 186,623; or
ddddd. the sense strand comprises the nucleotide sequence of SEQ ID NO: 359, and the antisense strand comprises the nucleotide sequence of SEQ ID NO: 186,624.

50. The dsRNA of claim 49, wherein:
a. the sense strand comprises the nucleotide sequence of SEQ ID NO: 186,507, and the antisense strand comprises the nucleotide sequence of SEQ ID NO: 186,543;
b. the sense strand comprises the nucleotide sequence of SEQ ID NO: 186,512, and the antisense strand comprises the nucleotide sequence of SEQ ID NO: 538;
c. the sense strand comprises the nucleotide sequence of SEQ ID NO: 186,514, and the antisense strand comprises the nucleotide sequence of SEQ ID NO: 186,561;
d. the sense strand comprises the nucleotide sequence of SEQ ID NO: 361, and the antisense strand comprises the nucleotide sequence of SEQ ID NO: 186,562;
e. the sense strand comprises the nucleotide sequence of SEQ ID NO: 361, and the antisense strand comprises the nucleotide sequence of SEQ ID NO: 186,563;
f. the sense strand comprises the nucleotide sequence of SEQ ID NO: 364, and the antisense strand comprises the nucleotide sequence of SEQ ID NO: 186,593;
g. the sense strand comprises the nucleotide sequence of SEQ ID NO: 364, and the antisense strand comprises the nucleotide sequence of SEQ ID NO: 186,600;
h. the sense strand comprises the nucleotide sequence of SEQ ID NO: 356, and the antisense strand comprises the nucleotide sequence of SEQ ID NO: 186,603;
i. the sense strand comprises the nucleotide sequence of SEQ ID NO: 356, and the antisense strand comprises the nucleotide sequence of SEQ ID NO: 186,605;
j. the sense strand comprises the nucleotide sequence of SEQ ID NO: 186,532, and the antisense strand comprises the nucleotide sequence of SEQ ID NO: 186,606;
k. the sense strand comprises the nucleotide sequence of SEQ ID NO: 356, and the antisense strand comprises the nucleotide sequence of SEQ ID NO: 186,607;
l. the sense strand comprises the nucleotide sequence of SEQ ID NO: 359, and the antisense strand comprises the nucleotide sequence of SEQ ID NO: 186,614;
m. the sense strand comprises the nucleotide sequence of SEQ ID NO: 186,536, and the antisense strand comprises the nucleotide sequence of SEQ ID NO: 536; or
n. the sense strand comprises the nucleotide sequence of SEQ ID NO: 186,537, and the antisense strand comprises the nucleotide sequence of SEQ ID NO: 186,617.

51. The dsRNA of claim 46, wherein the dsRNA has any one or more of the following characteristics compared to the corresponding unmodified dsRNA or to the dsRNA sequence used as the starting dsRNA:
a. Improved efficacy;
b. Improved potency;
c. Improved stability; and/or
d. Reduced off-target effects.

52. (canceled)

53. The dsRNA of claim 46, wherein each of the sense strand and/or the antisense strand:
a. is conjugated to one or more lipophilic moieties; and/or
b. is packaged in a lipid nanoparticle (LNP).

54. (canceled)

55. A synthetic antisense oligonucleotide (ASO) targeting MUC5B, wherein the oligonucleotide comprises a nucleotide sequence:
a. having at least 90% nucleotide sequence identity to any one of SEQ ID NOs: 709-802;
b. comprising at least 15, 16, or 17 contiguous nucleotides differing in sequence by no more than 3 nucleotides from any one of SEQ ID NO: 709-802;
c. comprising at least 15, 16, or 17 contiguous nucleotides differing in sequence by no more than 3 nucleotides from any one of SEQ ID NO: 803-896; or
d. comprising at least 15, 16, or 17 contiguous nucleotides differing in sequence by no more than 3 nucleotides from any one of SEQ ID NO: 147,339-186,429.

56.-59. (canceled)

60. The dsRNA of claim 1, or the synthetic antisense oligonucleotide of claim 57, wherein at least one of the nucleotides is modified, wherein the modification is selected from a deoxy-nucleotide, a 3′-terminal deoxy-thymine (dT) nucleotide, a 2′-O-methyl modified nucleotide (2′-OMe), a 2′-fluoro modified nucleotide (2′-F), a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a 5′-phosphorothioate group, a phosphorothioate linkage, a nucleotide comprising a 5′-methylphosphonate group, a nucleotide comprising a 5′ phosphate or 5′ phosphate mimic, a nucleotide comprising vinyl phosphonate, a nucleotide comprising adenosine-glycol nucleic acid (GNA), a nucleotide comprising thymidine-glycol nucleic acid (GNA) S-Isomer, a nucleotide comprising 2-hydroxymethyl-tetrahydrofurane-5-phosphate, a nucleotide comprising 2′-deoxythymidine-3′ phosphate, a nucleotide comprising 2′-deoxyguanosine-3′-phosphate, a 2′-0 hexadecyl nucleotide, a nucleotide comprising a 2′-phosphate, a cytidine-2′-phosphate nucleotide, a guanosine-2′-phosphate nucleotide, a 2′-O-hexadecyl-cytidine-3′-phosphate nucleotide, a 2′-O-hexadecyl-adenosine-3′-phosphate nucleotide, a 2′-O-hexadecyl-guanosine-3′-phosphate nucleotide, a 2′-O-hexadecyl-uridine-3′-phosphate nucleotide, a 5′-vinyl phosphonate (VP/(e.g., (vin-u)), a 2′-deoxyadenosine-3′-phosphate nucleotide, a 2′-deoxycytidine-3′-phosphate nucleotide, a 2′-deoxyguanosine-3′-phosphate nucleotide, a 2′-deoxythymidine-3′-phosphate nucleotide, a 2′-deoxyuridine nucleotide, a terminal nucleotide linked to a cholesteryl derivative, a dodecanoic acid bisdecylamide group and a C3 spacer; and combinations thereof.

61.-69. (canceled)

70. A pharmaceutical composition comprising the dsRNA of claim 1, further comprising a lipid delivery vehicle, wherein the lipid delivery vehicle is a lipid nanoparticle (LNP) comprising one or more of a cationic lipid, a non-cationic lipid, a cholesterol-based lipid, a PEG-modified lipid, an amphiphilic block copolymer and/or a polymer, or a combination thereof.

71.-72. (canceled)

73. A vector comprising one or more of the dsRNA of claim 1.

74. A cell containing the dsRNA of claim 1.

75.-76. (canceled)

77. A device for inhalation administration comprising the dsRNA of claim 1.

78. (canceled)

79. A method of inhibiting expression of a MUC5B gene in a cell, the method comprising: contacting the cell with the dsRNA of claim 1.

80.-82. (canceled)

83. A method for treating a human subject having a lung disease or a subject at risk of developing a lung disease, comprising administering to the subject in need thereof a therapeutically effective amount of the dsRNA of claim 1.

84.-91. (canceled)
